(19) United States
(12) Patent Application Publication
(10) Pub. No.: US 2025/0144187 A1
(43) Pub. Date: May 8, 2025

(54) COMBINATION OF HYDROLASES AND ANTI-MICROORGANISM DRUGS AND USES THEREOF

(71) Applicants: CENTRE FOR EYE AND VISION RESEARCH LIMITED, Hong Kong (CN); THE HONG KONG POLYTECHNIC UNIVERSITY, Hong Kong (CN)

(72) Inventors: Qian ZHAO, Hong Kong (CN); Qi ZHAO, Hong Kong (CN)

(21) Appl. No.: 18/888,874

(22) Filed: Sep. 18, 2024

Related U.S. Application Data
(60) Provisional application No. 63/590,783, filed on Oct. 16, 2023.

(51) Int. Cl. A61K 38/47; A61K 31/4439; A61K 31/496; A61K 31/53; A61K 31/546; A61K 31/7135; A61P 31/04

(52) U.S. Cl. CPC A61K 38/47; A61K 31/4439; A61K 31/496; A61K 31/53; A61K 31/546; A61K 31/7135; A61P 31/04

(57) ABSTRACT

The disclosure provides a combination of one or more hydrolases and one or more anti-microorganism drugs, and a pharmaceutical composition or a kit comprising the combination. The disclosure also provides a method for treating a microorganism infection by using a therapeutically effective amount of the combination, pharmaceutical composition or kit. The combination of this disclosure is safer, and more economical, and can re-sensitize a drug against lots of bacteria at less dosage in clinical and avoid antimicrobial resistance to extend the lifespan of the drug.

### TECHNICAL FIELD

The present disclosure belongs to the field of medicine. In particular, the present disclosure relates to a combination of hydrolases and anti-microorganism drugs and ues thereof.

### BACKGROUND

Since the clinical introduction of penicillin in the 1940s, antimicrobial resistance (AMR) has been a worldwide crisis because it is very difficult to treat and poses a huge threat to human health. To cope with the crisis, scientists must develop more antimicrobials, but the development speed is recently very slow due to the limited resources producing antimicrobials in nature. Compared with the development of new antimicrobial or combination of multiple antimicrobials, combination therapy comprising an existing antibiotic and a non-antibiotic (such as resistance breaker) is a current popular alternative as safer, more economical, and effective solution.

### BRIEF SUMMARY OF THE DISCLOSURE

In the present disclosure, following technical solutions are provided:

In the first aspect, a combination of one or more enzymes and one or more anti-microorganism drugs is provided.

In a specific embodiment, compared with the anti-microorganism drug alone, said combination results in at least a 10-fold, 50-fold, 00-fold, 150-fold, 20-fold, 250-fold, 300-fold, 350-fold, 400-fold, 450-fold, 500-fold, 550-fold, 600-fold, 650-fold, 700-fold, 750-fold, 800-fold, 850-fold, 900-fold increase in the sensitivity of a microorganism to the anti-microorganism drug.

In a specific embodiment, the fractional inhibitory concentration index (FICI) value of the combination is not higher than 0.5; preferably not higher than 0.3; move preferably not higher than 0.1; most preferably not higher than 0.03.

In a specific embodiment, the microorganism is a bacterium, fungus, virus, or parasite; preferably, a bacterium.

In a specific embodiment, the microorganism is a drug-resistant bacterium.

In a specific embodiment, the bacterium is a Gram-positive bacterium, or Gram-negative bacterium; and preferably, the bacterium is a Gram-positive bacterium.

In a specific embodiment, the Gram-positive bacterium includes but is not limited to methicillin-resistant Staphylococcus aureus (MRSA), methicillin-resistant Staphylococcus epidermis (MRSE), Enterococcus faecalis, Enterococcus faecium, Streptococcus mutans, Corynebacterium pseudodiphtheriticum, Streptococcus pyogenes or Streptococcus pneumoniae, Propionibacterium acnes.

In a specific embodiment, wherein the Gram-positive bacterium is methicillin-resistant Staphylococcus aureus (MRSA) or methicillin-resistant Staphylococcus epidermis (MRSE).

In a specific embodiment, the enzyme is a hydrolase.

In a specific embodiment, the hydrolase is selected from the group consisting of lysozyme, an isoform and homologue thereof, lysin and lysomtaphin; and preferably, the hydrolase is lysozyme or an isoform or homologue thereof.

In a specific embodiment, the lysozyme is of C-type (from, for example human or chicken), G-type (from, for example goose), T-type (from, for example 14 bacteriophage) or bacterial-type (from, for example bacteria, such as Bacillus subtilis.

In a specific embodiment, the anti-microorganism drug is a drug with an inhibitory or killing activity against the microorganism.

In a specific embodiment, the anti-microorganism drug is a drug approved by a local drug regulatory authority.

In a specific embodiment, the local drug regulatory authority is the FDA in the USA, EMA in EP, PMDA in JP, or NMPA in CN.

In a specific embodiment, the anti-microorganism drug is selected from the group consisting of FDA-approved drugs.

In a specific embodiment, wherein anti-microorganism drug is selected from an oxaxolidinone class antibiotic, a β-lactam class antibiotic or anti-bacterial compound.

In a specific embodiment, the oxazolidinone class antibiotic is an oxazolidinone with following core structure:

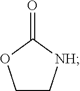

preferably, the oxazolidinone class antibiotic is an oxazolidinone with following core structure:

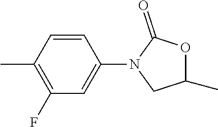

In a specific embodiment, the anti-microorganism drug is selected from the group consisting of Auranofin, Cefdinir, Tedizolid Phosphate, Chlorhexidine 2HCl, Tilmicosin, Mezlocillin Sodium, Bardoxolone Methyl, Cefdinir, Cefoperazone, Nitazoxanide, Carmofur, Doripenem Hydrate, Sultamicillin, Cefmetazole sodium, Benzylpenicillin potassium, Mitomycin C, Ceftriaxone Sodium, Cefradine, Meropenem Trihydrate, Ceftazidime Pentahydrate, Cefoselis Sulfate, Luliconazole, Cefcapene Pivoxil Hydrochloride, Ceftazidime Sulfate, TD52 (dihydrochloride), SCP1-IN-1, Rifamycin S, Paroxypropione, Ethyl 3,4-dihydroxybenzoate, Delafloxacin (meglumine) 2,4-Diacetylphloroglucinol, Sertaconazole (nitrate), delpazolid phosphate (DP), eperezolid phosphate (EP) and a pharmaceutically acceptable salt thereof.

In a specific embodiment, the anti-microorganism drug is selected from the group consisting of Auranofin, Cefdinir, Tedizolid Phosphate, Chlorhexidine 2HCl, Tilmicosin, Mezlocillin Sodium, Bardoxolone Methyl, Cefoperazone, Nitazoxanide, Carmofur, Doripenem Hydrate, delpazolid phosphate (DP), eperezolid phosphate (EP) and a pharmaceutically acceptable salt thereof.

In a specific embodiment, wherein the anti-microorganism drug is selected from the group consisting of Auranofin, Cefdinir, Tedizolid Phosphate, delpazolid phosphate (DP), and eperezolid phosphate (EP).

In a specific embodiment, wherein the combination further comprises an autolysis promoter or simulator, including but not limited to a walR inhibitor or antagonist, or a N-acetylmuramoyl-L-alanine amidase domain-containing protein agonist; or
an LTA/WTA synthesis promoter or simulator, including but not limited to a promoter or simulator up regulating the expression of an enzyme selected from the group consisting of TarA, TarB, TarD, TarF, TarL, TarS, TagH and FmtA.

In the second aspect, a pharmaceutical composition ii provided, wherein the pharmaceutical composition comprises the combination of the first aspect and a pharmaceutically acceptable excipient.

In a specific embodiment, the pharmaceutical composition is in a dosage form for systemic or topical administration.

In a specific embodiment, the systemic dosage form is selected from the group consisting of a tablet, granule, capsule, pill, solutions, emulsion, suspension, injection.

In a specific embodiment, wherein the topical dosage form is selected from the group consisting of a drop, cream, ointment, lotion, liniment, suppository, paste, and patch.

In the third aspect, a kit is provided, wherein the kit comprises one or more container for accommodating the combination of the first aspect or the pharmaceutical composition of the second aspect.

In a specific embodiment, the kit further comprises an instruction on how to use the combination or composition for treating or preventing microorganism infections.

In a specific embodiment, the microorganism infection is an eye infection, skin infection, or an infection in wound.

In the fourth aspect, a use of a combination of the first aspect, or a pharmaceutical composition of the second aspect, or a kit of the third aspect is provided in the preparation of a medicament for treating or preventing microbial microorganism infections.

In a specific embodiment, the microorganism infection is an eye infection, skin infection, or an infection in wound.

In the fifth aspect, a combination of the first aspect, or a pharmaceutical composition of the second aspect, or a kit of the third aspect for use in a method for treating or preventing microorganism infections is provided.

In a specific embodiment, wherein the microorganism infection is an eye infection, skin infection, or an infection in wound.

In the sixth aspect, a method for treating a microorganism infection is provided, wherein the method includes a step of giving a therapeutically effective amount of a combination of the first aspect, or a pharmaceutical composition of the second aspect, or a kit of the third aspect to a subject in need thereof.

In a specific embodiment, the subject is a mammal.

In a specific embodiment, the subject is a house pet, a racing animal, a domestic animal, or a primate.

In a specific embodiment, the subject is a cat, dog, horse, pig, cattle, COW, goat, sheep, rabbit, or human; preferably, a human.

In a specific embodiment, the microorganism infection is an eye infection, skin infection, an oral infection, a respiratory system infection, a digestive system infection or an infection in wound.

It should be understood that, within the scope of the present invention, each technical feature of the present invention described above and, in the following, (as examples) may be combined with each other to firm a new or preferred technical solution, which is not listed here one by one.

### DESCRIPTION OF DRAWINGS

FIG. 1A to FIG. 1F: Combination therapy efficiently inhibited the growth and resistance evolution in MRSA. FIG. 1A: Representative heat-map of MRSA treated with combination therapy. FIG. 1B: Time-kill curves showing the growth of log-phase MRSA under treatment with lysozyme, auranofin and their combination. FIG. 1C: Resistance acquisition curves during 16 passages in MRSA under the sub-inhibitory concentration of auranofin and its combination therapy with fixed concentration of lysozyme. FIG. 10D Heat map of mutant frequencies of MRSA exposed to lysozyme, auranofin and their combination. FIG. 1F: Mutation prevention index (MPI) of auranofin used in combination with lysozyme. FIG. 1F: Crystal violet absorbance assays showing these effects on biofilm of MRSA under treatment with lysozyme, auranofin and their combination.

FIG. 2A to FIG. 2F: Morphological change of MRSA in the absence or presence of drugs. Scanning electron microscopy images of FIG. 2A: MRSA itself and those incubated with FIG. 2B: 0.5 mg/ml lysosyme. FIG. 2C and FIG. 2E: 0.17 or 0.34 μg/ml auranofin alone or FIG. 2D and FIG. 2F: their combination for 5 hours. White arrowheads indicated damaged cell wall and cell autolysis. Assays were performed in triplicate.

FIG. 3A to FIG. 3D: Proteomic analysis of MRSA exposed to lysozyme alone or the combination of lysozyme plus auranofin. FIG. 3A: Volcano plot and FIG. 38: GO annotation analysis of the differential expression proteins in MRSA after exposing lysozyme (0.5 mg/ml) or the combination of lysozyme (0.5 ng/ml) plus auranofin (034 μg/ml) for 5 hours. The dotted lines in x- and y-axes reflected an adjusted p-value (<0.05, student's t-test) and fold change (>1.2), respectively. FIG. 3C: These changes in expression levels of several interesting proteins were detailed and FIG. 3D: PRM analysis on N-acetylmuramoyl-L-alanine amidase domain-containing (UniProt ID: Q2G222). The inserted peptide was the unique validated sequence in PRM study.

FIG. 4A to FIG. 4B: Auranofin showed great combination effect with lysozyme in ARPE-19 cell-based infection model. FIG. 4A: Survival ratio and FIG. 4B: bacterial load of ARPE-9 cells infected with or without MRSA were shown. In general, the combination between lysozyme and auranofin significantly reduced the bacterial load and increased the survival ratio of ARPE-19 cell after MRSA infection.

FIG. 5: Inoculation effects affect the MIC₉₀ of auranofin and lysozyme against MRSA. Herein, MRSA was insensitive to lysozyme, in which 150 mg/ml lysozyme hardly killed MRSA no matter how many the innoculated density was. All assays were performed in triplicate.

FIG. 6A to FIG. 6B: Auranofin synergized with lysozyme against MRSA. FIG. 6A: Fractional inhibitory concentration (FIC) curves. FIG. 6B: Representative images showing the inhibited effect on MRSA under treatment with 0.5 mg/ml lysozyme, 0.34 μg/ml auranofin alone or their combination.

FIG. 7A to FIG. 7B: Proteomic analysis of MRSA exposed to lysozyme in the absence or presence of 0.17 μg/ml auranofin. FIG. 7A: Volcano plot and FIG. 7B: (0 annotation analysis of the differential expression proteins in MRSA exposed to lysozyme (0.5 mg/ml) or the combination of lysozyme (0.5 mg/ml) plus auranofin (0.17 μg/ml) for 5 hours. The dotted lines in x- and y-axes reflected an adjusted p-value<0.05 (Student's t-test) and |Fold change|>1.2, respectively.

FIGS. 8A to FIG. 8G: PRM analysis validated the change of FIG. 8A: CopZ (uniprot ID: A6QK48. FIG. 8B: probable copper-transporting P-type ATPase H (uniprot ID: A8YZ02). FIG. 8C: thiol peroxidase (uniprot ID: Q5MHF61). FIG. 8D: GTP cyclohydrolase FolE2 (uniprot ID: Q2G0L1), FIG. 8E: 30S ribosomal protein S13 (uniprot ID: Q6GEK7), FIG. 8F: transcriptional regulatory protein WalR (uniprot ID: Q7A8E1) and FIG. 8G: α-hemolysin (uniprot ID: Q2G1X0) in protein levels. These inserted peptides in each protein maps were validated peptides in PRM studies.

FIG. 9: ATP level was significantly reduced when MRSA was treated with combination therapy of 0.5 mg/ml lysozyme and 0.34 μg/ml auranofin. Assays were performed in triplicate and results were expressed as average±SD, ***p<0.001.

FIG. 10: Toxicity analysis of auranofin on ARPE-19 cells by using CCK-8 kit. Herein, the IC₅₀ of auranofin was figured out as 2.21±0.34 μg/ml.

FIG. 11A to FIG. 11I: High-throughput screening explores potential of cefdinir to re-sensitize lysozyme against broad-spectrum gram-positive pathogens. 4989 compounds comprising of 6 types FIG. 11A: at fixed concentrations (2.5 μM) were screened in combination with 1 mg/ml human lysozyme against FIG. 11B: MRSA at 1.0×10⁶ CFU/ml. FIG. 11C: Representative heat-map of MRSA in checkerboard microbroth-dilution assay and FIG. 11D: Isobolograms of combination therapies against 8 different gram-positive pathogens. FIG. 11F: Fractional inhibitory concentration (FIC) index analysis on MRSA between cefdinir and 5 subtypes of lysozyme, FIG. 11F; Time-kill curves for log-phage growing MRSA under treatment with lysozyme, cefdinir and their combination for up to 16 hours. FIG. 11G to FIG. 11H: Heat map of mutant frequencies of MRSA exposed to lysozyme, cefdinir and their combination at different concentrations and mutation prevention index (MPI) of cefdinir used in combination with lysozyme. FIG. 11I: Crystal violet assays showing the pro-biofilm of MRSA under treatment with lysozyme, cefdinir and their combination. The result indicated combination therapy has great inhibition effect on formation of MRSA biofilm.

FIG. 12A-1 to FIG. 12C: Combination therapy significantly aggravated the damage degree of cell wall. FIG. 12A-1 to FIG. 12A-2: Permeability and morphological analysis of MRSA in the absence or presence of drugs. White arrowheads in diffused SYBR gold dye and scanning electron microscopy images of MRSA under combination therapy indicated damaged cell wall with numerous pits and holes. FIG. 12B: The HADA-based synthesis analysis of nascent cell wall of MRSA exposed to 0.5 mg/ml lysozyme, 0.5 μg/ml cefdinir and their combination for different tissue. Remarkably, intact bacteria with or without 100 μM HADA treatment served as control or no-drug groups, respectively. FIG. 12C: The digestion rate study on cell wall of MRSA under different treatments. HADA signal from digested supernatant indicated that cefdinir re-sensitized lysozyme against cell wall of MRSA.

FIG. 13A to FIG. 13G: Proteomic analysis on MRSA under different treatments uncovered increased LTA/WTA synthesis facilitated the interaction with lysozyme. FIG. 13A: Volcano plot showing changed proteins in MRSA exposed to cefdinir (0.5 μg/ml), lysozyme (0.5 mg/ml) or their combination for 1 hour. The x- and y-axes in FIG. 13A represent the expression changes and corresponding statistically significant degree, respectively. An adjusted p-value<0.05 (Student's t-test) and |Fold change|>1.5 were applied as the cutoff for significant DEPs. FIG. 13B: Differential expression of selected proteins associated with the LTA and WTA synthesis. FIG. 13C: Diagram to show the pathway to synthesize LTA/WTA in MRSA. All observed related enzymes were merged into red arrowheads. The insert data indicated its fold-change in combination-treated MRSA if compared with that treated with lysozyme only. FIG. 13D: PRM-based analysis on several representative enzymes, i.e., Tar F, Tar L, Tar S and TagH. These inserted sequences were the unique peptides used in PRM analysis. FIG. 13E: Cytochrome C binding analysis on MRSA under different treatment. The lower intensity at 530 nm clearly indicated cefdinir treatment led to stronger interaction between cell wall and positive charge lysozyme. FIG. 13F: FICI analysis on MRSA with or without the treatment of inhibitor of the synthesis of WTA (Targocil) or LTA (LtaS-IN-1). The increased FICI values indicated the overexpression of LTA/WTA did work in combination therapy. FIG. 13G: ATP levels in MRSA exposed to cefdinir (0.5 μg/ml), lysozyme (0.5 mg/ml) or the combination for 1 hour.

FIG. 14A to FIG. 14E: Cefdinir shows gat combination effect with lysozyme in relieving the symptoms of rats in skin infection model. FIG. 14A: A flow chart showing the detail on infected rat in skin infection model. FIG. 14B: Bacterial load in wound of rats infected with MRSA. FIG. 14C: Representative hematoxylin and cosin (H-E) staining images from wounds of infected rats. Black arrowheads clearly showed reduced denser inflammatory cells with blue nucleus, more new blood vessels and smoother edges in combination therapy groups. FIG. 14D to FIG. 14E: Normalized levels of FIG. 14D: ROS or FIG. 14E: IL-6 in wounds of infected rats under different treatments. *p<0.05, ***p<0.001.

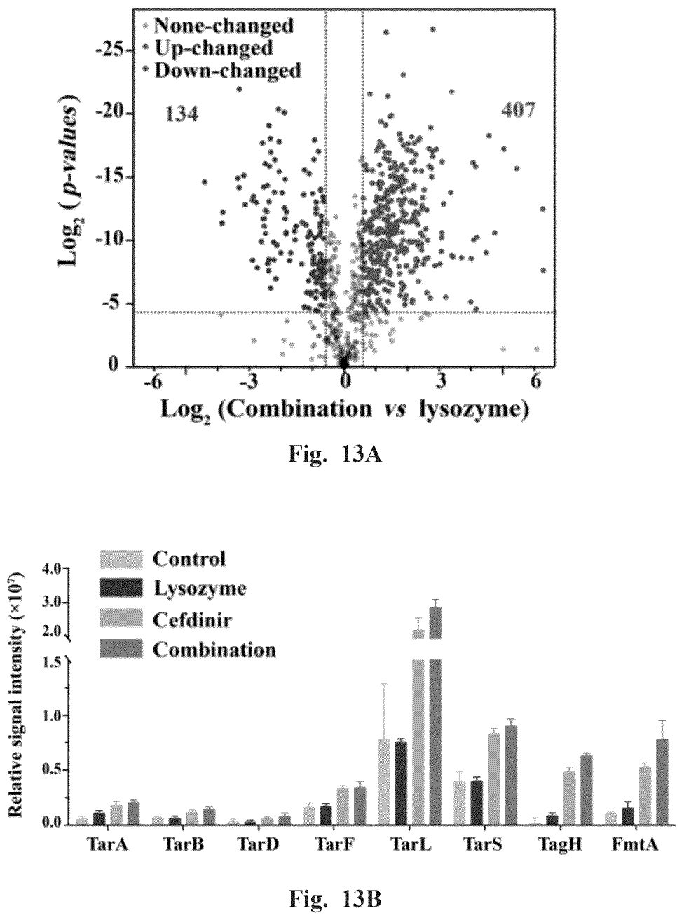

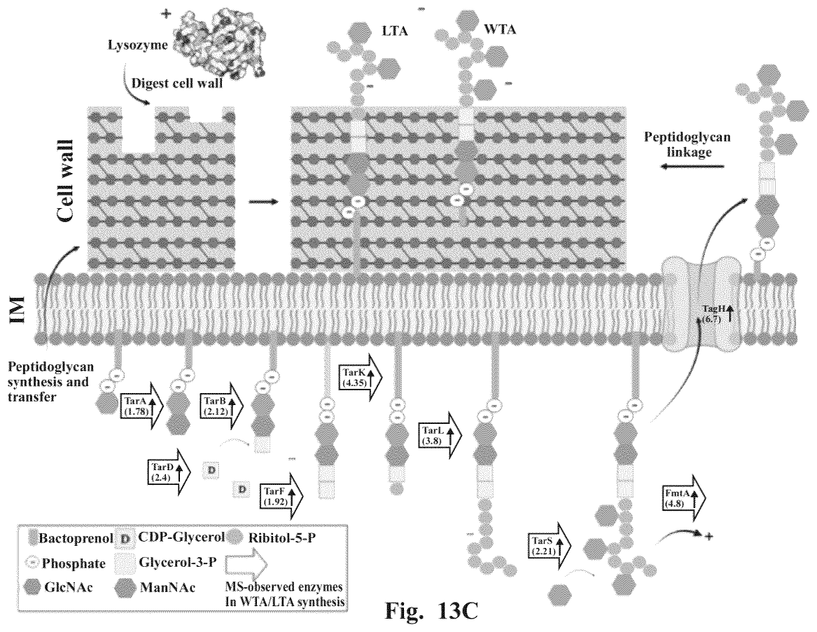

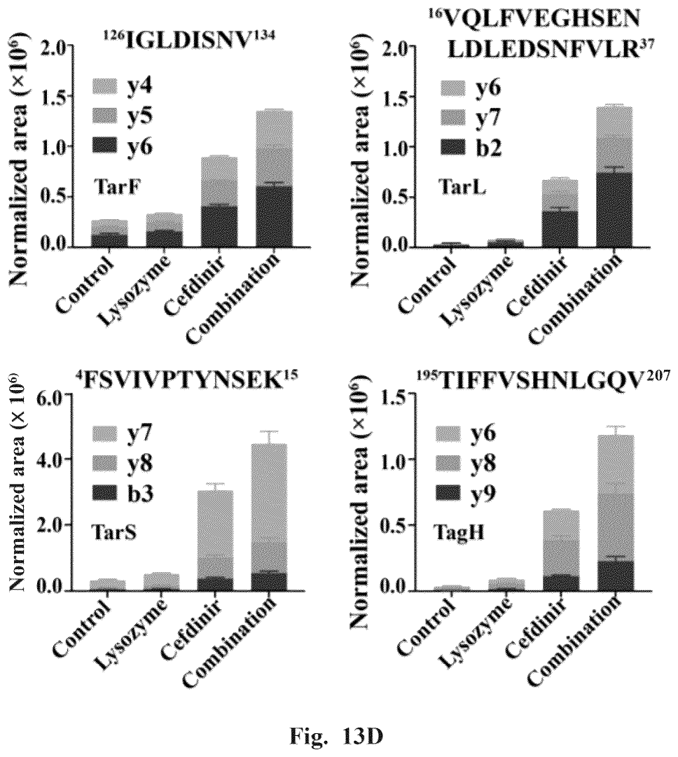

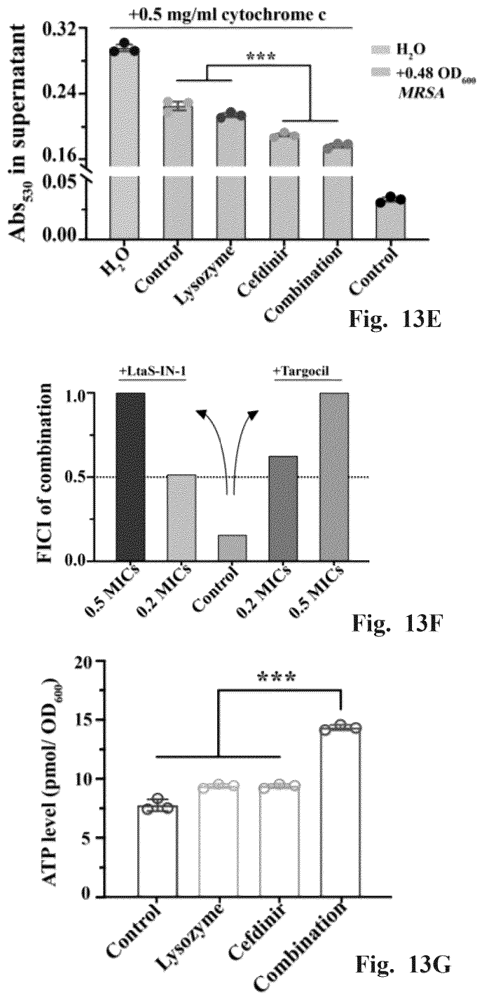

FIG. 13: Scheme to screen the novel lysozyme adjuvants. 4989 compounds from our drug library were screened against MRSA or MRSE in the absence or presence of 1 mg/ml lysozyme. Those without treatment of lysozyme served as control groups. The real-time bacterial growth curves were monitored for 24 hours. The inhibition ratio (%) was calculated as (ODcontrol−ODsample)/(ODcontrol−ODbackground)×100%. Here, the absorbance of bacterial suspension without any treatment served as ODcontrol. Synergy effect was defined as the inhibition ratio of ≥90%.

FIG. 16A to FIG. 16B: Dose-dependent inhibition against FIG. 16A: MRSA or FIG. 16B: MRSE under the treatment of lysosyme alone or its combination with several drug candidates including curcusone B, α-mangostin and tanshinone IIA.

FIG. 17A to FIG. 17H: Cefdinir had the broad-spectrum combination effect with lysozyme. FIG. 17A: The MIC₉₀ of lysozyme and cefdinir against 8 gram-positive pathogens. FIG. 17B to FIG. 17H: Representative heat-maps of com-bination therapies against 8 gram-positive pathogens. Inhibition ratios of bacterial growth were indicated by upper heat-map columns.

FIG. 18A to FIG. 18B: G-type lysozyme was purified from eggs of Chinese goose (Answer cygnoides). FIG. 18A: The coomassie blue staining gel showed the increasing purity of lysozyme from goose egg white treated with isoelectric precipitations at pH 6.0, 4.0, 7.0 and then cation exchange chromatography (SP column) (lysozyme was confirmed by using in-gel digestion mass spectrometric identification, data not shown). FIG. 18B: The curve of gel filtration chromatography to purify lysozyme from SP elution.

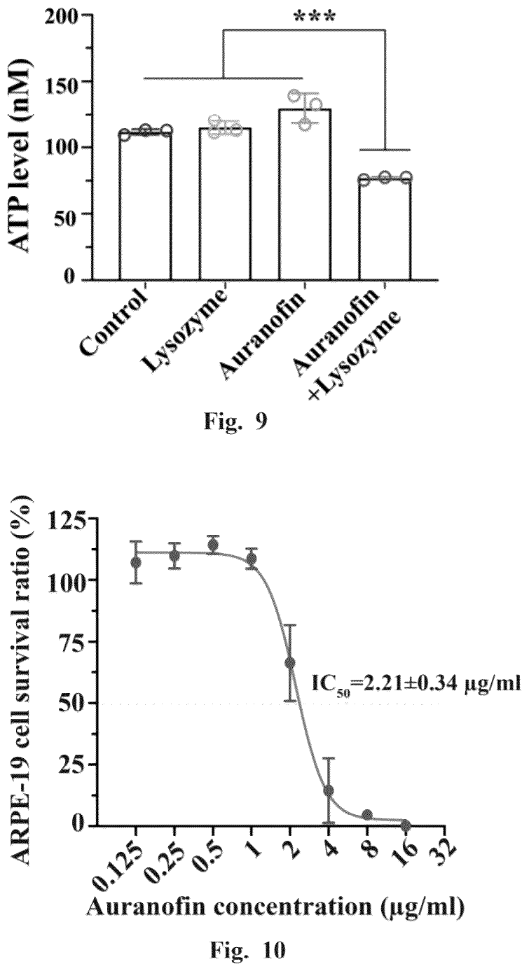

FIG. 19A to FIG. 19D: (Partial) synergy effects were observed in combination therapies consisting of cefdinir and one of several lysozymes, including FIG. 9A: chicken-source lysozyme. FIG. 19B: T4 phage-source lysozyme. FIG. 19C: Bacillus subtilis-source lysosyme and FIG. 19D: goose-source lysozyme. Their representative heat-maps were shown, Inhibition ratios of bacterial growth were indicated by right heat-map columns.

FIG. 20A to FIG. 20B; FIG. 20A: Representative images reflecting the time-killing effect on MRSA growth under the treatment of lysozyme, cefdinir alone or in combination. FIG. 20B; Biofilm-based absorbance intensities reflecting the effects of different treatments on biofilm after biofilm had appeared.

FIG. 21A to FIG. 21B: FIG. 21A: The damage evaluation of cell membrane under combination therapy effects MRSA was exposed to 0.5 mg/ml lysozyme, 0.5 μg/ml cefdinir and their combination for 1 hour. Triton-X100/NP-40 was used to break cell membrane in the positive group. FIG. 21B: The HADA-based signal intensities of cell wall under treatment of 0.5 mg/ml lysozyme for 16 hours. All cell walls of MRSA under different treatments were collected by ultrasonication. Bacteria treated with 0.05 μg/ml cefazolin (0.5 MIC, a random-selected representative β-lactams antibiotic) served as the negative control group. Cell walls from MRSA treated with cefdinir were more sensitive to lysozyme if com-pared with those from other groups.

FIG. 22A to FIG. 22C: Combination therapy had no effect on acetylation modification on cell wall. FIG. 22A: Diagram to show the 4-MU-Ac-based reaction catalyzed by several typical enzymes involved in acetylation modification on cell wall. FIG. 22B: Fluorescent curves analysis on 4-MU-Ac contents, prompting that the linear range in absorption intensity. FIG. 22C: Fluorescent spectrum of reaction mixture consisting of 0.5 mM 4-MU-Ae and 0.5 μg cell lysis supernatant from MRSA under different treatments.

FIG. 23A to FIG. 23F: Proteomic analysis on MRSA under different treatments. FIG. 23A: Feat-map of total protein levels. FIG. 23B: (K) annotation between combination and lysozyme only group. FIG. 23C and FIG. 23E: Volcano plot and FIG. 23D and FIG. 23F: GO annotation analysis when cefdinir only group was compared with FIG. 23C and FIG. 23D: combination group or FIG. 23E and FIG. 23F control group, respectively.

FIG. 24A to FIG. 24D: Heat maps showing effects of LTA synthesis inhibitor (LtaS-IN-1, FIG. 24A to FIG. 240) and WTA synthesis inhibitor (Targocil, FIG. 24C to FIG. 24D) on combination therapies against MRSA. Inhibition ratios of bacterial growth were indicated by right heat-map columns.

FIG. 25A to FIG. 25D: Proteomic analysis on MRSA under different treatments. The relative levels of FIG. 25A: ATP synthase subunit c, subunit β and FIG. 23D ADP-dependent (S)-NAD)(P)H-hydrate dehydratase. PRM-based analysis on FIG. 258: ATP synthase subunit c and FIG. 25C: ATP synthase subunit f. The inserted sequence in each data was the unique peptide in PRM analysis.

FIG. 26A to FIG. 26G: Proteomic analysis on MRSA under different treatments, FIG. 26A: Fold-changes in proteins in MRSA treated with lysozyme only or combination therapy. FIG. 26B to FIG. 266: PRM-based analysis on these selected proteins. These inserted sequences were corresponding unique peptides in PRM analysis,

FIG. 27A to FIG. 27D: Analysis on the levels of FIG. 27A: pyruvate, FIG. 27B: the total contents of NAD+ and NADH, FIG. 27C: relative levels of NADH and FIG. 27D: the ratio between NAD+ and NADH in MRSA under different treatments for 1 hour.

FIG. 28A to FIG. 280; Cefdinir showed great combination effect with lysozyme in ARPE-19 cell infection model. The toxicity analysis of FIG. 28A: lysozyme or FIG. 28B: cefdinir on ARPE-19 and 293T cells. FIG. 24C: Survival ratios and FIG. 28D: bacterial loads of ARPE-19 cells under different treatments were shown. In general, the combination composing of lysozyme and cefdinir in safe dosages significantly reduced the bacterial loads and increased the cell survival ratios of ARPE-19 cells infected by MRSA.

FIG. 29: Representative images showing these wounds of rats under different treatments for 3 days. Compared with those in other groups, rats treated with combination therapy had more relieving scars.

FIG. 30A to FIG. 30I: Tedizolid phosphate showed great combination effect with lysozyme against broad spectrum grain-positive pathogens. FIG. 30A to FIG. 30B: Representative heat-maps indicating the inhibited growth of FIG. 30A; MRSA and FIG. 30B: MRSE under combination therapies. FIG. 30C: Time-kill curves for log-phage MRSE under different treatments for up to 22 hours. FIG. 30D: Isobolograms of combination therapies against 8 different gram-positive pathogens. FIG. 30E: Fractional inhibitory concentration (FIC) index analysis on MRSA between tedizolid phosphate and 5 subtypes of lysozymes. FIG. 30F Resistance acquisition curves during 12 passages in either MRSA or MRSE treated with the sub-inhibitory concentration of tedizolid phosphate or combination therapy with identical concentration of lysozyme. The gaps among curves indicated that combination therapy efficiently suppressed the development of tedizolid phosphate resistance. FIG. 30G: Representative heat maps of mutation frequencies of MRSA exposed to lysozyme, tedizolid phosphate or their combination at different concentrations and FIG. 30I: mutation prevention concentrations (MPC) of tedizolid phosphate used in combination with lysozyme. FIG. 30I: Crystal violet assays on MRSA and MRSE, indicating that combination therapies were not only able to inhibit the form of biofilm, but also remove the biofilm thoroughly even when biofilm had appeared. TP: tedizolid phosphate. ***p≤0.001.

FIG. 31A to FIG. 31H: Proteomic analysis of MRSA under different treatments and follow-up effects on energy metabolism. FIG. 31A: The total heat-map of changed proteins in MRSA under different treatments, indicating the high similarity between the combination group and the 40 μg/ml tedizolid phosphate group. FIG. 31B: Volcano plot. FIG. 31C: GO (gene ontology) annotation and FIG. 31D: KEGG (Kyoto Encyclopedia of Genes and Genomes) analyses of the differential expression proteins in MRSA under different treatments for 1 hour. The dotted lines in x- and y-axes of volcano plot reflect an adjusted p-value<0.05 (Student's t-test) and |Fold change|>1.5, respectively. FIG. 31E: The diagram shows the inhibited glycolysis pathway and TCA (Tricarboxylic Acid) cycle, in which the up-:down-regulated proteins are highlighted in red*blue areas Numbers in the highlighted areas indicate the fold-change of proteins in these cycles. FIG. 31F to FIG. 31H: Analyses on the levels of FIG. 31F; NAD+/NADH, FIG. 31G: pyruvate and FIG. 31H: ATP in MRSA under different treatments for 1 hour. TP: tedizolid phosphate. **p<0.01, ***p<0.01,

FIG. 32A to FIG. 32H: Combination therapies accelerated the intracellular accumulation of tedizolid phosphate by alkalizing the cellular micro-environment, activating alkaline phosphatase. FIG. 32A: The synthesis of nascent proteins in MRSA under different treatments for 1 hour. The dotted line indicates half inhibitory concentrations of tedizolid phosphate. FIG. 321k Time-dependent or FIG. 32C: dose-dependent analysis on intracellular accumulation of tedizolid phosphate in MRSA, indicating that combination therapies significantly increased the content of intracellular tedizolid phosphate. FIG. 32D: The FICI analysis on combination therapies against MRSA exposed to different ion-targeting inhibitors. The increasing FICI in the CCCP-treatment group indicated that H+ might play a role in the combination therapy. FIG. 32E: Analyses on membrane potentials in MRSA and MRSE under different treatments for 1 hour. The higher fluorescence ratio (green/red) indicated the weaker membrane potential. Bacteria treated with CCCP, in which proton gradient can be eliminated, served as the positive control group. FIG. 32F: Fluorescence signal intensities at 520 nm from a pH-sensitive fluorescence probe BCECF-AM significantly increased if MRSE was treated with combination therapies for 1 hour, indicating a higher alkaline cellular pH level in the combination group. FIG. 32G: The rate studies of alkaline phosphatase extracted from MRSA under different treatments to hydrolyze tedizolid phosphate in vitro. FIG. 32H: Representative micrographs showing the catalytic activity of alkaline phosphatase. The dark pigment shows the product (NBT-formazan) catalyzed by alkaline phosphatase.

FIG. 33A to FIG. 33D: Comparison of FICIs for several combination therapies, where lysozyme was used in combination with each compound. FIG. 33A: Comparison of FICIs between single drug and combination therapies and the synergy effects wen confirmed when FICI<0.5 (dah line). T: tedizolid; TP: tedizolid phosphate; D: delpazolid; DP: delpazolid phosphate; E: eperezolid; EP: eperezolid phosphate. FIG. 33B: Structures and the FICI values of tested compounds and their combinations with lysozyme. These structures of new synthesized pro-drugs with phosphorylation were highlighted and the pan in blue box indicated the core structure of oxazolidinone-class antibiotic. FIG. 33C: The BCECF-AM-based fluorescence signal from MRSA to indicate intracellular pH of bacteria. FIG. 33D: The conditions to synergize the antibiotic with lysozyme.

FIG. 34A to FIG. 34L: Urea cycle and cell division were targeted in MRSA under combination therap. FIG. 34A: Diagram showing urea cycl, in which up-/down-regulated proteins were highlighted in red/blue areas. These changes indicate increased levels of ammonia and thus an alkalized cellular pH level. FIG. 34B: Heat maps and old-changes of FIG. 34C; arginine deiminase. FIG. 34D: urease subunit alpha, and FIG. 34E: omithine carbamoyl transferase in MRSA under different treatments for 1 hour. FIG. 34F: The FICI analysis on combination therapies against MRSA exposed to urease and/or arginine deiminase inhibitors. The increasing FICIs in inhibitors-incubation groups indicated that the aggravated alkaline pH level was partly relieved. FIG. 34G: Successful observation of synergistic effect of combination therapy composed of tedizolid phosphate and poly-L-lysine, a randomly selected representative of a compound with considerable positive charge in physiological conditions. FIG. 34H; Morphological change of MRSA exposed to lysozyme (0.5 mg/ml), tedizolid phosphate (0.25 or 0.5 μg/ml) or their combination for 1 hour. Compared with other scanning electron microscopy images of MRSA, those under combination therapies bad numerous clearly observable bubble-like divisions (white arrowheads). FIG. 34I to FIG. 34K: Analysis of fold-change in levels of three critical enzymes in cell division, i.e., FIG. 34I: OatA, FIG. 34J: FtsA and FIG. 34K: SepF in proteomics studies. TP: tedizolid phosphate. FIG. 34L: lasso-like Z-rings observed at the unequal bubblelike junction in contrast to the equal division site.

FIG. 35A to FIG. 35E: Combination therapy effectively relieved the symptoms of rats in a skin infection model. FIG. 35A: Flow-process diagram shows the details in rot skin infection model. FIG. 35B: Bacterial load in wounds of infected rats under different treatments for 7 days. FIG. 35C: The representative hematoxylin-cosin (H-E) staining images of wounds of rat in the skin infection model. Two magnifications (upper: 40× and lower: 100×) were available, Less dense inflammatory cells with blue nuclei (black arrowheads) and smoother edges of slides were clearly observable in the combination therapy group. FIG. 35D to FIG. 35E: The normalized levels of FIG. 35D: IL-6 or FIG. 35E: ROS in wounds of rats under different treatments for 7 days. TP: tedizolid phosphate, *p<, 0.05,***p<0.001.

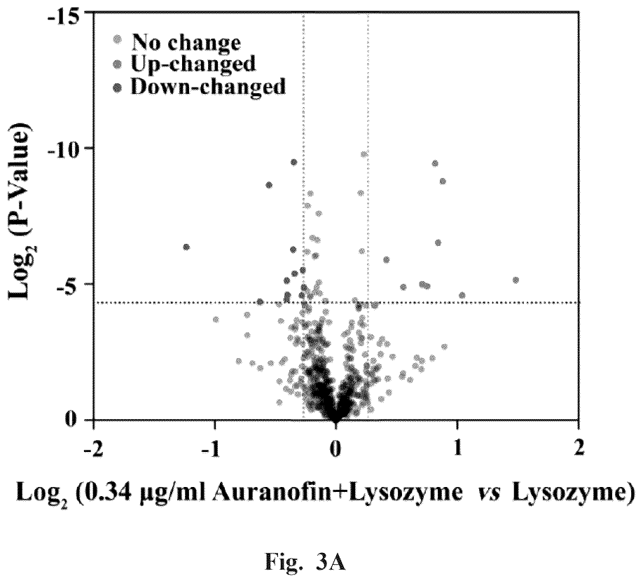

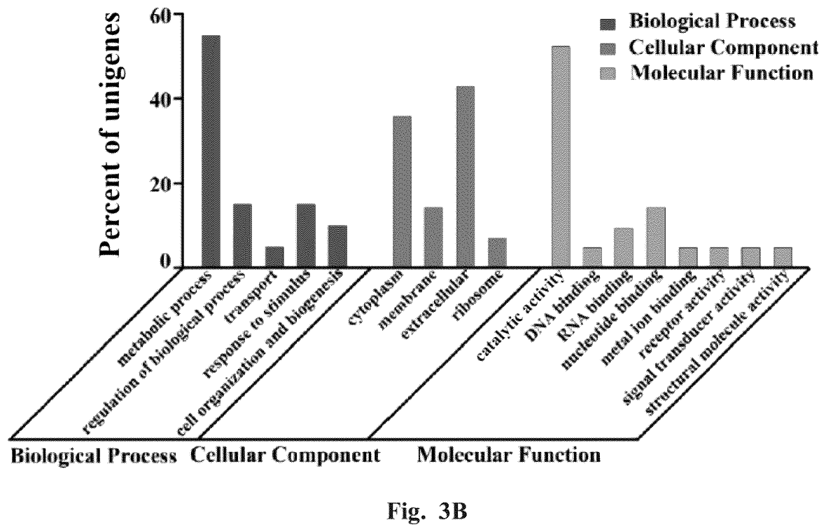

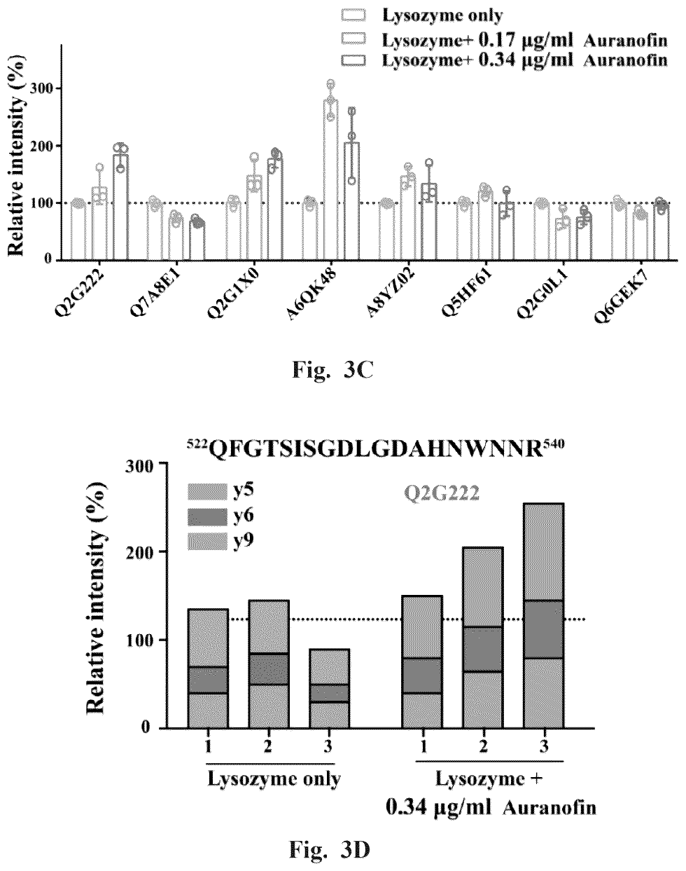

FIG. 36A to FIG. 36F: Tedizolid phosphate had a strong synergistic effect with lysozyme to treat ocular infection FIG. 36A to FIG. 36B: Bacterial loads in FIG. 36A: tear fluids and FIG. 36B: ocular homogenates of C57BL/6 mice under different treatments in the ocular infection model. The dashed line indicates the lowest detection limit in bacterial load in ears. FIG. 3C: The percentages of invaded epithelial cells in tear fluids of mice under different treatments, in which invaded epithelial cells are defined as those with ≥1 bacterium in the hema-3 staining image (×400 magnification). The red dots in the inserts show the average number of bacteria per epithelial cell, in which similar values indicated minimal effects on infectivity of the bacterium itself under combination therapy if compared with those in the uninfected group. FIG. 36D: The normalized expression levels of SP-D in tear fluids and ocular homogenates. GAPDH was used to normalize SP-D levels in ocular homogenates. FIG. 36E: Representative fluorescein images showing the stained corneas of mice under different treatments for 48 hours. Write arrows indicate the injured corneal integrities. All eye injuries were scored according to the ophthalmology scoring standard, FIG. 36F: Representative H-E staining images of eyeballs from C57BL/6 mice under different treatment. Two magnifications (upper: 40× and lower: 20×) were available. Black arrowheads indicate infiltrating inflammatory cells and edges of lens. Notably, ganglion cells in the ganglionic layer of retina were also “irregularly” observed. Compared with eyeballs in the combination therapy group, those in other infected groups were observed stronger inflammatory response and irregular retinas. TP: tedizolid phosphate; GAPDH: glyceraldehyde 3-phosphate dehydrogenase, *p<0.05, **p<0.01, **p<0.001.

FIG. 37A to FIG. 37I: Combination therapy successfully re-built the ocular bacterial communities in tear. FIG. 37A: Venn diagram showing the detected OTUs in tears of mice under different treatments. 575 core OTUs were shared among different groups. FIG. 37B: Composition analysis on bacterial communities in tears from healthy mice (left) or infected mice under combination therapy (right). FIG. 37C: Heat-maps showing the relative abundance of detected bacterial genera, which clearly indicated that combination therapy had successfully re built bacterial communities in teams. FIG. 37D: Change in proportions of several bacteria in communities in tears of mice under different treatments. Here, combination therapy not only reduced pathogens, such as Staphylococcus, but also increased commensal probiotics including Prevotella, Anaerostipes and Propionibacterium. FIG. 37E; The partial least squares-discriminant analysis on bacterial communities. FIG. 37F to FIG. 37G: The alpha and beta diversity analyses on different bacterial communities, prompting community diversities were minimally of affected if different therapies were applied. The whisker lines of each diversity boxplot indicate the minimum, median and maximum values, respectively. FIG. 37H: Graphlan map showing the evolutionary branching trees of detected genera. The heat-cycles in outer layers show the relative genus-associated abundance in different groups highlighted by different colors. FIG. 37I: Heat-maps showing the correlation coefficient among different genera; red and blue indicate the co-existence and inter-exclusion, respectively. TP: tedizolid phosphate. *p<0.05, **p<0.01, ***p<0.001.

FIG. 38: The scheme to screen novel lysozyme adjuvants from FDA approved drugs. 1953 compounds were screened against MRSA or MRSE in combination with 1 mg/ml lysozyme. Here, LB medium without bacteria served as background group and bacteria under monotherapy of compound served as control group of each compound. Bacterial optical density at 60 m was real-time recorded to draw bacterial real-time growth curves within 24 hours. The relative inhibition ratio was calculated as (ODcontrol−ODcombination)/(ODcontrotl−ODbackground)×100%. Synergy effect was defined as the relative inhibition ratio of ≥90%.

FIG. 39A to FIG. 39B: The growth of bacteria exposed to drugs in the absence and presence of lysozyme. Op-tical densities at 60) nm of FIG. 39A: MRSA or FIG. 39B: MRSE under the treatments with several interesting drugs alone or their combination with 1 mg/ml lysozyme in high-throughput screening. Tedizolid phosphate was able to be observed in both MRSA and MRSE groups.

FIG. 40: Synergy analyses on combination therapies between lysozyme and several hits. Representative heat-maps of MRSA or MRSE under the combination therapies consisting of lysozyme and several candidate drugs, i.e., chlorhexidine, tilmicosin, mezlocillin, carmofur, cefoperazone, doripenem, nitazoxanide and bardoxolone.

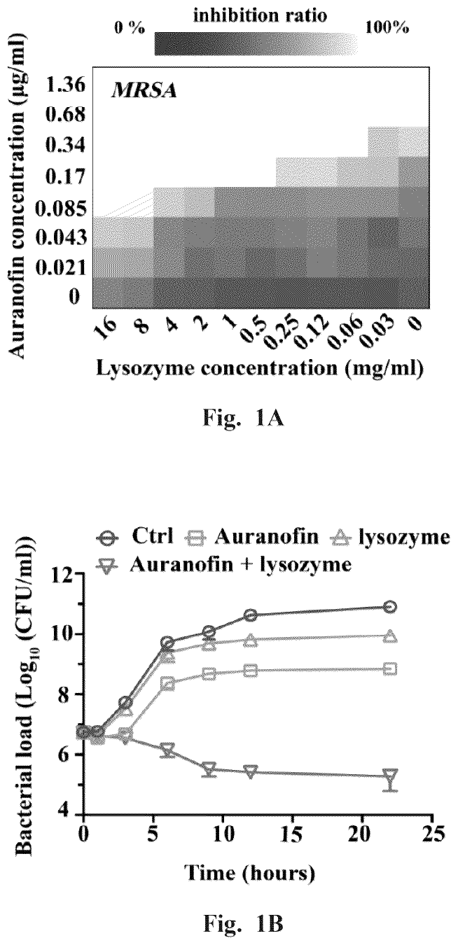

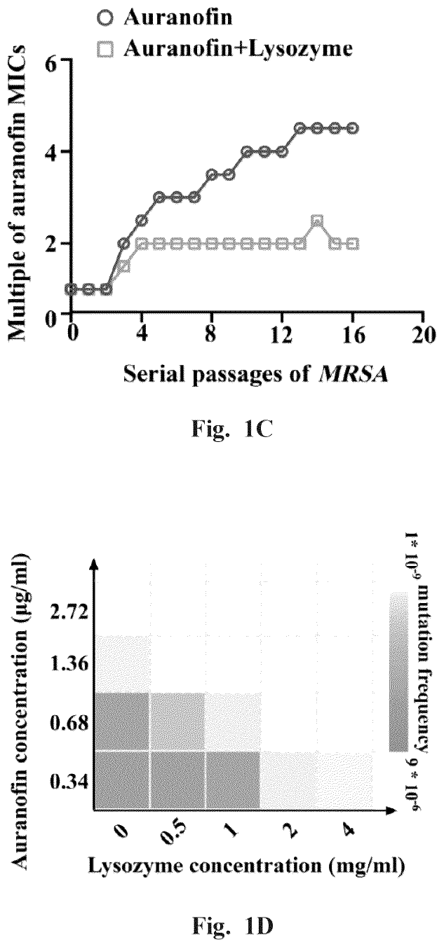

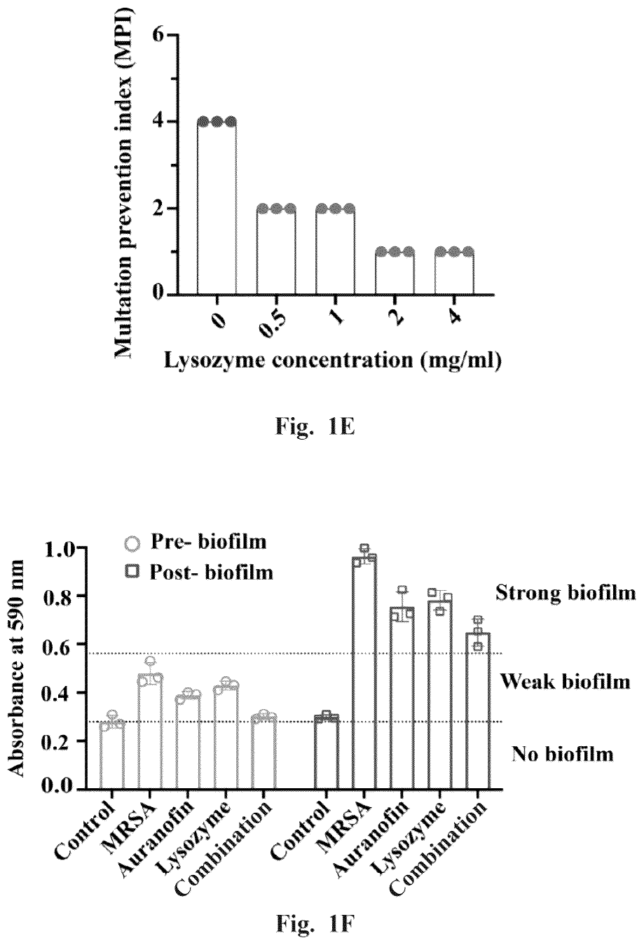

FIG. 41A-1 to FIG. 41C: Several synthesized oxazolidinone-class antibiotics. FIG. 41A-1 to FIG. 41A-2: Nuclear magnetic resonance and LC-MS spectroscopies showing validities and purities of two synthesized pro-drugs (delpazolid phosphate and eperezolid phosphate) with phosphorylation modifications. FIG. 41B: The synthesis routes of delpazolid phosphate and eperezolid phosphate, and their structural comparison, with other oxazolidinone-class antibiotics, where their core structures were highlighted in the blue box. FIG. 41C; Isobolograms of the combination of lysozyme and several oxazolidinone-class antibiotics against MRSA or MRSE. The cyan dash line indicated ideal isobole, where drugs acted additively and independently. Data points below this line indicated synergism.

FIG. 42A to FIG. 42C: The dynamic killing effects on MRSA and MRSE. FIG. 42A Representative time-kill curves of MRSA under different treatments for 22 hours, and these pictures of bacterial cultures showing inhibited growth of FIG. 42; MRSE and FIG. 42C; MRSA under different treatments at the end of experiments.

FIG. 43A to FIG. 43B: G-type lysozyme was purified from eggs of Chinese goose (Anser cygnoides). FIG. 43A: The elution peak of purified lysozyme by using cation exchange chromatography. The coomassie blue staining gels were inserted. FIG. 43B: The gel filtration chromatography curve of purified lysozyme and a representative gel image showing the purity of lysozyme.

FIG. 44A to FIG. 44C The combination effects on bacterial mutation and biofilm. FIG. 44A: Heat map showing mutation frequencies of MRSE under different treatments and FIG. 44B: the mutation prevention concentrations (MPC) of tedizolid phosphate in the absence or presence of lysozyme. FIG. 44C: Biofilm-based absorbance analysis on MRSA or MRSE under different treatments. The significant reduce in absorbance at 600 nm indicated that combination therapy was able to strongly inhibit the bacterial growth even if biofilm had appeared. All assays were performed in triplicate. ***P<0.001.

FIG. 45A to FIG. 45C: The effects of optical density, cell membrane and ceil wall of bacteria under different treatments. FIG. 45A Optical densities of MRSA treated with tedizolid phosphate in the absence or presence of 0.5 mg/ml lysozyme for 1 hour. FIG. 45B: Permeability analysis on cell membrane of MRSA under different treatments. Hem, bacteria treated with 0.5% Triton-X100 served as positive control group. FIG. 45C; Evaluation on cell wall integrity in MRSA under different treatments. Mid-log-phase MRSA was cultured in UL medium supplied with tedizolid phosphate, lysozyme alone or their combination for 1 hour, Bacteria without any drug treatment were re-suspended in digestion buffer (0.2% EDTA+0.5 ng/ml lysozyme) to serve as positive control group. Remarkably, diffused fluorescence signal from SYBR™-Gold dyes (white arrow heads) was clearly observed in positive control group but not in other groups suggesting that cell wall wasn't damaged under combination therapy.

FIG. 46A to FIG. 46B: Pearson correction coefficient (R²) and principal component analysis (PCA) maps for changed proteins in MRSA treated with 40 μg/ml tedizolid phosphate or combination therapy. FIG. 46A: Heat map of correction coefficient among different groups. Value in each combination-box indicated relative Pearson correction coefficient. FIG. 46B: Two principal components were defined to analyze changed proteins in proteomics study. Here, higher similarities between combination therapy group and 40 μg/ml tedizolid phosphate group were clearly observed.

FIG. 47A to FIG. 47I: Proteomic analysis on MRSA exposed to lysozyme, tedizolid phosphate alone or their combination for 1 hour. Volcano plot of proteins, GO (gene ontology) and KEGG (Kyoto encyclopedia of genes and genomes) annotation analysis on differential expression proteins in MRSA under treatments with FIG. 47A to FIG. 47C: 0.5 μg/ml tedizolid phosphate or combination therapy. FIG. 47D to FIG. 47F: 40 μg/ml tedizolid phosphate or 0.5 μg/ml tedizolid phosphate. FIG. 47G to FIG. 47I: 40 μg/ml tedizolid phosphate or combi-nation therapy. The total numbers of up-changed or down-changed proteins were highlighted by red or cyan in the related volcano plot maps,

FIG. 48A to FIG. 48V: No synergistic effect was observed if TP was used in combination with several inhibitors. FIG. 48A; Minimum inhibitory concentrations (MIC₉₀) of 21 representative transporters (or pumps) inhibitors against MRSA, and FIG. 48B their FICI in combination with tedizolid phosphate according to FIG. 48C to FIG. 48V their representative heat-maps of combination therapies.

FIG. 49A to FIG. 49P: Combination effects on MRSA or MRSE exposed to several inhibitors. FIG. 49A: Classification diagrams of 14 ions-targeting inhibitors and FIG. 498 to FIG. 49P: their representative heat-maps of combination therapies.

FIG. 50A to FIG. 50C: Combination therapy alkalized MRSA, leading to more reaction products catalyzed by activated alkaline phosphatase. FIG. 50A: Combination therapy against MRSA aggravated alkaline cellular pH level, as judged from increased fluorescence signal intensities at 520 nm from pH-sensitive fluorescence probe BCECF-AM. Three thin lines per treatment indicated three independent repeats. FIG. 50H: Activated alkaline phosphatase extracted from MRSA under combination therapy resulted in more catalytic products (NDT-formazan) with optical absorbance at 560 nm. FIG. 50C: Intra-cellular accumulation of tedizolid in MRSA exposed to 0.2 μg/ml tedizolid in the absence or presence of lysozyme for 0.5 hour.

FIG. 51A to FIG. 51D: Inhibitors targeting urea cycle relieved cytoplasmic alkaline level in MRSA. Representative heat-maps of MRSA treated with combination therapies in the presence of inhibitors against FIG. 51A: arginine deiminase (0.5 MIC BB-Cl-amidine). FIG. 51B: urease (0.5 MIC acetohydroxamic acid) or FIG. 51C: both enzymes (0.25 MIC BB-Cl-amidine+0.25 MIC acetohydroxamic aid). FIG. 51D: The pH-dependent fluorescence signal intensities at 520 nm were significantly reduced when urea cycle was inhibited, indicating that the cytoplasmic alkaline level in MRSA did be relieved.

FIG. 52: Morphological change of MRSE under different treatments. Compared with scanning electron microscopy images of MRSE with or without the treatment of tedizolid phosphate or lysozyme alone, those in combination groups were clearly observed numerous cytolysis sites or bubble-like humps. White arrowheads indicated damaged areas.

FIG. 53A to FIG. 53H: Tedizolid phosphate showed great combination effect with lysozyme in cell-based infection model. The survival ratios of FIG. 51A and FIG. 53D: ARPE-19 cells, FIG. 538 and FIG. 53E; NIH/3T3 cells and FIG. 53C and FIG. 53F: 293T cells exposed to lysozyme or tedizolid phosphate for 12 and 24 hours. Number of cells with-out any drug treatment was normalized for 100% survival ratio. FIG. 53G: Bacterial load in ARPE 19 cells and FIG. 53H survival ratios of ARPE-19 cells after ARPE-19 cells were infected by mid-log-phase MRSA at multiplicity of infection (MOI) of 10 for 20 hours. All assays were performed in triplicate.

FIG. 54: Representative gram-staining images of MRSA in wounds of rats under different treatments. These images clearly indicated that combination therapy was able to significantly reduce the bacterial load in infected wound. Each blue point reflected the bacterium. Three images per group were randomly selected.

FIG. 55A to FIG. 558: The schematic diagram in ocular bacterial infection model and the ocular anatomy structure of C57BL/6 mouse. FIG. 55A: A flow chart showing the details on infected mice in ocular infection model. FIG. 55B The structure of mouse eye. The middle intraocular cartoon showed physiological positions of several labeled tissues. Sub-structure of retina was detailed in right diagram.

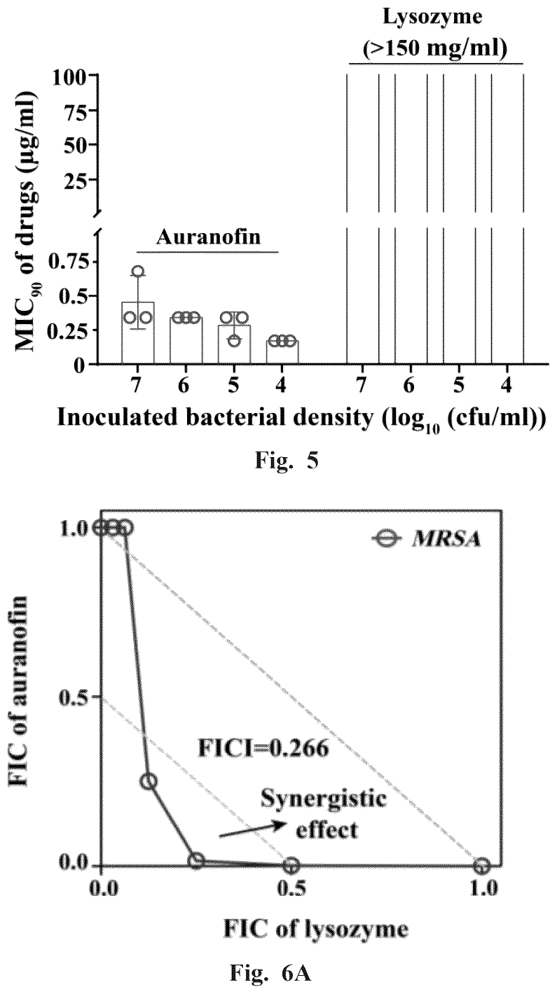

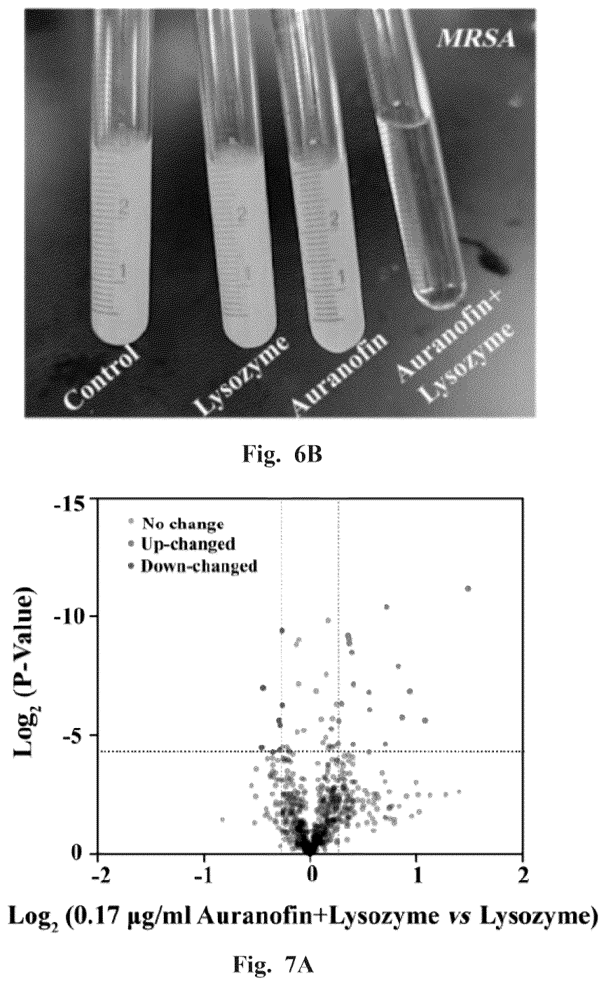

FIG. 6: Representative images showing epithelial cells in tears of mice under different treatments. Black arrows indicated the invading bacteria.

FIG. 57: Representative fluorescein images showing the stained corneas of mice under different treatments for 24 hours. Write arrows indicated the injured corneal integrities. These eye injuries were scored according to described scoring standard. Compared with other infected mice, that under combination therapy showed less injury.

FIG. 58A to FIG. 58D: The reliability assessment of 16S rRNA analysis. The species accumulation curves (FIG. 58A and FIG. 58C) and OTU rank curves (FIG. 58B and FIG. 58D) when bacterial communities in wears (FIG. 58A and FIG. 58B) or ocular homogenates (FIG. 58C and FIG. 58D) were analyzed by performing 16S rRNA analysis.

FIG. 59: The composition analysis on bacterial communities in tears from infected mice with-out any treatment (left), those treated with TP (middle) or lysozyme (right). The inserted values indicated corresponding composition percentages. Herein, Staphylococcus (MRSA) was highlighted by orange yellow.

FIG. 60A to FIG. 60C: Composition analysis on ocular bacterial microbiome of mice under different treatments. FIG. 60A: The composition analysis on bacterial communities in tears. FIG. 60B: Composition analysis on bacteria in ocular homogenates and FIG. 60C: several representative bacteria with the change in pro-portions in these bacterial communities of mice under different treatments, Bacteria were descripted at the genus level. *P<0.05, **P<0.01, ***P<0.001.

FIG. 61A to FIG. 61C: The analysis on bacterial communities in ocular homogenates of mice. FIG. 61A: Venn diagram showing the detected OTUs in ocular homogenates of mice under different treatments. 35 core OTUs were shared among different groups. FIG. 61B: The composition analysis on bacterial communities in tears from healthy mice (upper left), infected mice without any treatments (middle left) or infected mice treated with TP (middle right), lyosome (lower left) or their combination therapy (tower right). FIG. 61C: The heat-maps showing the relative abundance of detected bacterial genus in different groups.

FIG. 62A to FIG. 62C: Analyses of similarities and diversities. FIG. 62A: The principal component analysis on bacterial communities in ocular homogenates. FIG. 62B: The alpha and FIG. 62C: beta diversity analyses on bacterial communities in ocular homogenates of mice under different treatments, prompting bacterial com-munity diversities were recovered in infected mice if combination therapy was applied. These bot-tom-top lines of each diversity boxplot indicated minimum, median and maximum values, respectively.

FIG. 63A to FIG. 63B: The relationship among bacteria in communities in ocular homogenates. FIG. 63A: Graphlan map showing the evolutionary branching trees of detected genera. The heat-cycles in outer layers showed relative genus-associated abundances in different groups highlighted by different colors. FIG. 63R The heat-maps showing the correlation coefficient among different genera. Hemin, red and blue indicated the co-existence and inter-exclusion, respectively.

FIG. 64A to FIG. 64F: Representative heat maps indicating the inhibited growth of Enterococcus faecalis, Enterococcus faecium, Streptococcus mutans, Streptococcus pyogenes, Streptococcus pneumonia or Corynebacterium pseudodiphtheriticum under combination therapies.

FIG. 65A to FIG. 65D: Representative heat maps indicating the inhibited growth of MRSA treated with TP in the presence of chicken lysozyme, goose lysozyme, T4-phage lysozyme and bacterial lysozyme, respectively.

### DETAILED DESCRIPTION OF THE DISCLOSURE

### I. General

In the present disclosure, combination therapies from over thousands of clinical drugs have been successfully discovered and show, that hydrolase increases the cellular uptake of antimicrobial drugs and re-sensitizes them against different bacteria. The excellent combination effects have been validated in in-vitro assays and in-vivo animal studies. It is the same important that long-term usage of the developed combination therapies did not lead to drug resistance. Meanwhile, the mechanisms of the effects of combination therapies have also been uncovered.

### II. Definitions

Unless otherwise defined, all technical and scientific terms used herein have the same meanings as commonly understood by those skilled in the art. For clarity, some Kerns of the present invention are defined below.

As used herein, the term “combination” has the same or similar meanings as commonly understood by a skilled person and refers to a collection of things that have been combined, Therefore, a skilled person will appreciate that a combination will comprise two or more components, and the components of a combination can be used together or separately.

As used herein, the term “hydrolase” has the same or similar meanings as commonly understood by a skilled person and is a generic term for a class of enzymes that catalyze hydrolysis reactions using water as a receptor for the transferred group.

As used herein, the term “microorganism” has the same or similar meanings as commonly understood by a skilled person and is a collective term for a group of organisms, including bacteria (which can be further divided into, for example Gram-positive bacteria and Gram-negative bacteria), viruses, fungi, parasites and the like. In a preferable embodiment, the bacteria are Gram-positive bacteria.

As used herein, the term “drug resistance” has the same or similar meanings as commonly understood by a skilled person and refers to the tolerance of microorganisms, parasites and tumor cells to the effects of chemotherapeutic drugs. Once resistance has developed, the chemotherapeutic effect of the drug is significantly reduced.

As used herein, the term “anti-microorganism drug” has the same or similar meanings as commonly understood by a skilled person and refers to a drug with an inhibitory or killing activity against the microorganism. When anti-microorganism drugs are applied for a long period of time, the sensitive strains that make up the majority of the population air killed, and drug-resistant strains will replace the sensitive strains and increase the rate of bacterial resistance to the drug, that is, drug resistance as said above.

As used herein, the term “pharmaceutical composition” has the same or similar meanings us commonly understood by a skilled person and refers to a composition comprising one or e pharmaceutically active components in specified amounts and a pharmaceutically acceptable excipient. By “pharmaceutically acceptable”, it is meant the excipient must be compatible with the other ingredients of the pharmaceutical composition and not deleterious to the recipient thereof.

As used herein, the term “pharmaceutically acceptable excipient” has the same or similar meanings us commonly understood by a skilled person and refers to a substance that aids the administration of an active agent to and absorption by a subject. Pharmaceutical excipients useful in the present disclosure include, but are not limited to, binders, fillers, disintegrants, lubricants, coatings, sweeteners, flavors and colors. Pharmaceutical excipients useful in the present disclosure for transdermal/topical delivery include, but are not limited to, enhancers, solubilizers, antioxidants, plasticizers, thickeners, polymers, and pressure sensitive adhesives. A skilled person will recognize that other pharmaceutical excipients are useful in the preset disclosure.

As used herein, the term “kit” has the same or similar meanings as commonly understood by a skilled person and are used to hold chemical reagents for testing chemical composition, drug residues, virus types and other chemical reagents. A kit is generally used for therapeutic, diagnostic or testing purposes.

As used herein, the terms “treat”, “treating” and “treatment” have the same or similar meanings as commonly understood by a skilled person and refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating: improving a patient's physical or mental well-being. The treatment or amelioration of symptoms can be based on objective or subjective parameters, including the results of a physical examination, neuropsychiatric exams, and/or a psychiatric evaluation.

As used herein, the term “subject” has the same or similar meanings as commonly understood by a skilled person and refers to a living organism suffering from or prone to a disease or condition that can be treated by administration of a pharmaceutical composition as provided herein. Non-limiting examples include humans, other mammals, cats, dogs, horses, pigs, cattle, cows, goats, sheep, rabbits, and the like. In some embodiments, the subject is human.

As used herein, the term “therapeutically effective amount” has the same or similar meanings as commonly understood by a skilled person and refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science aid Technology of Pharmaceutical Compounding (1999: Pickar, Dosage Calculations (1999); and Remington: The Science and Practice of Pharmacy, 20th Edition. 2003, Gennaro, E. Lippincott, Williams & Wilkins).

As used hemin, the term “fractional inhibitory concentration index (FICI) value” has the same or similar meanings as commonly understood by a skilled person and can be calculated according to the equation: FICI=MIC_{AB}/MIC_{A}+MIC_{BA}/MIC_{B}=FIC_{A}+FIC_{B}, in which MIC, is the MIC of compound A alone; MIC_{AB} was the MIC of compound A in combination with compound B; MIC_{B} M was the MIC of compound 8 alone; MIC_{BA} was the MIC of compound B in combination with compound A: FIC_{A} was the FIC of compound A; and FIC_{B} was the FIC of compound B. Fractional inhibitory concentration index (FICI) value can be used to evaluate synergy. Notably, in a standard 2-fold dilution assay, each drug in the combination has a series of diluted concentrations. Thus, there would be a series of FICI values. In most of cases, the lowest one among these values was listed to reflect the best achieving degree of combination therapy. In terms of the lowest FICI, And synergy, part synergy and non-synergy are defined as FICI≤0.5, 0.5<FICI<1 and FICI≥1, respectively.

As used herein, the term “promoter” or “simulator” has the same or similar meaning as understood by a skilled person, which refers to an agent up-regulating or facilitating the expression of a gene or enzyme.

As used herein, the term “inhibitor” or “antagonist” has the same or similar meaning as understood by a skilled person, which refers to an agent down-regulating or inhibiting the expression of a gene or enzyme.

### III. Combination of this Disclosure

It is well-known to a skilled person that antimicrobial resistance (AMR) has always been a worldwide crisis. Since 2014, an FDA-approved drug has been FDA-approved. However, up to now, no related products can be found although combination therapies between a antimicrobial drug and another antimicrobial have been reported (so it is hardly to avoid antimicrobial resistance mentioned above) (such as Smith J R et al., Antimicrob, Agents Chemother. 2018, 62(5): e0101-18). Furthermore, there are not any publications about combination therapy between un antimicrobial drug and hydrolase.

In the present disclosure, a combination of one or more enzymes and one or more anti-microorganism drugs is provided. By using combination therapy consisting of an antimicrobial drug and hydrolase, an antimicrobial drug can be re-sensitized against lots of bacteria at less dosage in clinical (less than 1/500-fold, FICI=0.025) in Win) and in vivo. In a specific embodiment, compared with the anti-microorganism drug alone, the combination of this disclosure results in at least a 10-fold, 50-fold, 100-fold, 150-fold, 200-fold, 250-fold, 30-fold, 350-fold, 400-fold, 450-fold, 500-fold, 550-fold, 600-fold, 650-fold, 700-fold, 730-fold, 800-fold, 850-fold, 900-fold increase in the sensitivity of a microorganism to the anti-microorganism drag. Therefore, the combination of this disclosure can avoid antimicrobial resistance and extend the lifespan of a drug.

In the combination of this disclosure, hydrolases arm used in combination with anti-microorganism drugs. It is well-known to a skilled person that hydrolases are naturally abundant and safe enzymes, many of which can kill bacteria by hydrolyzing bacterial cell walls. Indeed, the combination of this disclosure can also reduce the cost since the hydrolase can be produced by the subject himself or herself (free).

In a specific embodiment, lysozyme, an isoform or homologue thereof, lysin, or lysostaphin is used as the hydrolase in the combination therapy with lysozyme or an isoform or homologue thereof being preferred. And lysozyme is naturally abundant in tears (>1 mg/ml for adults) and is defined as “natural” antimicrobial (safer and cheaper).

Moreover, in the prior art, the hydrolase has not been used to enhance antimicrobial activity, and a higher dose of the anti-microorganism drug are required, so that serious side effects will be caused. Compared with the prior art, with the addition of hydrolase, such as lysozyme, the antimicrobial efficacy can be enhanced by more than 500 folds, therefore, the dosage and overall adverse effects can be reduced.

Without being limited to specific theory, it is believed that the hydrolase (such as lysozyme) can alkalize the cellular micro-environment to activate the alkaline phosphatase, which is responsible for potential effects on an antimicrobial drug and its metabolite and uptake.

In a specific embodiment, the lysozyme can be a C-type lysozyme, for example a lysozyme from human or chicken; G-type lysozyme, for example a lysozyme hmm goose; 1-type lysozyme, for example a lysozyme from T4 bacteriophage: or bacterial-type lysozyme, for example a lysozyme from bacteria, such as Bacillus subtilis.

From the teachings of this disclosure, a skilled person will appreciate that the combination has the broad-spectrum effect among different bacteria, for example gram-positive pathogens and gram-negative pathogens, including but not limited to methicillin-resistant Staphylococcus aureus (MRSA), methicillin-resistant Staphylococcus epidermis (MRSE), Enterococcus faecalis, Enterococcus faecium, Streptococcus mutans, Corynebacterium pseudodiphtheriticum, Streptococcus pyogenes or Streptococcus pneumoniae, Propionibacterium acnes. In a preferred embodiment, then Gram-positive bacteria is methicillin-resistant Staphylococcus aureus (MRSA) or methicillin-resistant Staphylococcus epidermis (MRSE).

The combination of this disclosure can be used to rescue a subject from bacterial infection including but not limited to ocular and skin bacterial infection. And the combination of this disclosure can also be used to reduce the bacterial infection accidence in animal breeding and save cost in animal husbandry. Therefore, the combination of this disclosure can be used in a subject, which can be a mammal. In some embodiments, the subject can be a house pet, a racing animal, a domestic animal, or a primate. In some specific embodiments, the subject can be a cat, dog, horse, pig, cattle, ow, goat, sheep, rabbit, or human. In a preferred embodiment, the subject is a human.

From the teachings of this disclosure, a skilled person will appreciate that lots of drugs with inhibitory or killing activities against the microorganism can be used in the combination of this disclosure. And at present, more than 2000 drugs in FDA-approved library are screened to uncover the excellent combination therapy. In particular, the anti-microorganism drug can be an oxazolidinone class antibiotic, a β-lactam class antibiotic or anti-bacterial compound. And the inventors unexpected found that the anti-microorganism drug is an oxazolidinone with following core structure:

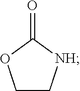

preferably, the oxazolidinone class antibiotic is an oxazolidinone with following core structure:

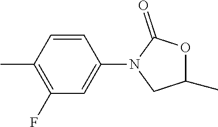

In a specific embodiment, the drug is selected from the grp consisting of Auranofin, Cefdinir, Tedizolid Phosphate, Chlorhexidine 2HCl, Tilmicosin, Mezlocillin Sodium, Bardoxolone Methyl, Cefdinir, Cefoperazone, Nitazoxanide, Carmofur, Doripenem Hydrate, Sultamicillin, Cefmetazole sodium, Benzylpenicillin potassium, Mitomycin C, Ceftriaxone Sodium, Cefradine, Meropenem Trihydrate, Ceftazidime Pentahydrate, Cefoselis Sulfate, Luliconazole, Cefcapene Pivoxil Hydrochloride, Cefoselis Sulfate, TD52 (dihydrochloride), SCP1-IN-1, Rifamycin S, Paroxypropione, Ethyl 3,4-dihydroxybenzoate, Delafloxacin (meglumine), 2,4-Diacetylphloroglucinol, Sertaconazole (nitrate) and a pharmaceutically acceptable salt thereof. In a preferred embodiment, the drug is selected from the group consisting of Auranofin, Cefdinir, Tedizolid Phosphate, Chlorhexidine 2HCl, Tilmicosin, Mezlocillin Sodium, Bardoxolone Methyl, Cefoperazone, Nitazoxanide, Carmofur, Doripenem Hydrate and a pharmaceutically acceptable salt thereof. In a more preferred embodiment, the drug is selected from the group consisting of Auranofin, Cefdinir, Tedizolid Phosphate.

In the present disclosure, the inventors also identified several potential targets for preventing or treating microorganism infections, such as walR, N-acetylmuramoyl-L-alanine amidase domain-containing protein, enzymes involving in LTA/WTA synthesis, and the like. Therefore, in a specific embodiment, the combination of the present disclosure may further comprise an autolysis promoter or simulator, including but not limited to a walR inhibitor Or antagonist, or a N-acetylmuramoyl-L-alanine amidase domain-containing protein agonist, a LTA/WTA synthesis promoter or simulator. In a further embodiment, the LTA/WTA synthesis promoter or simulator can be an agent up regulating the expression of an enzyme selected from the group consisting of TarA, TarB, TarD, TarF, TarL, TarS, TagH and FmtA.

By using multiple bio-chemical/bio-physical methods, a clear mechanism and excellent combination effects in vitro and in viva are shown, which facilitate the future drug development.

### IV. Pharmaceutical Composition of this Disclosure

Based on the combination of this disclosure, a pharmaceutical composition comprising the combination and a pharmaceutically acceptable excipient is provided.

In a specific embodiment, the pharmaceutical composition is in a topical dosage form, including (but not limited) a drop, cream, ointment, lotion, liniment, suppository, paste, and patch.

### V. Kit

Based on the combination or pharmaceutical composition of this disclosure, a kit is provided, wherein the kit comprises one or more container w accommodating the combination or the pharmaceutical composition. And for using the combination or composition to treat microorganism infections, the kit can further comprise an instruction.

### VI. Treatment method

Based on the combination, pharmaceutical composition or kit of this disclosure, a method for treating a microorganism infection, such as an eye infection, skin infection, an oral infection, a respiratory system infection, a digestive system infection or an infection in wound, is provided, and the method includes a step of giving a therapeutically effective amount of the combination, pharmaceutical composition or the kit to a subject in need thereof.

According to the teachings of this disclosure, a skilled person will appreciate that each component of the combination, pharmaceutical composition or kit of this disclosure can be administered to a subject together or separately. That is, all the component of the combination, pharmaceutical composition or kit of this disclosure can be administered to a subject concurrently or sequentially. And the time interval and dosage of each administration can be routinely determined by a physician.

### Advantages of the Present Disclosure

1. The combination therapy comprising an available antibiotic and a non-antibiotic of this disclosure is safer, more economical, and effective alternative than the development of new antimicrobial or combination of multiple antimicrobials.
2. The combination therapy of this disclosure can re-sensitize a drug against lots of bacteria at less dosage in clinical (less than 1/500-fold, FICI=0.025), and avoid antimicrobial resistance to extend the lifespan of the drug; and
3. The combination therapy of this disclosure provide the referred basis for future drug development and avoid the antimicrobial resistance crisis in future.

### Example 1. Auranofin Restores Lysozyme Susceptibility Against Methicillin-Resistant Staphylococcus aures

### 1. Materials and Methods

1) Check board broth micro-dilution assays According to standard two-fold dilution method [H. Sun et al., (2020) Nat Commun 11:5263], auranofin at different concentration was well-mixed with fresh Luria-Bertani (LB) medium containing human lysozyme in 96-well plate. Then, about 5.0×10⁵ colony-forming units (CFUs) of methicillin-resistant Staphylococcus aures (MRSA, USA300 strain) in log phase were added into each well for overnight culture. Bacterial growth inhibition was monitored by optical density at 600 nm (OD₆₀₀) and colony units in serial dilution in LB agar plate. Well without any drug served as growth control, and well without bacteria served as background control. The inhibition ratio was calculated as 1−[(ODₛₐₘₚₗₑ−OD_{background})]×100%. CFUs were counted by 10-fold serial dilution in phosphate buffered saline (PBS) and 10 μl of dilutions were spotted in LB agar plate. The minimum inhibitory concentration (MIC) was determined as the lowest concentration of a drug that could inhibit the 90% growth of microorganism by both visual CFUs and OD₆₀₀-Fractional inhibitory concentration (FIC) and fractional inhibitory concertation index (FICI) values were determined by standard methods [N. Abdul Rahim et al., (2015) J Antimicrob Chemother 70: 2589-2397]. Briefly, FICI=MIC_{AB}/MIC_{A}+MIC_{BA}/MIC_{B}=FIC_{A}+FIC_{B}. MIC₄ is the MIC of compound A alone; MIC_{AB} was the MIC of compound A in combination with compound B; MIC_{B} was the MIC of compound B alone; MIC_{BA} was the MIC of compound B in combination with compound A; FIC_{A} was the FTC of compound A; and FIC_{B} was the FIC of compound B, Notably, in a standard 2-fold dilution assay, each drug in the combination has a series of diluted concentration. Thus, there would be a series of FICI values. In most of cases, the lowest one among these values was listed to reflect the best achieving degree of combination therapy. In terms of the lowest FICI, FICI≤0.5, 0.5<FICI<1 or FICI≥1, would indicate whether the combination has synergy, part synergy or non-synergy effects, respectively. Besides, to better highlight the inhibition degree of bacterial growth in combination therapy or identify whether combination therapy has synergy effect, results might be shown by heat-maps or line-charts containing different FICI values, although they were performed by using same 2-fold dilution method. All assays were performed in triplicate.

2) Time-killing curves According to standard method [K. R. V. Thappeta et al., (2020) ACS Infect Dis 6: 1228-1237], over-night cultured MRSA was diluted and aliquoted into new sterile tubes at 1:1000 ratio. Then, the bacterial suspension was co-cultured with 034 μg/ml auranofin, 0.5 mg/ml lysozyme or their combination at 37° C. with shaking at 200 rpm. Similar bacterial suspension without any additions served as control group. Then, 20 μl of each culture was extracted at time intervals of 0, 1, 3, 6, 9.12 and 22 hours to determine CFUs by spotting 10 μl of 10-fold dilution on the LB agar plates and incubating at 37° C. for 24 hours prior to enumeration. All assays were performed in triplicate.

3) Biofilm assays According to described previously method [K. R. V. Thappeta et al., (2020) ACS infect Dis 6: 1228-1237], biofilm inhibition was assessed by using an adherence assay on 96-well tissue culture plates. Briefly, bacterial suspensions (1×10⁶ (CFU/ml) were exposed to 0.34 μg/ml auranofin in LB medium in the absence and presence of 0.5 mg/ml lysozyme. LB medium with or without bacteria served as negative or positive control group, respectively. After overnight incubation at 37° C. bacterial suspensions were removed, and the wells were gently washed twice with sterile PBS to remove exclusively non-adherent bacteria. The adherent biofilms were fixed by using 95% methanol at 60° C. for 15 minutes. Then, biofilms were stained with 1% crystal violet (100 μl/well) at 37° C. for 15 minutes, followed by gently washing with PBS to remove redundant crystal violet. Finally, the dye was solubilized with 150 μl of 95% ethanol per well at 37° C. for 30 minutes. The optical density of solution in each well was measured at 590 nm using a micro-plate reader. Herein, strong biofilm, weak biofilm and no biofilm wen defined as “ODₛₐₘₚₗₑ≥2 OD_{negative control}”, “OD_{negative control}<ODₛₐₘₚₗₑ<2 OD_{negative control}” and “ODₛₐₘₚₗₑ≤OD_{negative control}”, respectively. All assays were performed in triplicate.

4) Resistance development study. As described before [Y, Lau et al., (2020) Adv Sci 7:1902227]. MRSA at exponential phase was diluted (1:1000) into fresh LB medium containing different concentrations of auranofin, lysozyme or their combination. After 1 passage (24 hours) at 37° C., MIC of culture was determined by standard two-fold serial dilutions. Next, this culture with higher MIC was further diluted into similar LB medium but with higher concentrations of compounds for next passages. The process was repeated for 18 days. By analyzing initial MICs of auranofin or lysozyme, fold-changes in MI s of these compounds were calculated. Assays were performed in triplicate.

5) Mutation frequency and prevention concentration analysis. According to previously described method [H. Sun et al., (2020) Nat Commun 11: 5263; Q. Zhang et al., (2022) Proc Natl Acad Sci USA 119:e2110417119; Q. Zhang et al., (2023) J Biol Inorg Chem 28; 225-234] MRSA in log phase were collected and concentrated into bacterial suspension of about 1.0×10¹⁰ CFU/ml in PBS buffer. Then, 100 μl of diluted MRSA suspension were evenly applied onto agar plates with gradient auranofin concentrations (including 0.34, 0.68, 1.36 and 2.72 μg/ml) in the presence of lysozyme (including 0.5, 1, 2 and 4 mg/ml Diluted bacterial culture on agar plates with only auranofin served as control group, Initial MRSA suspension was also cultured on agar plate to figure out the initial bacterial load. After 48 hours incubation at 37° C. colony counts on agar plates were figured out. Herein, the bacterial mutation frequency was calculated as colony counts_{treated group}/colony counts_{initial supplied group}. Besides, the minimal concentrations of auranofin in combination with lysozyme in plates without any colony were recorded as mutation prevention concentrations (MPC) in this combination therapy and corresponding quotients between MPC and auranofin itself MIC in control group were figured as “mutation prevention index” (MPI), Assays were performed in triplicate.

6) Scanning Electron Microscope (SEM) analysis. According to standard method [N. Abdul Rahim et al., (2015) J Antimicrob Chemother 70; 2589-2597], MRSA single colony was picked up and cultured in LB medium. After 24 hours incubation at 37° C., bacterial suspension was inoculated into LB medium supplied with auranofin (0.17 or 0.34 μg/ml), lysozyme (0.5 mg/ml) alone or their combination. After 5 hours incubation at 37° C., these bacteria in log phase were collected by centrifugation at 3220 g for 10 minutes and fixed with 2.5% glutaraldehyde prior to being washed and re-suspended in PBS. After that, bacterial cultures were dehydrated by using increasing concentrations of ethanol in water (10%, 30%, 50%, 70%, 80%, 90% and 100%) for 10 minutes in each step. Then, 5 μl of bacterial suspension at 1.0×10⁹ CFU/ml was incubated on polyethylenimine-coated coverslips (22 mm×22 mm) and the coverslips were dried in a Balzers critical point dryer (Balzers, Liechtenstein, Germany) prior to mounting on 25 mm aluminum stubs with double-sided carbon tabs. The edges of the coverslips were treated with silver liquid, dried, and then gold-coated in an Edwards S150B sputter coater (Edwards High Vacuum, Crawley, West Sussex, UK). The cells were imaged with a TESCAN VEGA3 scanning electron microscope (TESCAN, Brno, Czech Republic) at a voltage of 20 kV.

7) Proteomics analysis and PRM-based validation. According to the previously described method [S. Zhang et al., (2021) Adv Sci 8:2100681], MRSA in log phase was treated with lysozyme (1 mg/ml) in combination of auranofin (0.17 or 0.34 μg/ml) at 37° C. Those exposed to lysozyme (1 mg/ml) alone served as control group. After 5 hours incubation, cells were harvested and the total proteins of samples were extracted using the multiple freezing-thawing at liquid nitrogen followed by sonication at lysis buffer (25 mM for HEPES-Na, 150 mM NaCl, 0.1% NP40, 4M urea and 1× protease inhibitor). Then, BCA kit (Thermo fisher, China) was used to quantity the extracted proteins for next digestion by trypsin at the ratio of 5:1. These overnight digested peptides were further labeled by TMT¹⁰plex before loading onto Orbitrap Exploris™ 480 Mass Spectrometer (Thermo fisher, China) coupled with an UltiMate 3000 UPLC System (Thermo Fisher Scientific) with C18 analytical column. Mobile phases A and B consisted of 0.1% FA in water and 0.1% FA in 100% ACN, respectively. Mobile phase B was increased to 6% at 12 minutes, 20% at 92 minutes, 30% at 92 minutes, 90% at 100 minutes and held for 5 minutes. Data was collected in data-dependent acquisition (DDA) mode with HCD fragmentation at TopN mode. The resolution was set at 60,000 for MS1 and 15,000 for MS2 with 30 ms maximum injection time. All resulted spectra were searched against UniProt Staphylococcus aureus (20,330 entries, accessed September 2019) using MaxQuant 1.5.8.2. The parameters for searching: a mass tolerance of 10 ppm for precursor ions; ±0.1 Da for-fragment ions, carbamidomethylation on cysteine was set as a fixed modification, oxidation on methionine and protein N-terminal acetylation was set as variable modifications. The enzyme was specified as trypsin with two missed cleavages allowed. False discovery rate for peptide spectral matches and proteins were set as 1%. The maximum number of modifications per peptide was three. Differentially expressed proteins at protein-levels were identified with p-value≤0.05 and fold change (F) values≤1.2 (log₂ FC≥0.26 or log₂ FC≤−0.26). The cuff-diff program was used to analyze differences between two treatments.

Similar protocol was performed in parallel reaction monitoring (PRM) analysis with minor revision [H. Guo et al., (2022) ACS Chem Biel 17:2003-2009]. Overnight digested label-free peptides were loaded onto Orbitrap Exploris™ 480 Mass Spectrometer (Thermo fisher, China). PRM acquisition methods were directly developed according to our DDA data. The DDA data was imported into Skyline to select targeted peptides from previously selected targets. The relative information including precursor m/z charge, retention time window of the selected peptides was exported from skyline into xcalibur software to edit the PRM method. The targeted MS parameters were as follows: resolution, 60,000; AGC target. 2.0×10⁵; and maximum injection time, 50 ms. PRM scanning was performed at 30,000 resolution. 1×10⁵ AGC target, 54 ms maximum injection time and 0.7 m/z isolation window with peptides in 5 minutes retention time windows. After PRM data acquisition, the data was imported into skyline for analysis to confirm the confidence of proteomics analysis results. To get reliable identification and quantification, idotp and dotp were >0.60. The top three product ions were summed up to represent the peptide abundance.

8) ATP level. According to the described method [S, Zhang et al., (2021) Adv Sci 8:2100681], MRSA in log phase was treated with lysozyme, auranofin or their combination at different concentrations. Those without any treatment served as control group. Alter 1 hour incubation at 37° C. all bacteria were collected and washed by cold PBS for 3 times. Then, these collected samples were further re-suspended in cold extracting buffer (Beyotime) to get bacterial suspension with an initial density of about 5.0×10⁶ CFU/ml, followed by sonication lysis for 40 minutes at 4° C., and centrifugation at 18,000 g at 4° C. for 20 minutes to collect these supernatants. Then, 20 μl of each supernatant were added white 96-well plate pre-reactivated by 100 μl of ATP test solution (Beyotime) to perform luminometer measurement. All assays were performed in triplicate and results were expressed as average±SD.

9) Cell-bused infection model. According to the previously described method [H. Sun et al., (2020) Nat Commun 11: 5263; Q. Zhang et al., (2022) Proc Natl Acad Sci USA 119: e2119417119], ARPE-19 cell was firstly cultured in gibco dulbecco's modified eagle medium: nutrient mixture F-12 (DMEM/F12) supplemented with 10% fetal bovine serum (FBS) and grown at 37° C. in 5% CO₂-humidified atmosphere for 7 days. Then, about 1.0×10⁴ collected ARPE-19 cells were seeded into per well of 96-well plates and incubated overnight to ensure their confluency. Logarithmic cultures of MRSA were washed with PBS for three times and re-suspended in DMEM/F12-10% FBS to get bacteria suspensions with initial density of about 5.0×10⁶ CFU/ml. Next, 10 μl of bacterial suspension were added to each well and incubated for another 3 hours, when bacterial infection had the multiplicity of infection (MOI) of 5. Next, infected levels were washed vigorously with PBS for six times and replenished with vulture medium to remove unbound bacteria. Herein, cell-associated bacteria were defined as bacteria that attach to, penetrate, or transcytose in ARPE-19 cells. Next, these ARPE-19 cells were exposed to either lysozyme, auranofin or their combination overnight under identical cell culture condition 1100 μl of per well), Cells in the absence of drugs served as control group. After 24 hours incubation, CCK-8 assays were performed to analyze the survival ratio of cells under different treatments, Specifically. 10 μl of CCK-8 solution per well was added and the cells were incubated for another 2 hours at 37° C. for chromogen development. The absorbance was measured at 450 nm, with 630 nm as reference wavelength. Herein, survival ratio=(Absₛₐₘₚₗₑ−Abs_{negative control})/(Abs_{positive control}−Abs_{negative control})×100%. The bacterial loads were examined by lysing ARPE-19 cells with 1% Triton X-100 in PBS and serially diluting the resulting lysates to enumerate bacterial colonics on agar plates. These assays were performed in triplicate, and results were expressed as average±SD.

10) Bacterial strains, compounds, reagents and instruments. Methicillin-resistant Staphylococcus aures USA 300 (MRSA, ATCC-BAA1556) was from our own collection. Human lysozyme, ampicillin, hydroxyethyl piperazine ethanesulfonic acid (HEPES), formaldehyde, crystal violet, sodium chloride (NaCl), LB both powder and PBS were supplied by Sigma-Aldrich. Bacterial incubator, plate-reader and high-throughput screening robust were purchased from Thermal Fisher Scientific. Deionized (DI) water (18.2 MΩ·cm) and filters (0.45 and 0.22 μm) were purchased from Millpore, USA. All other chemicals were purchased from MedChemExpress unless otherwise stated.

11) Data availability and statistical analysis. All assays were performed in triplicate and result were expressed as average±SD unless otherwise stated. All tests of significance were based on *p<0.05, **p<0.01 and ***p<0.001. Other data are available from the corresponding author upon reasonable request.

### 2. Results

### 2.1 Auranofin Re-Sensitizes Lysozyme Against MRSA.

The synergistic effect between lysozyme and auranofin, a well-known anti-rheumatic agent, was firstly measured by performing standard check board broth micro-dilution assays (FIG. 1A). Standard methicillin-resistant Staphylococcus aureus USA 300 (MRSA, ATCC-BAA1556) was used as the model strain, which is resistant to auranofin with the raging MICs from 0.17 to of 0.68 μg/ml due to the inoculum effect [M. Snoussi et al., (2018) eLife 7: e38174; S. Thangamani et al., (2016) Sci Rep 6: 22571] (FIG. 5), and successfully observed synergistic effect in the combination therapy (the fractional inhibitory concentration index (FICI) was 0.266). Specifically, it was found that MRSA could be re-sensitized against lysozyme from the level of ≥>150 to 1.0 mg/ml, in which 0.17 μg/ml auranofin was used in combination (FIG. 1A, FIG. 6A). Considering that lysozyme is abundant in tear and defined as “natural antimicrobial” the potential of auranofin as lysozyme adjuvant to overcome ocular bacterial infection in the subsequent studies was emphasized.

To collect more details of auranofin in restoring the activity of lysozyme, the time-killing experiments were then performed [H. Sun et al., (2020) Nat Common 11; 5263; Q Zhang et al. (2022) Proc Natl Acad Sci USA 119: e2119417119]. It was found that either 0.34 μg/ml auranofin or 05 mg/ml lysozyme have few inhibitions effect on the growth of MRSA at exponential phase. By contrast, their combination significantly reduced the bacterial load from the level of 10¹⁰ to 10⁵ CFU/ml (FIG. 18, FIG. 6B). Considering the resistance-development as always the tough problem m treatment of bacterial infection [L. Imamovic and M. O. A. Sommer (2013) Sci Transl Med 5: 204ra132; C. Munck et al., (2014) Sci Transl Med 6: 262ra156), the effect of combination therapy on the resistance development was further assessed. However, limited solubility of lysozyme (≤300 mg/ml) made it impossible to accurately assess lysozyme resistance (D. K. Sen and G. S. Sarin (1986) Br J Ophthalmol 70: 246-248]. Thus, serial passages assays on MRSA exposed to auranofin at increasing concentrations in the absence and presence of lysozyme were performed. Results showed combination therapy did suppress the development of auranofin resistance in MRSA if compared with that treated with auranofin alone (FIG. 1C). Further studies explored the combination therapy could also efficiently reduce the mutation prevention concentrations of drugs (FIG. 1D, FIG. 1E). Specifically, the gene mutation frequency of MRSA treated with 0.34 μg/ml auranofin was significantly narrowed from ≥9×10⁻⁶ to ≤1×10⁻⁹ when 2 mg/ml lysozyme (physiological concentration in tear) was used in combination (FIG. 1E) These results suggested that the combination therapy could effectively minimize the de novo emergence of resistance development.

Remarkably, the emergence of bacterial biofilm seriously limited clinical effect of antibiotics, especially in ocular treatment encountering the blood-eye barrier [D. Qu et al., (2020) Sci Adv 6: eaay9597]. Crystal violets assays on bacteria at early and late exponential phases were subsequently preformed, corresponding to pre-biofilm and post-biofilm stage, to assess whether biofilm could be inhibited or not. Interestingly, compared with the treatment of auranofin or lysozyme alone to slightly reduce the biofilm-based absorbance at 590 nm, the combination therapy perfectly decreased it into the same level with that in control group, indicating the biofilm had been absolutely removed (FIG. 1F). Nevertheless, the decrease was not observed after biofilm had appeared (FIG. 1F). The difference may be attributed to the difficulties for drugs to penetrate into EPS. [D. Qu et al., (2020) Sci Adv 6: eaay9597].

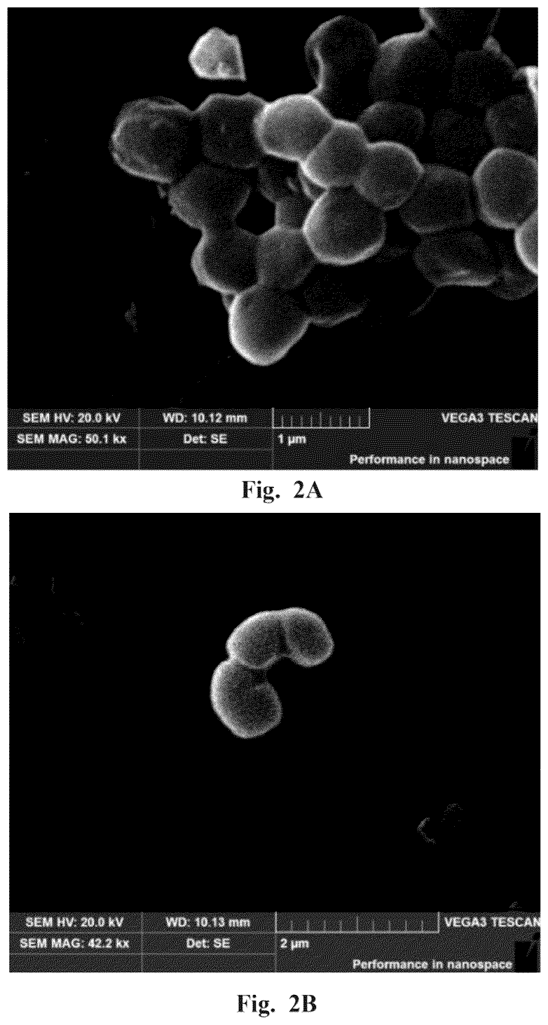

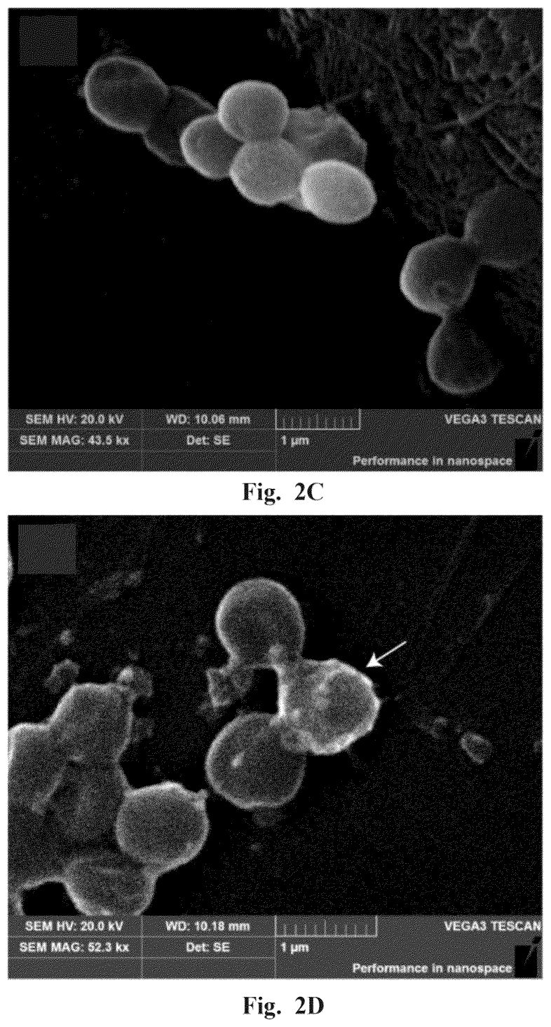

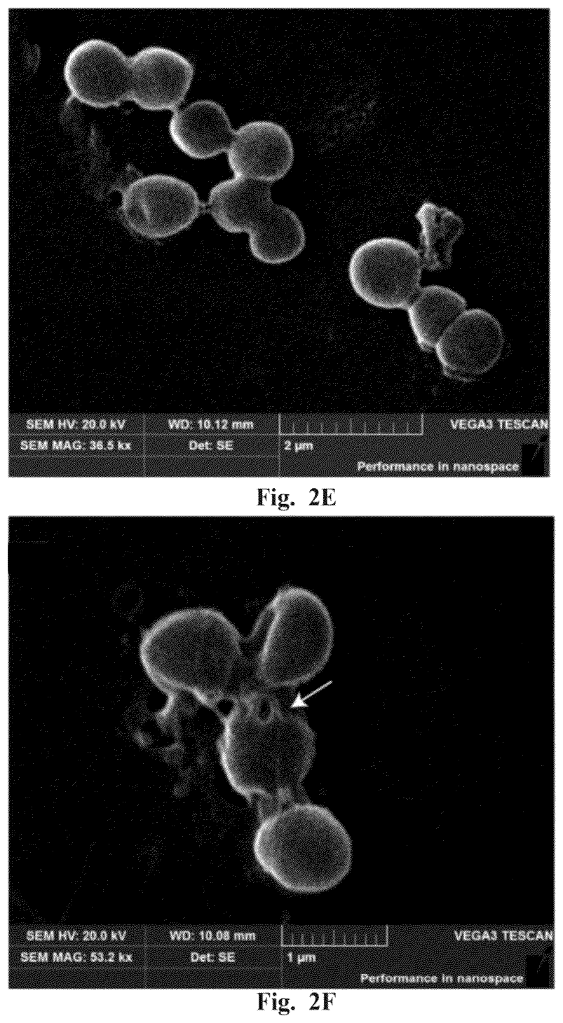

Next, the morphological analysis was further performed by comparing the scanning electron microscope (SEM) imaging of MRSA under treatment of auranofin (0.34 or 0.17 μg/ml), lysozyme (0.5 mg/ml) alone or their combination. As shown in FIG. 2A to FIG. 2F, the cell surface in untreated group was relatively smooth. Such a phenomenon could be also found in those treated with auranofin or lysozyme alone. However, it was clearly observed that MRSA under combination treatment had severe cell autolysis-like surface damage. (Conceivably, the damage might be so severe that bacteria were hardly to self-pair and died. On the whole, these results together indicated that auranofin was able to efficiency re-sensitize lysozyme against MRSA.

### 3.2 Proteomics Studies Explored Potential Targets in MRSA.

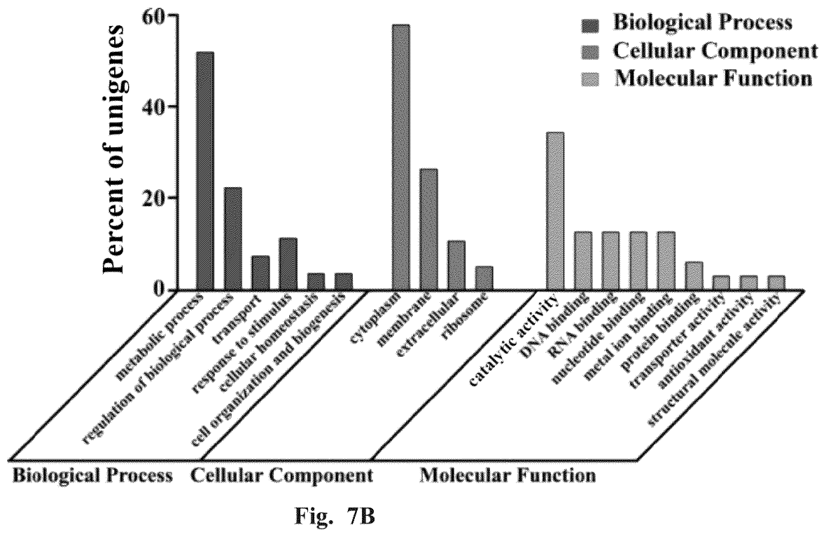

To explore the mechanism of auranofin to re-sensitize lysozyme against MRSA, the proteomic study was next performed, which is an effective approach to uncover effect of drugs against bacteria in protein level [H. Guo et al., (2022) ACS Chen Biol 17:2003-2009]. Unfortunately, so far, the proteomics change in MRSA treated with superstar “auranofin” retained unknown. By performing tandem mass tag (TMT) labeling quantitative proteomics, the significant up-regulation of 11 and down-regulation of 9 differentially expressed proteins (DEPs) were observed in MRSA under treatment of combination and lysozyme alone for 5 hours (p<0.05, FIG. 3A, FIG. 7A). Gene ontology (GO) annotation analysis showed that these DEP, were mainly correlated with catalytic reaction and nucleotide binding (molecular functions), played their roles in metabolic process and regulation of biological process (biological processes), and in cytoplasm (cellular components) (FIG. 38. FIG. 7B).

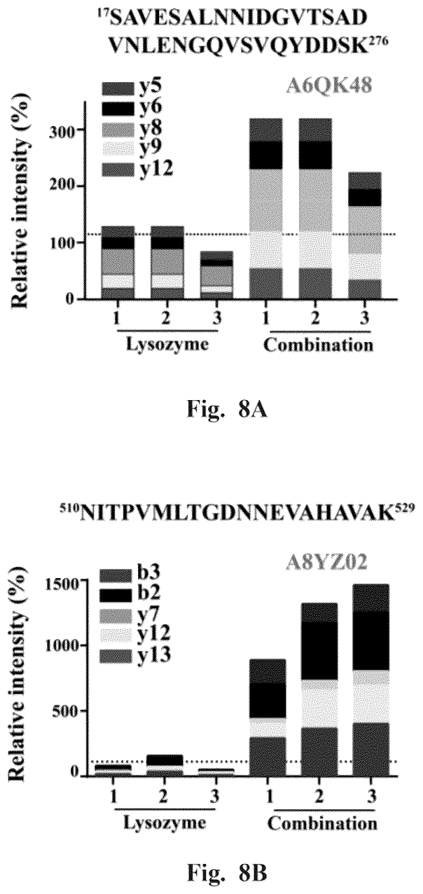

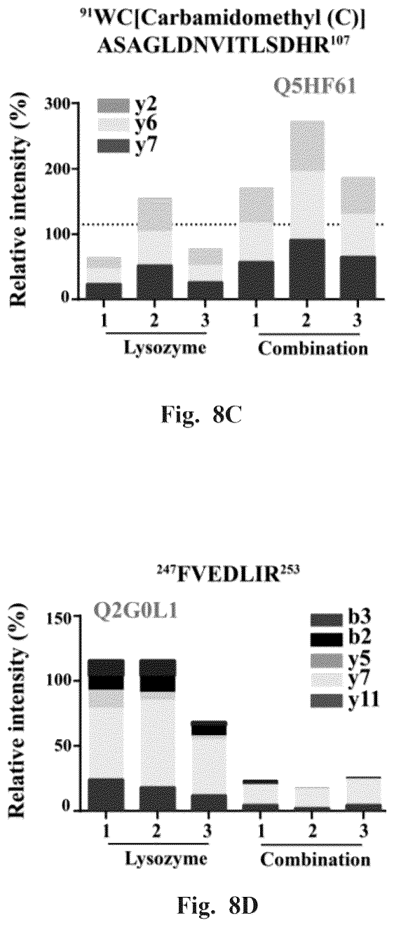

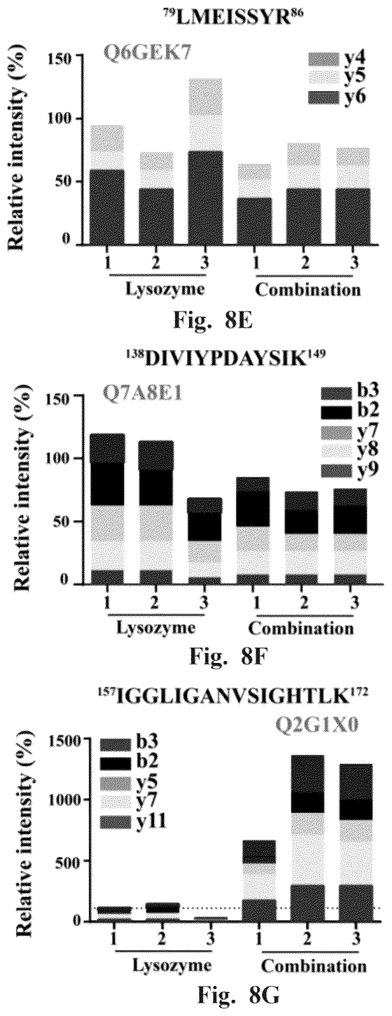

Interestingly, some interesting proteins showed significant changes when MRSA was under combination treatment (FIG. 3C). A typical example was CopZ (UniProt ID: A6QK48, FIG. 8A), chaperone that served for the intracellular sequestration and transport of Cu. Its over-expression (21-fold) was clearly observed in combination therapy group. Such a phenomenon was also observed in probable copper-transporting P-type ATPase B (Uniprot ID: A8YZO2, FIG. 8B). The change was reasonable because auranofin inevitably resulted in intracellular accumulation of metal (Au in MRSA and thereby MRSA, in turn, had to over-express existing transporters for other similar essential elements in cellular, such as Cu⁺, to export these metals [Y. Zhou et al., (2021) In: E. C. Constable, G. Parkin and L Que Jr (eds) Comprehensive coordination chemistry iii. Elsevier, Oxford, pp. 680-705]. Thiol peroxidase (UniProt ID: Q5HF61, FIG. 8C), an enzyme protecting cell against oxidative stress by detoxifying peroxides, was another overexpressed candidate, which might be attributed to cell stress response. By contrast, GTP cyclohydrolase FolE2 (UniProt ID: Q2G0L1, an enzyme converting GTP to 7,8-dihydroneopterin triphosphate for cofactor biosynthesis. FIG. 8D) and 30S ribosomal protein S13 (UniProt ID: Q6GEK7, structural constituent of ribosome for RNA and rRNA binding, FIG. 8E) were in the decreasing lists. Indeed, they might be the following-up effects because it was clearly observed that ATP level was significantly reduced in MRSA under combination treatment (FIG. 9).

Interestingly, it was unexpectedly found the overexpressed enzyme domain of N acetylmuramoyl-L-alanine amidase (UniProt ID: Q2G222) (nearby 1.84-fold, FIG. 3C), which was consistent with the parallel reaction monitoring (PRM) results (FIG. 3D Considering that the enzyme was able to remove L/D-alanine from wall teichoic acids (WTA) and caused the autolysis, it was plausible that its overexpression would inevitably facilitate cell lysis. Moreover, the protein level of walR (UniProt ID: Q7A8E1) also significantly reduced (nearby 0.68-fold) in combination treatment groups (FIG. 3C, FIG. 8F). As the member of two-component regulatory system walK/walR, walR worked in regulating genes involved in cell wall metabolism, virulence regulation, biofilm production, oxidative stress resistance and antibiotic resistance via direct or indirect regulation of autolysins, Thus, such a change would disrupt normal regulatory system in cellular and cause the autolysis. All in all, these effects synergized with lysozyme digestion of cell wall to strengthen the combination ability in killing MASA (FIG. 3A to FIG. 3D).

### 3.3 Combination Therapy Reduced the Bacterial Load in Cell Infection Model.

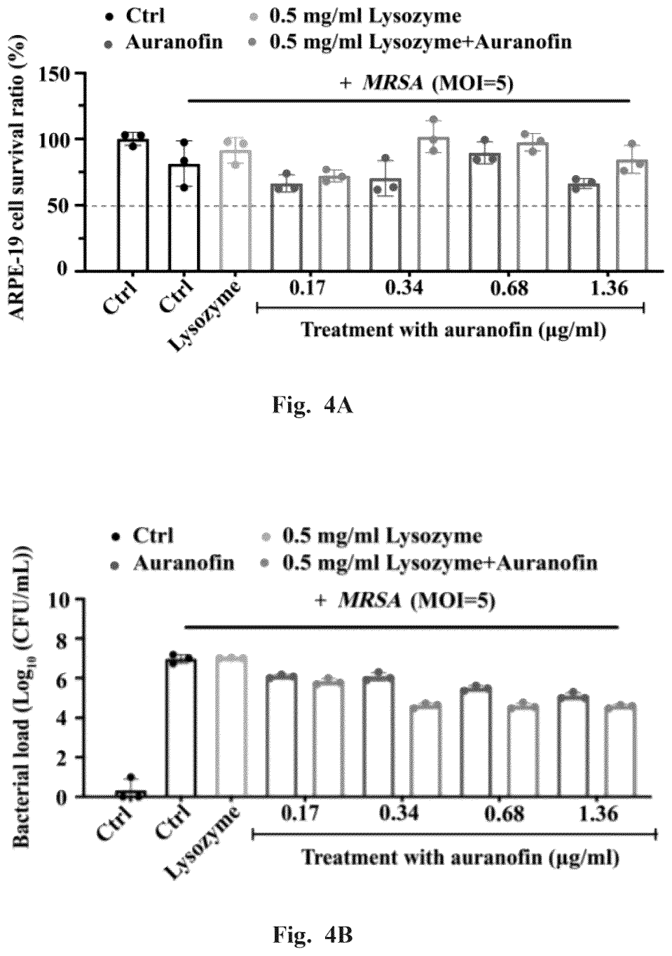

The ability of combination treatment to kill MRSA in mammalian cell was then investigated. Considering lysozyme was abundant in human tear, the bacterial infection assays was performed on ARPE-19 cell, a spontaneously arising retinal pigment epithelia (RPE) cell. It was found that, besides self-secreted lysozyme, auranofin also had negligible effect on ARPE-19 cell (IC₅₀˜2.21±034 μg/ml, FIG. 10). Remarkably, MRSA infection would result in about 25% ARPE-19 cells death (FIG. 4A), Interestingly, with the increase in auranofin's concentration, increasing survival ratio of infected cells and reducing bacterial load in cellular under the combination treatment were clearly observed. Specifically, the bacterial load significantly dropped into a level of 10⁵ CFU/ml (10²-fold, when ≥0.34 μg/ml auranofin was combined with 0.5 mg/ml lysozyme and maintained until the end of analyzed points (the highest concentration of auranofin was 1.36 μg/ml) (FIG. 4B). However, the higher concentration of auranofin in combination therapy would increase the death of infected ARPE-19 cells, which might attribute to the increased expression of α-hemolysin (UniProt ID: Q2G1X0) uncovered in our previous proteomics studies (FIG. 8C). As a toxin, it was expired to bind to the membrane of eukaryotic cells, resulting in the release of low-molecular weight molecules and an eventual osmotic lysis. Collectively, it was demonstrated that antimicrobial efficacy of auranofin and lysozyme combination therapy in vitro could be well-translated into in cellular efficacy. However, considering the significance and complexity of in vi) study, more studies are required to validate it before future clinical application.

### 4. Conclusions and Discussion

In the post-antibiotic era, the emergence of antibiotics-resistant pathogens and their recurrent infections seem to be getting mote and more uncontrollable, especially in ocular infections [M. Z. David and R. S. Daum (2010) Clin Microbiol Rev 23: 616487; A, S Lee et al., (2018) Nat Rev Dis Primers 4: 18033], because the development of novel antibiotics are suffering a serious setback [H. Sun et al., (2020) Nat Commun 11; 5263; J. H. Kwon and W. G. Powderly (2021) Science 373: 471]. Human eyes are expected to have great ability to avoid bacterial infection due to their abilities to secret many natural antimicrobials, such as immunoglobulin and lacritin [P. M. Tiffany (2003) Eye 17: 92.3-926. Unfortunately, nearly 10 million people have been visually impaired because of ocular infectious diseases ID. Miller (2017) Middle East Afr J Ophthalmol 24: 3042; G. B, a. V. I. C. V. L. E. G. o. t. G. B. o. D. Study. (2021) Lancet Glob Health 9: e130-e143]. Ocular infection has been a global concern to individuals and to communities.

In order to treat these ocular infections, antibiotics are frequently applied. However, the emergence of AMR has resulted in poor therapeutic effect. Nowadays, combination therapy is receiving more and more attentions in treatment of bacterial infection because loner dose of antibiotic will be enough in clinical [H. Sun et al., (2020) Nat Commun 11: 5263]. Lysozyme is one of the most famous enzymes and selected as the model protein in solving protein structure, Indeed, its mechanism to kill gram-positive bacteria by hydrolyzing 1,4-β-linkages between N-acetyl-muramic acid and N-acetyl-D-glucosamine inhibiting bacterial growth has been well known. However, due to the blood-eye barrier [J. G. Cunha-Vaz (1997) Doc Ophthalmol 93:149-157], the study on lysozyme used in combination remains limited. Notably, biofilm formation on the surfaces of implants significantly challenges the treatment for device-related ocular infection Indeed, after biofilm form on the surface of indwelling devices, such as contact lenses, it eventually leads to chronic infection [J. H. Mah-Sadorra et al., (2005) Cornea 24: 51-58; K. Srivastava et al., (2019) J Bone Joint Surg Am 101:14-24]. In this disclosure, it is reported that the combination of low-doe auranofin and lysozyme can inhibit the adherence of bacteria before the biofilms appear. The weaker ability to remove post-biofilm may attribute to the difficulties for auranofin/lysozyme to penetrate into EPSs [M. Vert et al., (2012) Pure Appl Chem 84; 377-410], which urges suitable drug delivery system to improve the antibacterial effects in future.

In the post-antibiotic era, proteomics analysis has received moov and more attentions in exploring potential mechanism of drugs in treatment of bacterial infection, Interesting, many drugs as panaceas are believed to have multiple targets and effects in bacterial pathogens [H. Sun et al., (2020) Nat Commun 11:5263; Q. Zhang et al., (2(122) Proc Natl Acad Sci USA 19:e2119417119]. Thus, it is urgent and significant to analyze these targets to explore the mechanism of these panaceas. Among them, auranofin is a typical drug which has great effect on the anti-rheumatoid arthritis (RA) (14. Sun et al., (2020) Nat Commun 11:5263; N. S. Abutaleb and M. N. Seleem (2020) Sci Rep 10:77011, bacterial infection as well as SARS-CoV2 (COVID 19) [H. A. Rothan et al., (2020) Virology 547:7-11]. Auranofin has thioredoxin reductase (TrxR) as its mainly target in gram-positive bacteria but lacks complete proteomics analysis in critical pathogens, such as MRSA. Herein, auranofin proteomics map was analyzed and it was observed that it could not only reduce the expression of walR, a member of the two-component regulatory system walK/walR, but also increase the expression of proteins working in autolysis: N-acetylmuramoyl-L-alanine amidase domain-containing protein. Alt results provide a crucial reference for mechanism study of auranofin in MRSA-causing ocular infections.

In summary, auranofin shows the potential as lysozyme adjuvant to overcome the ocular bacterial infection. Nevertheless, it should be refined to enlarge its clinical application window and more prospective clinical trials are still required to verify the combination effect in vivo.

### Example 2. Repurposing Lysozyme Against Broad-Spectrum Gram-Positive Pathogens

### 1. Materials and Methods

1) High-throughput screening. According to standard compounds-screening method [Sun H et al., Nat Commun 11, 5263 (2020); Zhang Q et al., Proc Natl Acad Sci USA 119, e2119417119 (2022)], 4989 compounds (containing 109 natural products or their analogs) from ourselves chemical library at a fixed concentration of 2.5 μM were screened against methicillin-resistant Staphylococcus aures (MRSA) or Staphylococcus epidermis (S. ep. MRSE) in combination with human lysozyme. Specifically, these compounds were added into bacterial suspensions (1×10⁶ CFU/ml) in the 6-well microliter plate, in which each well contained 50 μl of lysogeny broth (LB) medium with or without 1 mg/ml lysozyme. LB medium in the absence of bacteria served as background group. Then, the real-time growth curves of MRSA were monitored per hour and for 24 hours. Experiments were performed with the biological replicates. Fly analyzing the absorbance 600 nm (OD₆₀₀) of bacterial culture at the time point of 24-hour, the inhibition ratio (%) was calculated according to (OD_{control}−ODₛₐₘₚₗₑ)/(OD_{control}−OD_{background})×100%. Here, the relative and apparent inhibition ratios were figured out by using the absorbance of bacterial suspension only containing compound and that without any drug treatment as OD_{control}, respectively. The relative and apparent inhibition ratios indicated the combination effect and potential toxicity of compound in combination effect. Synergy effect was defined as the relative inhibition ratio of ≥90%.

2) Check board broth micro-dilution assays. According to standard two-fold dilution method [Sun H et al., Nat Commun 11, 5263 (2020)], cefdinir at different concentration was added into fresh LB medium (or trypticase soy broth medium with 5% defibrinated horse blood) containing different concentration of human lysozyme in 96-wells plate. After well mixed, about 20 μl of 1.0×10⁷ CFU/ml logarithmic cultures of MRSA, MRSE, Streptococcus pyogenes (S. py), Streptococcus mutans (S. mu), Enterococcus faecium (E. faecium), Enterococcus faecalis (E. faecalis) and Corynebacterium pseudodiphtheriticum (C. ps) were added into each well of the plate und co-incubated overnight, Wells without any drugs served as growth controls, and wells without bacteria served as background controls. The inhibition ratio was calculated as [1−(ODₛₐₘₚₗₑ−OD_{background})/(OD_{control}−OD_{background})]×100%. Besides, bacterial growth inhibition was also monitored by serial dilution in LB-agar plate. CFU was counted by performing 10-fold serial dilution in PBS and then spotting 10 μl of dilutions in LB-agar plate. The MIC was determined as the lowest concentration of a drug that could inhibit the 907% growth of microorganism by both visual CFU and OD₆₀₀ using a microtiter plate reader. Fractional inhibitory concentration (FIC) and fractional inhibitory concentration index (FICI) values were determined by standard method. Briefly, FICI=MIC_{AB}/MIC_{A}+MIC_{BA}/MIC_{B}=FIC_{A}+FIC_{B}. MIC_{A} was the MIC of compound A alone; MIC_{AB} was the MIC of compound A in combination with compound B; MIC_{B} was the MC of compound B alone; MIC_{BA} was the MIC of compound B in combination with compound A; FIC_{A} was the FIC of compound A; and FIC_{B} was the FIC of compound B. Notably, in a standard 2-fold dilution assay, each drug in the combination has a series of diluted concentrations. Thus, there would be a series of FICI values. In most of Cases the lowest one among these values was listed to reflect the best achieving degree of combination therapy. In terms of the lowest FICI, FICI≤0.5, 0.5<FICI<1 or FICI≥1, would indicate the combination has synergy, part synergy or non-synergy effects, respectively.

Besides, to better highlight the inhibitor degree of bacterial growth in combination therapy or identify whether combination therapy has synergy effect, results might be shown by heat-maps or line-charts containing different FICI values, respectively. Each test was performed in triplicate.

3) Time-killing curves. According to standard method [Thappeta K R V et al., ACS Infect Dis 6, 1228-1237 (2020)], over-night cultured MRSA was diluted and aliquoted into new sterile 50 ml tubes at 1:1000 ratio. Then, the bacterial suspension was cultured in LB medium containing 0.5 or 2.0 μg/ml cefdinir, 0.5 mg/ml lysozyme or their combination at 37° C. with shaking at 250 rpm. Bacterial suspension without any drugs served as control group. To measure their kinetic time-killing curves, twenty micro liters of each culture was extracted at time intervals of 0, 2, 4, 6, 9 and 16 h for measurement of the OD₆₀₀ and colony-forming units. Ten micro liters of each dilution was spotted on the LB-agar plates and incubated at 37° C. for 24 hours prior to enumeration. All assays were performed in triplicate.

4) Biofilm assays. According to reported method [Thappeta K R V et al., ACS Infect Dis 6, 1228-1237 (2020); Merritt J H et al., Curr Protec Microbiol Chapter 1, Unit 1B.1 (2005): O'Toole G A. J Vis Exp 47, 2437 (2011)], biofilm inhibition was assessed by using an adherence assay on 96-well tissue culture plates. Briefly, cefdinir at final concentration of 0.5 μg/ml was added into bacterial suspension (1×10⁶ (CU/ml) in LB medium in the absence or presence of 0.5 mg/ml lysozyme. LB medium with or without bacteria served as negative or positive control group, respectively. After overnight incubation at 37° C., the bacterial suspension was removed, and the well was gently washed twice with sterile phosphate buffered saline (PBS) to remove exclusively non-adherent bacteria. In terms of analysis on post-biofilm assay, 100 μl of fresh LB medium with or without drugs was added for overnight incubation at 37° C. The biofilm-based optical density was measured to evaluate the dug's effect on the growth of biofilm-based bacteria. In terms of effect on pre-biofilm, the mentioned adherent biofilms were directly fixed by using 95% methanol at 60° C. for 15 minutes. Then, biofilms were stained with 1% crystal violet (100 μl/well) at 37° C. for 15 minutes and the wells gently washed with PBS to remove redundant crystal violet. Finally, the dye was solubilized with 150 of 95% ethanol per well at 37° C. for 30 minutes. The optical density of solution in each well was measured at 590 nm using a micro-plate reader. Herein, strong biofilm, weak biofilm and no biofilm were defined as “ODₛₐₘₚₗₑ≥2OD_{negative control}”, “OD_{negative control}<ODₛₐₘₚₗₑ<2OD_{negative control}” and “ODₛₐₘₚₗₑ≤OD_{negative control}”. All assays were performed in triplicate.

5) Mutation frequency and prevention concentration analysis. According to previously described method [Sun H et al., Nat Commun 11, 5263 (2020)], MRSA culture in log phase were collected and concentrated into bacterial suspension of about 1.0×10¹⁰ CFU/ml in PBS buffer. Then, 100 μl of diluted MRSA suspension was evenly applied onto agar plates with gradient cefdinir concentrations (including 1, 2, 4 and 8 μg/ml) in the presence of lysozyme (including 0.5, 1, 2 and 4 mg/ml). Diluted bacterial culture on agar plates with cefdinir alone served as control group. Initial diluted MRSA suspension was also cultured on agar plate without any additions to figure out accurate bacterial concentrations. After 48 hours incubation at 37° C., colony counts on agar plates were figured out. Herein, the bacterial mutation frequency was calculated as colony counts_{treated group}/colony counts_{initial supplied group}. Besides, the minimal concentrations of cefdinir combined with lysozyme in plates without any colony were recorded as mutation prevention concentrations (MPC) in combination therapy and corresponding quotients between MPC and cefdinir itself MIC in control group were figured as “mutation prevention index” (MP). All assays were performed in triplicate.

6) Scanning Electron Microscope (SEM) analysis. According to standard method [Abdul Rahim N et al., J Antimicrob Chemother 70, 2589-2597 (2015).], a MRSA single colony was picked up and cultured in LB medium. After 24 hours incubation at 37° C., bacterial suspension was inoculated into LB medium containing cefdinir (0.5 μg/ml), lysozyme (0.5 mg/ml) alone or their combination. After 5 hours incubation in a shaking at 250 rpm at 37° C. these MRSA in log phase were collected by centrifugation at 3220 g for 10 minutes and fixed with 2.5% glutaraldehyde prior to being washed and re-suspended in PBS. After that, bacterial suspensions were dehydrated by using increasing concentrations of ethanol in water (10%, 30%, 30%, 70% 0%, 90% and 100%) for 10 minutes in each step. Then, 5 μl of bacterial suspension at 1.0×10⁹ CFU/ml was incubated on polyethylenimine-coated coverslips (22 mm×22 mm and the coverslips were dried in a Balzers critical point dryer (Balers. Liechtenstein, Germany) prior to mounting on 25 mm aluminum stubs with double-sided carbon tab Silver liquid was applied to the edges of each coverslip, and these were then dried, and gold coated in an Edwards S150B sputter coater (Edwards High Vacuum, Crawley, West Sussex, UK). The cells were imaged with a TESCAN VEGA3 scanning electron microscope (TESCAN, Brno, Czech Republic) at a voltage of 20 kV.

7) Determination of cell wall and membrane integrity. According to the previously described method [Liu V et al., Adv Sci 7, 1902227 (2020); Santiso R et al., BMC Microbiol 11, 191 (2011)], to evaluate the integrity of cell wall, an aliquot of each sample was diluted to a concentration of 5×10⁶ CFU/ml in LB medium. Then, a tube containing 0.5 ml of low-melting point agarose was placed in a water bath at 90-100° C. for about 5 minutes to melt the agarose completely and then placed in a water bath at 37° C., 25 μl of the diluted bacterial sample was added into the tube and mixed with the melted agarose. A 20 μl aliquot of the sample-agarose mixture was pipetted onto a precoated slide, and the sample was covered with a 22×22 mm coverslip. The slide was placed on a cold plate in the refrigerator (4° C.) for 5 minutes to allow the agarose to produce a microgel with the trapped intact cells inside. The coverslip was removed gently, and the slide was immediately immersed horizontally in 10 ml of the lysing solution (25 mM HEPES-Na, 150 mM NaCl, 0.5% NP40 and 0.5% Triton-X100) for minutes at 37° C. for MRSA. Those treated with control buffer (25 mM HEPES-Na, 150 mM NaCl, 0.2% EDTA and 0.5 mg/ml lysozyme) served as positive control group. The slide was washed horizontally in a tray with abundant distilled water for 3 minutes, dehydrated by incubating horizontally in cold (−20° C.) ethanol of increasing concentration (70%, 90% and 100%) for 3 minutes each, and air-dried in an oven. The dried slide was incubated in a microwave oven at 750 W for 4 minutes and the DNA was stained with 25 μl of the fluorochrome SYBR Gold (Thermo fisher, China, diluted 1:400) in TBE buffer (0.09 M Tris-borate, 0.002 M EDTA, pH 7.5) for 2 minutes in the dark. After a brief wash in phosphate buffer pH C88 (Merck, Darmstadt, Germany) a 24×40 mm coverslip was added, and the slide was visualized under fluorescence microscopy.

According to reported method [Liu Y et al., Adv Sci 7, 1902227 (2020)] with some modifications, a permeability assay was performed to analyze the integrity of cell membrane. Specifically, about 50 μl of 10⁹ CFU/ml mid-log-phase MRSA with different pre-treatments were incubated with propidium iodide (PI) at final concentration of 0.5 μM. As a red-fluorescent DNA dye, PI can only cross the plasma membrane of nonviable but not live cells. After incubation for 30 minutes in dark, the fluorescence intensity (λₑₓ=35 nm, λₑₘ=615 nm) was determined using an infinite M200 microplate reader. Those treated with 0.5% Triton-X100 or 1×PBS served as positive or negative control group, respectively. All assays were performed in triplicate and results were expressed as average±SD.

8) Synthesis and digestion of cell wait. According to the reported method [Kuru E et al., ACS Chem Biol 14, 2745-2756 (2019)], over-night cultured MRSA was inoculated into LB medium and cultured at 37° C. with shaking at 250 rpm until when its OD₆₀₀ reached 0.6. Then, these mid-log-phase MRSA was treated with cefdinir (0.5 μg/ml), lysozyme (0.5 mg/ml) alone or their combination. Those treated with mezlocillin (1.0 μg/ml) and without any treatment served as antibiotic-control and untreated group, respectively. Meanwhile, MRSA suspension in control group was further divided into two groups. i.e., control group and no drug group, Next, 10 μM HCC-Amino-D-alanine (HADA) was added into all samples except those in no drug group, in which HADA was replaced by PBS. After 2- or 4-hour incubation, all bacteria were collected by centrifugation at 3220 g for 10 minutes and then washed by cold PBS for 3 times to remove redundant HADA. Then, all bacteria were normalized according their OD₆₀₀ and 100 μl of bacterial suspension was added into each well of 96-well plate before the plate was put into M200 micro-plate reader to record their fluorescence intensities (λₑₓ=385 nm, λₑₘ=461 nm).

To evaluate the digestion rate of lysozyme against cell wall, similar bacterial treatment was performed but further sonication was performed to collect the cell wall by centrifugation at 3220 g for 10 minutes. All cell walls from different treatment groups were re-suspended and normalized in digestion buffer (50 mM NaCl, 10 mM Tris-HCl, pH 8.0). Then, 0.5 mg/ml lysozyme was added into tubes for next digestion reaction at 37° C. After 2 hours incubation, 100 μl of digestion supernatant was separated from undigested cell wall by centrifugation at 3220 g for 10 minutes and recorded its fluorescence intensity (λₑₘ=461 nm). The step was repeated at time intervals of 4, 8 and 16 h. All assays were performed in triplicate.

9) Acetylation modification analysis. According to the reported reference [Brott A S et al., Assays for the enzymes catalyzing the O-acetylation of bacterial cell wall polysaccharides. In: Bacterial Polysaccharides: Methods and Protocols (ed Brockhausen I). Springer New York (2019); Yadav A K et al., Front Microbiol 9, 2064 (2018)], acetylation on cell wall was the mot frequently reported modification in lysozyme resistance. To analyze the acetylation potential of cell wall in different group, mid-log-phase MRSA with OD₆₀₀ of 0.4 to 0.6 was treated with 0.5 mg/nm lysozyme, 0.5 μg/ml cefdinir or their combination. Those without any treatment seed as control group. After 1 hour incubation at 37° C. equal bacteria (1 ml bacteria were collected when the OD₆₀₀ was 1) were collected, washed by cold PBS for 3 times and centrifuged at 12000 g for 3 minutes to collect the supernatant. According to the described method [Brott A S et al., Assays for the enzymes catalyzing the O-acetylation of bacterial cell wall polysaccharides. In: Bacterial Polysaccharides: Methods and Protocols (ed Brockhausen I). Springer New York (2019); Yadav A K et al., Front Microbiol 9, 2064 (2018)] and previous pre-assay, 1.5 μg cell supernatant was incubated with 0.5 mM of 4-Methylumbelliferone (4-MU). After 3 hours incubation at 37° C. 200 μl of the mixture in each group was added into each well to analyze the fluorescence intensity (λₑₓ=372 nm, λₑₓ=450 nm). All assays were performed in triplicate and results were expressed as average±SD.

10) Proteomics analysis ad PRM-based validation. According to the previously described method [Guo H. et al., ACS Chem Biol 17, 2003-2009 (2022)], MRSA in exponential phase was exposed to lysozyme (0.5 mg/ml), cefdinir (0.5 μg/ml) alone or their combination at 37° C. Similar treatment was performed in mezlocillin (1.0 μg/ml) group, Control group was defined as those without any drug treatment. After incubation for 1 hour, all cells were harvested mid the total proteins of samples were extracted using the multiple freezing-thawing at liquid nitrogen followed by sonication at lysis buffer (25 mM HEPES-Na, 150 mM NaCl, 0.1% NP40, 4M urea and 1× protease inhibitor). Then, BCA kit (Theme fisher, China) was used to quantify the extracted proteins for next digestion by trypsin at the ratio of 1:3. These overnight digested peptides were further labeled by TMT-16^{plex} before loading onto Orbitrap Exploris™ 480 Mass Spectrometer (Thermo fisher, China) coupled with an UltiMate 3000 UPLC System (Theme Fisher Scientific) with C18 analytical column. Mobile phases A and B consisted of 0.1% FA in water and 0.1% FA in 100% ACN, respectively. A 120-minute gradient at a flow rate of 300 nl/minute was used. Mobile phase B was increased to 6% at 12 minutes, 20% at 82 minutes, 30% at 92 minutes, 90% at 100 minutes and held for 5 minutes. Data was collected in data-dependent acquisition (DDA) mode with HCD fragmentation at TopN mode. The resolution was set at 60,000 for MS1 and 15,000 for MS2 with 30 ms maximum injection time. All resulted spectra were searched against UniProt Staphylococcus aures (20,330 entries, accessed September 2019) using MaxQuant 1.5.8.2. The parameters for searching: a mass tolerance of 10 ppm for precursor ions; ±0.1 Da for-fragment ions, carbamidomethylation on cysteine was set as a fixed modification, oxidation on methionine and protein N-terminal acetylation was set as variable modifications. The enzyme was specified as trypsin with two missed cleavages allowed. False discovery ratio for peptidic spectral matches and proteins were set as 1%. The maximum number of modifications per peptide was three. Differentially expressed proteins at protein-levels were identified with p-values≤0.05 and fold change (FC) values ≥1.5 (log₂ FC≥0.58 or log₂ FC≤−4.58). The cuff-diff program was used to analyze differences between two treatments.

Similar protocol was performed in parallel reaction monitoring (PR) analysis with minor revision [Guo H et al., ACS Chem Biol 17, 2003-2009 (2022)]. Overnight digested label-free peptides were loaded onto Orbitrap Exploris™ 490 Mass Spectrometer (Thermo fisher. China). PRAM acquisition methods were directly developed based on our DDA data. The DDA data was imported into skyline to select targeted peptides from previous selected targets. The relative information including precursor m/z charge, retention time window of the selected peptides was exported from skyline into xcalibur software to edit the PRM method. The targeted MS1 parameters were as follows: resolution, 60,000; AGC target. 2.0×10⁵; and maximum injection time, 50 ms. PRM scanning was performed at 30,000 resolution, 1×10⁵ AGC target, 54 ms maximum injection time and 0.7 m/z isolation window with peptides in 5 minutes retention time windows. Fragmentation was performed with normalized collision energy of 32. After PRM data acquisition, the data was imported into skyline for analysis to confirm the confidence of proteomics analysis results. To get reliable identification and quantification, idotp and dotp were >0.60. The top three product ions were summed up to represent the peptide abundance.

11) Cytochrome C binding assay. According to the reported method [Palacios L et al., PLoS One 9, e93830 (2014); Yang S J et al., Antimicrob Agents Chemother 54, 3079-3085 (2010)], overnight cultured MRSA was inoculated into LB medium at the ratio of 1:100. After 3.5 hours incubation at 37° C., mid-log-phase MRSA (OD₆₀₀˜0.6) was exposed to cefdinir (0.5 μg/ml), lysozyme (05 mg/ml) alone or their combination. Those without any treatment served as control group. After 1 hour incubation at 37° C., all bacteria were washed twice in deionized water and suspended in the same solution until the final OD₆₀₀ of 2.4. The suspension was then incubated with 0.5 mg/ml of cytochrome c. After 10 minutes incubation at rooms temperature, the supernatant was collected by centrifugation at 12000 g for 3 minutes. MRSA-free deionized water with or without 0.5 mg/ml of cytochrome c served as positive control and background group, respectively, 200 μl of supernatant in each group was added into each well to analyze the content of unbound cytochrome c by measuring the absorption at 530 nm. The lower intensity at 530 nm indicated more positive charge of cell wall and stronger interaction with positive charge lysozyme. All assays were performed in triplicate and results were expressed as average±SD.

12) ATP, NADH and pyruvate levels. According to the described method [Liu Y et al., Adv Sci 7, 1902227 (2020)], mid-log-phase MRSA with an OD₆₀₀ of 0.4 to 0.6 was treated with lysozyme, cefdinir or their combination at different concentrations. Those without any treatment served as control group. After 1 hour incubation at 37° C. bacteria with similar load (according to OD₆₀₀, 1 ml bacteria were collected if the OD₆₀₀ was 1) were collected, washed by cold PBS for 3 times. To evaluate the ATP and NADH level, these collected samples were further re-suspended in cold extracting buffer (Beyotime. Co. Ltd. China) to get bacterial suspension with an initial density of about 5.0×10⁶ CFU/ml, followed by sonication lysis for 40 minutes at 4° C. and centrifugation at 18,000 g at 4° C. for 20 minutes to collect these supernatants. Here, 20 μl of each supernatant were added each well of white 96-well plate, in which each well had been pre-reactivated by 100 μl of ATP test solution (Beyotime. Co. Ltd, China), followed by luminometer measurement. To analyze the NAD⁺/NADH ratio [Xue C et al., Front Pharmacol 12, 6000296 (2021)], half of lysis supernatants were frozen at −20° C. immediately and the rest half were firstly heated at 60° C. for 30 minutes in PCR machine to decompose NAD⁺ if had, Next, these heated supernatants were further centrifuged at 18,000 g at 4° C. for 20 minutes to collect their supernatants. Then, 10 μl of staining solution (Beyotime. Co, Ltd. China) was added to react with these supernatants from frozen group and heated group at 37 XC, respectively. After 30 minutes incubation in the prevention of light, orange formazan was observed and then the absorbance at 450 nm was recorded. The intensities indicated the level of NADH itself or NADH+NAD⁺. Then, the ratio of NAD⁺/NADH was figured out accordingly. To evaluate the pyruvate level [De Palma S et al., PLoS One 8, 656716 (2013)], all samples were similarly re-suspended in cold extracting buffer (Solarbio. Co Ltd. China) to get bacterial suspension with n initial density of about 5.0×10⁶ CFU/ml, followed by sonication lysis for 40 minutes at 4° C. and centrifugation at 18,000 g at 4° C. for 20 minutes to collect these supernatants. Then, 75 μl of each supernatant were incubated with 25 μl of reaction I, followed by continuous incubation with 125 μl of reaction II. By recording the absorbance at 520 nm, the pyruvate level in each sample was figured out according to the standard formula. By defining that in control group as 100%, other were normalized. All assays were performed in triplicate and results were expressed as average±SD.

13) Purification of G-type lysozyme. Lysozyme can cleave β-1,4 glycosidic bond of peptidoglycan in bacterial cell wall. Widely used lysozyme in nature generally belongs to: C-type (from human or chicken), G-type (from goose), T-type (from T4 bacteriophage) and bacterial-type (from bacteria, such as Bacillus subtilis) [Ganz T. Lysozyme, In: Encyclopedia of Respiratory Medicine (eds Laurent G J, Shapiro S D). Academic Press (2006)].

According to a referable protocol with refinement [Thammasirirak S et al., Biosci Biotechnol Biochem 65, 584-592 (2001)], goose egg white (from Anser cygnoides egg) was firstly separated, diluted with two volumes of 50 mM phosphate buffer (PBS, pH 7.0) and stirred for 30 minutes at 4° C., followed by centrifugation at 12,000 g at 4° C. for 15 minutes to collect the solvable supernatant. Next, the crude extract was further treated with isoelectric precipitation at pH 4.0, 6.0 and 7.0. At each step of pH treatment, the solution was adjusted to the desired pH by using 1 M HCl or NaOH for next incubation at 4° C. for 1 hour. After that, the crude extract would be further purified by centrifuging at 12,000 g at 4° C. for 30 minutes and filtering under 0.45 μm filter to remove insoluble protein precipitation. Next, all soluble supernatant was applied to a 5 ml HiTrap SP-FF cation exchange rein column (GE Healthcare) pre-washed by five column volumes of 50 mM PBS (pH 7.0) at a rate of 2 ml/min. Then, column was washed with the same buffer and the targeted lysozyme was eluted with a linear gradient of NaCl concentration from 100 mM to 500 mM in the same buffer at the flow rate of 1.5 ml/min. Next, the purified lysozyme was pooled and uploaded onto Superdex 75 column (GE Healthcare) equilibrated with running buffer (10 mM NH₄NO₃, pH=7.0). These target lysozyme fractions (lysozyme was confirmed by using in-gel digestion mass spectrometric identification, data not shown) were collected and then concentrated to 80 mg/ml for further analysis by using SpeedVac rotary evaporator at 4° C. for 5 hours, followed by aliquoting for long-term storage at −80° C.

14) Toxicities analysis on lysozyme and cefdinir. Toxicities analysis of lysozyme and cefdinir were performed on ARPE-19 and 293T cells [Guo H et al., ACS Chem Biol 17, 2003-2009 (2022)]. Drugs at different concentrations were added into the plates, in which cells had 80% confluency. Those without any drug's treatment served as control group. Ater 24 hours culture at 37° C., survival cells were counted by using CCK-8 kit, 100% survival ratio (no toxicity) was defined as those had same number of survival cells in control group. These assays were performed in triplicate and results were expressed with as average±SD.

15) Cell-based infection model. According to the previously described method [Wang R et al., Nat Commun 9, 439 (2018)], ARPE-19 cell was firstly cultured in gibco dulbecco's modified eagle medium: nutrient mixture F-12 (DMEM/F12) supplemented with 10% fetal bovine serum (FBS) and grown at 37° C. in 5% CO₂-humidified atmosphere for 7 days. Then, about 1.0×10⁴ collected ARPE-19 cells were seeded into per well of 96-well plates and incubated overnight to ensure their confluency. Logarithmic cultures of MRSA were washed with PBS for three times and resuspended in DMEM/F12-10% FBS to get bacteria suspensions with an initial density of about 1.0×10⁸ CFU/ml. Next, 10 μl of bacterial suspension was added into each well, followed by incubation for another 4 hours until multiplicity of infection (MOI) of bacterial infection was 50. Infected cells were then washed vigorously with PBS tor six times and replenished with culture medium to remove unbound bacteria. Herein, cell-associated bacteria were defined as bacteria that attach to, penetrate, or transcytose in ARPE-19 cells. Next, these ARPE-49 cells were exposed to either lysozyme, cefdinir or their combination overnight under identical cell culture condition (100 μl of per well). Cell in the absence of any drug served as control group. After 24 hours incubation, CCK-8 assays were performed to analyze the survival ratio of cells under different treatments. Specifically, 10 μl of CCK-8 solution per well was added mid the cells were incubated for another 1 hour at 37° C. for chromogen development. The absorbance was measured at 450 nm, with 630 nm as reference wavelength. Herein, survival ratio=(Absₛₐₘₚₗₑ−Abs_{negative control})/(Abs_{positive control}−Abs_{negative control})×100%. The bacterial loads were examined by lysing ARPE-19 cells with 1% Triton X-100 in PBS and serially diluting the resulting lysates to enumerate bacterial colonies on agar plates. These assays were performed in triplicate, and results were expressed as average±SD.

16) Rat skin Infection model. According to the reported method [Zhang Q et al., Proc Natl Acad Sci USA 119, e2119417119 (2022)], 25 Sprague Dawley (SD) male rats (6-7 weeks old) were divided into 5 groups (5 rats per group), 5 rats served as uninfected group. Others were infected by about 5×10¹¹ CFU of mid-log phase MRSA from our own collection. Briefly, all rats were firstly pre-depilated by using 10% Na₂S to get an uncovered back area of 4 cm×4 cm prior to 24 hours for the anesthetization on rats. Then, the skin of anesthetized rat was removed until fascial region under sterile condition, followed by bacterial infection by spraying 5×10¹¹ CFU of MRSA suspension on wound (3 cm×3 cm). To collect these bacterial suspensions, mid-log phase MRSA would be collected by centrifugation at 4500 g for 15 minutes washed with PBS for 3 limes and re-suspended in cold PBS to get bacterial suspension with an initial density of about 4.0×10¹¹ CFU/ml according to pre-experiment. Next, 1.25 ml of bacterial suspensions was inoculated into wound, Normal saline was used to replace bacterial suspension in uninfected group. After 1 hour infection, wound of infected rat was covered by sterile medical gauze. All sterile medical gauzes were 0.2 g/cm², which were further covered by two layers of sterile gauzes and fixed by sterile tapes. After that, rats were monitored until they were fully awake, followed by return to their cages. After 24 hours infection, these sterile medical gauzes were replaced with new sterile medical gauzes. Specifically, rats in lysozyme only and combination groups were treated with new sterile medical gauze soaking with lysozyme (14 mg/ml, 500 μl). However, rats in other groups kept same sterile medical gauzes, i.e., those soaking with normal saline. Meanwhile, cefdinir was prepared in 0.5% methylcellulose, and was further administered orally (123 mg/kg) to rats in cefdinir only and combination groups. All drug treatment was repeated at 72-hr post-inoculation. At 168 hours post-inoculation, all rats were sacrificed to collect their wounds. These collected tissues were fixed in 10% formalin for at least 24 hours and embedded in paraffin. About 4-μm sections was analyzed by using the hematoxylin and eosin (H-E) staining. The rest of wounds were placed into 1 ml sterile PBS on ice, and then homogenized to detect the levels of ROS or IL-6. The rest of homogenates were immediately stored in sterile tube at −80° C. for further analysis.

17) Animals, cells, bacterial strains, drugs, reagents and instruments. Methicillin-resistant Staphylococcus aures USA 300 (MRSA, ATCC-BAA1556), Staphylococcus epidermis (MRSE, ATCC35984), ARPE-19 and 293T cells in the experiment were from our own collection. Corynebacterium pseudodiphtheriticum (CGMCC code: 1.592), Streptococcus pyogenes (CGMCC code: 1.8868), Streptococcus pneumoniae (CGMCC code: 18722), Enterococcus faecalis (CGMCC code: 1.10682), Enterococcus faecium (MCC code: 115321), Streptococcus mutans (CGMCC code: 1.2494) and Propionibacterium acnes (CGMCC code: 1.5003) were purchased from China general microbiological culture collection center (CGMCC) Competent cells (TOP10, BL21 (DE3) and DH5a (DE3) were supplied by TianGen, China. Egg of Chinese goose was ordered from Etsy, Hong Kong. Sprague-Dawley (SD) male rats in six to seven weeks old were purchased in rat-skin infection studies from ZHBY Biotech Co. Ltd. All experiments were approved by and performed in accordance with the guidelines approved by committee on the use of live animals in teaching and research (CULATR) (Ref No.: ARSA-22127-OTH-ABCT), The Hong Kong Polytechnic University (Shenzhen Research Institute). Human lysozyme (C-type), chicken lysozyme (C-type), T4 phage lysozyme (T-type), Bacillus subtillis lysozyme (bacterial-type), ampicillin sodium salt, hydroxyethyl piperazine ethanesulfonic acid (HEPES), imidazole, thrombin, ammonium nitrate (NH₄NO₃) formaldehyde, crystal violet, sodium chloride (NaCl), 4-Methylumbelliferone, cytochrome c, phosphate buffered saline (PBS) and Luria-Bertani (LB) broth powder were supplied by Sigma-Aldrich. ATP assay kit, NAD⁺/NADH assay kit, pyruvate level kit, Tryptic Soy Broth (TSB) medium, defibrinated horse blood were obtained from Solarbio. Co. Ltd. China. Methanol, glutaraldehyde. NP40, urea and protease inhibitor, TMT-16^{plex}, C18 column, propidium iodide (PI) Triton-X100, ethanol, SYBR gold dye, DMEM/F12, fetal bovine serum (FBS), CCK-N assay kit, BCA assay kit, ROS kit, IL-6 kit, hematoxylin and cosin (H-E) staining kit, plate-reader, bacterial incubator and high-throughput screening robust were purchased from Thermo fisher, China. Fast performance liquid chromatograph (FPLC), ITC 2009, PCR machine, dialysis membrane and centrifugal filters (Ultra-15 (10 kDa, 30 kDa)) were purchased from GE, USA. Ultra sonicator and centrifuges were supplied by Beckman (Germany). Deionized (DI) water (18.2 MΩ·cm) and filters (0.45 and 0.22 μm) were purchased from Millpore, USA. All other chemicals/compounds were purchased from MCE (China) unless otherwise stated.

18) Data availability and statistical analysis. All assays were performed in triplicate and results were expressed as average±SD unless otherwise stated. All tests of significance were based on *p<0.05, **p<0.01 and ***p<0.001. Other data were available from the corresponding author upon reasonable request.

### 2. Results and Conclusion

### 2.1 High-Throughput Screening Explores Broad-Spectrum Synergy Effect Between Cefdinir and Lysozyme.

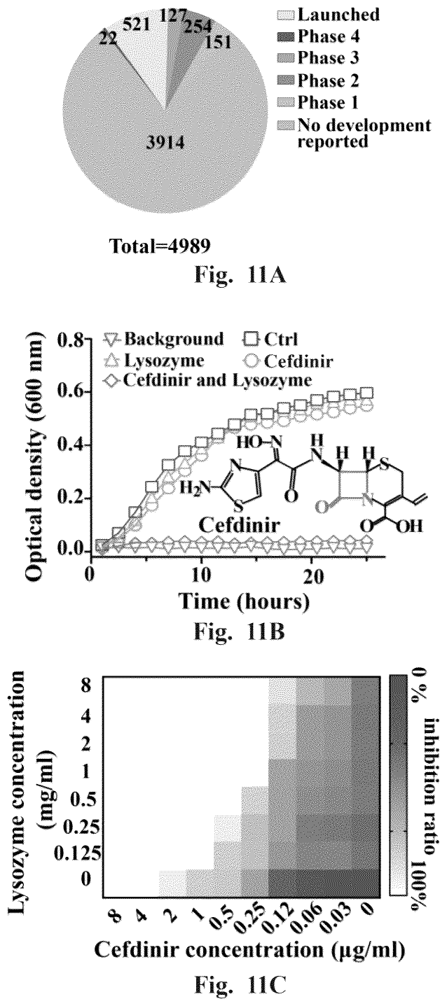

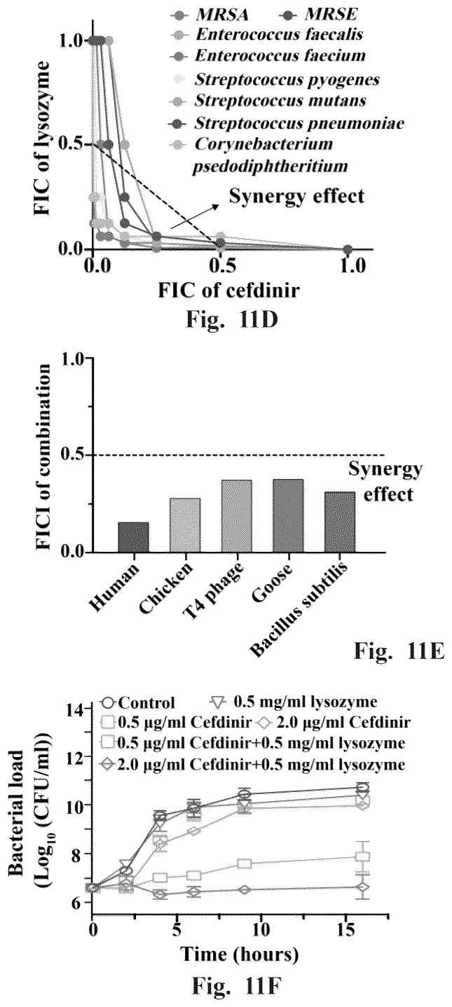

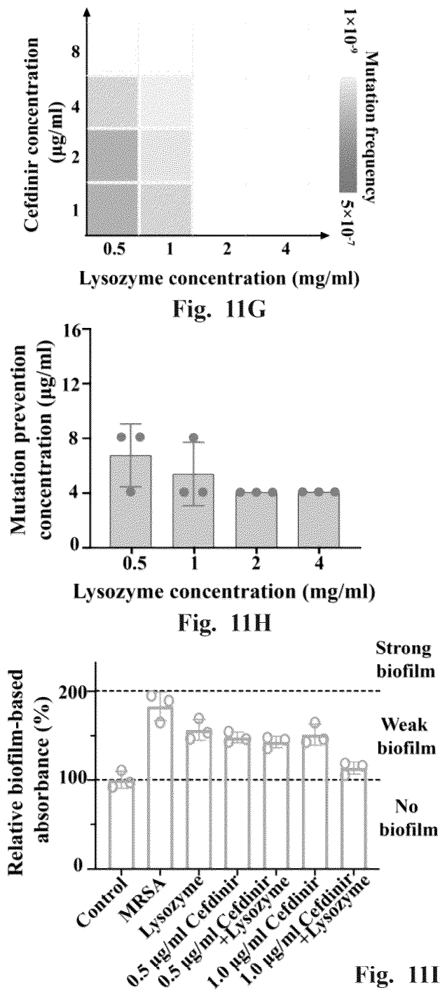

Using standard methicillin-resistant Staphylococcus aureus USA 300 (MRSA, ATCC-BAA1556) and Staphylococcus epidermidis (S. ep, MRSE, ATCC35984) as these model strains, which are the most representative pathogens in hospital acquired infection [Ziebuhr W et al., Int J Antimicrob Agents 28, 14-20 (2006); Sebola D C et al., Front Vet Sci 9, 108752 (2023)] and resistant to human lysozyme with the minimum inhibitory concentration (MIC) at the level of >150 mg/ml (FIG. 15 to FIG. 17H), the synergistic activity between lysozyme and 4989 compounds (six sub-classes, FIG. 11A, FIG. 15, FIG. 16A to FIG. 16B) at the fixed concentrations was measured by monitoring bacterial growth curves during 24 hours (FIG. 11B) The primary screening identified several hits (see following table) that had better inhibition activity in combination with lysozyme against MRSA and MRSE, respectively (FIG. 11B, FIG. 16A).

| | MRSA | | | MRSE | | |
|---|---|---|---|---|---|---|
| | Drug | | Inhibition | Drug | | Inhibition |
| Compounds name | only | Combination | ratio (%) | only | Combination | ratio (%) |
| TD52 (dihydrochloride) | 0.6512 | 0.0926 | 93.07 | 0.402 | 0.1057 | [symbol] 4.42 |
| Se [symbol] aconazole (nitrate) | 0.593 | 0.2603 | 61.38 | 0.4977 | 0.079 | 93.73 |
| SCP1 [symbol] IN [symbol] 1 | 0.1762 | 0.0766 | 79.55 | 0.1091 | 0.0698 | 67.64 |
| Rifamycin S | 0.1545 | 0.074 | 77.78 | 0.5935 | 0.0967 | 91.58 |
| Paroxypropione | 0.3624 | 0.0655 | 95.34 | 0.5254 | 0.0445 | 101.37 |
| Ethyl 3,4-dihydroxybenzoate | 0.5805 | 0.064 | 97.54 | 0.6328 | 0.0703 | 96.68 |
| Delafloxac [symbol] n [symbol] meglumine) | 0.177 | 0.0629 | 90.56 | 0.1374 | 0.0639 | [symbol] 5.07 |
| 2,4-Diacetylphloroglucinol | 0.2097 | 0.0588 | 95.09 | 0.09 | 0.0686 | 54.87 |
| Ce [symbol] dinir | 0.6975 | 0.0564 | 99.16 | 0.6206 | 0.0528 | 99.68 |

[symbol]

indicates data missing or illegible when filed

Of these hits, cefdinir, a semi-synthetic antibiotic belonging to the third generation of the cephalosporin class and widely used to treat common bacterial infections especially for skin infections [Tack K J et al., Antimicrob Agents Chemother 41, 739-742 (1907); Giordano P A et al., Curr Med Res Opin 22, 2419-242K (2006)] (FIG. 11B), was found to exhibit the obvious, broad-spectrum synergistic effect (both inhibition ratios were above 90% against two pathogens) in combination with 1 mg/ml lysosyme, whereas cefdinir or lysozyme alone displayed few direct inhibition activity (FIG. 11C, FIG. 17B).

Then, standard checkerboard microbroth-dilution assay was performed in LB medium to monitor the interaction between cefdinir and lysozyme against several gram-positive pathogens, including MRSE, Streptococcus pyogenes (S. py), Streptococcus mutans (S. mu), Streptococcus pneumoniae (S. pn), Enterococcus faecium (E. faecium), Enterococcus faecalis (E. faecalis) and Corynebacterium pseudodiphtheriticum (C. ps) (FIG. 11D). The synergistic effects were observed in all combination treatments, as judged from fractional inhibitory concentration index (FICI) values from 0.094 to 0.281 (FICI≤0.5 is defined as synergism, FIG. 11D, FIG. 17C to FIG. 17H). Similarly, such a phenomenon was also observed between cefdinir and other sub-types of lysozymes in nature, i. e., C-type (from human or egg), G-type (from goose), T4-type (from T4 phage) and bacterial-type (from bacteria, such as Bacillus subtilis lysozymes (FIG. 11E, FIG. 18A to FIG. 19D). Specifically, it was found that cefdinir at concertation of ea. 0.75 μg/ml (equivalent to 1.89 μM) could re-sensitize MRSA to human lysozyme (C-type lysozyme) with the MIC value of lysozyme dropped by 1200 folds from 1200 folds from 150 mg/ml to 0.125 mg/ml (FIG. 11C. Considering MRSA has been the most notorious superbug, the potential of combination therapy against MRSA in the subsequent studies was thus focused on.

To collect more details of cefdinir in restoring the activity of lysozyme, the time-killing assay was subsequently performed. It was found that neither 0.5 mg/ml lysozyme nor 0.5 μg/ml cefdinir (1.26 μM, even 2.0 μg/ml cefdinir) has any inhibition effect on the growth of MRSA at exponential phase. By contrast, their combination significantly reduced the bacterial load from the level of 10¹⁰ to 10⁷ CFU/ml after 16 hours incubation (FIG. 11F. FIG. 20A). Furthermore, considering that the resistance-mutation is always the tough problem in bacterial infection [Imamovic L et al., Sci Transl Med 5, 204ra132 (2013); Abreu A C et al., Nat Prod Rep 29, 10007-1021 (2012)], the effect of combination therapy on the frequency of resistance mutations was thus assessed. Specifically, the mutation frequency of cefdinir resistance on MRSA exposed to cefdinir in the presence and absence of lysozyme at the increasing concentrations was evaluated, Results showed combination therapy would not induce the surged mutation frequency and the prevention concentrations of cefdinir fluctuated from 8 to 10 μg/ml against MRSA when it was treated with increasing concentrations in combination therapies (FIG. 11G to FIG. 11H). These results suggested that the combination of lysozyme and cefdinir could effectively minimize the de no emergence of resistance development.

Considering the emergence of bacterial biofilm often resulted in invalid treatment effect, especially in skin and ocular infection caused by MRSA [Qu D et al., Sci Adv 6, eaay9597(2020); Zhao F et al., Advanced Materials 35, 2208069 (2023)], crystal violets assays on bacteria at early and late exponential phases were subsequently preformed, corresponding to pro-biofilm and post-biofilm stage, to assess whether biofilm would be inhibited. Interesting, although the treatment of both cefdinir and lysozyme alone slightly reduced the biofilms which could be reflected by absorbance at 590 nm, the combination therapy perfectly decreased into at the san level of control group, indicating the biofilm had been absolutely removed (FIG. 11I) Similarly, the inhibition was also clearly observed after biofilm appeared when such treatment also had the significant inhibition effect on the biofilm-related absorbance, especially when more cefdinir (≥1.0 μg/ml) were added (FIG. 20B). All results prompted the great combination effect between cefdinir and lysozyme against broad-spectrum gram-positive pathogens.

### 2.2 Permeability and Morphological Analysis Indicated Cell Wall Integrity was Damaged Under Combination Therapy.

To explore the mechanism of cefdinir to re-sensitize lysozyme against MRSA, the permeability analysis on MRSA treated with or without cefdinir (0.5 μg/ml), lysozyme (0.5 mg/ml) alone or in combination was firstly preformed. It was found propidium iodide (PI) [Liu Y et al., Adv Sci 7, 1902227 (2020)], a red-fluorescent DNA dye which can only cross the plasma membrane of nonviable but not live cells, couldn't cross the plasma membrane of MRSA in all groups, suggesting no cell membrane was damaged in all groups (FIG. 21A). However, when fluorochrome SYBR gold [Santiso R et al., BMC Microbiol 11, 191 (2011)], a nucleic acid dye, was mixed with MRSA-agarose mixture and pipetted on the precooled slides, the diffusion of nucleic acid was clearly observed in combination groups but not in control and drug alone groups (FIG. 12A-1 to FIG. 12A-2). Such a phenomenon indicated the cell wall but not cell membrane had been seriously damaged under combination treatment.

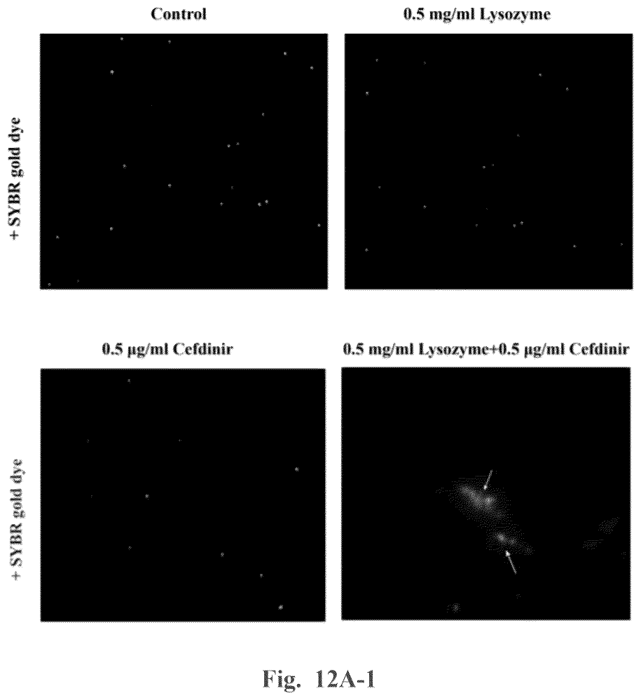

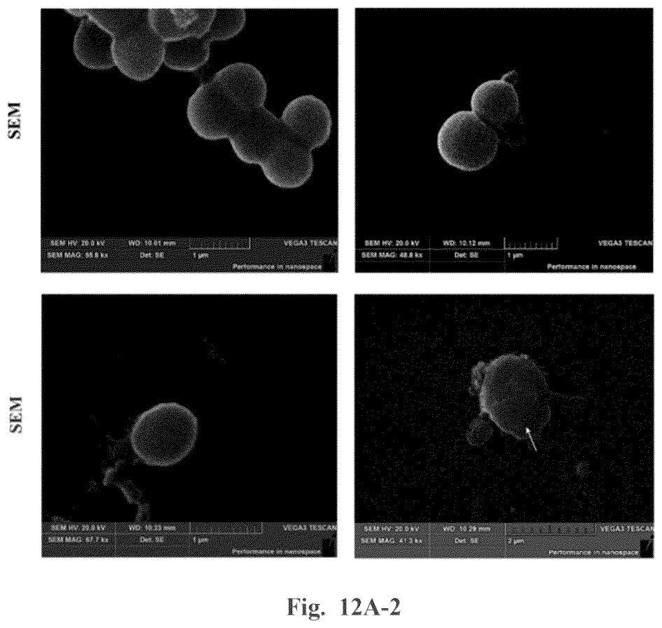

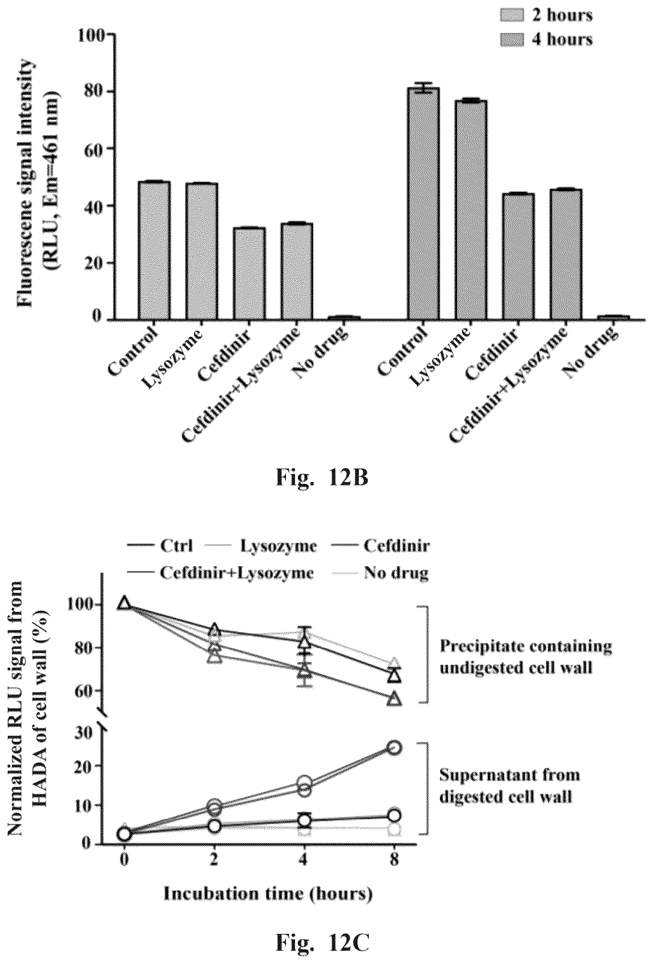

Considering cell wall is the well-known target of both lysozyme [Choi Y et al., Science 335, 319-324 (2012); Oliver W T et al., J Anim Sci Biotechnol 6, 35 (2015)] and cefdinir [Srivastava S et al., Front Pharmacol 12, 677005(2021)], the scanning electron microscope (SEM) imaging analysis was then performed to directly uncover the morphological change of MRSA treated with or without cefdinir (0.5 μg/ml), lysozyme (0.5 mg/ml) alone or in combination. As shown in SEM images in FIG. 12A-I to FIG. 12A-2, the cell surface in untreated group was relatively smooth. Similarly, treatments with either cefdinir or lysozyme alone had few effects on MRSA in morphological level. Interestingly, cells under combination treatment did show obvious digestion-like roughness (FIG. 12A-1 to FIG. 12A-2), which might attribute to the ability of lysozyme to digest the existing cell wall. Conceivably, the damage might be so severe that bacteria were hardly to self-repair and survived.

Then, to explore whether the digestion of cell wall or the inhibited synthesis of nascent cell wall was the reason of the digestion-like roughness. HCC-Amino-D-alanine (HADA, λ_{inf}˜461 nm) was used [Williams Me al., Mbio 12, (2021)], the analog of D-alanine composing of nascent cell wall to evaluate the inhibition effect on synthesis of nascent cell wall under combination treatment. Specifically, ˜40% reduction in HADA signal intensity from nascent cell wall was observed in MRSA under either cefdinir alone or combination treatment for 2 hours (FIG. 12B). Such a phenomenon was more clearly observed in MRSA treated with longer time. Indeed, the decrease in the synthesis of nascent cell wall was expected because cefdinir was one of the typical β-lactam antibiotics. Unexpectedly, when these nascent cell walls were normalized and then digested by lysozyme, compared with MRSA in the control and lysozyme alone groups, the faster digestion rate to release HADA from cell wall of MRSA under cefdinir treatment was clearly observed, which might be attribute to the more effective digestion of cell wall under these treatments (FIG. 12C), Nevertheless, no difference was observed in MRSA treated with other β-lactam antibiotic, such as cefazolin (FIG. 21B), indicating the higher sensitivity might account for cefdinir itself but not as β-lactam antibiotic. Indeed, the higher sensitivity of cell wall against lysozyme might facilitate the irreparable digestion in cell wall, meanwhile cefdinir itself an reduce the synthesis of nascent cell wall, leading to the final bacterial death.

2.3 Proteomics Analysis Indicated that Cefdinir Induced the Overexpression of Enzymes in WTA/LTA Synthesis Pathway, Leading to Stronger Interaction Between Cell Wall and Positive Charge Lysozyme.

Considering that modification on cell wall was the most reported cause of lysozyme resistance and O-acetylation of MurNAc/N-deacetylation of GlcNAc held its dominant position [Bera A et al., Mol Microbiol 5, 778-787 (2005); Davis K M et al., Infect Immun 79, 562-570 (2011); Yadav A K et al., Front Microbiol 9, 2064 (2018)], to explore whether there were any difference in acetylation level on cell wall of MRSA under different treatments, activities of several representative enzymes in acetylation modification, such as OatA and pgdA, was firstly evaluated by using 4-methylumbelliferyl acetate (4-MU-Ac) as their common substrate [Brott A S et al., Assays for the enzymes catalyzing the O-acetylation of bacterial cell wall polysaccharides. In: Bacterial Polysaccharides: Methods and Protocols (ed Brockhausen I), Springer New York (2019)]. With the transfer of acetyl group into MurNAC or GlcNAC, 4-MU-Ac (λₑₓ=360 nm, λₑₘ=499 nm) was finally catalyzed into 4-MU (λₑₓ=372 nm, λₑₘ=445 nm), leading to the significant increase in fluorescence intensity at 450 nm. Unexpectedly, if 0.5 mM 4-MU-Ac was incubated with cell lysis supernatant of MRSA under different treatments at 37° C. for 3 hours, no obvious difference in their fluorescent spectroscopy was observed, prompting that the re-sensitization of MRSA to lysozyme might attribute to other factors but not the modification on cell wall (FIG. 22A to FIG. 22C).

To this end, the proteomics study was then performed to uncover potential change in protein level in MRSA treated with or without lysozyme and/or cefdinir combination for 3 hours. By performing tandem mass tag (TMT) labeling quantitative proteomics analysis on MRSA under different treatments, samples between lysozyme only and combination therapy groups were compared and revealed the significant (p<0.05) up-regulation of 407 and down-regulation of 134 differentially expressed proteins (DEP (FIG. 13A, FIG. 23A to FIG. 23F). Gene ontology (GO) annotation analysis showed that these DEPs are mainly correlated with carbohydrate derivative biosynthesis process (biological processes), small molecule binding (molecular functions) and located in cell periphery (cellular components). Similar results were also found in those with and without treatment with cefdinir (FIG. 23A to FIG. 23F). Unexpectedly, it was clearly observed that the general over-expression of enzymes, including TarA, TarB, TarD, TarF, TarL, TarS, TagH and FmtA, in the pathway to synthesize wall teichoic acids (WTA) and lipoteichoic acids (LTA) (FIG. 13B to FIG. 13C). Remarkably, WTA and LTA were dominative acids to adjust charge of cell wall in gram-positive pathogens [Schneewind O et al., Lipoteichoic acid synthesis and function in gram-positive bacteria. In: Biogenesis of Fatty Acids, Lipids and Membranes (ed Geiger O). Springer International Publishing (2016); van Dalen R et al., Trends Microbiol 28, 985-998 (2020)]. To further confirm these changes in protein level, these interesting candidates in list with targeted mass spectrometry approach were re-analyzed, namely parallel reaction monitoring (PRM). Specifically, some unique sequences were selected during accurate quantification studies. Despite with differential MS responses and intensities, all selected peptides showed the similar trends with those in previous proteomics analysis (FIG. 13D). Thus, it was believable that more LTA/WTA acids on cell wall inevitably facilitated stronger interaction between negative charge cell wall and positive charge lysozyme.

To further validate it, a cytochrome C binding assay was next performed [Yang S J et al., Antimicrob Agents Chemother 54, 3079-30M5(2010)], on MRSA under different treatments. As a colorful protein with positive charge in physiological condition [Ow Y-L P et al., Nature Reviews Molecular Cell Biology 9, 532-542 (2008)], cytochrome C could bind into cell wall, which depended on the degree of negative charge in cell wall [Yang S J et al., Antimicrob Agents Chemother 54, 3079-3085 (2010)]. By incubating 0.5 mg/ml) free cytochrome C with 0.5 OD₆₀₀ MRSA at 37° C. for 10 minutes, it was found that cefdinir treatment led to the less free cytochrome C, indicating that such MRSA had more positive charge of cell wall and thus possibility to stronger interaction with positive charge lysozyme (FIG. 13E) Meanwhile, by adding LTA/WTA synthesis inhibitor [Santa Maria J P et al., Proc Natl Acad Sci USA 111, 12510-12515 (2014)] into bacterial suspension, whether combination effect on MRSA would be disrupted was further explored. As shown in FIG. 13F and FIG. 24A to FIG. 24D, it was fund that both LTA synthesis inhibitor (LtaS-IN-1) and WTA synthesis inhibitor (Targocil) did be able to abolish the combination effect, as judged from the increasing FICI values in combination therapy. All results confirmed that the more synthesis of LTA/WTA led to stronger interaction between cell wall and positive charge lysozyme, facilitating the rapider digestion of cell wall by lysozyme in combination therapy.

Considering that many changed proteins in volcano plot had high correlation with energy metabolism, such as ATP synthase subunit c (UniProt ID: Q6G7K2) (FIG. 25A to FIG. 26G), to evaluate the possible side-effect on MRSA under different treatments, the levels of ATP, NADH/NAD⁺ and pyruvic acid were then analyzed. As shown in FIG. 27A, no decrease in pyruvic acid level was observed, which indicated few effects happened on the upstream energy metabolism. Nevertheless, compared with the unobserved change in total level of NAD⁺+NADH (FIG. 27B), the level of NADH was significantly reduced in MASA under cefdinir treatment (FIG. 27C). Thus, cefdinir led to 1.3-fold increase in the ratio of NAD⁺/NADH (FIG. 27M). Remarkably, the ratio of NAD⁺/NADH played the critical role in oxidation reaction, especially for bacterial energy metabolism [Spaans S K et al., Front Microbial 6, 742(2015)]. Furthermore, the increasing ATP level in MRSA under the combination therapy (1.5-fold, FIG. 13G) was also found which might be partly attribute to the overexpression of ATP-associated enzymes as well as its saved consumption in the inhibited synthesis of cell wall and side-effects from following cell death.

All in all, these results clearly uncovered that cefdinir could synergize lysozyme to digest cell wall by up regulating the expression of enzymes, including TarA, TarB, TarD, TarF, TarL, TarS, TagH and FmtA, in LTA/WTA synthesis to facilitate the interaction between cell wall and lysozyme, leading to strengthened abilities in digesting/killing MRSA under combination therapy.

### 2.4 Combination Therapy Significantly Reduces the Bacterial Load in Cell Infection Model.

Next, the potential of combination therapy in cell-based infection model was further evaluated. Considering lysozyme was abundant in human eye, ARPE-19 cell was thus selected [Fasler-Kan E et al., Methods Mol Biol 1745, 305-314 (2018)], a spontaneously arising retinal pigment epithelia (RPF) cell, to perform the bacterial infection assay. It was found that neither cefdinir (c 64 μg/ml) nor lysozyme (<16 mg/ml) at the certain safe concentration had any effect on cell survival ratios of uninfected ARPE-19 cells (FIG. 28A to FIG. 28B). However, if cells were infected by MRSA at the multiplicity of infection (MOI) of 50.80% infected cells were dead after 24 hours. Interestingly, with the increasing concentration of cefdinir in combination therapy with 0.5 mg/ml lysozyme, survival ratio of infected cells increased and bacterial load in cellular reduced (FIG. 28C to FIG. 28D). Specifically, the bacterial load significantly dropped into a level of 10³ CFU/ml (10²-fold) when 0.25 μg/mli cefdinir was combined with 0.5 mg/m lysozyme and maintained until the end of analyzed points (the highest concentration of cefdinir was 4 μg/ml) (FIG. 28D). Collectively, it was demonstrated that antimicrobial efficacy of cefdinir and lysozyme combination therapy in vitro can be well-translated into in cellular efficacy.

### 2.5 Combination Therapy Effectively Relieves the Symptoms of Rats in Skin Infection Model.

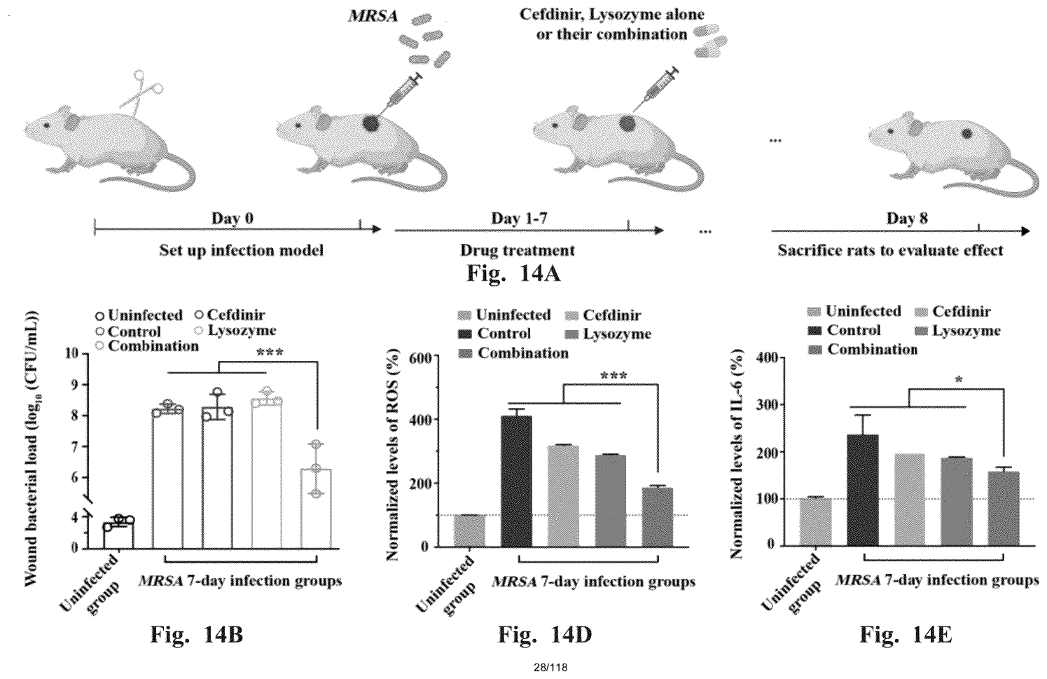

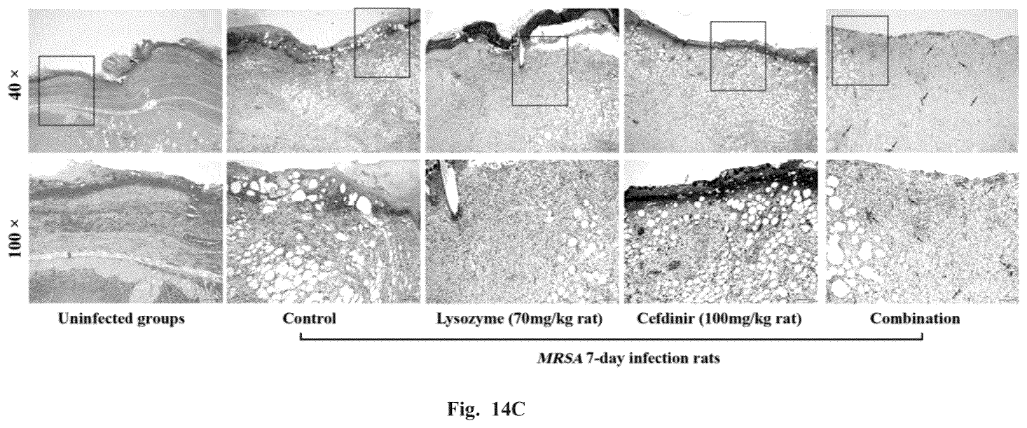

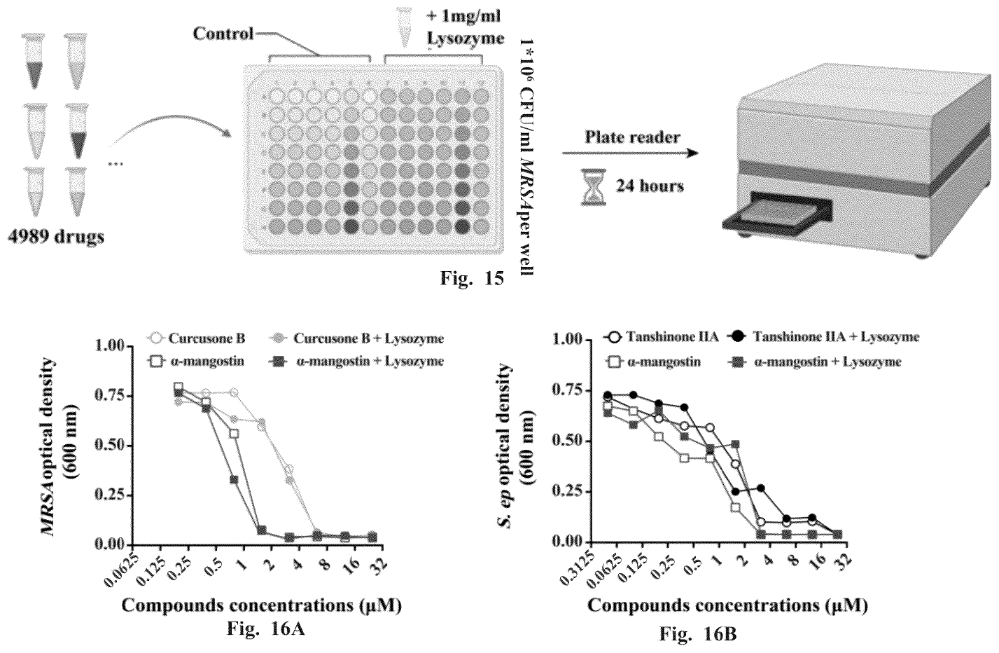

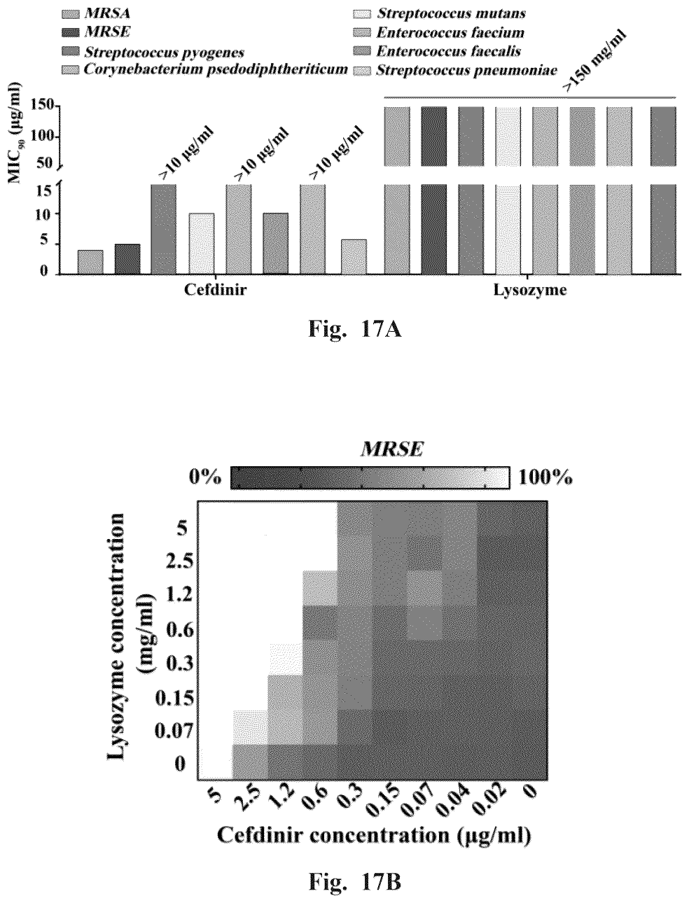

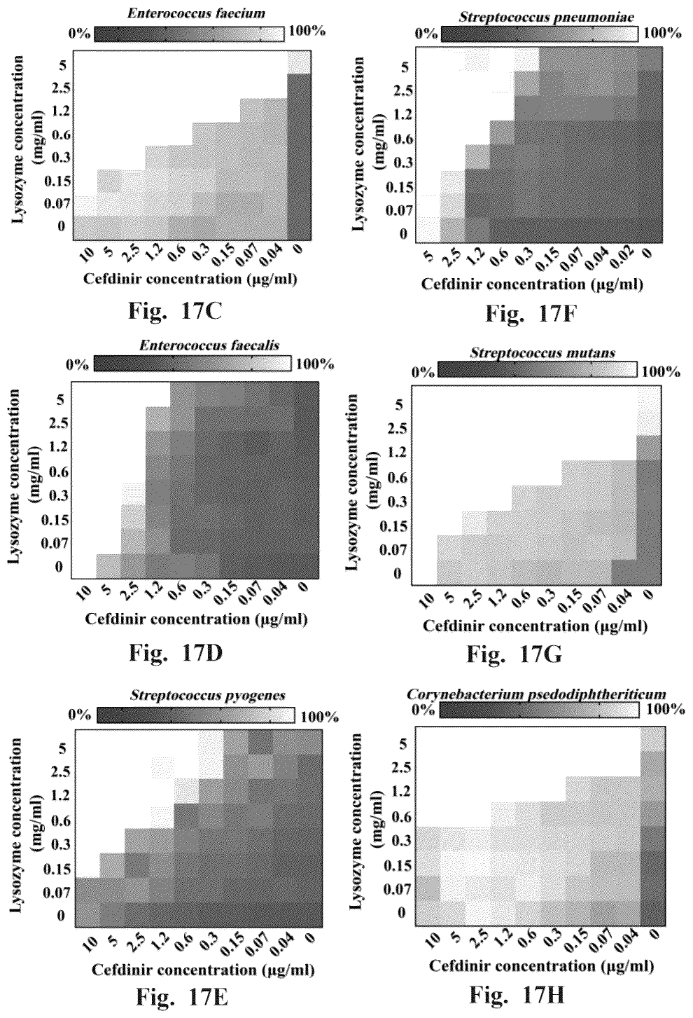

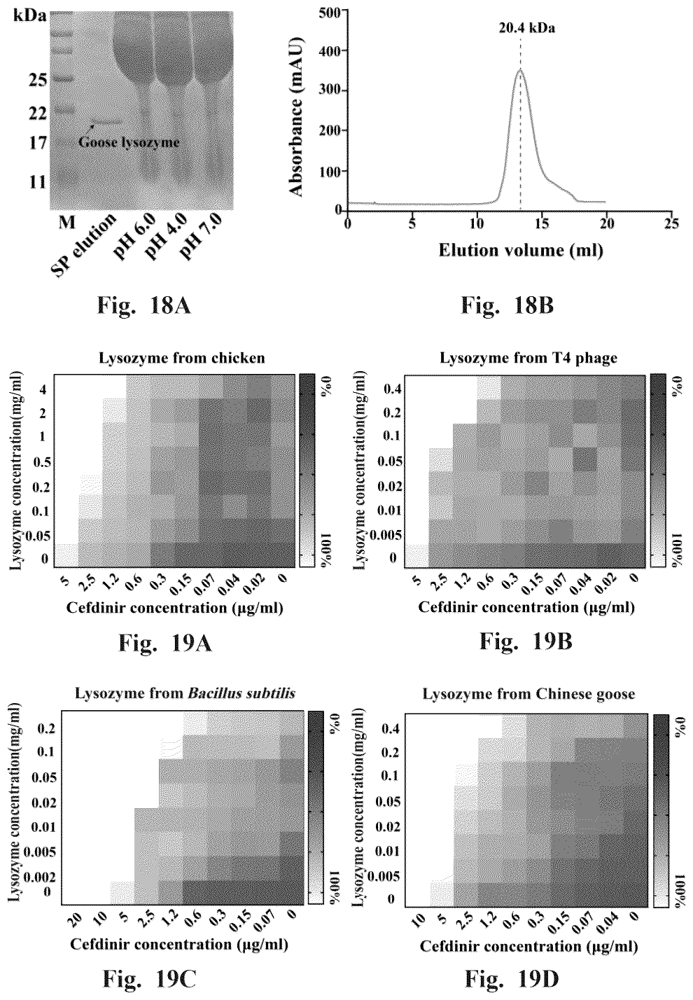

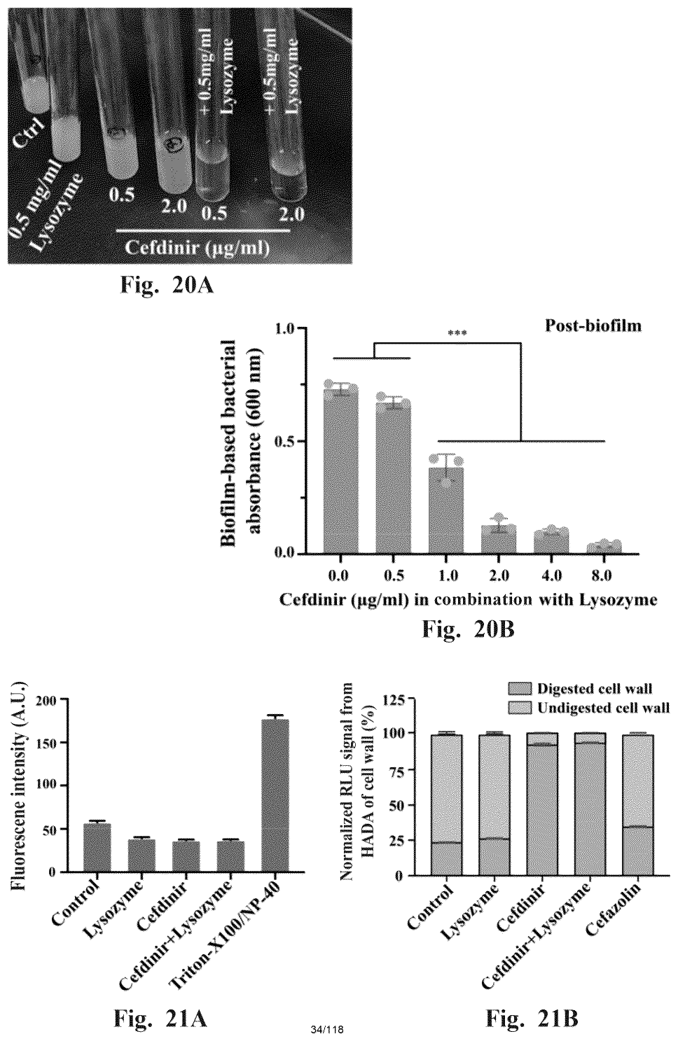

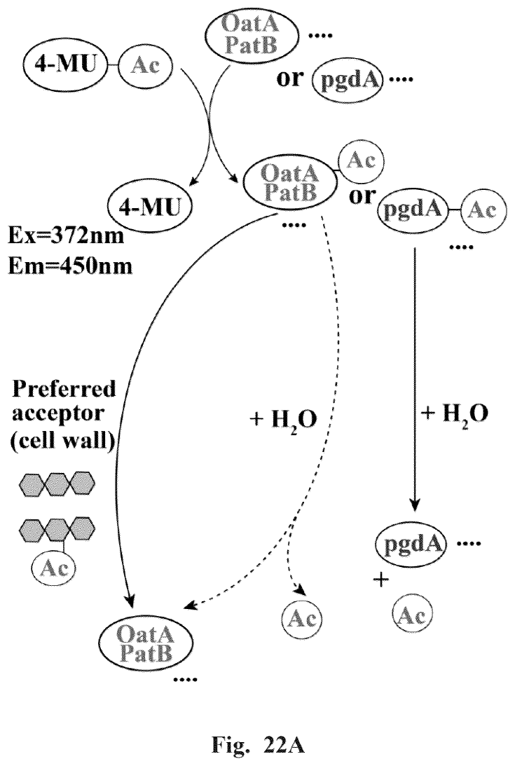

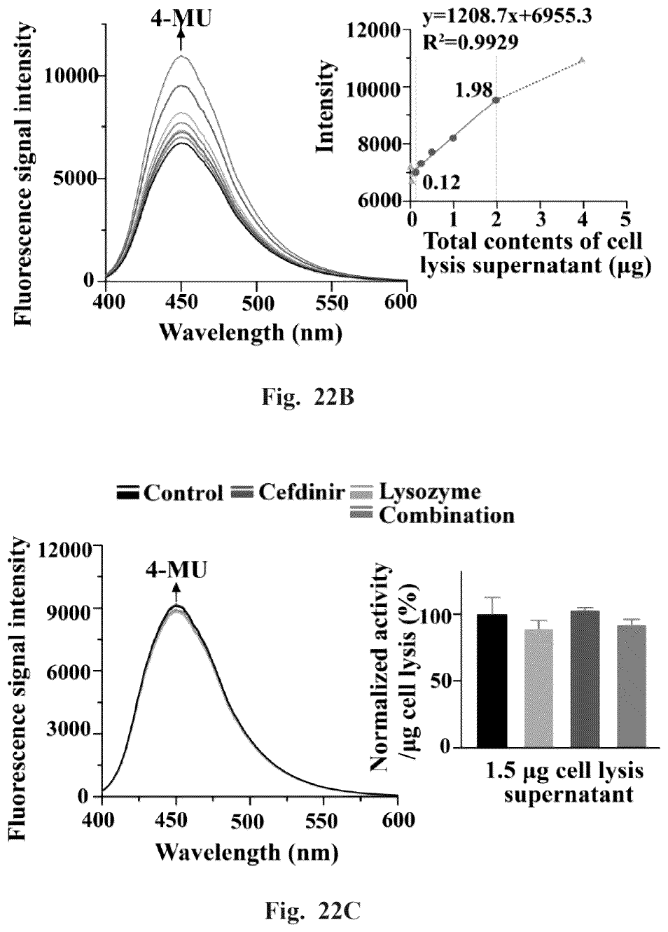

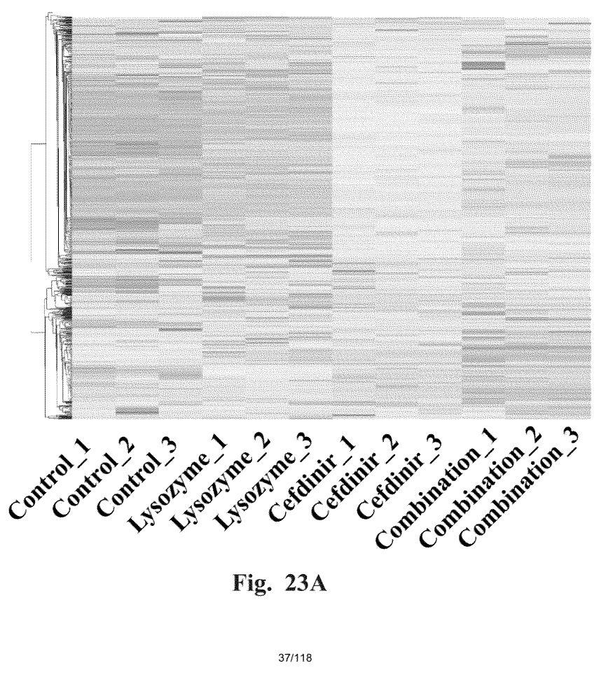

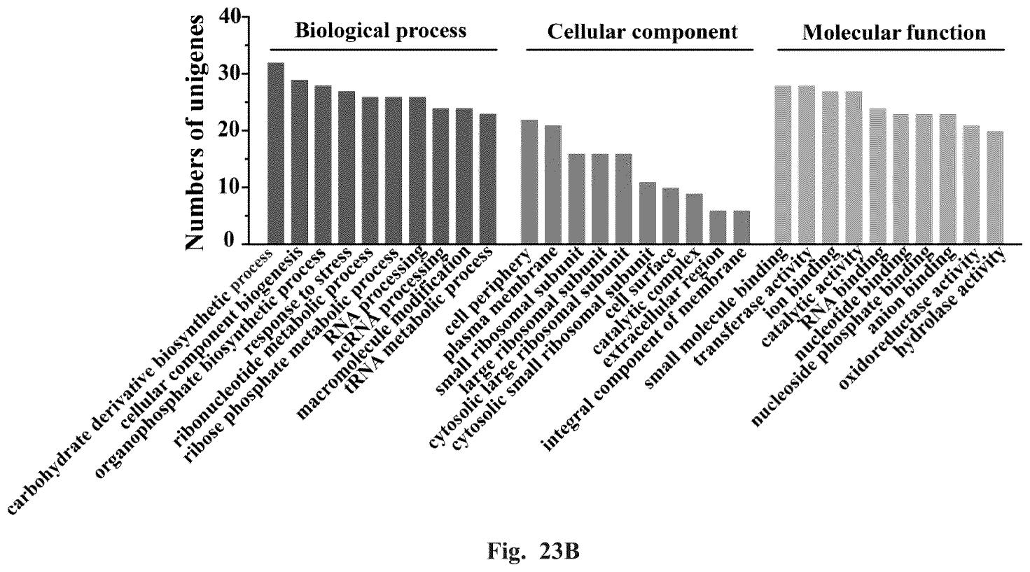

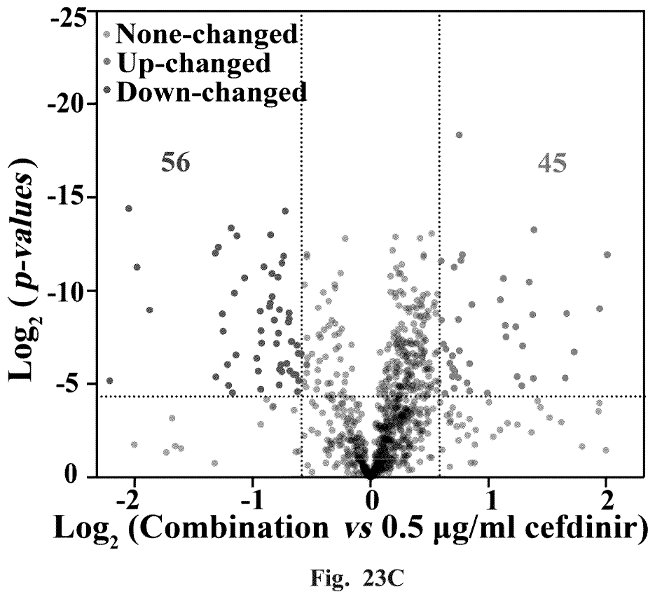

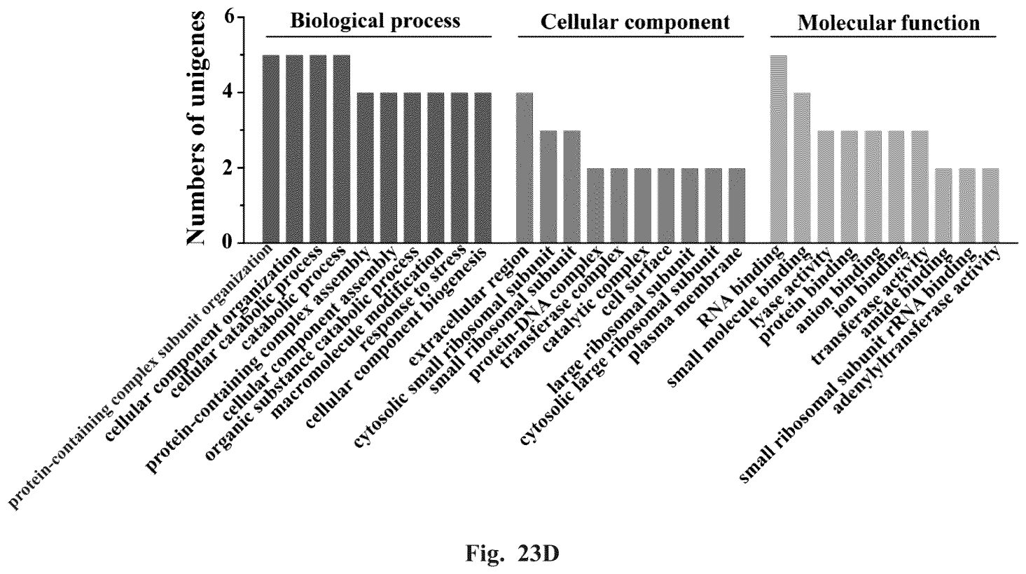

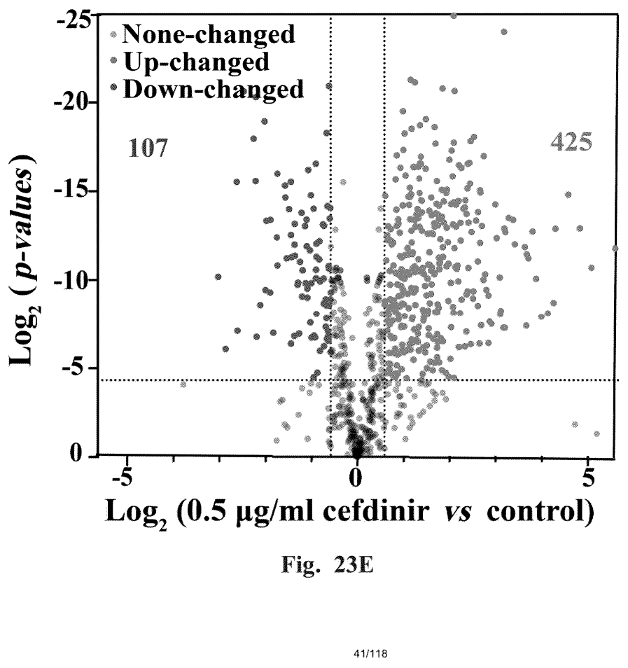

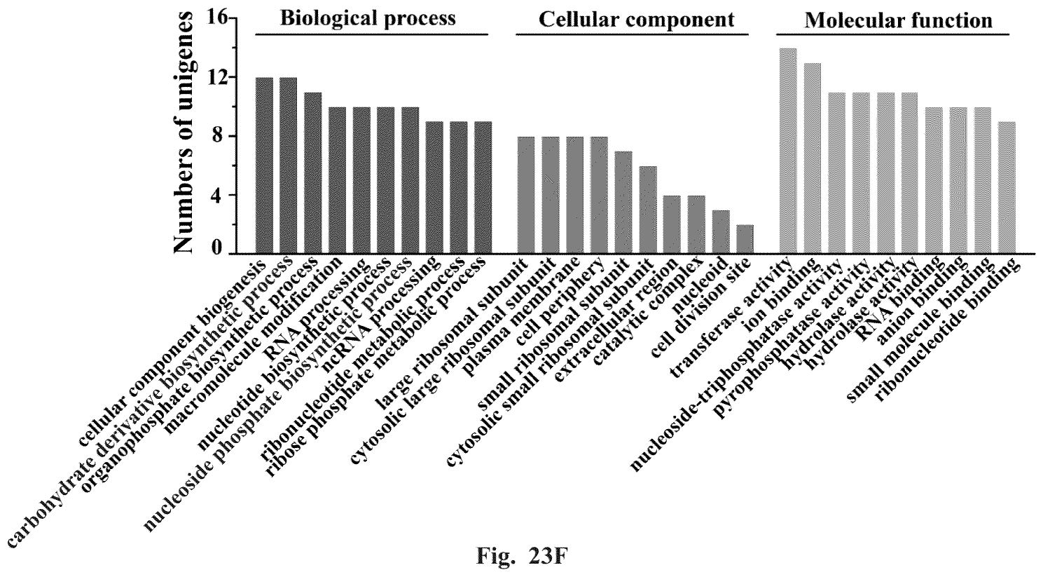

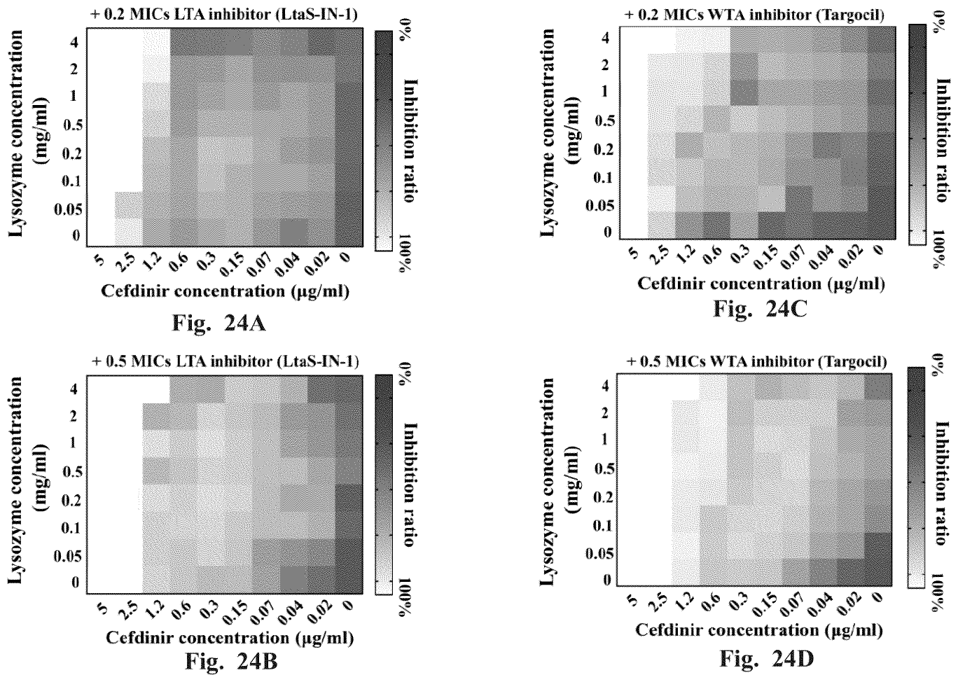

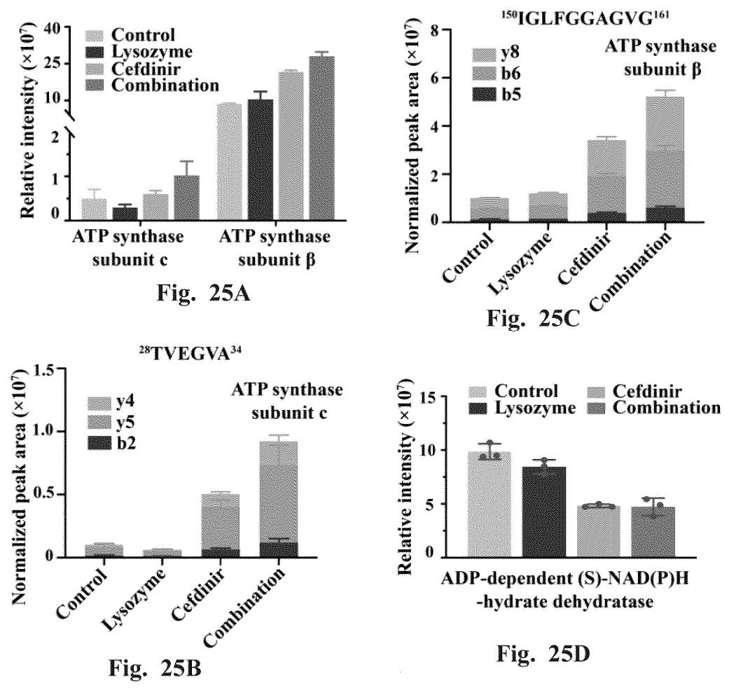

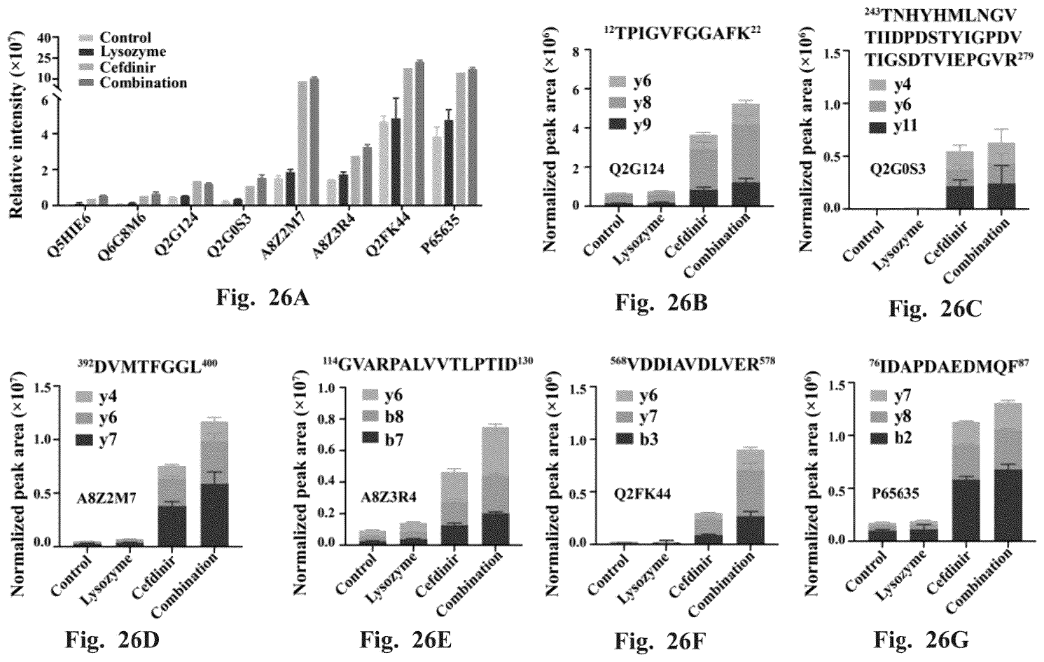

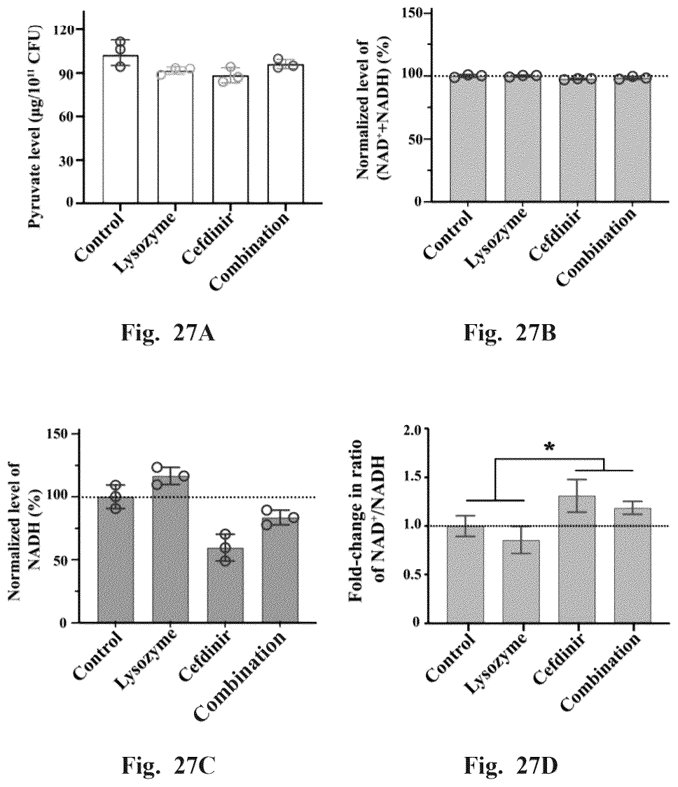

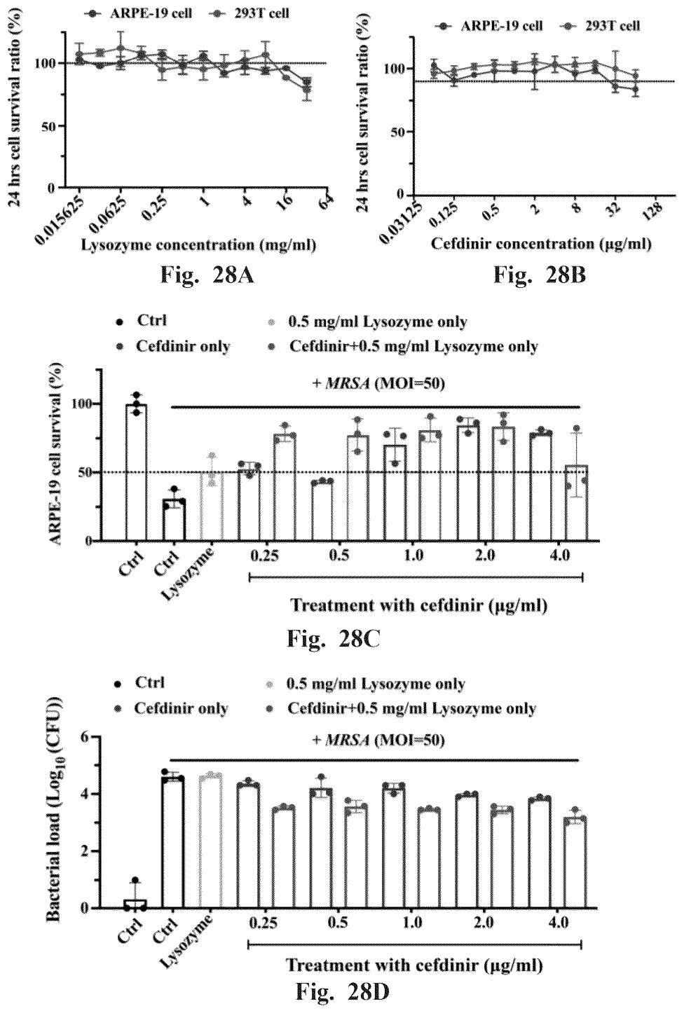

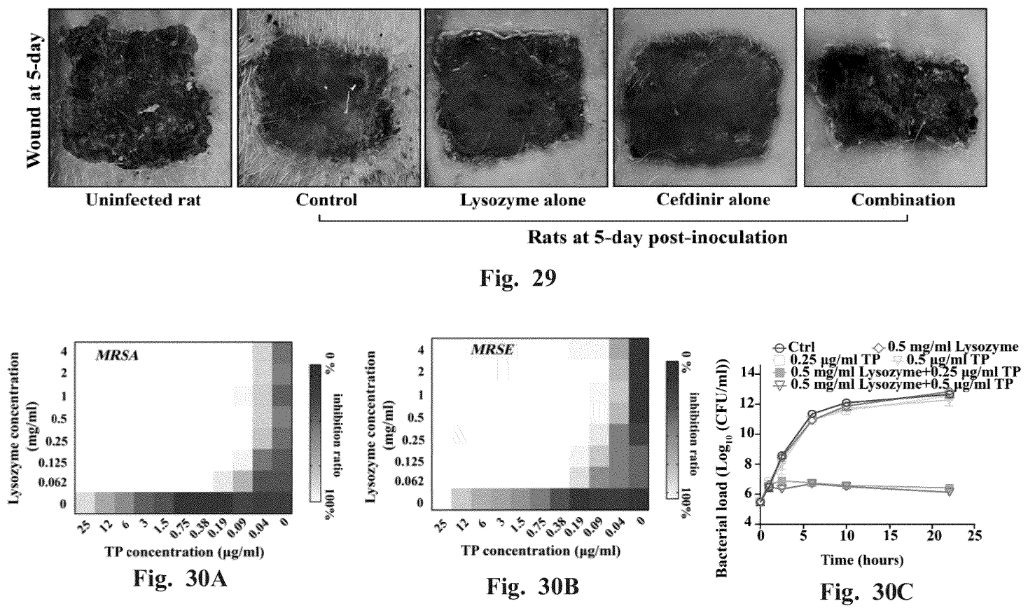

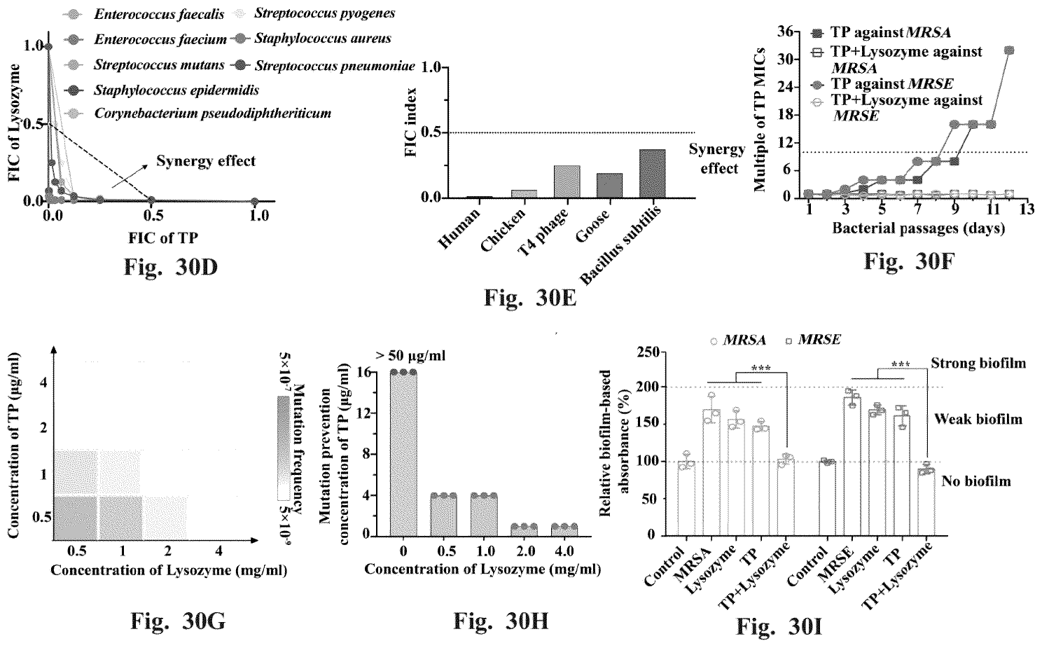

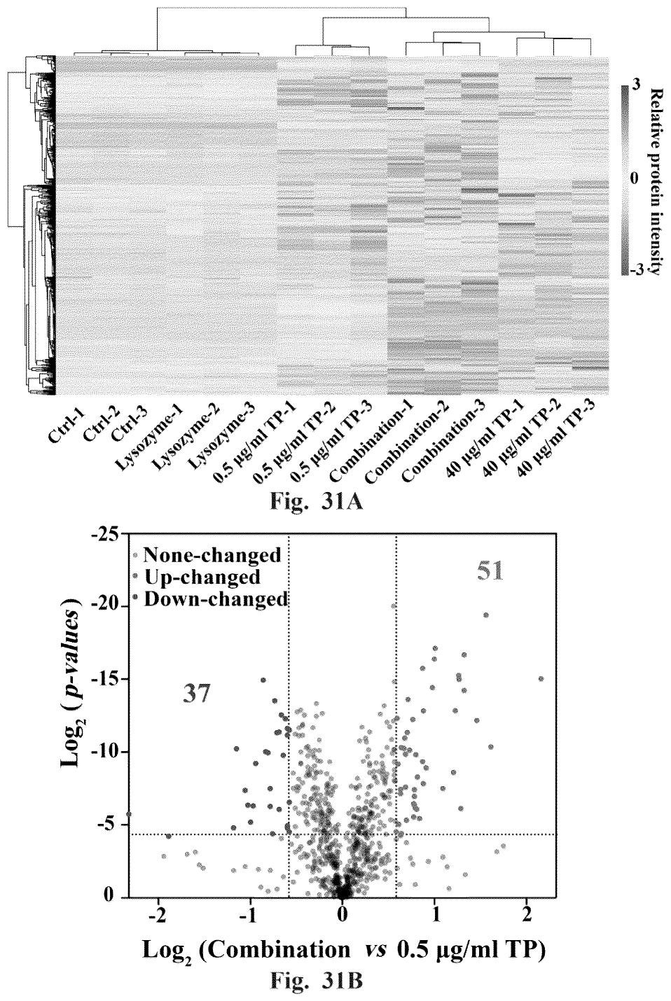

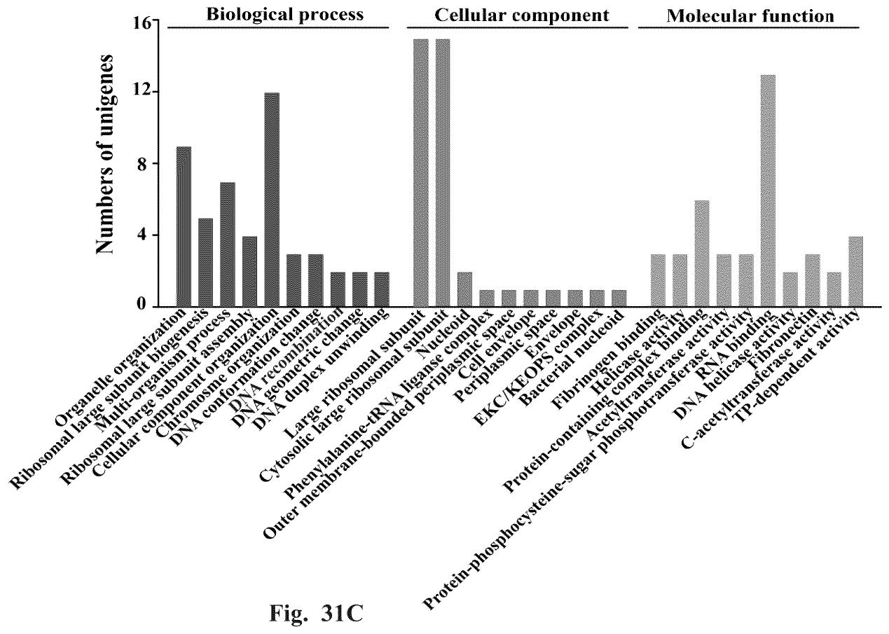

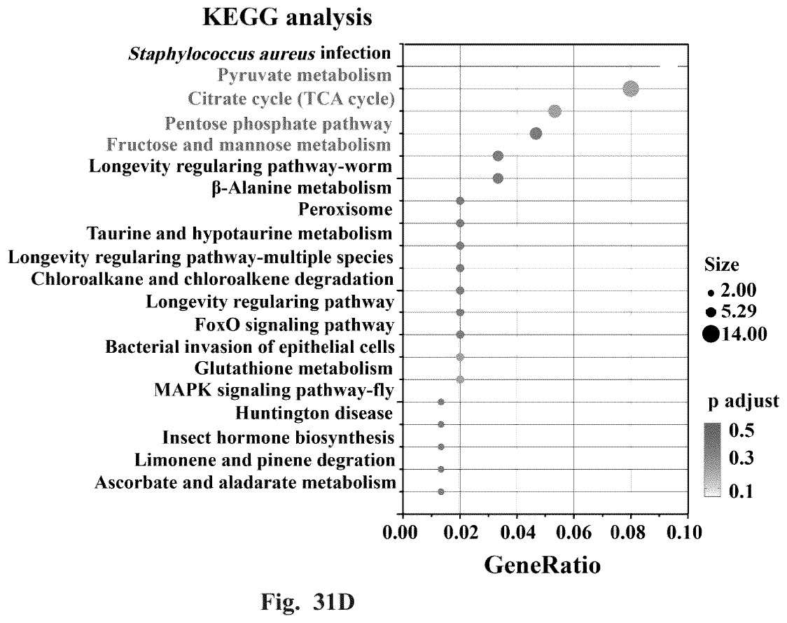

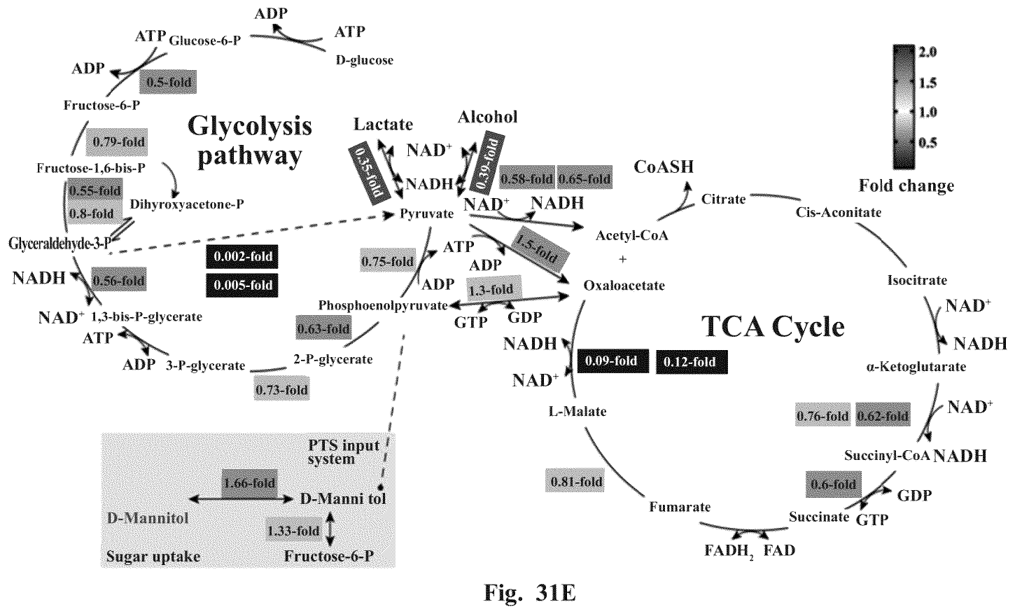

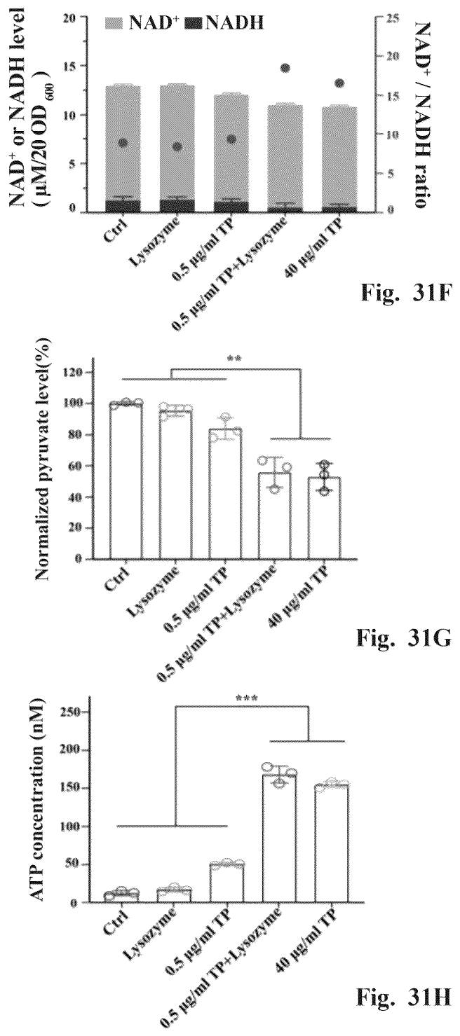

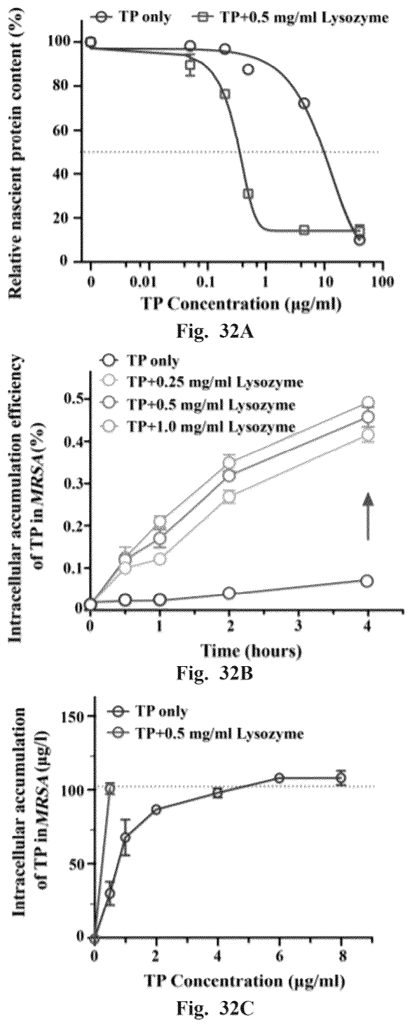

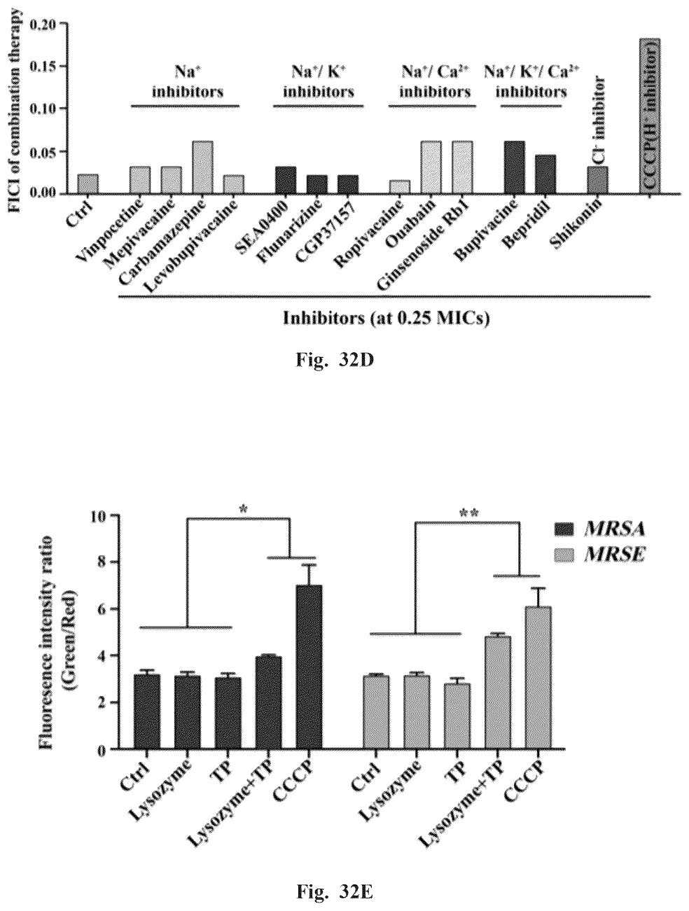

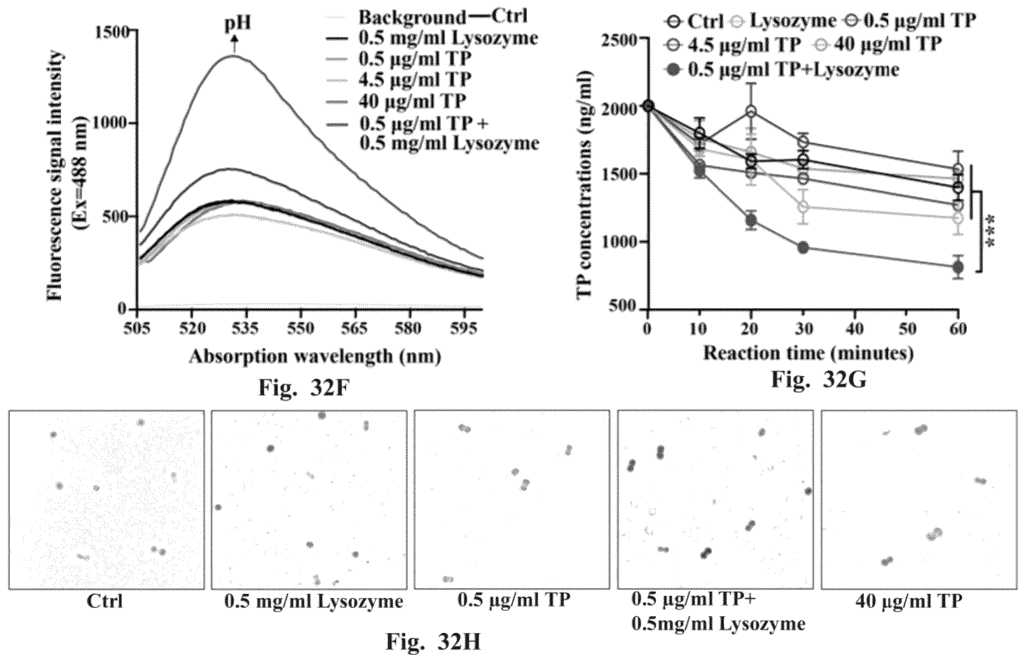

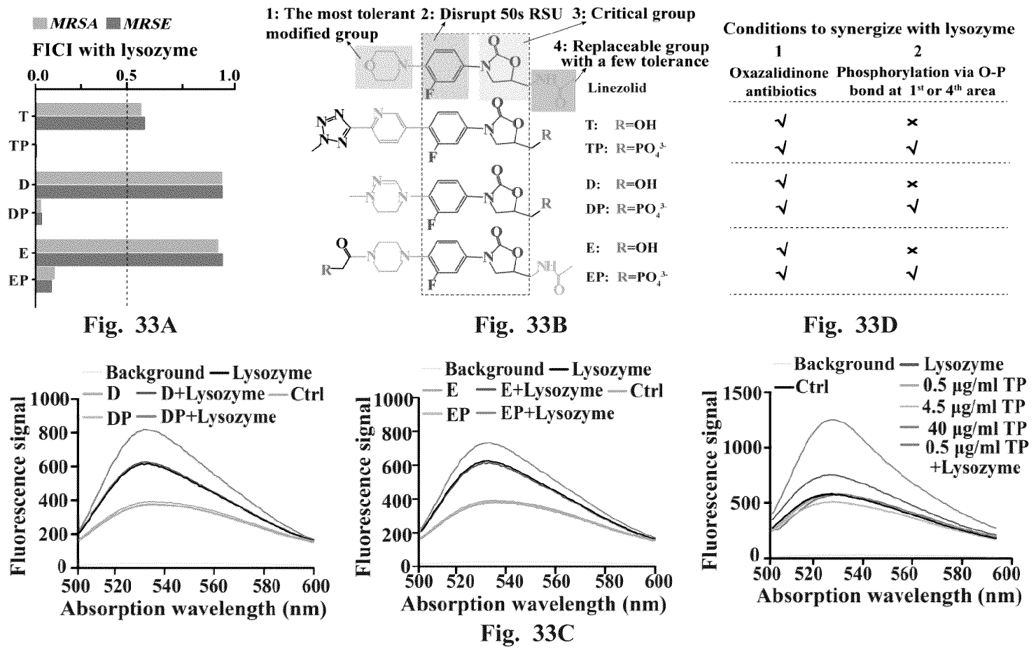

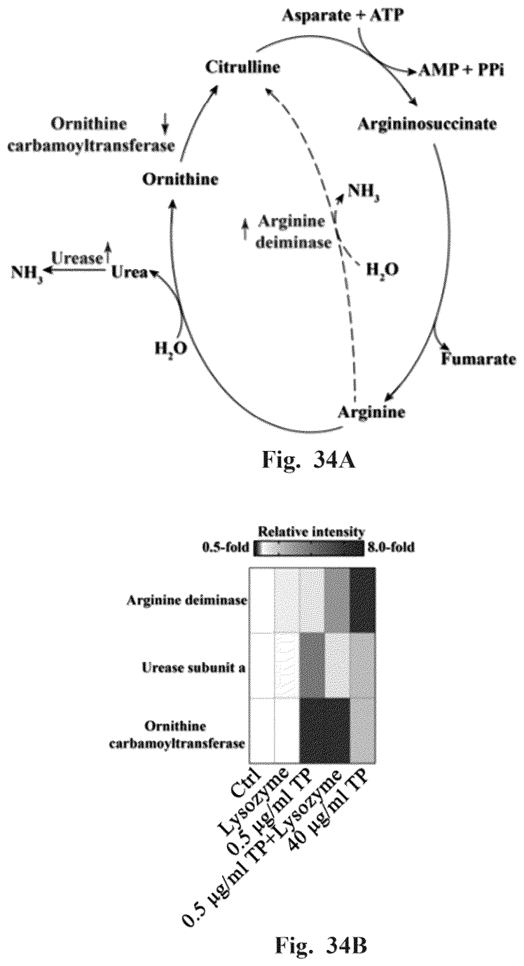

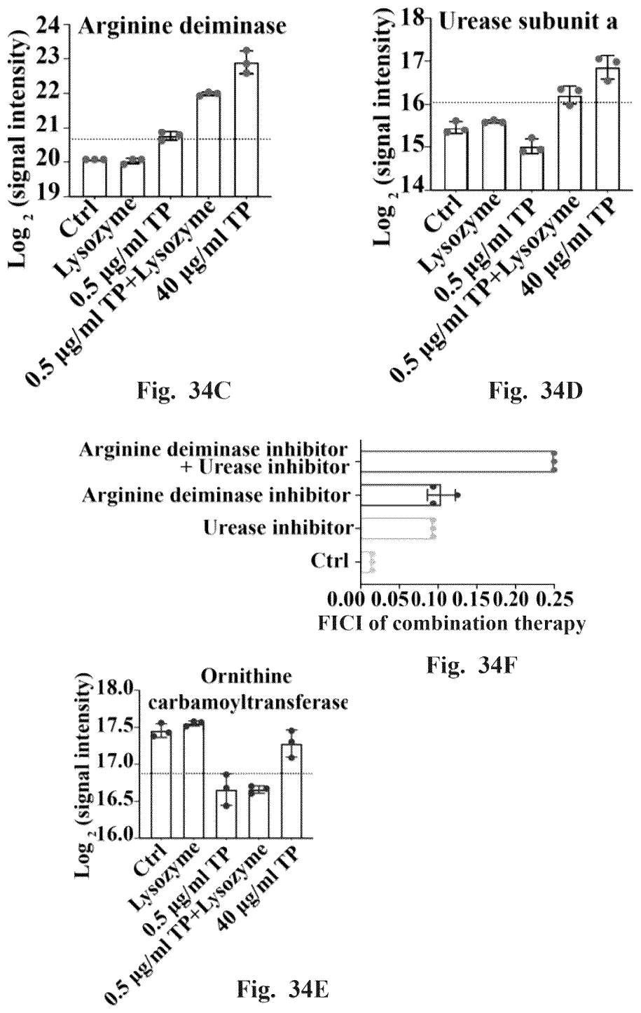

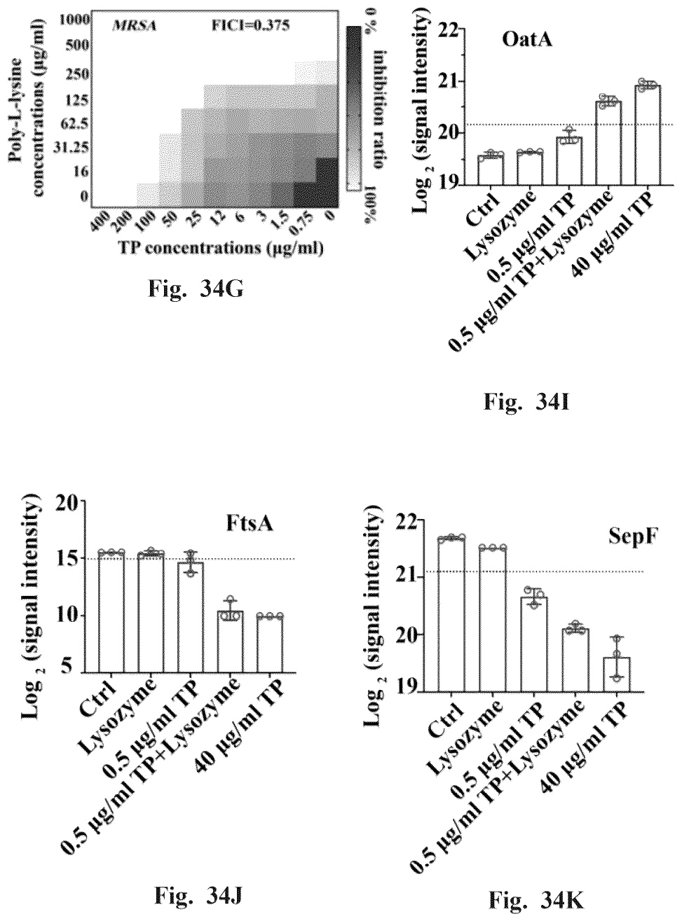

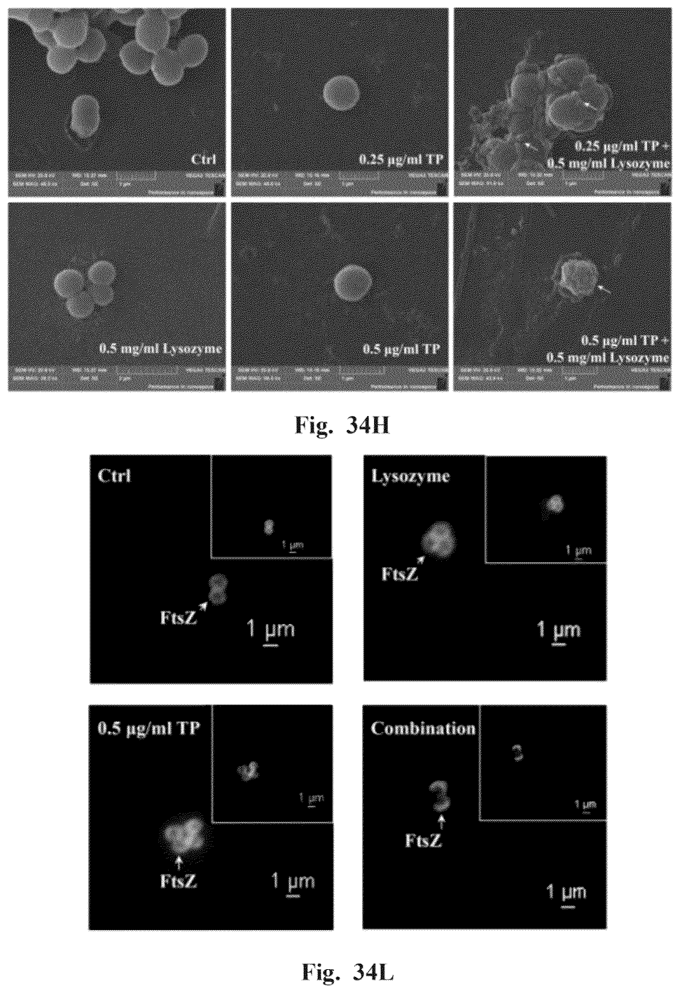

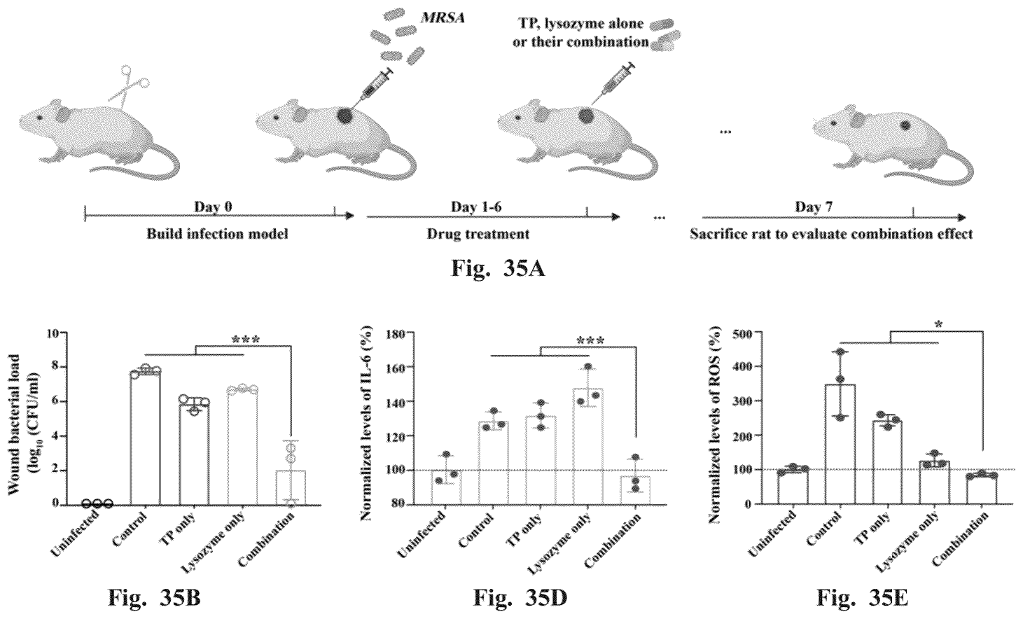

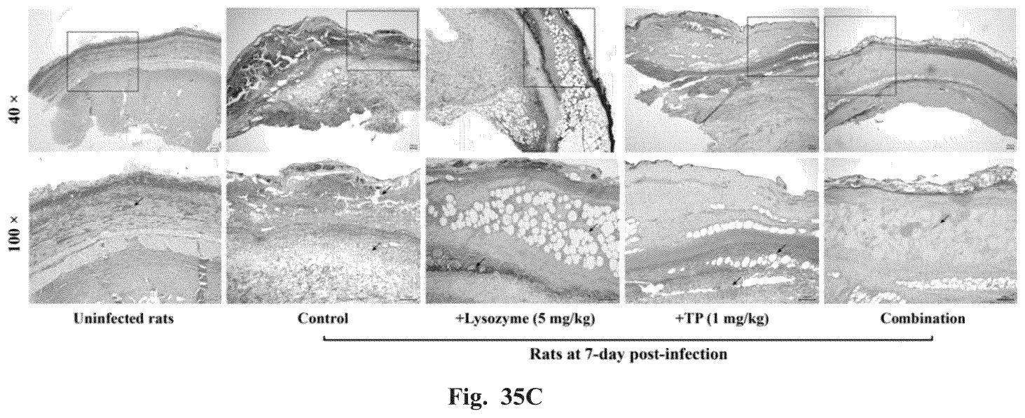

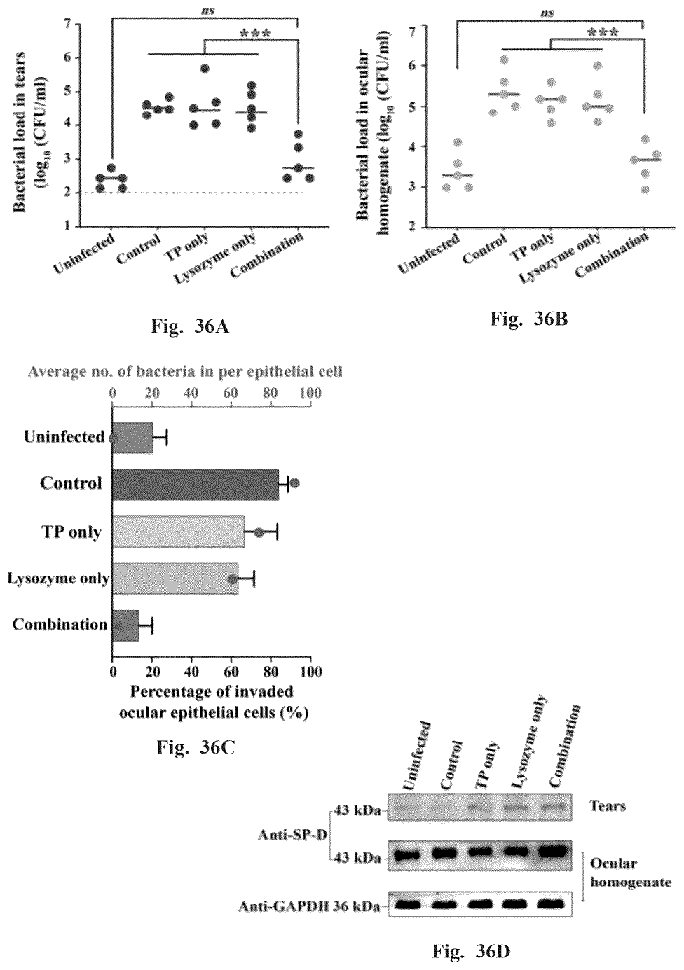

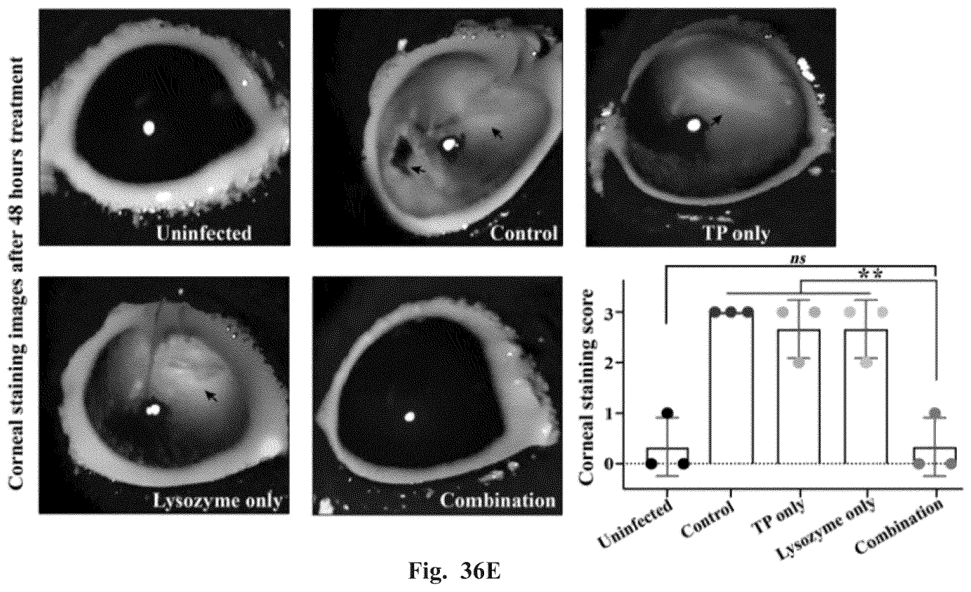

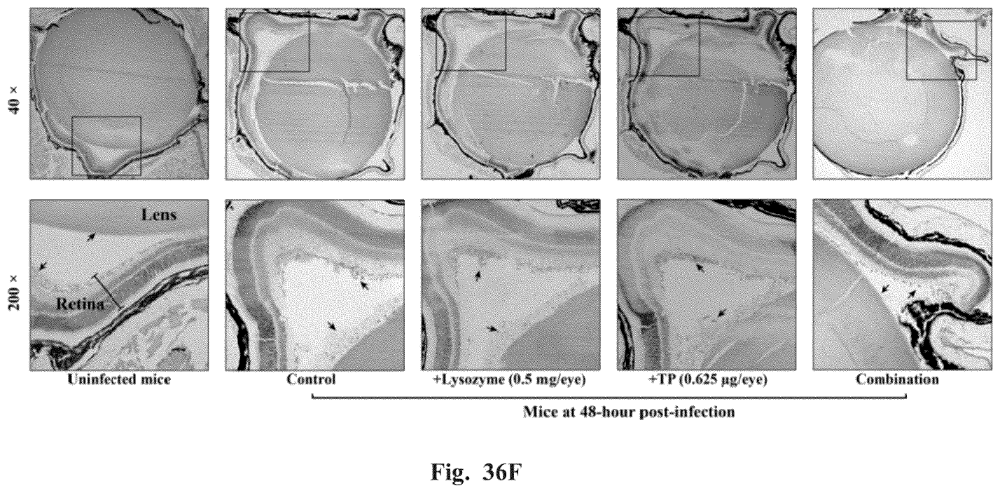

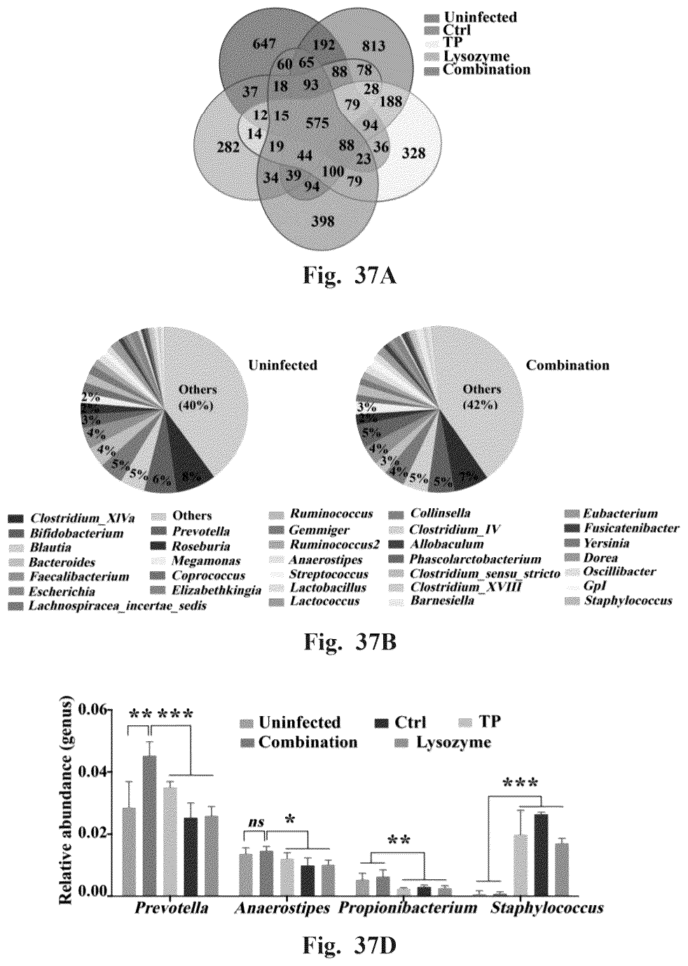

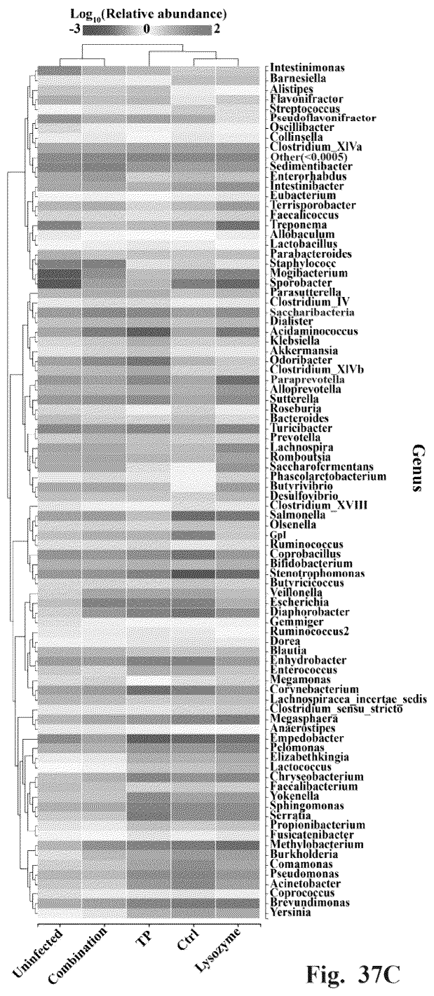

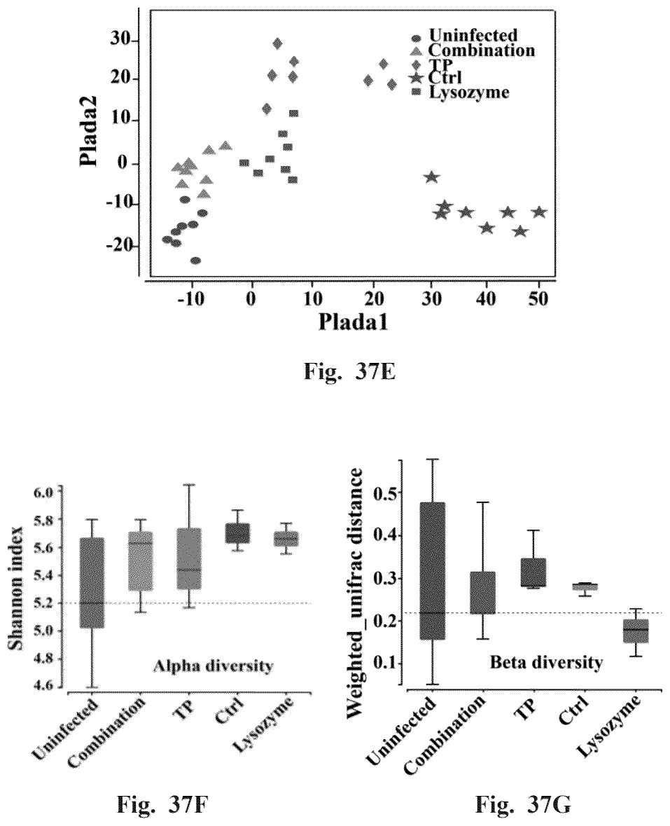

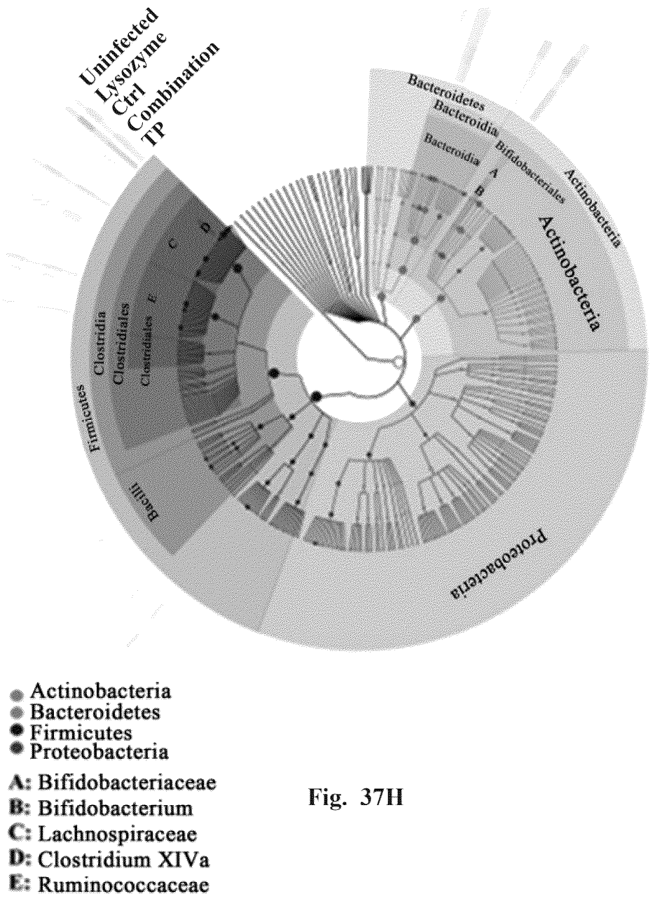

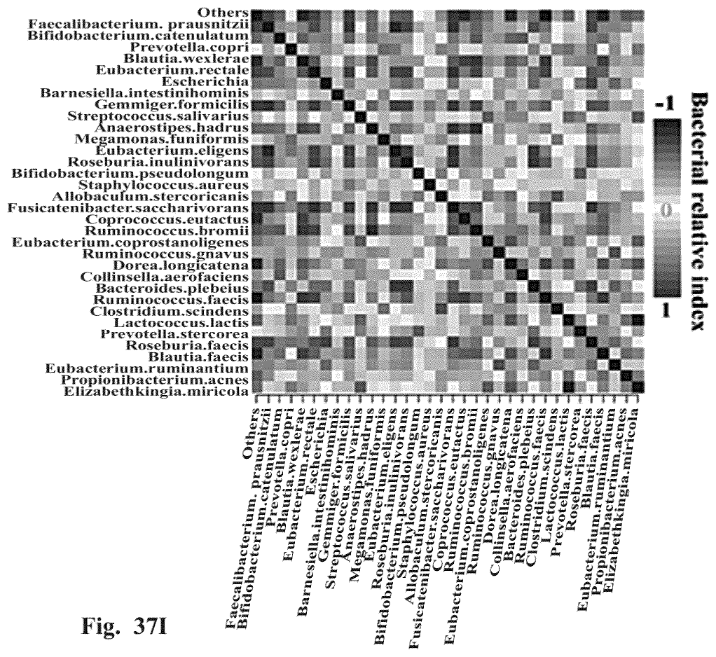

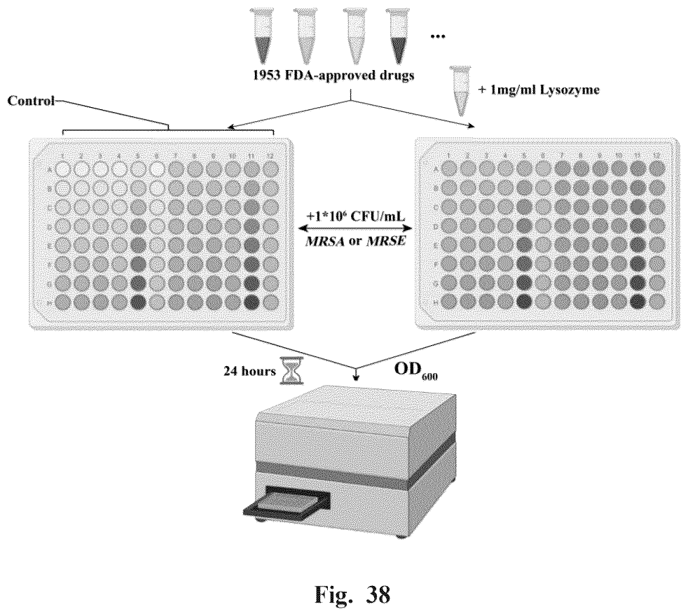

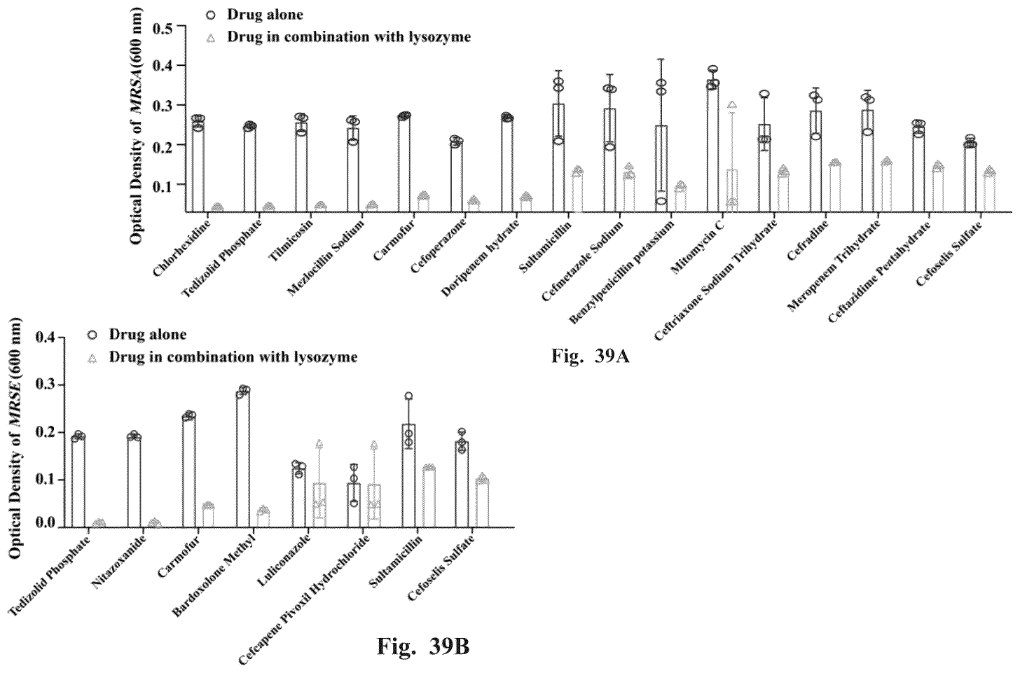

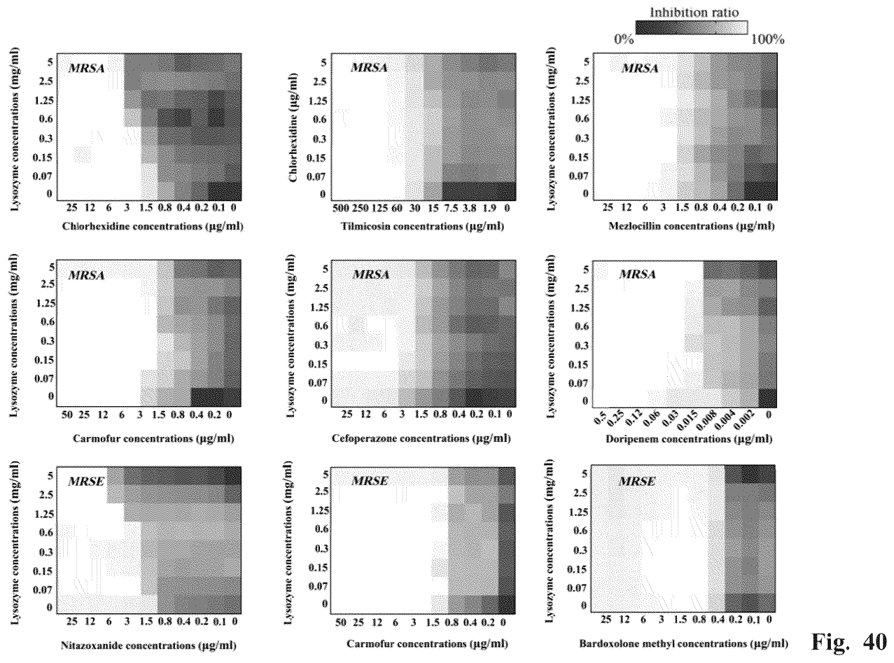

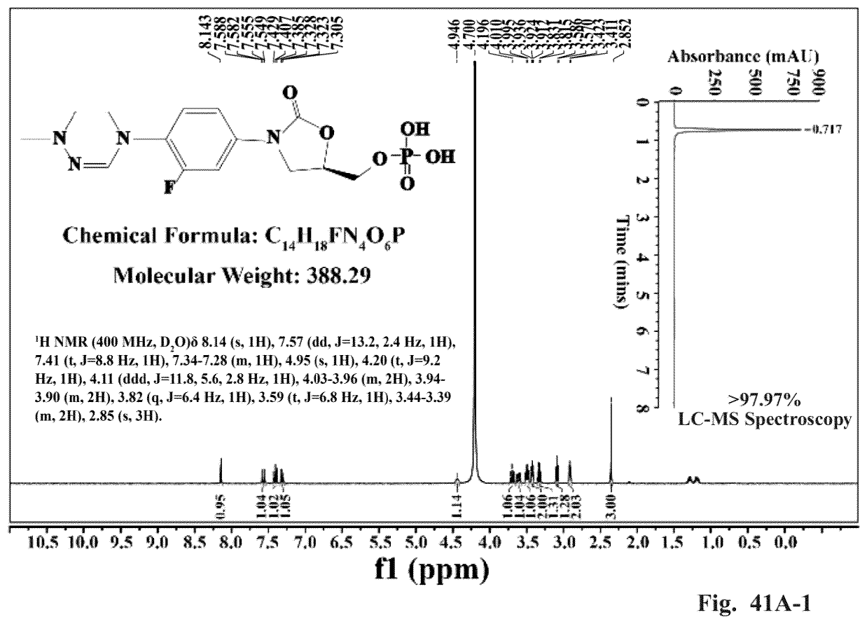

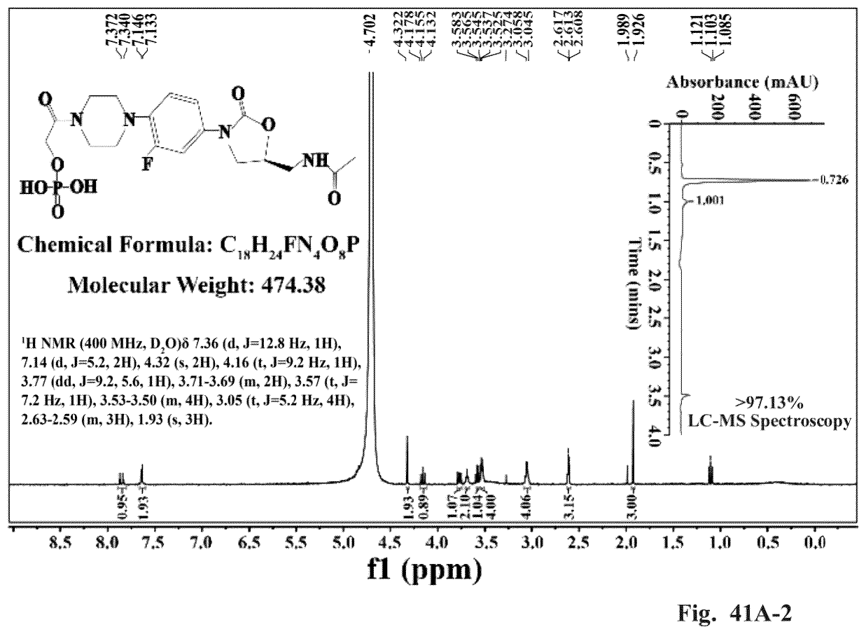

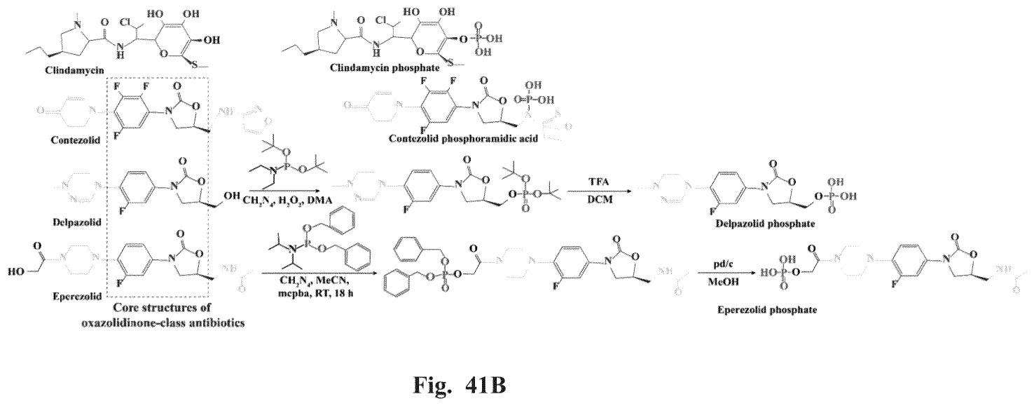

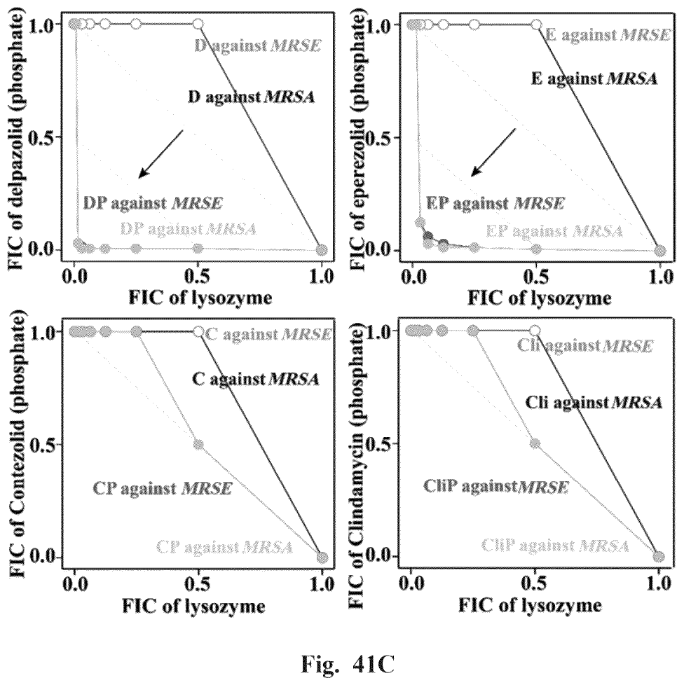

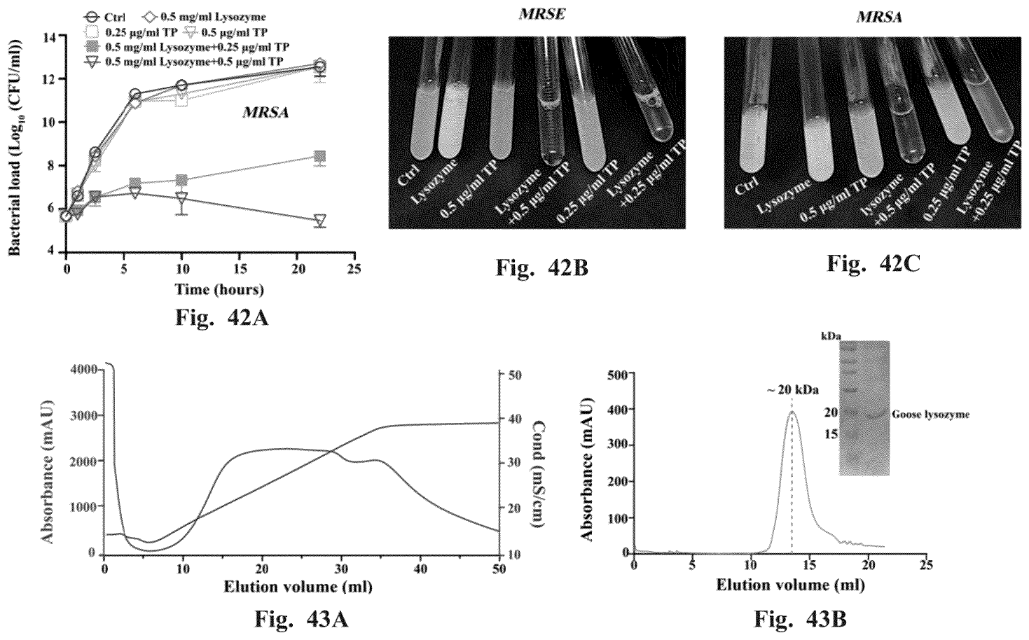

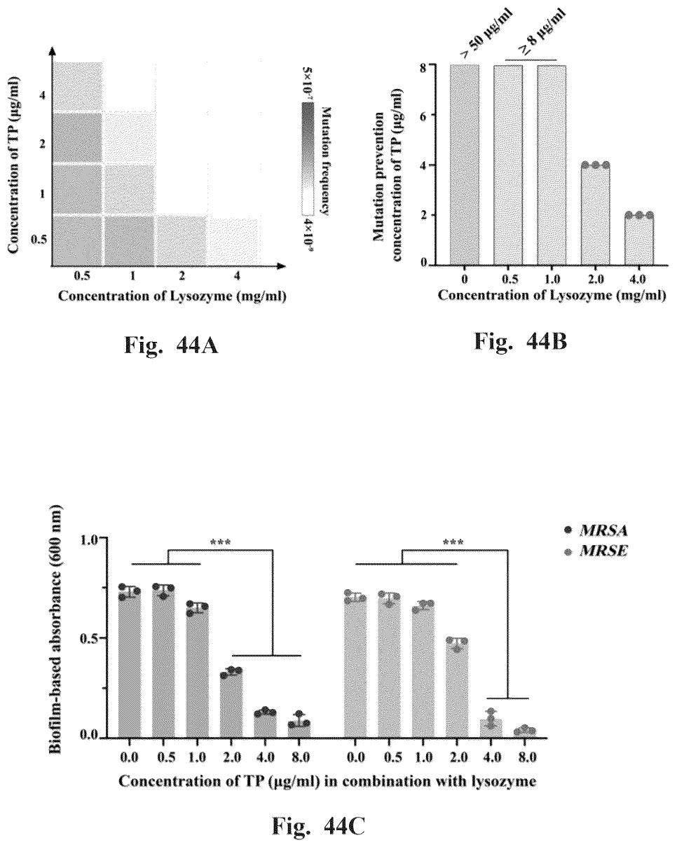

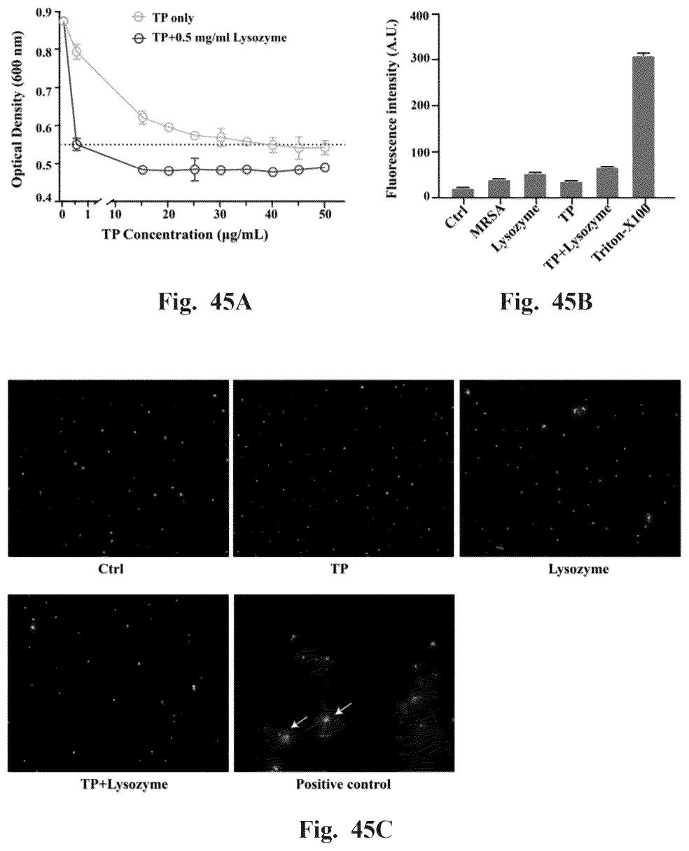

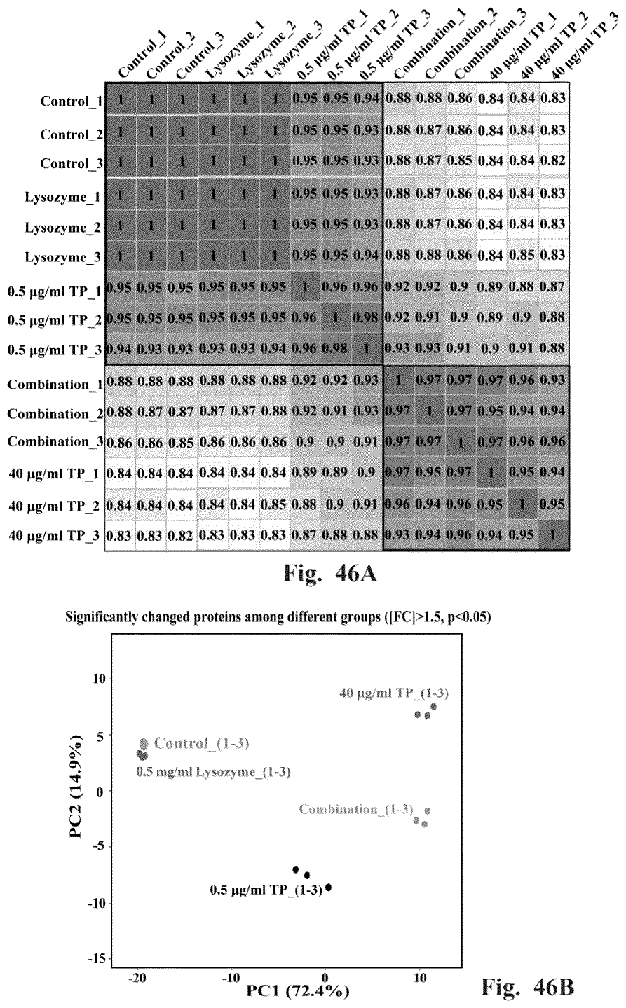

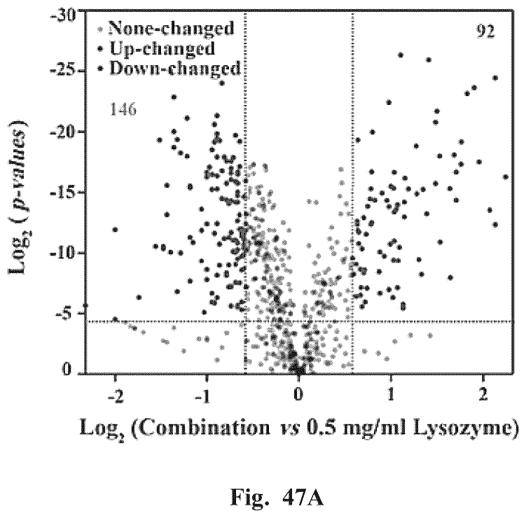

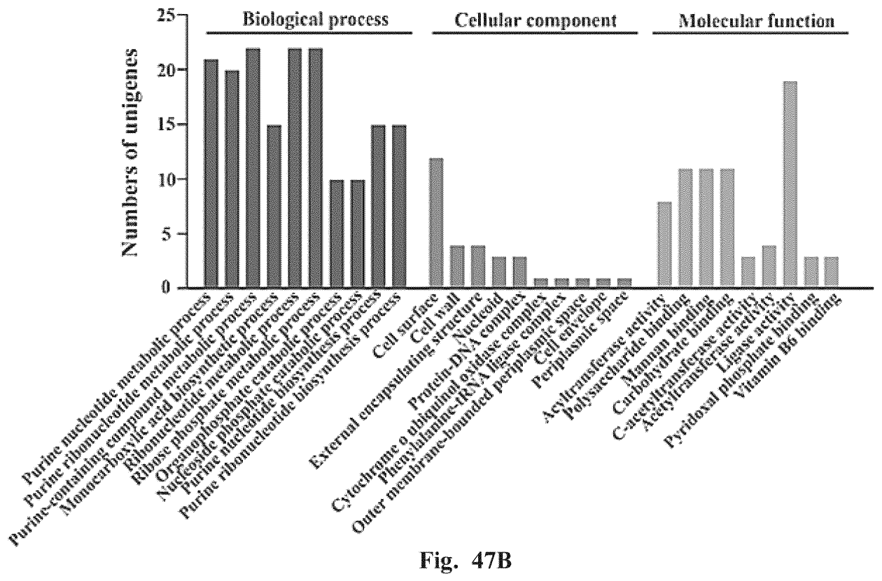

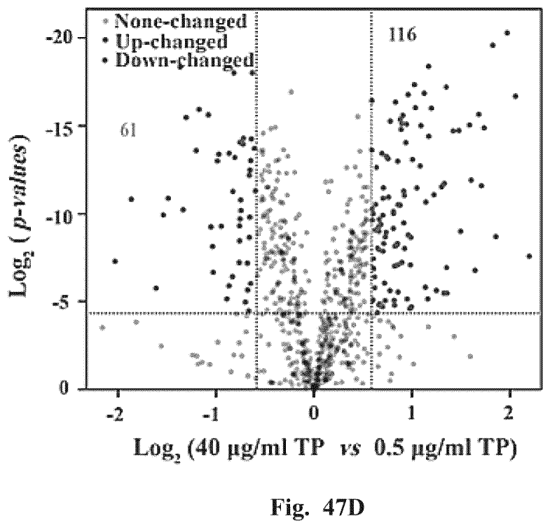

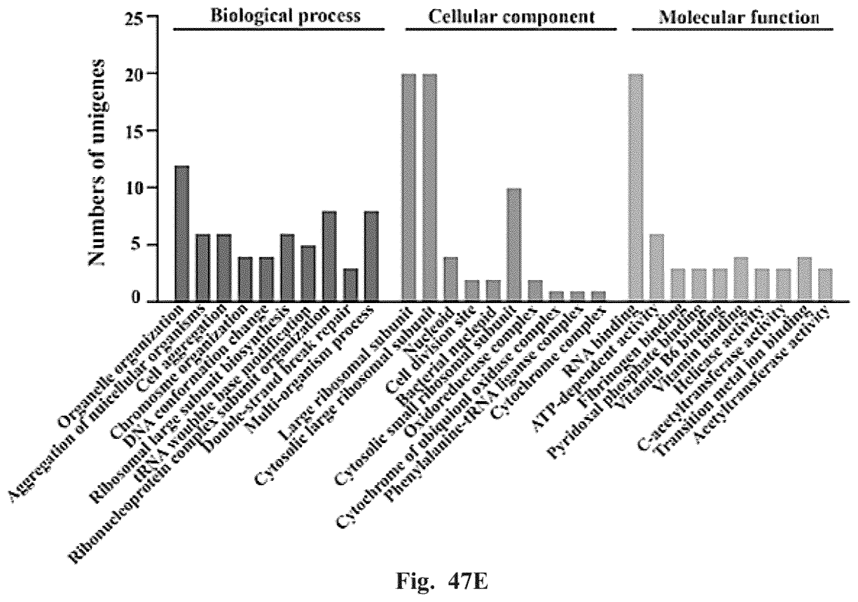

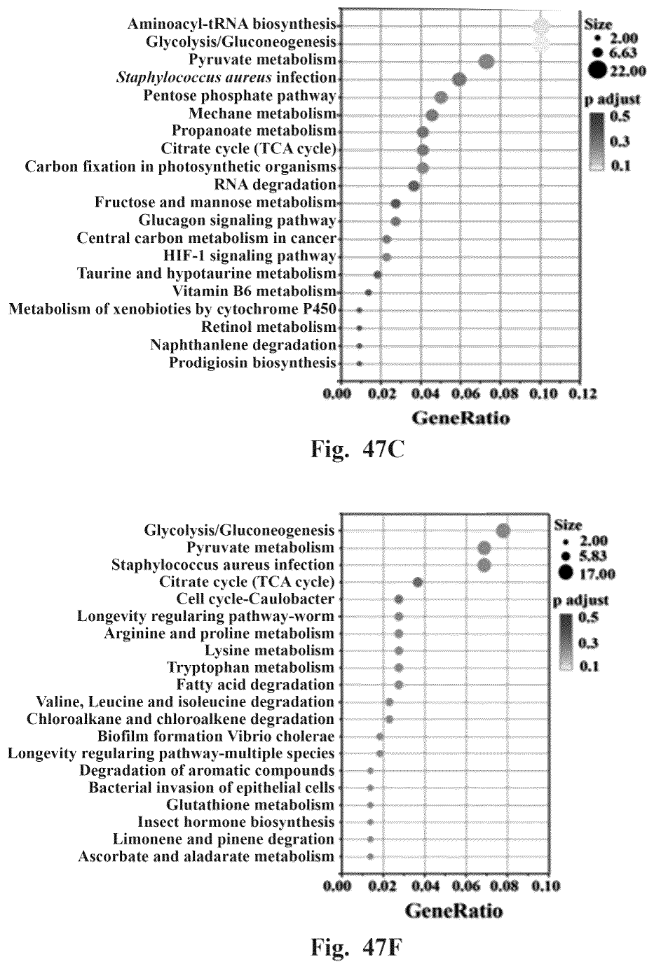

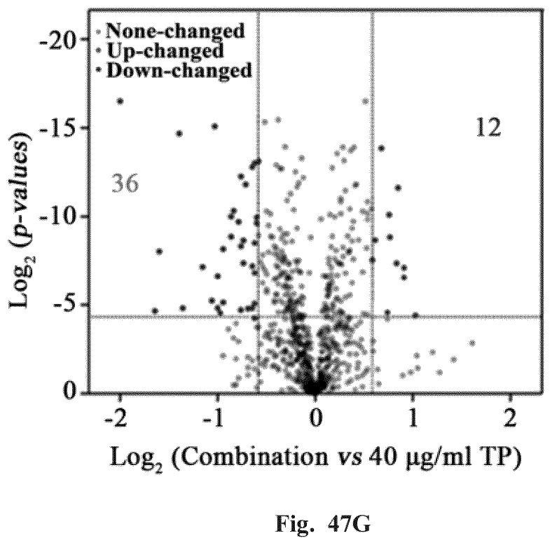

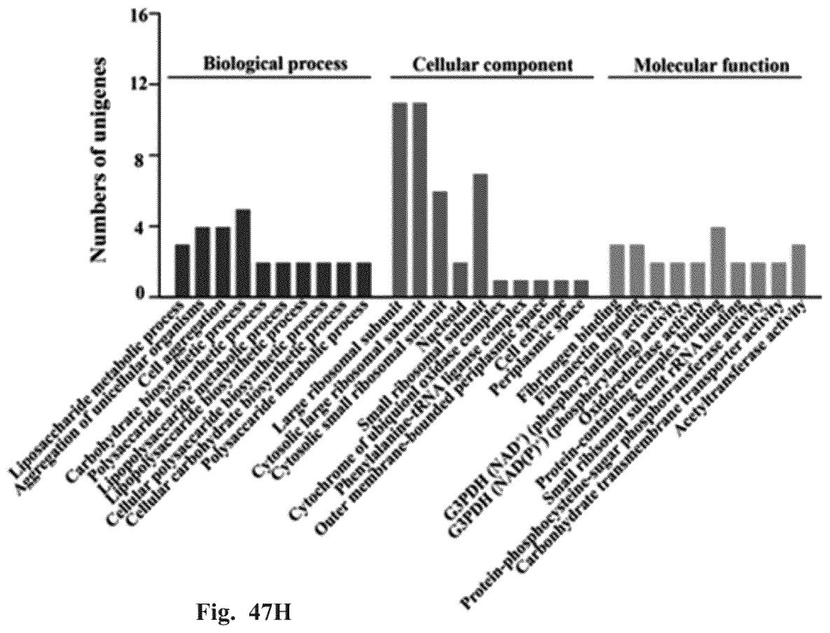

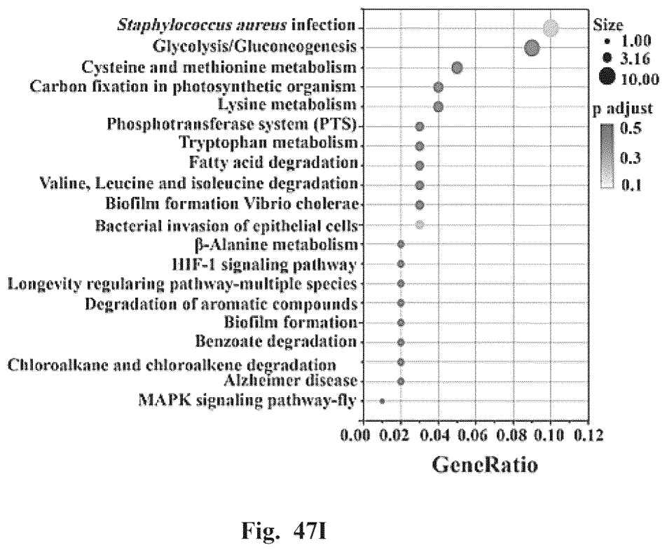

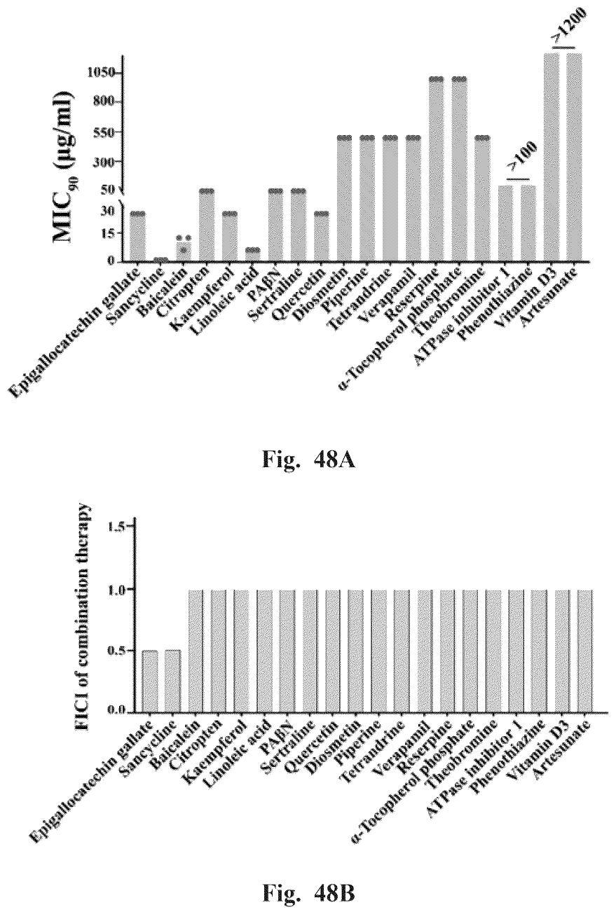

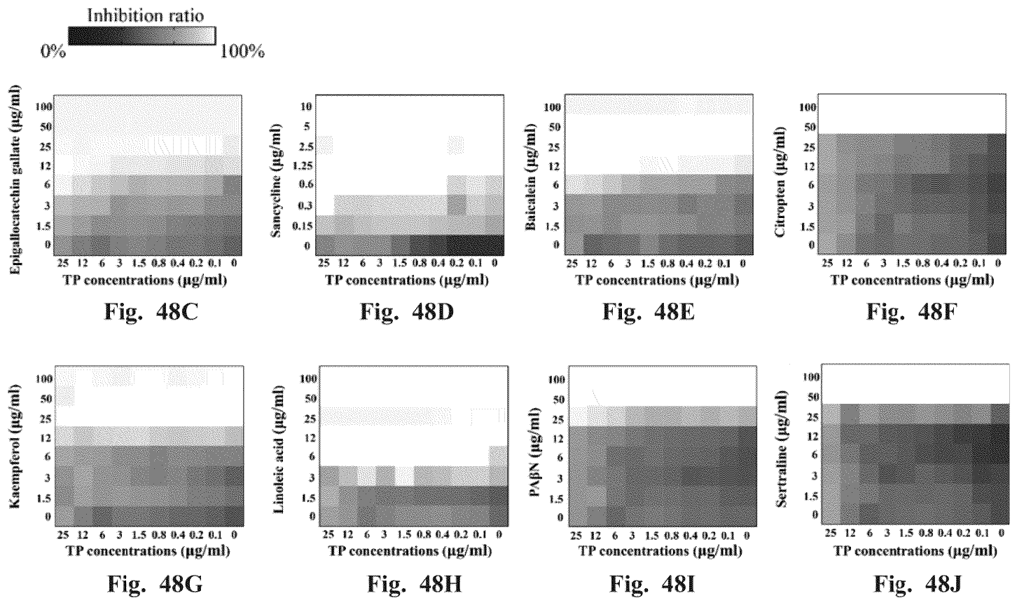

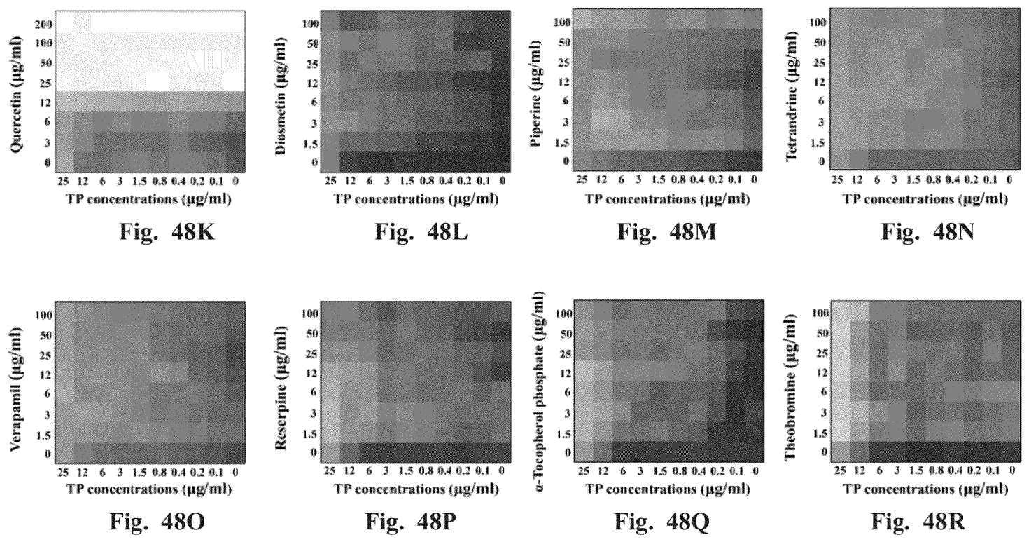

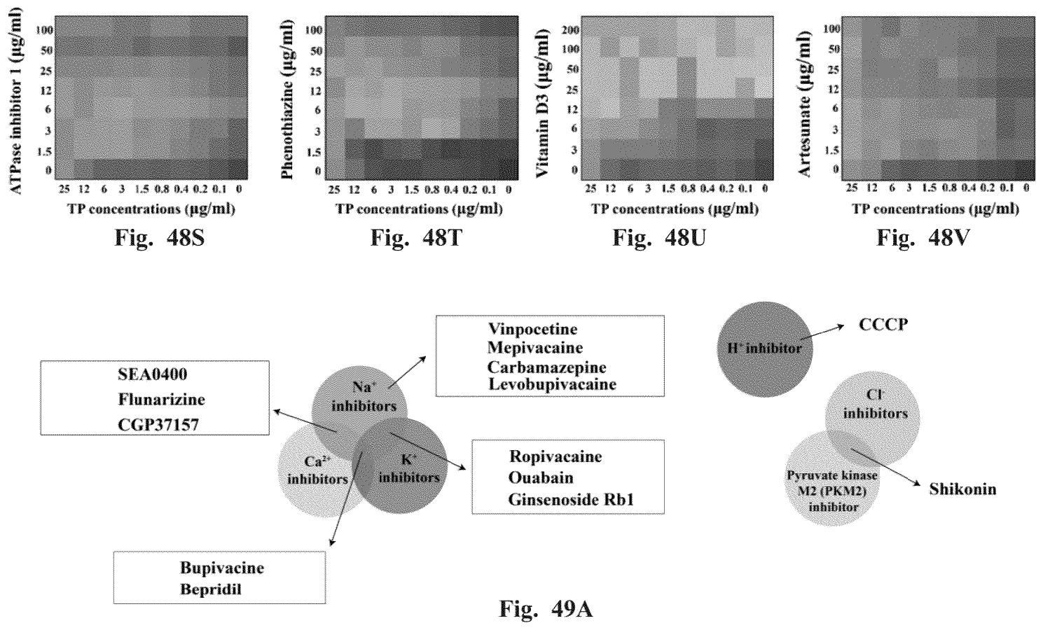

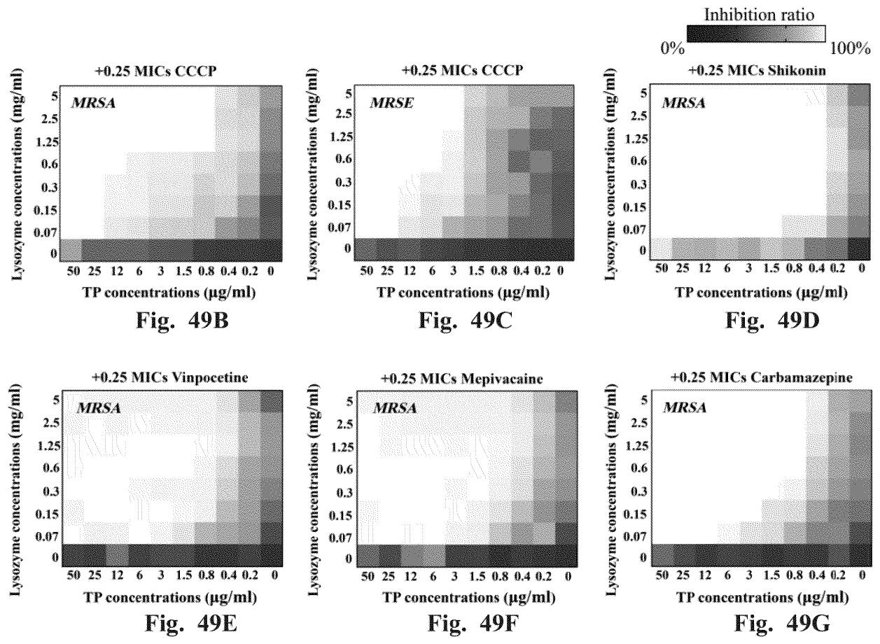

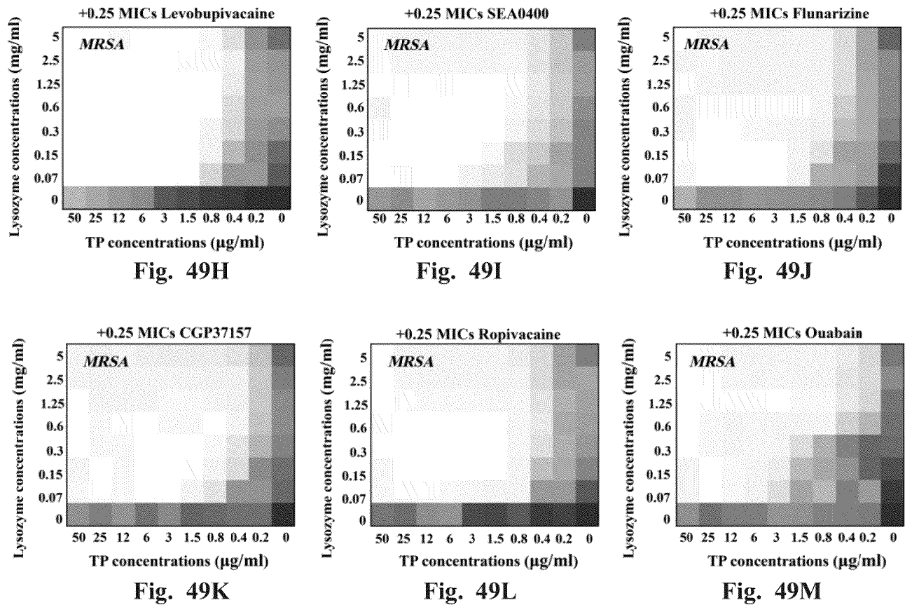

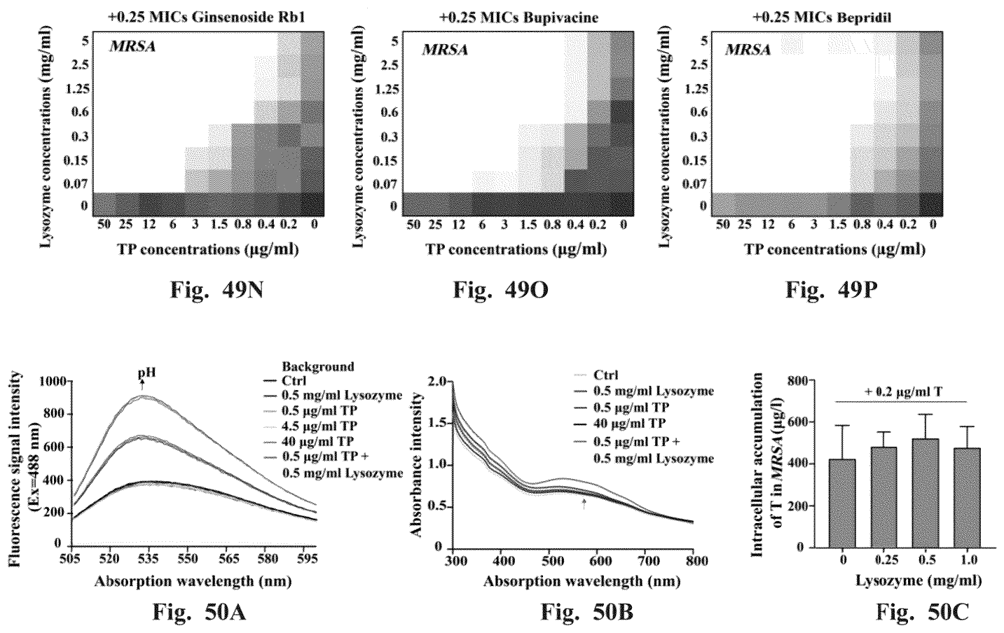

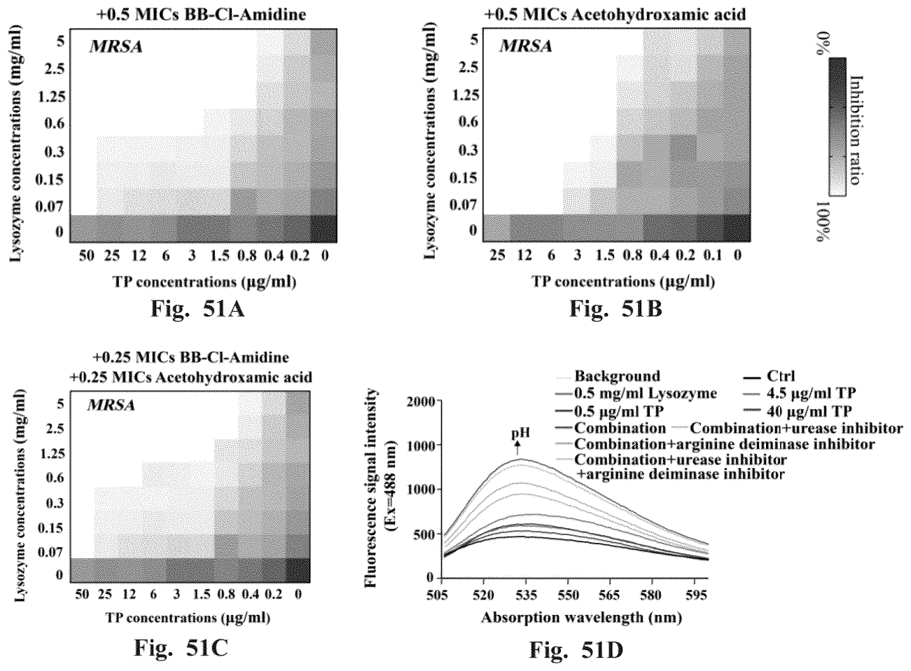

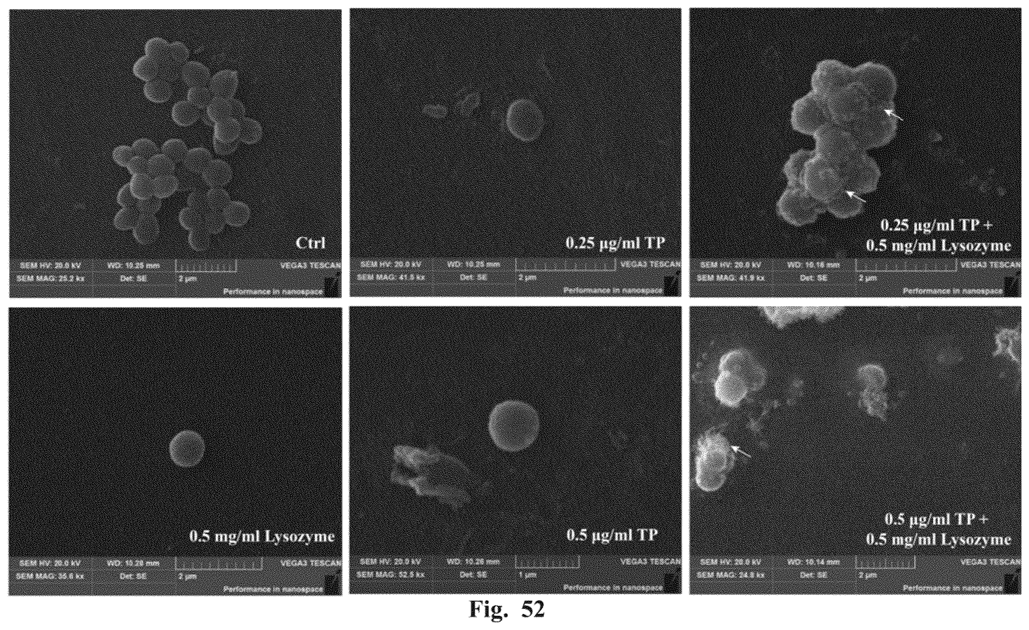

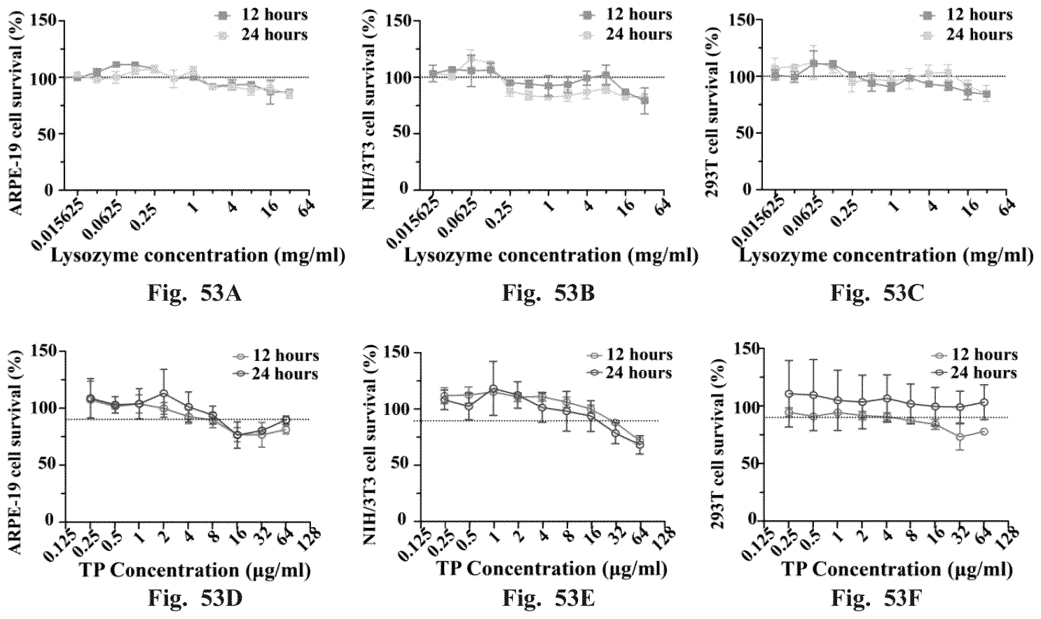

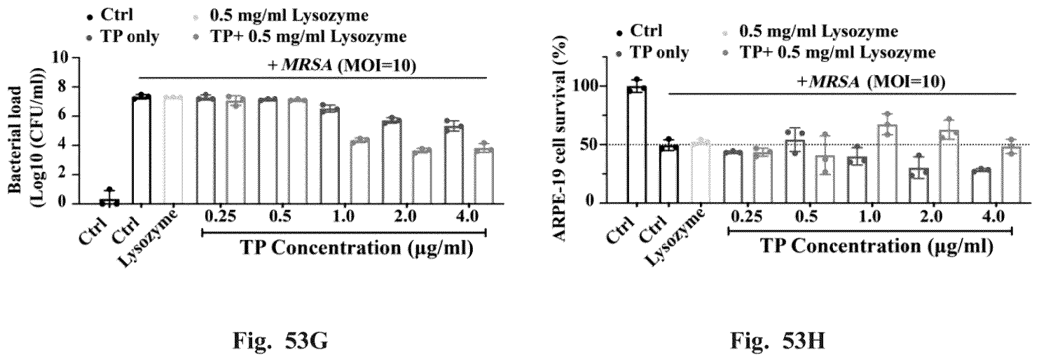

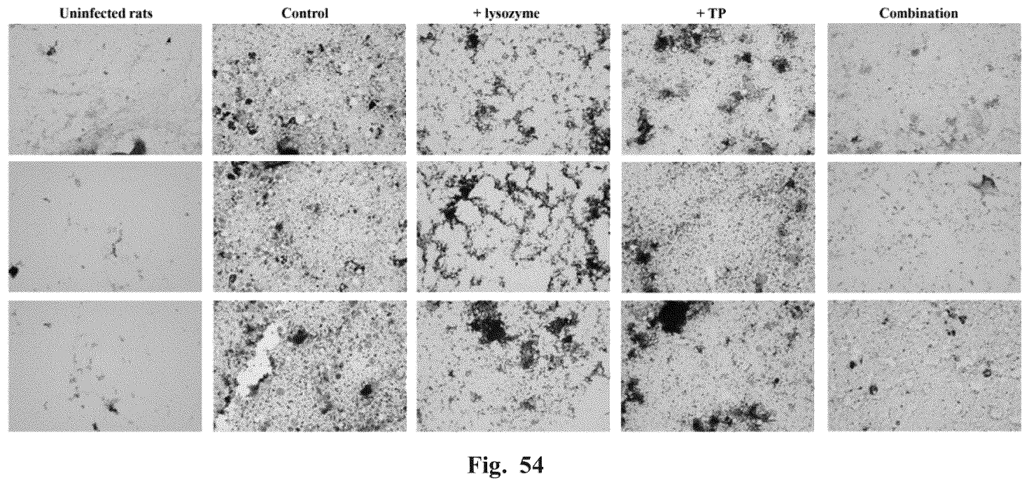

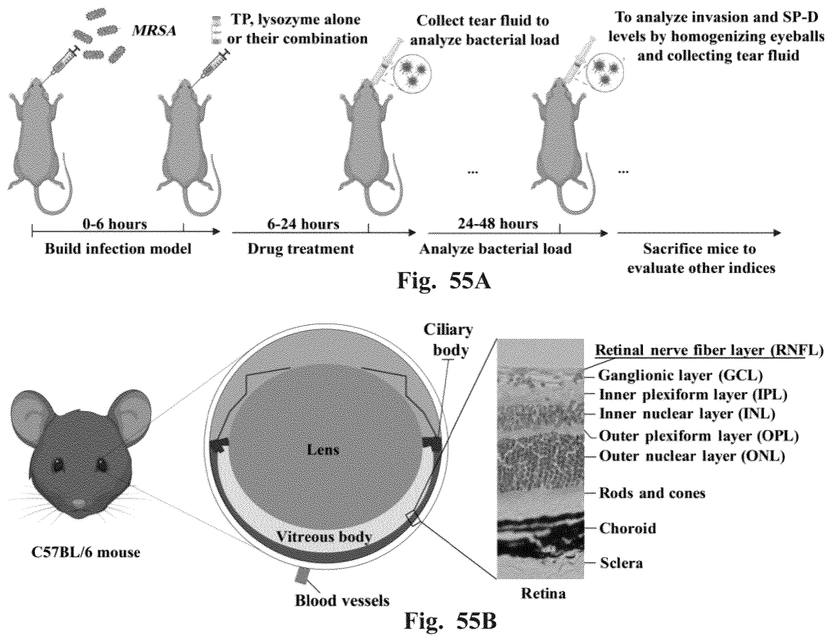

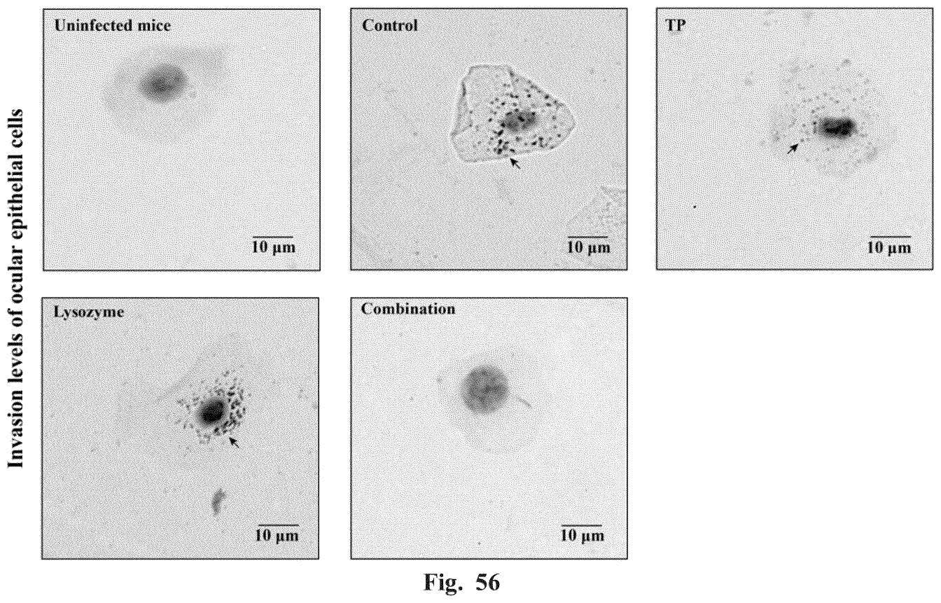

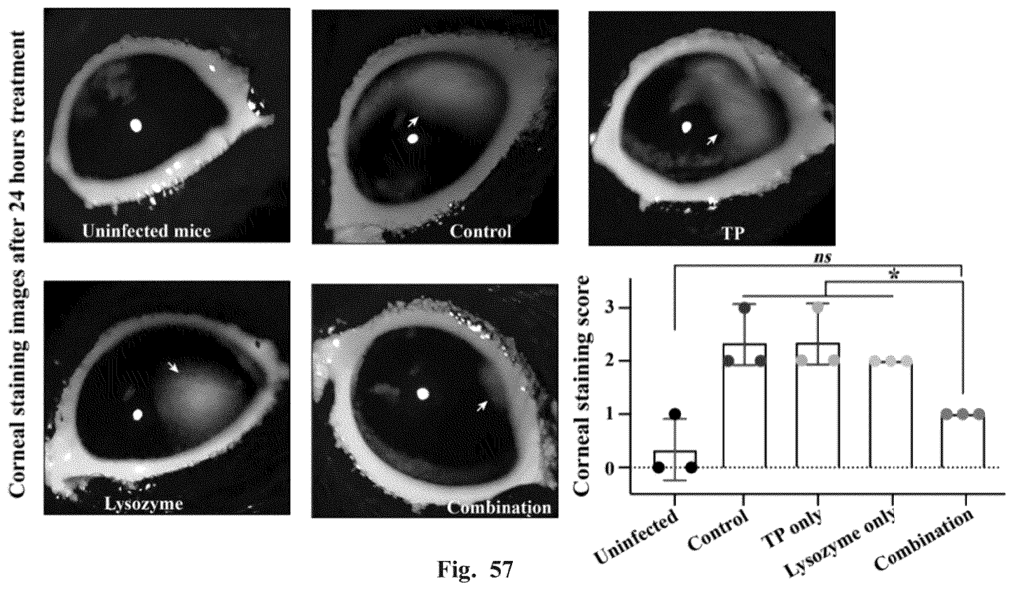

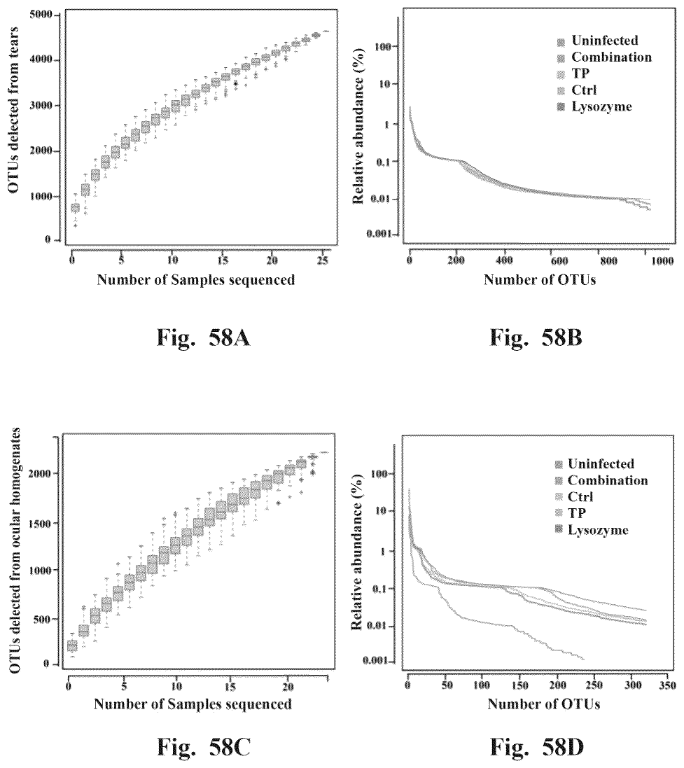

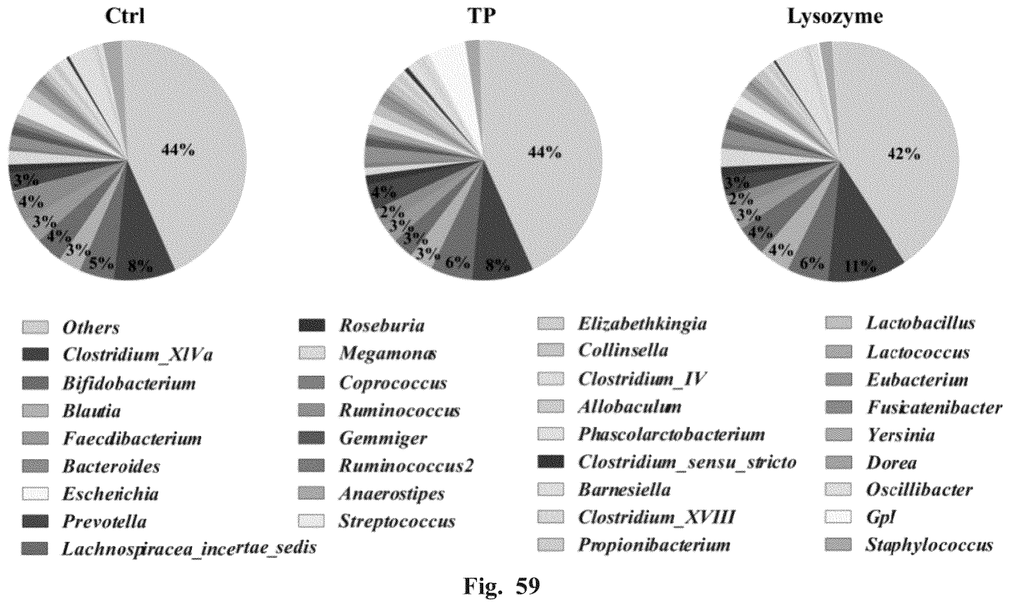

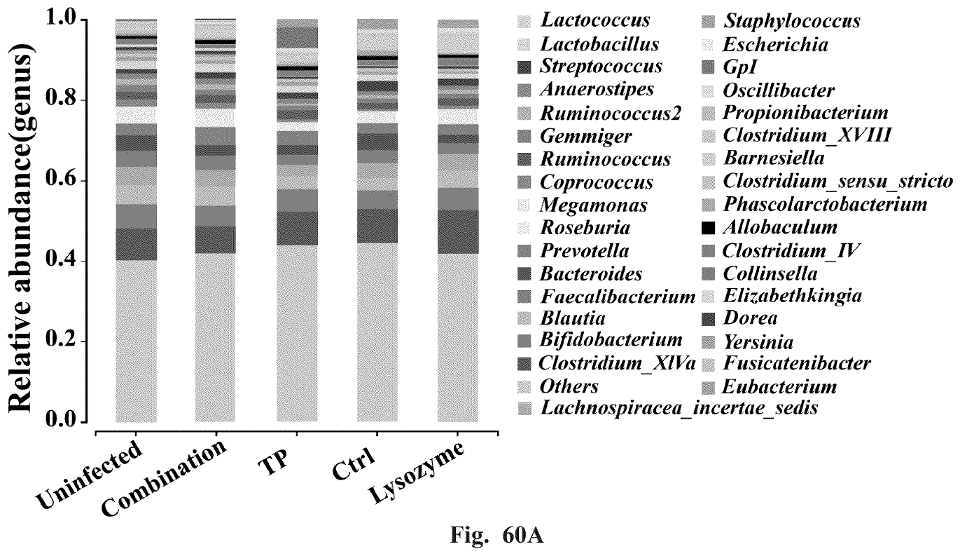

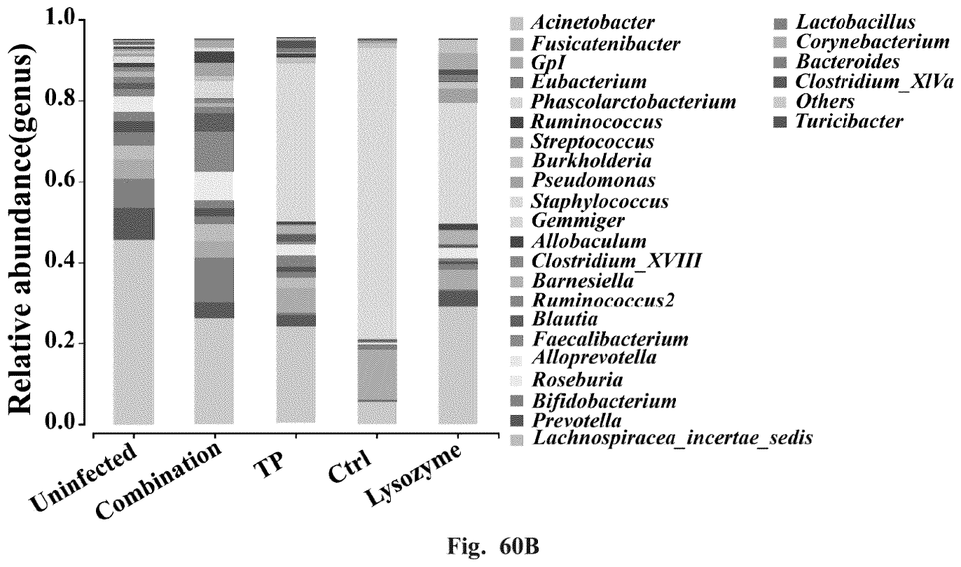

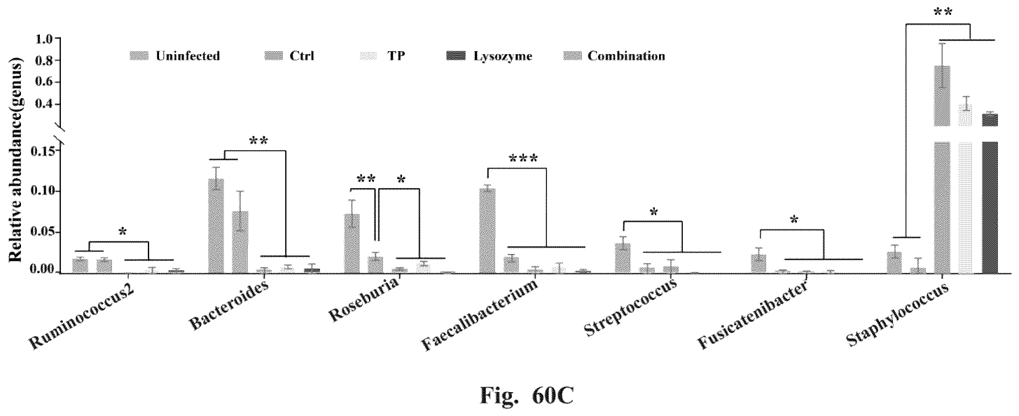

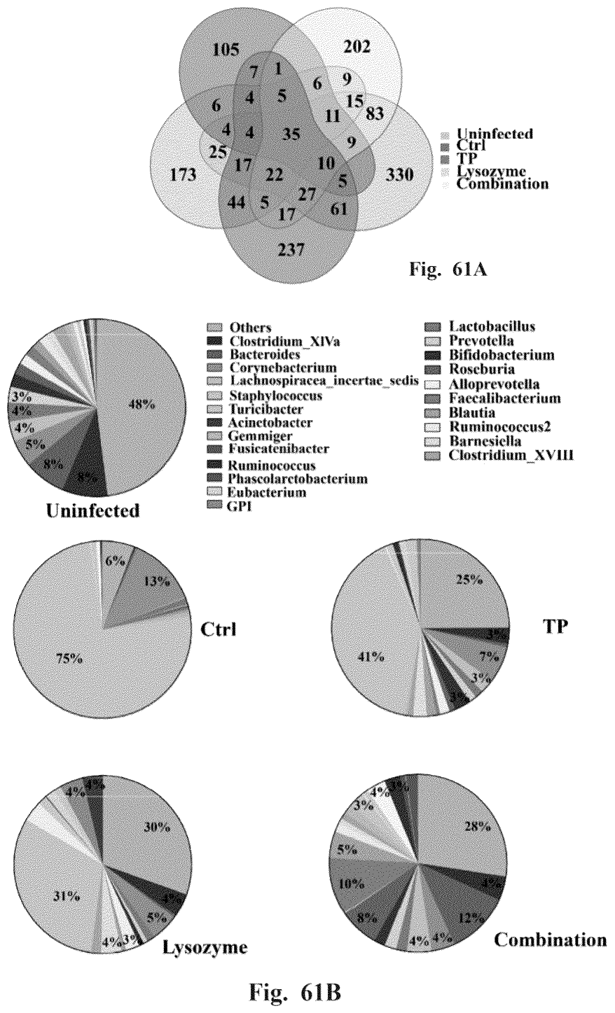

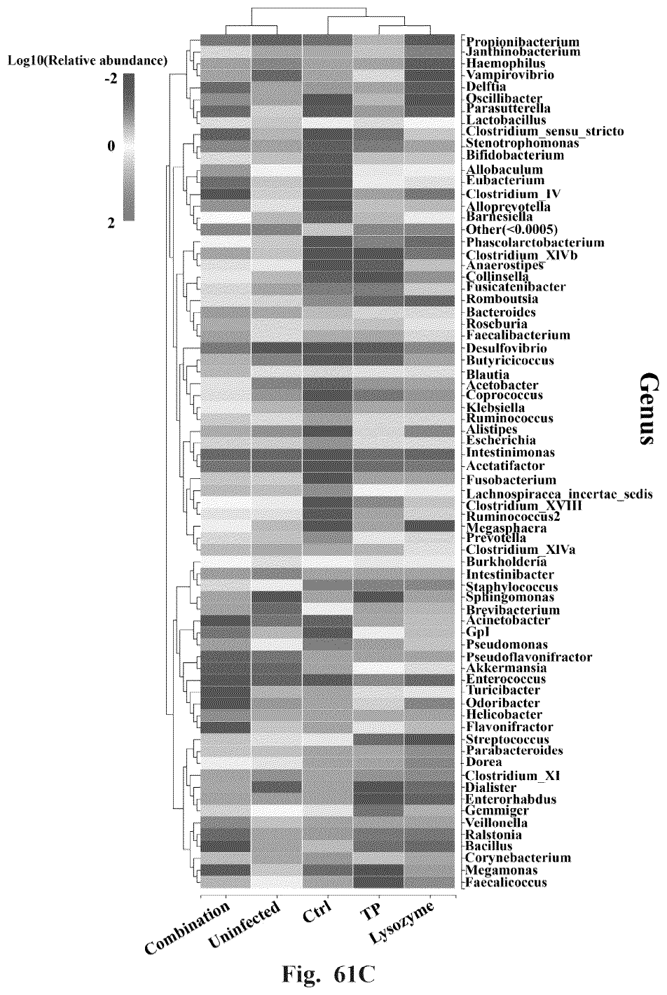

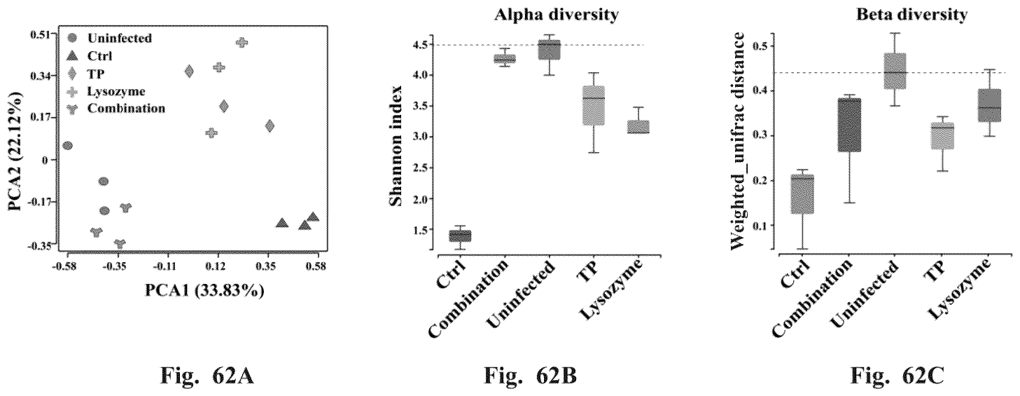

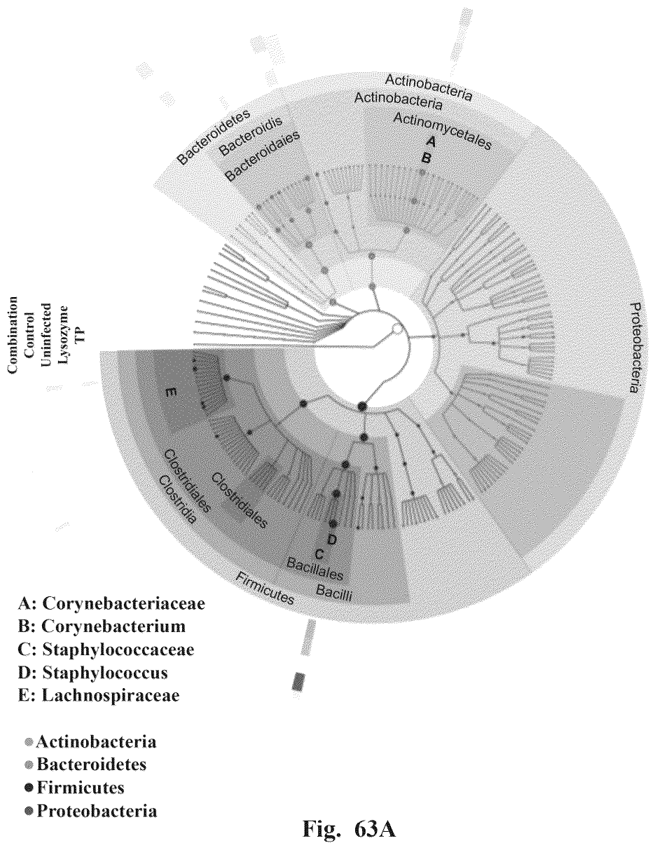

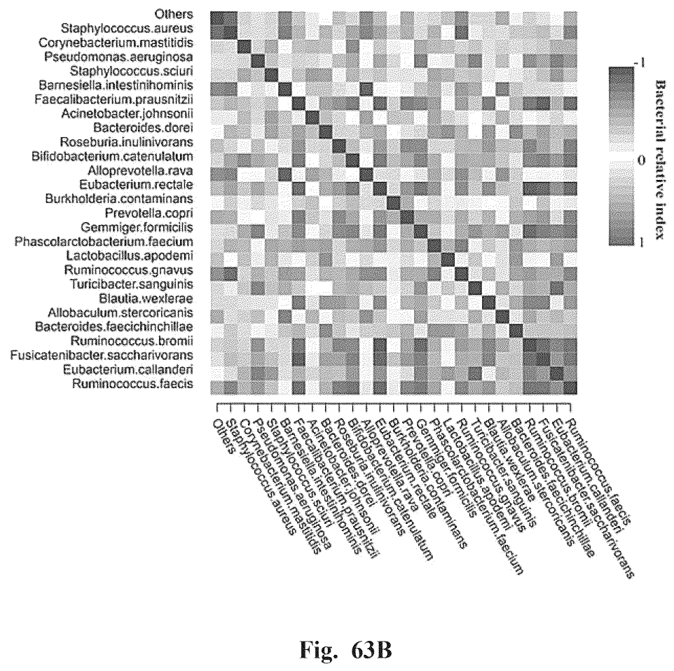

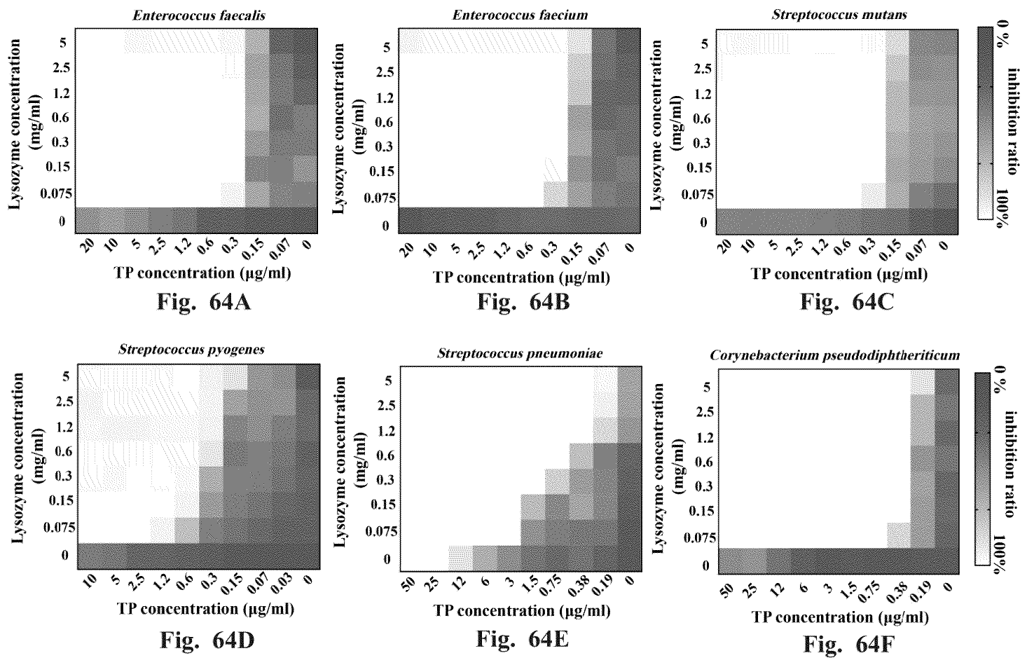

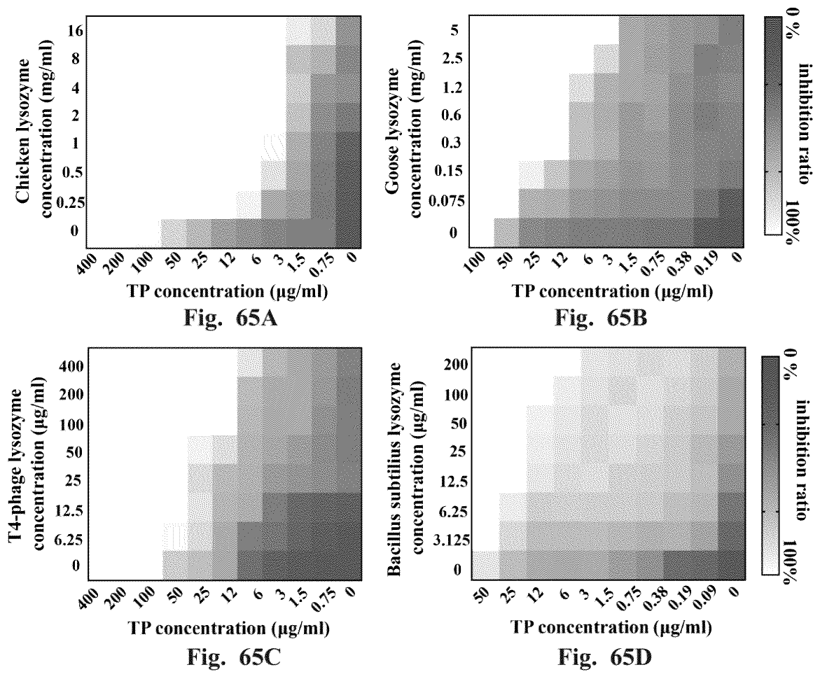

Considering lysozyme was currently widely applied in external bacterial infection studies [Oliver W T et al., J Anim Sci Biotechnol 6, 35 (2015); Wu T et al., Food Chen 274, 698-709 (20191)], rat skin infection model was thus used to explore whether the excellent combination effect can be well-transferred into in vivo studies (FIG. 14A), 1.25 ml of 5.0×10¹³ CFUs MRSA suspension was sprayed on skin wound (3 cm×3 cm) of each anesthetized rat, followed by treatment by sterile medical gauze soaking with normal saline (for control and cefdinir only groups) or lysozyme (14 mg/ml, 1 ml, for lysozyme and combination groups). Meanwhile, rats in cefdinir only and combination groups were further administered orally (25 mg/kg cefdinir). At 168 hours post-inoculation, all rats were sacrificed to collect their wounds. The bacterial load was found to be significantly reduced in wound from rats under the combination therapy if compared with drug along and/or control groups (FIG. 14H). Importantly, their inflammatory symptoms were effectively relieved according to hematoxylin and eosin (H-E) staining analysis. As shown in FIG. 14C, the denser inflammatory cells with blue nucleus were clearly observed in control, cefdinir and lysozyme groups but few in combination therapy groups, More importantly, more new blood vessels were built in wounds of rats under combination therapy, prompting that stronger reconstruction happened. Indeed, the smooth edges of slides from rats in combination therapy groups also indicated the better recovery, which were consistent with their recovered wound images (FIG. 14C, FIG. 29). Meanwhile, these details were further confirmed by two inflammatory indices, i. e., reactive oxygen species (ROS) and interleukin 6 (IL-6), It was observed that, compared with those from control or mono-therapy groups, samples from rats under combination therapy clearly showed more than 36% and 16% decrease in the levels of ROS and IL-6 respectively (FIG. 14D to FIG. 14E) In conclusion, these results clearly showed combination therapy effectively did relieve the symptoms of rats in skin infection model and excellent combination effect could be well-transferred into in via studies,

### 3. Discussion

The emergence and rapid spread of drug-resistant gram-positive pathogens including MRSA pose a severe threat for public health worldwide [David M Z et al., Clin Microbiol Rev 23, 616-687 (2010); Lee A S et al., Nat Rev Dis Primers 4, 18133 (2018)]. Currently, the antimicrobials resistance in pathogens and bacterial recurrent infections are drawing more and more attention, especially in the post-antibiotic era when the development of novel antibiotic is suffering a serious setback [Sun H. et al., Nat Commun 11, 5263 (2020); Kwon J H et al., Science 373, 471 (2021)]. Indeed, the misuse, overuse and prolonged use of antimicrobials inevitably accelerated the coming of superbugs. Thus, it is urgently needed to avoid antimicrobial resistance in the anti-infection treatment.

Human eyes are expected to avoid bacterial infection due to its ability to secrete many natural antimicrobials, such as immunoglobulin and lacritin [Tiffany J M, Tears in health and disease. Eye 17, 923-926 (2003)]. Among them, lysozyme should be the most famous enzyme. However, due to the lysozyme barrier [Cunha-Vaz J G, Doc. Ophthalmol 93, 149-15711997)] and the property of lysozyme as protein to be digested in oral application [Wu T et al., Food Chem 274, 698-709 (2019)], the anti-bacterial role of lysozyme in traditional combination therapy is very limited, but promising in external infection, such as ocular surface or skin infection. In this disclosure, cefdinir used in combination to re-sensitize lysozyme against the broad-spectrum gram-positive pathogens was reported. Mechanism studies indicated that cefdinir facilitated the re-sensitization of lysozyme against cell wall by up regulating the expression of enzymes including TarA. TarB. Tar), TarF, TarL, TarS, TagH and FmtA in LTA/WTA synthesis pathway, as well as the expression of proteins, such as ATP synthase subunit c, to disrupt the energy metabolism, leading to strengthen the each ability to kill MRSA in combination therapy, More importantly, excellent combination effect can be also well-transferred into in vivo studies.

Notably, compared with in vitro, bacterial infection has the more complicated effect on infected organism. Indeed, besides bacterial load, other factors, such as bacterial virulence [Gao P et al., Proc Natl Aca Sci USA 115, 8003-8008(2018)] and host's immunomodulatory activity [da Silva R A G et al., Sci Adv 9, eadd9280 (2023)], have great effects on recoveries in rats under different drug treatments. A representative example in this disclosure was rat under monotherapy in skin infection model. Here, neither lysozyme nor cefdinir at applied dosages significantly killed bacteria in wounds of infected rats, as judged from unchanged bacterial loads. Nevertheless, monotherapies seemed to work in the recovering progress of infected rats because these levels of inflammatory indices, especially ROS, significantly reduced in mono-therapy groups. Such a phenomenon might attribute to the activated immune modulation or weakened virulence of pathogen treated with drug, such as lysozyme, which has been reported in previous studies [Murphy T K et al., J Child Adolese Psychopharmacol 25, 57-64 (2015); Vanderkelen L et al. PLoS One 7, e45934 (2012); Ragland S A et al., PLOS Pathog 13, e1006512 (201: Jiang L et al., Front Pharmacol 12, 767642 (2021); Brott A S et al., Antibiotics 8, (2019)], although more evidences were required to validate it.

Furthermore, in the post-antibiotic era, proteomics analysis on pathogens and superstar drugs have been particularly important because it provides the referred basis to explore potential mechanism of drugs in treating bacterial infection [Tsakou F et al., Pharmaceuticals 13, (2020)]. As the best-sold semi-synthetic antimicrobial and famed self-secreted enzyme, cefdinir and lysozyme still lack complete proteomics data of targets in critical pathogens, such as MRSA. Herein, it was firstly explored that cefdinir and lysozyme proteomics map, which undoubtedly provides crucial information for development of antimicrobial drug in clinical.

In summary, versatile cefdinir coupled with satisfied safety in drug resistance, suggests that cefdinir represents a promising natural antimicrobial adjuvant of lysozyme to tackle the ocular associated pathogenic bacteria. Nevertheless, more prospective clinical trials to verify the potentiation activity of cefdinir in combination with lysozyme in clinical are still required.

### Example 3. Re-Sensitizing Broad-Spectrum Gram-Positive Pathogens to Tedizolid Phosphate by Lysozyme

### 1. Materials and Methods

1) High-throughput screening. According to standard screening method [Zhong. Q et al., Proc. Natl. Acad. Sci. U.S.A. 119, e2119417119 (2022); Sun, 14 et al., Nat. Commun. 11, 523 (2020)], 1953 FDA-approved compounds from our drugs library at a fixed concentrations of 25 μM were screened against methicillin-resistant Staphylococcus aureus (MRSA) and Staphylococcus epidermis (a coagulase negative Staphylococci, MRSE), in combination with human lysozyme. Specifically, these compounds were added into bacterial suspensions (1×10⁵ CFU/ml, in LB medium) in the absence or presence of 1 mg/ml lysozyme. LB medium without bacteria served as background group. Then, the real-time growth curvet of MRSA were monitored per hour and for 24 hours. Absorbance at 600 am (OD₆₀₀) of bacterial culture at the time point of 24-hour was also measured. The inhibition ratio (%) was calculated as (OD_{control}−ODₛₐₘₚₗₑ)/(OD_{control}−OD_{background})×100%. Notably, the relative and apparent inhibition ratios were figured out by defining OD_{control} as the OD₆₀₀ of bacterial suspensions only containing compounds and those without any treatments, respectively. Herein, the relative and apparent inhibition ratios indicated the combination effect and potential toxicity of compound, respectively. Synergy effect was defined as the relative inhibition ratio of ≥90%. All assays were performed in triplicate.

2) Check board broth micro-dilution assays. According to standard two-fold dilution method [Sun. H et al., Nat. Commun. 11, 5263 (2020) tedizolid phosphate at different concentrations were added into fresh LB medium (or trypticase soy troth medium with 5% defibrinated horse blood) containing different concentrations of human lysozyme in 96-well plate. Then, 20 μl of 1.0×10⁷ CFU/ml logarithmic cultures of MRSA or MRSE were added into each well of the plate and co-incubated overnight. Drug-free LB medium in the absence or presence of bacteria served as background control and growth control group, respectively. By measuring the OD₆₀₀ of bacterial culture, the inhibition ratio (%) was calculated as (1−(ODₛₐₘₚₗₑ−OD_{background})/(OD_{control}−OD_{background})]×100%. Colony-forming units were counted by 10-fold serial dilution in PBS to spray 10 μl of dilution onto LB-agar plate. The MIC (minimum inhibitory concentration) was defined as the lowest concentration of drug that inhibited 90% growth of microorganism by both visual CFU and OD₆₀₀. Fractional inhibitory concentration (FIC) and fractional inhibitory concentration index (FICI) were determined by standard method³. Briefly, FICI=MIC_{AB}/MIC_{A}+MIC_{BA}/MIC_{B}=FIC_{A}+FIC_{B}. MIC_{A} was the MIC of compound A aloe; MIC_{AB} was the MIC of compound A in combination with compound B; MIC_{B} was the MIC of compound B alone; MIC_{BA} was the MIC of compound B in combination with compound A; FIC_{A} was the FIC of compound A in combination with compound B; FIC_{B} was the FIC of compound B in combination with compound A. Notably, in a standard 2-fold dilution assay, each drug in the combination has a series of diluted concentrations. Thus, there would be a series of FICI values. In most of cases, the lowest one among these values was listed to reflect the best achieving degree of combination therapy. In terms of the lowest FICI, FICI≤0.5, 0.5<FICI<1 or FICI≥1, would indicate the combination has synergy, part synergy or non-synergy effects, respectively.

Besides, to better highlight the inhibition degree of bacterial growth in combination therapy or identify whether combination therapy has synergy effect, results might be shown by heat-maps or line-charts containing different FICI values, respectively. Each test was performed in triplicate.

3) Time-killing curves. According to standard method [Sun. H et al., Nat. Commun. 11, 5263 (2020); Thappeta, K. R. V., Vikke. Y. S., Yong. A. M. H., Chan-Park. M. B. Kline, K. A. Combined efficacy of an antimicrobial cationic peptide polymer with conventional antibiotics to combat multidrug-resistant pathogens. ACS Infect. Dis. 6, 1229-1237 (2020)], over-night cultured MRSA was diluted and aliquoted into new sterile 50 ml tubes at 1:1000 ratio. Then, the bacterial suspension was co-cultured with 0.25 or 0.5 μg/ml tedizolid phosphate, 0.5 mg/ml lysozyme or their combination at 37° C. with shaking at 50 rpm. Similar bacterial suspension without any additions served as control group. To measure their real bacterial loads to draw their kinetic time-killing curves, 20 μl of each culture was extracted at time intervals of 0, 1, 3, 6, 9, 12 and 22 hours, and then 10 μl of each sample (10-fold serial dilution) was applied onto LB-agar plate and incubated at 37° C. for 24 hours. All assays were performed in triplicate.

4) Biofilm assays. According to previously described method [Thappeta, K. R. et al., ACS Infect. Dis. 6, 1228-1237(2020); Merritt, J. H. et al., Curr. Protoc. Microbiol. Chapter 1, Unit 1B.1 (2005); O'Toole, G. A. et al., J, Vis. Exp. 47, 2437 (2011)], biofilm was assessed by using an adherence assay on 96-well tissue culture plates. Briefly, tedizolid phosphate at final concentration of 0.3 μg/ml was added into bacterial suspensions (1×10⁶ CFU/ml, in in LB medium) in the absence or presence of 0.5 mg/ml lysozyme. LB medium with or without bacteria served as positive or negative control group, respectively. After overnight incubation at 37° C., the bacterial suspension was removed, and well was gently washed twice with sterile PBS to remove exclusively non-adherent bacteria. The adherent biofilm was fixed by using 95% methanol at 60° C. for 15 minutes. Then, biofilm was stained with 1% Crystal violet (100 μl/well) at 37° C. for 15 minutes, Ater that, all wells were gently washed with PBS to remove redundant crystal violet. Finally, the dye was solubilized with 1.50 μl of 95% ethanol per well at 37° C. for 3 minutes. The optical density at 590 nm of solution in each well was measured using a micro-plate reader. Herein, strong biofilm, weak biofilm and no biofilm were defined as “ODₛₐₘₚₗₑ≤2OD_{negative control}”, “OD_{negative control}<ODₛₐₘₚₗₑ<2OD_{negative control}” and “ODₛₐₘₚₗₑ≤OD_{negative control}”. All assays were performed in triplicate.

5) Resistance development studies. As described before [Zhang, Q et al., Proc. Natl. Acad. Sci. U.S.A. 119, e2119417119 (2022); Sun. H et al., Nat Commun. 11, 5263 (2020)], MRSA at exponential phase was diluted (1:1000) into fresh LB medium containing different concentrations of tedizolid phosphate, lysozyme alone or their combination. After cultured at 37° C. for 24 hours, the MIC of culture was determined by standard two-fold serial dilutions in 96-well microtiter plates. Meanwhile, this culture with higher MIC was diluted into similar LB medium but with increasing concentrations of tedizolid phosphate for next passage. The process was repeated for 18 days, and the fold-change in MIC of tedizolid phosphate/lysozyme (relative to initial MIC) was calculated. All assays were performed in triplicate.

6) Mutation frequency and prevention concentration analysis. According to described method [Zhang, Q et al., Proc. Natl. Acad. Sci. U.S.A. 119, e2119417119 (2022); Sun, H et al., Nat. Commun. 11, 3263 (2020)], MRSA culture in log phase was collected and concentrated into bacterial suspension (about 1.0×10¹⁰ CFU/ml) in PBS buffer. Then, 100 μl of diluted MRSA suspension was evenly applied onto agar plates with gradient tedizolid phosphate amounts (including 0.5, 1, 2 and 4 μg/ml) in the presence of lysozyme (including 0.5, 1, 2 and 4 mg/ml). Diluted bacterial culture on agar plates with tedizolid phosphate only served as control group. Meanwhile, initial MRSA suspension was also cultured on agar plate to figure out accurate bacterial load. After 48 hours incubation at 37° C. all colony counts on agar plates were figured out. Herein, the bacterial mutation frequency was calculated as colony counts_{treated group}/colony counts_{initial supplied group}. Remarkably, the minimal concentrations of tedizolid phosphate when it was combined with lysozyme to kill all bacteria were recorded as mutation prevention concentrations (MPC) in combination therapy, and corresponding quotients between MPG and tedizolid phosphate itself MIC were defined as “mutation prevention index” (MPI). All assays were performed in triplicate.

7) Scanning Electron Microscope (SEM) analysis. According to reported method [Abdul Rahim, N et al., J. Antimicrob. Chemother, 70, 2589-2597 (2015)]. MRSA or MRSE single colony was picked up and cultured in LB medium. After 24 hours incubation at 37° C., bacterial suspension was inoculated into fresh LB medium supplied with tedizolid phosphate (0.25 or 0.5 μg/ml), lysozyme (0.5 mg/ml) alone or their combination to collect bacteria in log phase. After 3.5 hours incubation at 37° C. in a shaking at 250 rpm, these bacteria were collected by centrifugation at 3220 g for 10 minutes and fixed with 2.5% glutaraldehyde prior to being washed and re-suspended in PBS. After that, bacteria were dehydrated by using increasing concentrations of ethanol in water (10%, 30%, 50%, 70% 80%, 90% and 100%) for 10 minutes in each step. Then, 5 μl of bacterial suspension at 1.0×10⁹ CFU/ml was incubated on polyethylenimine-coated coverslips (22 mm×22 nu) and the coverslips were dried in balzers critical point dryer (Balzers, Liechtenstein, Germany) prior to mounting on 25 mm aluminium stubs with double-sided carbon tabs. Silver liquid was applied to the edges of each coverslip, and then dried and gold coated in an Edwards S150B sputter coater (Edwards High Vacuum, Crawley, West Sussex, UK). Next, bacteria were imaged with a TESCAN VEGA3 scanning electron microscope (TESCAN, Brno, Czech Republic) at a voltage of 20 kV.

8) Proteomics analysis. According to the previously described method [Guo. H et al., ACS Chet. Biol. 17, 2003-2009 (2022)], MRSA at exponential phase was exposed to lysozyme (0.5 mg/ml tedizolid phosphate (0.5 or 40 μg/ml alone or their combination for further culture for 1 hour at 37° C. Those without any treatment served as control group. Then, bacteria were harvested, and proteins of each sample were extracted using the multiple freezing-thawing at liquid nitrogen followed by sonication at lysis buffer (25 mM HEPES-Na, 150 mM NaCl, 0.1% NP40), 4M urea and 1× protease inhibitor). BCA kit (Thermo fisher, China) was used to quantify the extracted proteins for next digestion by trypsin at the ratio of 5:1. These overnight digested peptides were further labeled by TMT-16^{plex} before loading onto Orbitrap Exploris™ 480 Mass Spectrometer (Thermo fisher. China) coupled with an UltiMate 3000 UPLC System (Thermo Fisher Scientific) with C18 analytical column. Mobile phases A and 1) consisted of 0.1% FA in water and 0.1% FA in 100% ACN, respectively. A 120-minute gradient at a flow rate of 300 minute was used, Meanwhile, mobile phase B was increased to 6% at 12 minutes, 20% at 82 minutes, 30% at 92 minutes, 90% at 100 minutes and held for 5 minutes. Data was collected in data-dependent acquisition (DDA) mode with HCD fragmentation at TopN mode. The resolution was set at, 60,000 for MS1 and 15,000 for MS2 with 30 ms maximum injection time. All resulted spectra were searched against UniProt Staphylococcus aureus (20,330 entries, accessed September 2019) using MaxQuant 1.5.8.2. The parameters for searching: a mass tolerance of 10 ppm for precursor ions; ±0.1 Da for-fragment ions, carbamidomethylation on cysteine was set as a fixed modification, oxidation on Methionine and protein N-terminal acetylation was set as variable modifications. The enzyme was specified as trypsin with two missed cleavages allowed, False discovery ratio for peptide spectral matches and proteins were set as 1%. The maximum number of modifications per peptide was three. Differentially expressed proteins at protein-levels were identified with p-values≤0.05 and fold change (FC) values ≥1.5 (log₂ FC≥0.58 or log₂ FC≤−0.58). The cuff-diff program was used to analyze differences between two treatments.

9) Membrane potential analysis. Membrane potential assays were carried out according to the modified method described previously [Zhang. Q et al., Proc. Natl. Acad. Sci. U.S.A. 119, e2119417119 (2022); Sun, H et al., Nat, Commun. 11, 5263 (2020)]. About 10⁶ CFUs of mid-log-phase MRSA or MRSE pellets pre-incubated with lysozyme (0.5 mg/ml), tedizolid phosphate (0.5 μg/ml) or their combination at 37° C. for 1 hour were collected, washed by PBS for 3 times and then re-suspended in PBS supplemented with 3,3′-Diethyloxacarbocyanine iodides (DiOC₂(3), 30 μM) for further staining at 37° C. for 30 minutes, 10⁶ CFUs of MRSA or MRSE treated with carbonylcyanide3-chlorophenylhydrazone (CCCP, a proton ionophore, 5 μM) served as positive control group and those treated with PBS were used as negative control group. Then, these stained bacteria were uploaded into a flow cytometer (BD Accuri. ThermoFisher Scientific), and signals from FITC-A (fluorescein isothiocyanate-A, 488 nm, green channel) and PI-A (propidium iodide-A, 633 nm, red channel) were collected and analyzed by FlowJo v10 (Free Star Inc., Ashland. USA). All assays were performed in triplicate and results were expressed with fluorescence ratio (green/red) as average±SD.

10) Determination of cell wall and membrane integrity. According to the previously described method [(Liu. Y et al., Adv. Sci. 7, 1902227 (2020); Santiso, R et al., BMC Microbiol. 11, 191 (2011)], all samples under different treatments were firstly collected. To analyze the integrity of cell wall, an aliquot of each sample was diluted into fresh LB medium to be bacterial suspension (5×10⁶ CFU/ml). Meanwhile, a tube containing 0.5 ml of low-melting mint agarose was placed in a water bath at 90-100° C. for about 5 minutes to melt the agarose completely and then placed in a water bath at 37° C. 25 μl of the diluted bacterial sample was added into the tube and mixed with the melted agarose completely. After that, 20 μl aliquot of the sample-agarose mixture was pipetted onto a precoated slide, and the sample was covered with a 22-22 mm coverslip. The slide was placed on a cold plate in the refrigerator (4° C.) for 5 minutes to allow the agarose to produce microgel with the trapped intact cells inside. The coverslip was removed gently, and the slide was immediately immersed horizontally in 10 ml of the lysing solution (25 mM HEPES-Na, 150 mM NaCl, 0.5% NP40 and 0.5% Triton-X100) for 5 minutes at 37° C. These treated with control buffer 425 mM HEPES-Na, 150 mM NaCl, 0.2% EDTA and 0.5 mg/ml lysozyme) served as positive control group. The slide was washed horizontally in a tray with abundant distilled water 3 minutes, dehydrated by incubating horizontally in cold ethanol with increasing concentrations (70%, 90% and 100%) for 3 minutes each time and air-dried in an oven. Then, dried slide was incubated in a microwave oven at 750 W for 4 minutes, and the DNA was stained with 25 μl of the fluorochrome SYBR-Gold (Thermo fisher, China) diluted 1:400 in TBE buffer (0.09M Tris-borate, 2 mM EDTA, pH 7.5) for 2 minutes in the darkness. After a brief wash in PBS buffer, a 2460 mm coverslip was added, and the slides were visualized under fluorescence microscopy.

According to reported method with some modifications [Liu, Y et al., Adv. Sci. 7, 1902227 (2020)], a permeation assay was performed to evaluate the integrity of cell membrane, Specifically, about 50 μl of 10⁹ CFU/ml mid-log-phase MRSA under different pre-treatments wee incubated with propidium iodide (PI) at final concentration of 0.5 μM, As a red-fluorescent DNA dye, PI could only cross the plasma membrane of nonviable but not live cells. After incubation for 30 minutes in the darkness, the fluorescence intensity (λₑₓ535 nm, λₑₘ=615 nm) was determined using an infinite M200 microplate reader. Those treated with 0.5% Triton-X100 or 1×PBS served as positive or negative control group. All assays were performed in triplicate and results were expressed with fluorescence intensities as average±SD.

11) Antibiotics accumulation analysis. The accumulation of antibiotics in MRSA was determined by LC-MS/MS analysis according to a previous report [Liu, Y et al., Adv. Sci. 7. 1902227 (2020); Ong, V et al., Drug Metab. Dispos, 4127 (2014); Richter, M. F et al., Nature 545, 299-304 (2017)]. Briefly, 1.0 ml of overnight culture of MRSA was diluted into 100 in of fresh LB medium and cultured at 37° C. with shaking at 250 rpm until when bacteria OD₆₀₀ was about 0.4. Then, tedizolid phosphate (0.5 μg/ml) in combination with varying lysozyme (0, 0.25, 0.5 and 1.0 mg/ml) was added. After that, all samples were further cultured at 37° C. with shaking at 250 rpm for 0.5, 1.0, 2.0 or 4 hours, respectively. Then, all bacteria were pelleted by centrifuging at 13 000 g for 20 minutes. To lyse the bacteria, each pellet was dissolved in 400 μl of lysis buffer (150 mM NaCl, 1% Triton-X100, 1× proteinase inhibitor and 50 mM HEPES-Na, pH 8.0) and then subjected to sonication lysis, followed by three freeze-thaw cycles in liquid nitrogen. The lysates were pelleted at 13 000 g for 20 minutes and the supernatants were collected. All debris were re-suspended in 400 μl of methanol and pelleted as before, followed by 400 μl of acetonitrile treatment as before. These supernatants were combined with the previously collected supernatants, Residual debris was removed by centrifuging at 13 000 g for 20 minutes. Supernatants were analyzed by an Agilent 1260 Infinity HPLC system coupled to AD SCIEX QTRAP 6500 mass spectrometers (ABSciex, CA, USA). The liquid chromatography separation was performed on water HSS T3 column (2.1×100 mm, 3 μm) with mobile phase A (20 mM ammonium formate in water) and mobile phase B (methanol). The flow rate was 03 ml/min. The linear gradient was as follows: 0.1-1.0 minute, 98% A; 1.0-5.0 minutes, 98-10% A: 5.0-6.0 minutes, 10-0% A, 6.0-7.0 minutes, 0-98%, 7.0-8.0 minutes, 98% A. The injection volume was 2 μl, Considering the rapid metabolism of tedizolid phosphate into tedizolid in vivo [Ong, V et al., Drug Metab. Dispos. 42, 1275 (2014)], the intracellular level of tedizolid was also considered into the total content of tedizolid phosphate. The quantification determination of antibiotic uptake was performed by multiple reaction monitoring (MRM) with negative electrospray ionization using the m/z 451 transition for tedizolid phosphate [Ong, V et al., Drug Metab. Dispos. 42, 1275 (2014)]. Similar protocol was performed to analyze the intracellular uptake content of tedizolid in MRSA. Notably, the quantification determination of tedizolid uptake was performed by multiple reaction monitoring (MRM) with positive electrospray ionization using the m/z 371 transition for tedizolid¹⁰. All assays were performed in triplicate and results were expressed with average±SD.

12) Nascent protein synthesis analysis. According to the previously reported method [Forester, C. M et al., Proc. Nat. Acad. Sci. U.S.A. 115, 2353-2359 (2018); Liu, J et al., Proc. Natl. Acad. Sci. U.S.A. 109, 413-418 (2012)], Click-iT™ plus OPP Alexa Fluor™ 647 protein synthesis assay kit containing OPP (O-propargyl-puromycin) probe was used to evaluate the inhibited synthesis of nascent proteins. Specifically, mid-log-phase MRSA with OD₆₀₀ of 0.4 to 0.6 was treated with lysozyme, tedizolid phosphate or their combination at different concentrations at 37° C. for 1 hour. These bacteria served as related negative control groups. Positive control groups had similar protocols but with addition of 20 μM OPP. After 1 hour incubation, all bacteria were collected washed by cold PBS for 3 times and fixed by 3.7% formaldehyde in PBS, followed by re-suspension in lysis buffer containing 0.5% Triton® X-100. Then, these fixed and permeabilized cells were incubated with Click-iT™ plus OPP reaction cocktail according to the standard protocol [Forester, C. M et al., Proc. Nat. Acad. U.S.A. 115, 2353-2358 (2018)]. After 30 minutes incubation in the prevention of light at room temperature, the reaction cocktail was removed. Next, these fixed cells in 96-well plate were washed by using 200 μl/well of Click-iT® reaction rinse buffer, followed by further incubation with 1×HCS nuclear Mask™ blue stain working solution. After 30 minutes incubation in the prevention of light at room temperature, the nuclear Mask™ blue stain working solution was removed. Finally, these cells were washed twice with cold PBS before fluorescent analysis according to the individual excitation and emission maxima (Alexa Fluor™ 647 picolyl azide (λₑₓ=650 nm, λₑₘ=670 nm); nuclear Mask™ blue stain: (λₑₓ=350 nm, λₑₘ=451 nm)). Inhibition degrees of nascent proteins synthesis were expressed with fluorescence ratio (Alexa Fluor®/nuclear Mask™) as average±SD. All assays were performed in triplicate.

13) ATP, NAD⁺/NADH and pyruvate levels. According to the described method [Liu. Y et al., Adv. Sci. 7, 1902227(2020)], mid-log-phase MRSA with the OD₆₀₀ of 0.4 to 0.6 was treated with lysozyme, tedizolid phosphate or their combination at different concentrations. Those without any treatment served as control group. After 1 hour incubation at 37° C. all bacteria we collected and then washed by cold PBS for 3 times. To evaluate the pyruvate level, all samples were resuspended in cold extracting buffer (Solarbio, Co. Ltd, China) to get bacterial suspension with an initial density of about 5.0×10⁶ CFU/ml, followed by sonication lysis for 40 minutes at 4° C., and centrifugation at 18,000 g at 4° C. for 20 minutes to collect these supernatants. Then, 75 μl of each supernatant was incubated 25 μl of reaction I, followed by continuous incubation with 125 μl of reaction II. By recording the absorbance at 520 mu, the pyruvate level in each sample was figured out according to the standard formula. The intensity in control group was defined as 100% and others were normalized.

Similarly, to evaluate the intracellular ATP, NAD⁺ and NADH levels, these collected samples were re-suspended in cold extracting buffer (Beyotime Biotechnology, China) to get bacterial suspensions with an initial density of about 5.0×10⁶ CFU/ml, followed by sonication lysis for 40 minutes at 4° C. and centrifugation at 18,000 g at 4° C. for 20 minutes to collect these supernatants. Then, 20 si of each supernatant was added white 96-well plate pre-reactivated by 100 μl of ATP test solution (Beyotime Biotechnology, China), followed by luminometer measurement [Liu, Y et al., Adv. Sci 7, 1902227 (2020)]. To analyze the intracellular NAD⁺ and NADH levels [Xue, C et al., Front. Pharmacol. 12, 600296 (2021)], each lysis supernatant was divided into two tubes. One tube was heated at 60° C. for 30 minutes in PCR machine to decompose NAD⁺ if had. Then, this tube was further centrifuged at 18,000 g at 4° C. for 20 minutes to collect the supernatant. After that, 20 μl of supernatant from heated or unheated group was added white 96-well plate pre-reactivated by 90 μl/well of alcohol dehydrogenase solution (Beyotime Biotechnology, China) for 10 minutes incubation at 37° C. in the prevention of light, Next, 10 μl of staining solution (Beyotime Biotechnology, China) was added to react with product in previous reaction. After 30 minutes at 37° C. in the prevention of light, orange formazan was observed and then the absorbance at 450 nm was recorded. Intensities of those with and without heat treatment indicated the levels of NADH+NAD⁺ and NADH itself, respectively. Finally, according to the standard formula, the intracellular levels of NAD⁺, NADH and their ratio were figured out. All results were expressed as average±SD. Assays were performed in triplicate.

14) Enzyme activity of alkaline phosphatase. All enzyme activities of alkaline phosphatase were analyzed according to the modified method as previously described [Zhao, X.-y et al., Nature 46, 86-90 (2009); Zhang, XC e al., Anal. Chem. 92, 5185-5190 (2020); Rosch, J. & Caparon, M. Science 304, 1513-1151 (2004); Wang. W et al., PLoS One 10, e012403512013); Ghosh, S. S et al., Circ. Res. 128, 1646-1659 (2021)]. Briefly, mid-log-phase MRSA or MRSE was treated with lysozyme, tedizolid phosphate or their combination at different concentrations. Those without any treatment served as untreated group. After incubation at 37° C. for 1 hour, all samples were collected, washed by distilled water and re-suspended in distilled water to collect bacterial suspension with an initial density of about 5.0×10¹² CFU/ml according to pre-experiment. Then, sonication lysis for 40 minutes at 4° C. and centrifugation at 18,000 g at 4° C. for 20 minutes were performed to collect these supernatants. Next, 20 μl of each supernatant was incubated with 4 μg of tedizolid phosphate. After incubation at 37° C. for 1 hour, all samples were uploaded onto Agilent 1260 Infinity HPLC system coupled to an AB SCIEX QTRAP 6500 mass spectrometer (ABSciex, CA, USA) as described above. To visualize the difference in enzyme activities of alkaline phosphatase in different groups, above-described bacterial suspensions would be exposed to BCIP/NBT working solution (Beyotime Biotechnology. China) at 37° C. for 30 minutes in the prevention of light, Next, the staining was stopped by using distilled water to wash the cells for 3 times, and then re-suspended in 3.7% formaldehyde in PBS. After fixed at non temperature for 30 minutes, these bacteria were washed by cold PRS for 3 times. Then, 25 μl of each sample was mixed with the melted agarose. 20 μl aliquot of the sample-agarose mixture was pipetted onto a precoated slide, and then the sample was covered with a 22×22 mm coverslip. These slides were visualized under fluorescence microscopy and randomly selected bacteria were analyzed at ×1,500 magnification. To quantify the enzyme activity, similar protocol was performed on cell lysis supernatant instead of above-described bacterial suspension. Finally, absorption spectrums (300-800 nm) were collected and analyzed. All assays were performed in triplicate.

15) Purification of G-type lysozyme. Lysozyme can cleave β-1,4 glycosidic bond of peptidoglycan in bacterial cell wall. Widely used lysozyme in nature generally belongs to: C-type (from human or chicken), G-type (from goose), T-type (from T4 bacteriophage and bacterial-type (from bacteria, such as Bacillus subtilis) [Ganz, T. Lysozyme, in Encyclopedia of Respiratory Medicine (eds, Laurent, G. J. & Shapiro, S. D.) 6494-653 (Academic Press, Oxford, 2006)].

According to a referable protocol with refinement [Thammasirirak, S et al., Biosci. Biotechnol. Biochem. 65, 584-392 (2001)], goose egg white (from Anser cygnoides egg) was firstly separated, diluted with two volumes of 50 mM phosphate buffer (PBS, pH 7.0) and stirred for 30 minutes at 4° C., followed by centrifugation at 12,000 g at 4° C. for 15 minutes to collect the solvable supernatant. Next, the crude extract was further treated with isoelectric precipitation at pH 4.0, 6.0, and 7.0. At each step of pH treatment, the solution was adjusted to the desired pH by using 1 M HCl or NaOH for next incubation at 4° C. for 1 hour. After that, the crude extract would be further purified by centrifuging at 12,000 g at 4° C. for 30 minutes and filtering under 0.45 μm filter to remove insoluble protein precipitation, Next, all soluble supernatant was applied to a 5 ml HiTrap SP-FF cation exchange resin column (GE. Healthcare) pre-washed by five column volumes of 50 mM PBS (pH 7.0) at a rate of 2 ml/min. Then, column was washed with the same buffer and the targeted lysozyme was eluted with a linear gradient of NaCl concentration from 100 mM to 500 mM in the same buffer at the flow rate of 1.5 ml/min. Next, the purified lysozyme was pooled and uploaded onto Superdex 75 column (GE Healthcare) equilibrated with running buffer (10 mM NH₄NO₃, pH=7.0). These target lysozyme fractions (lysozyme was confirmed by using in-gel digestion mass spectrometric identification, data not shown) were collected and then concentrated to 80 mg/ml for further analysis by using SpeedVac rotary evaporator at 4° C. for 5 hours, followed by aliquoting for long-term storage at −80° C.

16) FtsZ immunofluorescence staining. According to the reported method for bacterial immunofluorescence staining [Park, S et al., Biochemistry 58, 4457-4465 (2009)], over-night cultured MRSA was inoculated into fresh LB medium at 1:1000 ratio. Next, the bacterial suspension was exposed to 0.5 μg/ml tedizolid phosphate, 0.5 mg/ml lysozyme alone or their combination. After 1 hour incubation at 37° C. in a shaking at 250 rpm, 10 ml of bacteria were collected by centrifugation at 4000 g for 5 minutes and washed twice with PBS. Then, bacteria were further re-suspended in 1 ml of 4% formaldehyde, followed by incubation at torn temperature for 34 minutes. Next, bacteria were washed twice with 1 ml of PBS, followed by re-suspension in 10 ml of 70% ethanol at room temperature for 1 hour. After that, all bacteria were washed twice with PBS and then incubated with permeabilization buffer (PBS containing 25 μg/ml lysozyme and 50 U/ml DNase 1) a 37° C. for 1 hour. Then, bacteria were re-suspended and incubated in blocking buffer (PBS containing 0.1% BSA, 0.05% Tween 20). After 1 hour treatment at room temperature, bacteria were further washed twice with PBS, followed by incubation with 10 ml of FtsZ antibody (0.2 μg/ml in PBS, Fab Gennix, USA). After 1 hour incubation at room temperature, the antibody/cell mixture were centrifuged at 1000 g for 5 minutes, followed by washed twice with PBS, Next. MRSA was further exposed to FITC labeled anti-rabbit second antibody (1:1000, in PBS) for 1 hour at room temperature. Then, further wash was performed, Similar protocol was performed in DAPI staining. Finally, 5 μl of bacterial suspension at 1.0×10⁹ CFU/ml was mixed with the melted agarose, and then 20 μl aliquot of the sample-agarose mixture were pipetted onto a re-coated slide. After a brief wash in PBS buffer, a 24×60 mm coverslip was added, and the slide was visualized under fluorescence microscopy. All assays were performed in triplicate and representative images were showed.

17) Toxicities analysis on lysozyme and several oxazolidinone-class antibiotics. It was well-known that lysozyme was a bio-safe protein [Ganz, T. Lysozyme, in Encyclopedia of Respiratory Medicine (eds. Laurent, G. J. & Shapiro, S. D.) 649-651 (Academic Press, Oxford, 2006); Wu, T et al., Food Chem. 274, 698-709(2019); Sen, D. K. & Sarin, G. S. Br. J. Ophthalmol 70, 246-248 (1986)] and tedizolid phosphate had been FDA-approved since 2014 [Rybak, J. M. & Roberts, K. Infect. Dis. Ther. 4, 1-14 (2015); Wilson, D. N et al., Proc, Natl. Acad. Sci. U.S.A. 10, 13339-13344 (2008)]. Nevertheless, the cellular toxicities of lysozyme and several oxazolidinon-class antibiotics including tedizolid phosphate were still evaluated by using several types of human cells, i. e., NIH/3T3, 293T and ARPE-19 cells. These cells at 80% confluency were cultured in medium with gradient amounts of tedizolid phosphate or lysozyme. Cells without any drug treatment served as control group. After 12 hours or 24 hours culture at 37° C., living cells were counted. Herein, 100% survival ratio (no toxicity) was defined as conditions, in which the number of living cells was equal with those in control group, Assays were performed in triplicate and results were expressed with as average±SD.

18) Cell infection model. According to the previously described method [Wang, R et. al., Nat. Commun. 9, 439 (2018)], ARPE-19 cell, a human retinal pigment epithelial cell line, was selected and cultured in gibco dulbecco's modified eagle medium (nutrient mixture F-12 (DMEM/F12) supplemented with 10% fetal bovine serum (FBS)) at 37° C. in 5% CO₂-humidified atmosphere for 7 days. Then, about 1.0×10⁴ collected ARPE-19 cells were seeded into per well of 96-well plate and incubated overnight to ensure 50% confluency. Logarithmic cultures of MRSA were washed with PBS for three times and re-suspended in DMEM/F12-10% FBS to get bacteria suspensions with an initial density of about 1.0×10² CFU/ml. Next, 10 μl of bacterial suspensions were mixed with ARPE-19 cells and incubated for 3 hours, in which multiplicity of infection (MOI) in bacterial infection was 10. Then, infected ARPE-19 cells were washed vigorously with PBS for six times and replenished with culture medium to remove these unassociated bacteria. Herein, cell-associated bacteria were defined as bacteria that attached to, penetrated, or transcytosed in ARPE-19 cells. Next, these ARPE-19 cells were exposed to lysozyme, tedizolid phosphate alone or their combination overnight under identical cell culture condition (100 μl of per well). Medium in the absence and presence of cells served as negative and positive control group, respectively. After 24 hours incubation, CCK-4 assays were performed to analyze the survival ratio of cells under different treatments. Specifically. 10 μl of CCK-8 solution per well was added, followed by incubation for another 2 hours at 37° C. for chromogen development. The absorbance at 450 nm was measured, with 630 nm as reference wavelength. Herein, survival ratio=(Absₛₐₘₚₗₑ−Abs_{negative control})/(Abs_{positive control}−Abs_{negative control})×100%. Meanwhile, bacterial leads were examined by lysing ARPE-19 cells with PBS containing 1% Triton X-100 and serially diluting the resulting lysates onto LB-agar plates. All assays were performed in triplicate and results were expressed as average±SD.

19) Skin infection model. According to the reported method [Zhang, Q et al., Proc. Natl. Acad. Sci. U.S.A. 119, e2119417119(2022)], 15 Sprague Dawley (SD) male rats 64 weeks old) were divided into 5 groups (3 rats per group. Except 3 rats in uninfected group, other 12 rats were infected by 5×10¹¹ CFUs of mid-log phase MRSA from our own collection. Briefly, all rats were firstly pec-depilated by using 10% Na₂S to get an uncovered back area of 4 cm×4 cm before 24 hours for the anesthetization on rats. Then, the skin of anesthetized rat was removed until fascial region under sterile condition, followed by spraying 5.0×10¹¹ CFUs of mid-log phase MRSA on wound (3 cm×3 cm). Bacterial suspensions were collected by below-described method [Zhang. Q et al., Proc. Natl. Acad. Sci. U.S.A. 119, e2119417119 (2022)]. Specifically, mid-log phase MRSA was collected by centrifugation at 4500 g for 15 minutes, washed with PBS for 3 limes and re-suspended in cold PBS to get bacterial suspension with an initial density of about 4.0×10¹¹ CFU/ml according to pre-experiment. Next, 1.25 ml of bacterial suspension was sprayed onto wound. Notably, normal saline was used to replace bacterial suspension in uninfected group, After 3 hours infection, wounds of all rats were covered by sterile medical gauze soaking with normal saline. All sterile medical gauzes were 0.2 g/cm², and further covered by two layers of sterile gauzes and fixed by sterile tapes. Next, rats were monitored until they were fully awake, and then returned to their cages. After 24 hours infection, these sterile medical gauzes were replaced with new sterile medical gauzes soaking different drugs, i.e., normal saline, tedizolid phosphate (0.167 mg/kg) or lysozyme (0.833 mg/kg), or their combination (0.167 mg tedizolid phosphates/kg rat+0.833 mg lysosyme/kg rat), respectively. Meanwhile, rats in uninfected group were treated with new sterile medical gauzes soaking normal saline. All steps were repeated at 48-, 72-, 96-, 120- and 144-hr post-inoculation. At 168 hours post-inoculation, all rats were sacrificed to collect their wounds. These collected tissues were fixed in 10% formalin for at least 24 hours and embedded in paraffin. Next, about 4-μm sections were analyzed by using the hematoxylin and eosin (H-E) staining kit. The rest of wound was placed into tube containing 1 ml of sterile PBS on ice, and then homogenized to detect the levels of ROS or IL-6. Meanwhile, this homogenate was also 10-fold gradient-diluted in 96-well plate to figure out the bacterial load in infected wound according to quantitative grown colony after overnight culture. The rest of homogenate was immediately stored in sterile tube at −80° C. for further analysis.

20) Ocular infection model. According to the reported method [Zhang, Z et al., Curt. Protoc, mouse Bot 7, 55-63 (2017)], 40 C57BL/6, male mice (64 weeks old) were used to evaluate the combination effect in the ocular infection model. Specifically, all mice were divided into 5 groups (4 infected groups and 1 uninfected group, 8 mice per group). Among than, 32 mice in infected groups were infected by slowly inoculating about 2.5×10⁷ CFUs of mid-log phase MRSA into each ocular surface. To collect these mid-tog phase ARA, a colony of MRSA cultured in LB-agar plate was firstly picked up and inoculated into fresh LB medium for overnight incubation at 37° C. Then, the overnight culture was diluted (1:300) into fresh LB medium for continuous culture until bacterial OD₆₀₀ was about 0.6. Next, these mid-log phase MRSA would be collected by centrifugation at 4500 g for 15 minutes, washed with PBS for 3 times and re-suspended in cold PBS to get bacterial suspension with an initial density of about 1.0×10¹⁰ CFU/ml according to pre-experiment. Next, 2.5 μl of bacterial suspensions were slowly inoculated into each ocular surface of anesthetized C57/BL6 mouse on the side of surgical bed. 2.5 μl of cold PBS were inoculated into each ocular surface of anesthetized C57/BL6 mouse in uninfected group. After 6 hours post-inoculation, these infected mice were further divided into 4 groups. Among them, 8 mice without any treatment served as control infected group. Meanwhile, these mice in other groups were treated with monotherapy of tedizolid phosphate (0.125 mg/ml, 2.5 μl/eye) or lysozyme (100 mg/ml, 2.5 μl/eye), or their combination therapy (0.125 mg/ml tedizolid phosphate+100 mg/ml lysozyme, 0.5 μl/eye in total). All C57/BL6 mice were fully anesthetized on the side of surgical bed until all treatments were finished. After that, they were repeated from the air-anesthetizing room and monitored until they were fully awake, followed by retuned to their cages. The step was repeated at 24-hr post-inoculation. Notably prior to the repeated step, 5 μl of PBS was administered to each ocular surface of anesthetized mouse on the side of surgical bed. After 30 sec, tear fluid was collected from the lateral canthus of the eye and transferred into a sterile tube on ice. Then, according to the standard protocol²⁸, 3 μl of tear fluid was gradient-diluted at 10-fold in 96-well plat to figure out the initial number of viable bacteria in tear fluid according to quantitative grown colony after overnight culture. The rest of tear fluid was immediately stored in sterile tube at −80° C. for further analysis. The step was repeated at 48-hr post-inoculation. Remarkably, at 24-hr and 49-hr post-inoculation, 3 anesthetized mice per group received the ocular injury analysis. Specifically, 1 μl of 1% sodium fluorescein was administered to the inferior-lateral conjunctival sac of anesthetized nose on the side of surgical bed. After 3 minutes staining, photograph of each cornea was be collected by using a slit-lamp bio-microscope under cobalt-blue light, According to reported method [Zhang, Z et al., Cur. Protoc, mouse Biol. 7, 55-43 (2017)], the staining of each corneal zone (i.e., superior, inferior, temporal, nasal and central) was scored as 0 (absent), 1 (regional or diffuse punctate staining and moderate stipple staining), 2 (heavy stippling, dense coalescent staining), or 3 (diffuse loss of epithelium). Finally, all unstained mice were sacrificed to collect their lacrimal glands, upper eye lids with conjunctiva and eyeballs for further analysis. Notably, at 48-hr pot-inoculation, both the percentage of invaded epithelial cells and average counts of bacteria in per epithelial cell in tear fluids were also determined. According to the reported method [Zhang, Z et al., PLoS One 10, e0138597 (2015)], 1-2 μl of collected tear fluids from mice in different groups at different time points were centrifuged at 4° C. at 250 g for 10 minutes to separate neutrophil and epithelial cells. Next, these cells were further washed three times with cold PBS, followed by sedimentation. The final suspensions were re-suspended in 5 μl of PBS, and then cells were mounted on slides by cytospin centrifugation. Next, these cells on slides were methanol-fixed and stained by using the Hema-3 stain kit according to the reported method [Zhang, Z et al., PLoS One 10, e0138597 (2015)]. Specifically, these methanol-fixed cells were firstly covered by 750 μl of Hema-3 stain solution I. After incubation with gentle agitation for 10 minutes, the Hema-3 stain solution I was removed by using distilled water. Then, these cells were further covered by 750 μl of Hema-3 stain solution II for 10 minutes, followed by further washing with distilled water twice before Ventilation drying in a laminar flow biosafety cabinet. Next, one hundred randomly selected epithelia cells per slide were analyzed at ×400 magnification under light microscopy. The invasion percentage of epithelial cell was defined as the percent of epithelial cells invaded by ≥1 bacterium if compared with all epithelial cells observed in slide. The ratios between numbers of observed bacteria and epithelial cells served as the average number of bacteria in per epithelial cell. To evaluate the combination effect on intact ability of host defense against the MRSA infection, the levels of surfactant D (SP-D) in tear fluid and ocular homogenate were also examined. Specifically, total content of proteins in above-collected tear fluid or ocular homogenate was firstly analyzed by standard BCA assay. Then, these samples were subjected to gel electrophoresis for western-blotting analysis on SP-D by using a specific SP-D antibody (diluted 1:1000) [Zhang, Z et al., PLoS One 10, e0138597(2015)]. To be consistent in contents of total proteins in ocular homogenate, the GAPDH level was analyzed and normalized by using anti-GAPDH antibody. To perform the H-E analysis on mice lacrimal glands, upper eye lids with conjunctiva and eyeballs, these collected tissues were firstly fixed in 10% formalin for at least 24 hours and then embedded in paraffin. About 4-μm sections from 3 mice per group were analyzed, Xylene deparaffinized sections were washed in 100% ethanol, rehydrated in graded ethanol, and incubated with H-E staining solution according to the reported method [Zhang, Z et al., PLoS One 10, e0138597 (2015); Zhang, S et al., Adv. Sci. 8, 2100681 (2021)].

21) Microbiome assay. According to the previously described method [Willis, K. A et al., Sci. Rep. 10, 12035 (2020); Deng, Y et al., Cell Discov. 7, 13 (2021); Xue, W et al., Front. Cell. Infect. Microbiol. 11, 759333 (2021); Ranjith, K et al., Exp. Eye Res. 205, 109476 (2021)], 40 C57BL/6 male mice (6-8 weeks old) were used to evaluate the combination therapy in the ocular infection model. At 24-hr and 48-hr post-inoculation, 3 μl of tear fluid (24-hr and 48-hr) and 100 μl of right ocular homogenate (48-hr) from each mouse were collected to perform the 16s rRNA analysis (BGI Genomics, China). The V3 and V4 regions of bacterial genome were specially amplified to analyze the contents of different microbes in microbial community [Bukin, Y. S et al., Sci. Data 6, 190007(2019); Chakravorty. S et al., J Microbiol. Methods 69, 330-339 (2007), Katiraei, S et al., Curr. Microbiol. 79, 276 (2022)]. All results were analyzed according to standard protocol [Johnson, J S et al., Nat. Commun. 10, 5029 (2019)] and expressed as average±SD.

22) Animals, cells, bacterial strains, compounds, reagents and instruments. NIH/3T3, 293T and ARPE-19 cells, methicillin-resistant Staphylococcus aures USA 300 (MRSA, ATCC-BAA1556) and Staphylococcus epidermis (MRSE, ATCC35984) were from our own collection. Streptococcus pyogenes (CGMCC code: 1.8868), Corynebacterium pseudodiphtheriticum (CGMCC code: 1.592), Streptococcus pneumoniae (CGMCC code: 1.8722) Enterococcus faecalis (CGMCC code: 1.10682), Enterococcus faecium (CGMCC code: 1.15321), Streptococcus mutans (CGMCC code: 1.2494) and Propionibacterium acnes (CGMCC code: 1.5003) were purchased from China general microbiological culture collection center (CGMCC). Competent cells (TOP10, BL21 (DE3) and DR5a (DE3) were supplied by TianGen, China. Egg of Chinese goose was ordered from Etsy, Hong Kong. C57/BL6 male mice in ocular infection model and microbiome assay were six to eight weeks old, male, 18-22 g of weight (from Charles River Laboratories, Inc). Sprague-Dawley (SD) male rats in skin infection model were six to seven weeks old and purchased from Charles River Laboratories, Inc. All experiments were approved by and performed in accordance with the guidelines approved by committee on the use of live animals in teaching and research (CULATR) (Ref No.: 21-22/262-ABCT-R-OTHERS), The Hong Kong Polytechnic University (Shenzhen Research Institute). Hunan lysozyme (C-type), Chicken lysozyme (C-type), T4 phage lysosyme (T-type), Bacillus subtilis lysozyme (bacterial-type), ampicillin sodium salt, hydroxyethyl piperazine ethanesulfonic acid (HEPES), imidazole, thrombin, ammonium nitrate (NH₄NO₃), formaldehyde, crystal violet, sodium fluorescein, sodium chloride (NaCl), Luria-Bertani (LB) both powder and phosphate buffered saline (PBS) were supplied by Sigma-Aldrich. ATP assay kit. NAD⁺/NADH assay kit, pH-sensitive fluorescence probe BCECF-AM, alkaline phosphatase activity kit, DiOC₂(3) were purchased from Beyotime. Pyruvate level kit, Tryptic Soy Broth (TSB) medium and defibrinated horse blood were obtained from Solarbio, Co. Ltd, China, Methanol, glutaraldehyde, NP40, urea and protease inhibitor, TMT-16^{plex}, C18 column, click-iT™ plus OPP alexa Fluor™ 647 protein synthesis assay kit, propidium iodide (PI), Triton-X100, ethanol, SYBR gold dye, DMEM/F12, fetal bovine serum (FBS), CCK-8 assay kit. BCA assay kit, ROS kit, IL-6 kit, SP-D antibody, GAPDH antibody, goat anti-rabbit IgG antibody, hematoxylin and cosin (H-E) staining kit, Hema-3 stain kit, plate-reader, slit-lamp bio-microscope, bacterial incubator and high-throughput screening robust were purchased from Thermo fisher, China, Hitra SP-PF cation exchange resin column and Superdex 75 column were ordered from GE Healthcare. Fast performance liquid chromatograph (FPLC), ITC 2009, PCR machine, dialysis membrane and centrifugal tilter (Ultra-15 (10 kDa)) were purchased from GE, USA. Ultra sonicator and centrifuges were supplied by Beckman (Germany). Deionized (DI) water (18.2 MΩ·cm) and filters (0.45 and 0.22 μM) were purchased from Milipore, USA. All other chemicals/compounds were purchased from MCE (China) unless otherwise stated.

### 23) Cellular pH Analysis

The cellular pH was analyzed by using pH-sensitive fluorescence probe BCECF-AM (Beyotime Biotechnology, China). Briefly, mid-log-phase MRSA or MRSE was treated with lysozyme, tedizolid phosphate or their combination at different concentrations. Those without any treatment served as untreated group. Meanwhile, pH-sensitive fluorescence probe BCECF-AM was also supplemented at the final concentration of 10 μM, LB medium exposed to 10 μM BCECF-AM served as negative control group. After incubation at 37° C. for 1 hour, 200 μl of each sample was added into the white 96-well plate to analyze the fluorescence absorbance. According to the reported method [Y. Liu et al., Adv. Sci. 7, 1902227 (2020)], the excitation wavelengths on the fluorescence spectrometry was set at 488 nm and the fluorescence emission absorbance spectra was recorded in the range of 550 nm to 600 nm. All assays were performed in triplicate.

24) Synthesis and characterization of delpazolid phosphate and eperezolid phosphate. The inventors firstly mixed delpazolid with phosphoramidic acid (Di-tert-butyl N,N-diethylphosphoramidite) at the 1:1 molar ratio in tetrazole (CH₂N₄) under H₂O₂ and dimethylacetamide (DMA) conditions, followed by further hydrolysis using trifluoroacetic acid (TFA) and dichloromethane (DCM). For the synthesis of eperezolid phosphate, eperezolid was mixed with phosphoramidous acid (Dibenzyl N,N-diisopropylphosphoramidite) at the 1:1 molar ratio in CH₂N₄ under acetonitrile (MCN) and meta-chloroperoxybenzoic acid (mepba) conditions. After 18 hours of reaction at room temperature, eperezolid phosphate was obtained by treating the mixture with palladium on carbon (pd/c) in methanol (MeOH). After these reactions, the resulting mixture was concentrated and subjected to flash column chromatography on silica gel using ethyl acetate/n-hexane (1:4) as the eluent to collect the desired products: delpazolid phosphate (white to off-white solid, 78% yield, >97% purity) and eperezolid phosphate (white to off-white solid, 44% yield, >97% purity). These products were then analyzed for purities (LC-MS: liquid chromatography-mass spectrometry) and validated (NMR: nuclear magnetic resonance).

Delpatzolid phosphate: 1H NMR (400 MHz, D2O) δ 8.14 (a, 1H), 7.57 (dd, J=13.2, 2.4 Hz, 1H), 7.41 (t, J=8.8 Hz. 1H), 7.34-7.28 (m, 1H), 4.95 (s, 1H), 4.20 (t, J=9.2 Hz, 1H), 4.11 (ddd, J=11.8, 5.6, 2.8 Hz, 1H), 4.03-3.96 (m, 2H), 3.94-3.90 (m, 2H), 3.82 (q, J=6.4 Hz, 1H), 3.59 (t, J=6.8 Hz, 1H), 3.44-3.39 (m, 2H), 2.85 (s, 3H).

Eperezolid phosphate: 1H NMR (400 MHz, D2O) δ 7.36 (d, J=12.8 Hz, 1H), 7.14 (d, J=5.2, 2H), 4.32 (s, 2H), 4.16 (t, J=9.2 Hz, 1H), 3.77 (dd, J=9.2, 5.6, 1H), 3.71-3.69 (m, 2H), 3.57 (t, J=7.2 Hz, 1H), 3.53-3.50 (m, 4H), 3.05 (t, J=5.2 Hz, 4H), 2.63-2.59 (m, 3H), 1.93 (s, 3H).

25) Data availability and statistical analysis. All assays were performed in triplicate and results were expressed as average±SD unless otherwise stat, Statistics are analyzed and graphed using Prism v9 (Graph Pad). After testing for normality, a two-tailed paired Student's t-test was used for pairwise statistical comparisons unless otherwise noted, Error bar in all figures represent means±SD. At least three biological replicates per experiment were carried out. Differences are significant at *P<0.05, **P<0.01, and ***P<0.001.

### 2. Results

### HTS Explores Synergistic Effects Between Lysosyme and Tedizolid Phosphate Against Broad-Spectrum Resistant Pathogens.

Using standard methicillin-resistant Staphylococcus aureus USA 300 (MRSA, ATCC-BAA1556) and Staphylococcus epidermis (MRSE, ATCC35984) as the model strains, which were both resistant to human lysozyme with the minimum inhibitory concentration (MIC) at the level of >150 mg/ml (FIG. 38 to FIG. 40), 1953 FDA-approved drugs were initially screened to evaluate their inhibition activities in the absence or presence of 1 mg/ml lysozyme (philological concentration in human tear) (FIG. 39), HTS successfully identified several hits that agents showed increased inhibitory activities in combination with lysosyme against MRSA and (or) MRSE (FIG. 39A to FIG. 40. Table below). Of these, tedizolid phosphate, an oxazolidinone class antibiotic, commercially available as SIVEXTRO™ (Merck & Co, Railway, NJ, USA) to treat gram-positive bacterial infections especially acute bacterial skin and kin structure infections (ABSSSI) [M. Bassetti et al., Core Evid. 14, 31-40 (2019)], showed exciting synergistic activities with lysozyme against both MRSA and MRSE (both inhibition ratios were above 90% FIG. 39A to FIG. 39B), against which tedizolid phosphate or lysozyme alone had little effect on bacterial growth (FIG. 39A to FIG. 39B).

Growth inhibition ratio
(mean ± SD, %)

| FDA-approved drugs | MRSA | MRSE |
|---|---|---|
| Chlorhexidine 2HCl | 94.16 ± 0.25 | 12.3 ± 18.27 |
| Tedizolid Phosphate | 92.61 ± 0.86 | 93.14 ± 5.17 |
| Tilmicosin | 91.59 ± 0.63 | 4.91 ± 8.1 |
| Mezlocillin Sodium | 90.46 ± 1.59 | 6.93 ± 8.22 |
| Bardoxolone Methyl | 29.95 ± 40.66 | 81.67 ± 1.61 |
| Cefdinir | 85.21 ± 9.84 | 20.31 ± 13.83 |
| Cefoperazone | 71.74 ± 5.27 | −16.2 ± 8.93 |
| Nitazoxanide | −34.29 ± 19.27 | 91.65 ± 7.12 |
| Carmofur | 82.5 ± 7.73 | 78.06 ± 11.38 |
| Doripenem Hydrate | 82.4 ± 5.63 | 11.18 ± 6.16 |
| Sultamicillin | 58.05 ± 16.75 | 45.94 ± 13.49 |
| Cefmetazole sodium | 58.25 ± 13.23 | 6 ± 29.05 |
| Benzylpenicillin | 48.39 ± 51.84 | 3.07 ± 4.93 |
| potassium | | |
| Mitomycin C | 66.02 ± 45.26 | 39.12 ± 43.57 |
| Ceftriaxone Sodium | 8.83 ± 1.45 | 34.56 ± 35.01 |
| Cefradine | 48.66 ± 13.26 | 33.52 ± 23.07 |
| Meropenem Trihydrate | 48.19 ± 11.14 | 20.45 ± 65.79 |
| Ceftazidime | 45.07 ± 6.86 | 34.84 ± 3.23 |
| Pentahydrate | | |
| Cefoselis Sulfate | 40.55 ± 6.2 | 51.56 ± 10.39 |
| Luliconazole | −9.69 ± 5.58 | 22.39 ± 10.76 |
| Cefcapene Pivoxil | 18.64 ± 7.49 | 3.23 ± 3.04 |
| Hydrochloride | | |
| Cefoselis Sulfate | 40.55 ± 6.2 | 51.56 ± 10.39 |

Then, standard checkerboard microbroth-dilution assays were performed in Luria-Bertani (LB) troth to confirm these combination effects (FIG. 40). Surprisingly, the fractional inhibitory concentration index (FICI) values [H. Sun et al., Nat. Commun. 11, 5263 (2020); Q. Zhang et al., Proc. Natl. Acad. Sci. U.S.A. 119, e2119417119 (2022)] between tedizolid phosphate and lysozyme against both MRSA and MRSE were so tiny (FICI<0.023, FICI≤0.5 is defined as synergism), indicating that their synergistic effects were extraordinary (FIG. 30A to FIG. 30B). Specifically, it was observed that tedizolid phosphate at a concentration of ca. 0.19 μg/ml (equivalent to 0.4 μM, 0.004 MIC) could re-sensitize MRSA to human lysozyme, resulting in a reduction of the MIC value of lysozyme by 1200-fold from >150 mg/ml to 0.1235 mg/ml (FIG. 30A to FIG. 30B). This phenomenon was also clearly observable in the time-kill curve [H. Sun et al., Nat. Commun. 11,263 (2020); Q. Zhang et al., Proc. Natl. Acad. Sci. U.S.A., 119, e2119417119 (2022)], an assay to evaluate the dynamic inhibition effect. As shown in FIG. 30C, neither 0.5 mg/ml lysozyme nor 0.25 μg/ml tedizolid phosphate (or even 0.5 μg/ml tedizolid phosphate) had any inhibitory effect on the growth of MRSA or MRSE in exponential growth phase. In contrast, bacterial growth was fully inhibited under combination treatment, as judged from bacterial load per ml culture dropping from the level of 10¹² to 10⁶ colony forming units (CFUs) after 22 hours incubation (FIG. 30C and FIG. 42 to FIG. 42C).

Following these findings, to determine the spectrum of the combination effect, it was tested against several gram-positive pathogens including Streptococcus pyogenes, Streptococcus mutans, Streptococcus pneumoniae, Enterococcus faecium, Enterococcus faecalis and Corynebacterium pseudodiphtheriticum (FIG. 30D and FIG. 64A to FIG. 64F). Synergistic effects were observed in all combination treatments with FICI values ranging from 0.015 to 0.156 (FIG. 30D). This phenomenon was also observed if tedizolid phosphate was used in combination with other sub-types of lysozymes, i. e., C-type, G-type, T4-type and bacterial-type lysozymes [L. Callewaert et al., J. Biosci. 35, 127-160 (2010)] (FIG. 30E, FIG. 43A to FIG. 43B and FIG. 65A to FIG. 65D). All indicated the combination therapy had a similar broad-spectrum effect.

Considering the resistance development is always the tough problem in treatment of bacterial infection [H. Sun et al., Nat. Commun. 11, 5263 (2020)], the effect of combination therapy on the resistance development was then assessed. Here, serial passages assays were performed on MRSA and MRSE exposed to tedizolid phosphate in the absence and presence of lysozyme at the increasing concentrations. The results revealed that combination therapy significantly suppressed the development of resistance to tedizolid phosphate if compared with that in the tedizolid phosphate alone group (FIG. 30F). Specifically, after 12-day serial passages, exposure to tedizolid phosphate alone led to 30-fold changes in the MIC level of tedizolid phosphate but remained unchanged under combination therapy. In addition, it was also observed that combination therapies could efficiently reduce the mutation prevention concentrations [H. Sun et al., Nat. Commun. 11, 5263 (2020); Q. Zhang et al., Proc. Natl. Acad. Sci. U.S.A. 119, e2119417119 (2022)] of drugs in both MRSA and MRSE (FIG. 30), with the Sent mutation frequencies [H. Sun et al., Nat, Commun. 11, 5263 (2020); Q. Zhang et al., Proc. Natl. Acad. Sci. U.S.A. 119, e2119417119 (2022)] of MRSA and MRSE being significantly narrowed from ≥5×10⁻⁷ to 2×10⁻⁹ when bacteria were treated with combination therapies (FIG. 30G and FIG. 44A). Also, the prevention concentrations of tedizolid phosphate were significantly reduced from >50 to ≤1 μg/ml in both MRSA and MRSE under combination treatments (FIG. 30H and FIG. 44B). These results indicated that the combination of lysozyme and tedizolid phosphate could effectively minimize de novo emergence of drug resistance.

The presence of biofilm increased the emergence of antibiotic resistance, thereby aggravating the antibiotic resistance crisis in the post-antibiotic era [D. Qu et al., Sci. Adv. 6, eaay9597 (2020)]. To determine the effects of the combination therapy against biofilm, crystal-violet assays [D. Qu et al., Sci, Adv. 6, eaay9597 (2020)] were subsequently performed on bacteria in their exponential growth phases, corresponding to the pre-biofilm stage, to assess whether biofilm formation could be inhibited. It was found that either tedizolid phosphate or lysozyme alone had only a minimum inhibitory effect on the biofilm formation, as judged fam a small reduction in absorbance at 590 nm. In contrast, biofilm was eliminated under combination treatment because absorbance was at the same level as that of the control group (FIG. 30I). Similarly, inhibition was also observed even after the initial appearance of biofilm, as biofilm-based bacterial growth in fresh LB medium was also thoroughly inhibited (FIG. 44C). All results pointed to the effectiveness of the lysozyme/tedizolid combination effect.

Proteomics Studies Prompted that Combination Therapy Resembled Treatment with Higher Dosage of Tedizolid Phosphate.

To explore the mechanism behind the combination effect, the inhibition effect on growth of log-phage MRSA treated with either combination therapy or increasing concentrations of tedizolid phosphate was evaluated by comparing their optical densities at 600 nm (OD As shown in FIG. 45A, bacteria treated with combination therapy or 40 μg/ml tedizolid phosphate had similar OD₆₀₀. The similarity indicated that combination therapy may work by amplifying the effect of tedizolid phosphate on the pathogen.

To validate this hypothesis, a proteomic analysis on MRSA under different treatments was subsequently performed. As it was considered that a proteomic study on MRSA treated by lysozyme was insufficient [X. Liu et al., Sci. Rep, 6, 19841 (2016)], it was necessary to firstly build a treated proteomic library. Similarities were observed in the heat-maps of changed proteins in those treated with combination therapy and 40 μg/ml tedizolid phosphate, as judged from the higher Pearson correction coefficient (R²˜0.95) [I. T. Jollife et al., Philos. Trans. Royal Soc. A 374, 2010202 (2016)] and principal component analysis (PCA) correlation [I. T. Jolliffe et al., Philos. Trans. Royal Soc. A 374, 20150202 (2016)] (FIG. 31A and FIG. 46A to FIG. 46). More importantly, by performing a tandem mass tag (TMT) labeling quantitative proteomics analysis [H. Guo et al., ACS Chem. Biol. 17, 2003-2009 (2022)] on MRSA, samples were further compared under different treatments for 1 hour (FIG. 31A to FIG. 31E and FIG. 47A to FIG. 47I). Specifically, it was observed that them were significant (p>0.05) up-regulation of 51 and down-regulation of 37 differentially expressed proteins (DEPs) in MRSA under combination therapy if compared with those noted in the 0.5 μg/ml tedizolid phosphate alone group (FIG. 31B), Gene ontology (GO) annotation analysis of these regulated proteins showed that these DEPs were mainly correlated with organelle organization/cellular component organization (biological processes), RNA binding/protein containing complex binding (molecular functions), and large ribosomal subunit/cytosolic large ribosomal subunit (cellular components) (FIG. 31C). These clusters in ribosome/protein synthesis were reasonable because tedizolid phosphate works as an oxazolidine class antibiotic inhibiting protein synthesis by disrupting ribosomal function. Interestingly, Kyoto Encyclopedia of Genes and Genomes (KEGG) enrichment analysis clearly demonstrated that these up-/down-regulated DEPs were significantly enriched in energy metabolism, i.e., pyruvate metabolism, citrate (TCA) cycle, pentose phosphate pathway and fructose/mannose metabolism (FIG. 31D). To visualize the effect on energy metabolism, an overall analysis was performed on these up/down-regulated proteins, especially those in the glycolysis pathway and TCA cycle. As shown in FIG. 31E, a great many crucial proteins, including phosphoglycerate kinase (Uniprot ID: Q5HQV3, 0.005 fold) and probable malate:quinone oxidoreductase I (Uniprot ID: P65421, 0.12 fold), were generally down-regulated, indicating that energy metabolism was greatly disrupted in MRSA under combination therapy.

To validate these findings, intracellular levels of three representative indices in energy metabolism, i.e., pyruvate, ATP and NAD⁺/NADH [A. Luengo et al., MoL Cell 81, 691-707.e696 (2021)] were subsequently analyzed. It was observed that the ratio of NAD⁺/NADH significantly increased from 8.84±1.89 to 18.43±4.42 if MRSA was treated with combination therapy for 1 hour, over which time both NAD⁺ and NADH decreased (FIG. 31M A similar change wats observed in the level of pyruvate (>40% decrease, FIG. 31G). Interestingly, combination therapy led to higher levels of ATP in MRSA, which was consistent with the level observed in cells treated with 40 μg/ml tedizolid phosphate alone (FIG. 31H), This increase might partly attribute to the ATP saved by the inhibition of synthesis of cell walls. In conclusion, together with the change of proteins in the energy pathway, cellular energy metabolism was strongly disrupted, which likely increased cell death of the pathogen under combination therapy.

### Combination Therapy Alkalized the Cellular pH Level to Significantly Increase Intracellular Accumulation of Tedizolid Phosphate by Activating Alkaline Phosphatase.

As the most typical effect of oxaxolidinone class antibiotics including tedizolid phosphate, is to inhibit bacterial protein synthesis [J. M. Rybak et al., Infect. Dis. Ther. 4, 1-14 (2015)], the degree of inhibited nascent protein synthesis was thus evaluated in MRSA treated with different concentrations of tedizolid phosphate in the absence or presence of 0.5 ng/ml lysozyme. As shown in FIG. 32A, the minimal dosage of tedizolid phosphate in monotherapy or combination therapy was 9.5 or 0.32 μg/ml if half of the nascent protein suffered from synthesis inhibition Given these similarities in overall effects and tedizolid phosphate-based protein synthesis inhibition, it was assumed that combination therapy increased the intracellular accumulation of tedizolid phosphate.

To verify this assumption, the intracellular level of tedizolid phosphate was firstly measured in MRSA exposed to different concentrations of tedizolid phosphate in the absence or presence of lysozyme by performing time-dependent LC-MS/MS analysis, Interestingly, a 10-fold increase in the level of intracellular tedizolid phosphate was observed in MRSA after 4 hours treatment if even a vey small concentration of lysozyme (≤0.25 mg/ml) was used in combination with tedizolid phosphate (FIG. 32B). Meanwhile, if MRSA was exposed to different concentrations of tedizolid phosphate for 1 hour, intracellular accumulation of tedizolid phosphate would reach a concentration of ca. 100 μg/l in MRSA under combination therapy. Such an accumulation could not be achieved unless ≥4.5 μg/ml tedizolid phosphate alone was added (FIG. 32C). Together these findings indicate that combination therapy can increase intracellular accumulation of tedizolid phosphate, leading to a strong bactericidal effect.

To further explore the mechanism of increased accumulation behind combination effect, whether combination therapy had any effects on the cell membrane and cell wall was subsequently evaluated. By performing permeability assays using propidium iodide (Pb, a red-fluorescent DNA dye, which can only cross the plasma membrane of nonviable cells [Y. Liu et al., Adv. Sci. 7, 1902227 (2020)], no difference was found in the PI-based fluorescence signal, indicating there was no damage to the cell membrane (FIG. 45B) To evaluate the integrity of the cell wall, fluorochrome SYBR gold, a nucleic acid dye [R. Santiso et al., BMC Microbiol. 11, 191 (2011)], was added to the MRSA-agarose mixture and pipetted onto precooled slides. However, it was hardly observed any diffusion of nucleic acid in combination group, indicating that combination therapy had little effect on the cell wall (FIG. 45C).

It is well-documented that efflux pumps or transporters on cell membrane play critical roles in pumping drugs into/out of cells and cell metabolism [A. Sharma et al., Med, Res. 149, 129-145 (2019); M. Putman et al., Microbiol. Mol. Biol. Rev. 64, 672-693 (2000)]. Thus, their potential effects on combination therapy were next investigated using representative inhibitors. However, no related differentially expressed proteins (DEPs), in particular membrane proteins, were identified in the proteomics analysis. Furthermore, no effect on the FICI of combination therapy was observed if MRSA was exposed to any of the 20 inhibitors targeting well-known efflux pumps/transporters [A. Sharma et al., Indian J. Med. Res. 149, 129-145 (2019)] (at 0.25 MIC, FIG. 48A to FIG. 48V). As ions-dependent transporters have been shown to be important in development of antibiotic resistance [M. Putman et al., Microbiol, Mol. Biol. Rev. 64, 672-693 (2000)], the FICI of combination treatment was next evaluated after MRSA was exposed to 14 inhibitors targeting typical ions, i.e., Na⁺, K⁺, Ca²⁺, Cl⁻ and H⁺ [J. Stautz et al., J. Mol. Biol 433, 16696 (2021)]. As shown in FIG. 32D, compared with other inns-targeting inhibitors, cells incubated with CCCP Carbonyl cyanide m-chlorophenyl hydrazone) [H. Sun et al., Nat. Commun. 11, 5263 (2020)], a H⁺ inhibitor led to the significant increase in FICI of combination therapy (FIG. 49A to FIG. 49P), indicating that H⁺ played a critical role in the re-sensitization of tedizolid phosphate against broad-spectrum gram-positive pathogens in combination therapy.

Following these studies, the membrane potentials of MRSA and MRSE, with or without combination therapies were next compared, in which bacteria treated with CCCP, a disruptor of potential by uncoupling of the proton gradient [H. Sun et al., Nat, Commun. 11, 5263 (2020); Q. Zhang et al., Proc. Nat. Acad. Sci. U.S.A., 119, e2119417119 (2022)], served as a positive control group. After 1 hour treatment, it was observed that combination therapy led to a reduced membrane potential if compared with those under other treatments, as judged by an increased fluorescence ratio (green/red) (FIG. 32E). This phenomenon was more obvious in MRSE than MRSA, which may be attributable to their different bacterial properties. Considering the role of proton/H⁺ in pH homeostasis of cellular micro-environment [N. Guan et al., Appl Microbiol. Biotechnol. 104, 51-65 (2020)], the pH level in MRSA under combination therapy was next determined using a pH-sensitive fluorescence probe BCECF-FAM (2′,7′-bis-(Carboxyethyl)-5-(and-6)-carboxyfluorescein) acetoxymethyl ester) [Y. Liu et al., Adv. Sci. 7, 1902227 (2020)]. It was found that combination therapy significantly alkalized the cellular pH level, as evidenced by the increased intensity at 535 nm if compared with other groups (FIG. 3F). Meanwhile, cells treated with 0.5 mg/ml lysozyme alone also had an elevated pH although it was far less than that observed in the combination group, indicating that combination therapy aggravated the alkaline degree of cellular micro-environment, Indeed, the alkalization was also successfully observed in MRSE (FIG. 50A).

As it had been well-documented that tedizolid phosphate was hydrolyzed into tedizolid by endogenous alkaline phosphatase in cytoplasm [M. Bassetti et al., Core Evid. 14, 31-41 (2019)], it was assumed that the alkalized cellular micro-environment would activate alkaline phosphatase to accelerate metabolism of tedizolid phosphate. To verify this, the in vitro hydrolysis rate of tedizolid phosphate by extracting alkaline phosphatase from sonication lysis of MRSA under different treatments was compared. It was observed that up to 65% of tedizolid phosphate in combination therapy group had been hydrolyzed after 1 hour, which was significantly faster than other treatments (only 25%) (FIG. 32G). To visualize the activated hydrolysis, the microscopy images of MRSA treated with BCIP/NBT working solution were analyzed by investigation of levels of brown-black NBT-formazan [L. Zhang et al., J. Biol. Chem. 280, 37289-37296 (2005)] (maximum absorption at 550 nm), a catalytic product of BCIP/NBT by alkaline phosphatase in cells. As shown in FIG. 32H, combination treatment led to more brown-black dots (each one representing one MRSA cell). Indeed, the normalized increase was also observed when the NBT-formazan-based absorbance was directly measured (FIG. 50B), indicating combination therapy did result in alkalization of the cellular pH level to activate alkaline phosphatase.

Notably, as one of promising strategies, pro-drug with phosphorylation modification is widely documented in the pharmaceutical industry due to its advantages such as reduced toxicity, enhanced bio-stability and improved lipid-solubility for efficient cell membrane penetration. Thus, we next selected two representative oxaxolidinone-class antibiotics and synthesized their pro-drugs by phosphorylating their hydroxyl groups located on both sides of their core structures (FIG. 33A to FIG. 33D and FIG. 41A to FIG. 41C). Excitingly, when combined with lysozyme, both delpazolid phosphate (DP) and eperezolid phosphate (EP) exhibited strong synergistic effects (FICI≤0.09373, slightly higher than TP's. FIG. 33A to FIG. 33D). This suggests that oxazolidinone-class antibiotics themselves may play an irreplaceable role in the combination with lysozyme. Consequently, TP was selected as the representative oxazolidinone-class antibiotic for further analysis of combination effect with lysozyme in subsequent studies.

Urea Cycle and Cell Division were Targeted in Combination Therapy

Further detailed analysis of the proteomics studies data revealed that combination therapy significantly increased the levels of arginine deiminase (Uniprot ID: Q5HKU2, 3.9-fold) and urease subunit alpha (Uniprot ID: A5IV71, 1,5-fold) but reduced expression of ornithine carbamoyltransferase (Uniprot ID: Q5HCR3, 0.5-fold) in MRSA compared with cells exposed to lysozyme alone (FIG. 34A to FIG. 34). These enzymes play a critical role in the urea cycle leading to production of urea and ammonia. Such change in this pathway and the endpoints would result in an alkalized cellular min-environment.

To validate this explanation, FICI analysis was subsequently performed on MRSA under combination therapies in the absence and presence of inhibitors targeting arginine deiminase (BB-Cl-Amidine [M. Quintero et al., J. Bacteriol. 182, 1008-1015 (2000)]) or urease (acetohydroxamic acid [Y. F. Rego et al., J. Adv. Res. 13, 69-100 (2018)]). As shown in FIG. 34F, the presence of inhibitors significantly increased FICI from 0.015 to 0.1 (up to 0.25 in cultures of both inhibitors), indicating that increased intracellular accumulation of tedizolid phosphate had been partly inhibited (FIG. 51A to FIG. 51C). A similar result was obtained from a parallel BCECF-AM-based pH-sensitive fluorescence assay (FIG. 51D).

Despite the effects of lysozyme, the intracellular pH was always tightly regulated to maintain cellular homeostasis [W. Aoi et al., BioMed Res. Int. 2014, 598986 (2014)]. Unlike eukaryotic cells which have a physiologically normal intracellular pH ranging between 7.0 and 74 [W. Aoi et al., BioMed Res. Int. 2014, 598986 (2014)], bacteria usually maintain their cytoplasmic pH in a narrow range (˜7.5-7.7) [T. A. Krulwich et al., Nat. Rev. Microbiol. 9, 330-343 (2011)] although some species can survive in severe pH environments. In addition to a challenging extracellular environment, some cellular metabolisms, including the urea cycle, increase the cell burden in pH homeostasis [M. Shibamura-Fujiogi et al., Commun. Biol. 5, 1284 (2022)]. However, the cellular acid-base regulatory system has limited ability to overcome dramatic change in pH, having strong capability around neutral pH but considerably weaker ability at pH levels increasingly different from the homeostatic pH [T. A. Krulwich et al., Nat, Rev. Microbiol. 9, 330-343 (2011)]. As basic residue-rich enzyme [J. M. Tiffany, Eye 17, 923-926 (2003)], lysozyme displays a strong positive-charge in the physiological environment, and thus intracellular pH homeostasis is inevitably disrupted when it is inserted into the cell wall/membrane or transported into cells [R. H. Ibrahim et al., Cur. Pharm. Des. K. 671-693 (2002)]. Thus, pH homeostasis would be more frangible after cell was treated with lysozyme, which might account for why pH homeostasis was easier to be disrupted in cells under treatments with combination therapy than tedizolid phosphate alone (FIG. 51D).

For further verification, the FICI of combination therapy was analyzed using a randomly selected poly-l-lysine with a high level of positive charge in physiological conditions in place of lysozyme [. Wang et al., Front. Bioeng, Biotechnol. 9, (2021)]. It was observed that this compound could successfully reproduce the expected synergistic effect in combination therapy, although the FICI was a little higher (FICI=0.375) (FIG. 34G). These studies provided strong supporting evidence for the suggested mechanism for the effectiveness of combination therapy.

Ultimately, scanning electron microscope (SEM) imaging was performed to visualize the morphological change in MRSA or MRSE under combination therapy. As shown in FIG. 34K and FIG. 52, pathogens under combination treatments showed obvious digestion-like cell lysis compared with those with or without treatment of tedizolid phosphate or lysozyme alone. Interestingly, numerous bubble-like protuberances appeared, especially in MRSE, if lysozyme was used in combination with ≥0.25 μg/ml tedizolid phosphate (FIG. 34H). These extraordinary morphological changes led to an in-depth analysis of these changed proteins in proteomics studies. It was observed them, was a significant increase in the level of OntA (Uniprot ID: Q2FV54, FIG. 34I, an O-acetyl transferase facilitating bacterial resistance against lysozyme by modifying the C₆-hydroxyl group of N-acetylmuramyl residues in the peptidoglycan of the cell wall [A. Bera et al., Mol. Microbiol. 55, 778-787 (2005)]). This overexpression may be the result of stress response to exposure to lysozyme. It has been previously reported that increased OatA production leads to aberrant cell division [E. Bernard et al., PLoS One 7, e47893 (2012)]. Interestingly, proteomics studies also revealed significantly decreases in the levels of FtsA (Uniprot ID: Q8NX33, 0.003-fold) and SepF (Uniprot ID: Q8NX33, 0.5-fold) (FIG. 34J to FIG. 34K.) FstA and SepF are very critical for FtsZ bundling, Z-ring tethering, and membrane r-shaping activities during cell division because down-regulation of anchors including SepF results in Z-ring assembly being unequal at the cell division site [D. W. Adams et al., Nat. Rev. Microbiol. 7, 642-653 (2009); A. Sogues et al., Nat. Commun. 11, 1641 (2020)]. This suggested that these bubble-like protuberances in SEM imaging were associated with changes in OatA, FtsA and SepF leading to faulty Z-ring assembly at unequal cell division sites, resulting in the presence of small nascent bacteria as bubble-like protuberances. To validate this explanation, immunofluorescence microscopy assays was subsequently performed on MRSA incubated with FtsZ antibody. As shown in FIG. 34L, lass-like Z-rings composed of FtsZ were observed at the unequal bubble-like junction (half-moon-shaped, combination group) in contrast to the equal division site (circle-like, other groups), indicating that combination therapy did lead to the unequal cell division. Such faulty cell division provides a reasonable molecular basis to explain these SEM results and further evidence for the ability of tedizolid phosphate (and lysozyme) to inhibit the growth of pathogens.

Combination Effect was Well-Transferred from Cell-Based Infection Model into Rat Skin Infection Model

Next, the application potential of combination therapy in vivo was further evaluated, Here, ARPE-19 cell (a spontaneously arising retinal pigment epithelial cell), NIH/3T3 cell (a fibroblast cell isolated from a mouse NIH/Swiss embryo), and 293T cell (an epithelial-like cell isolated from a human kidney) were firstly selected to analyze toxicities of tedizolid phosphate and lysozyme. All three cell lines were found to be insensitive to tedizolid phosphate and lysozyme, regardless of exposure time (12 hours and 24 hour), indicating that safe doses of combination therapy did not result in drug-induced cell death (FIG. 53A to FIG. 53F).

Then, considering lysozyme is abundant in human tears. ARPE-19 cell was selected to perform a cell-based bacterial infection assay. It had been observed that infection caused by MRSA at the multiplicity of infection (MOI) of 10 resulted in 50% cell death after 24 hours, and that neither tedizolid phosphate nor lysozyme alone could rescue these infected cells. However, cell death was prevented if 1.0 μg/ml tedizolid phosphate was used in combination with 0.5 mg/ml lysozyme, which was likely to be attributable to death of the bacteria under combination therapy (FIG. 53G to FIG. 53H), Indeed, the bacterial load was significantly reduced from the levels of 10⁷ to 10⁴ CFU/ml in ARPE-19 cells treated with ≥1.0 μg/ml tedizolid phosphate in the presence 0.5 mg/ml lysozyme (FIG. 53H).

As tedizolid phosphate is FDA-approved drug for treatment of acute bacterial skin and skin structure infections [J. M. Rybak et al., Infect Dis. Ther. 4, 1-14 (2015)], to validate whether the combination treatment would be effective in vivo, the relieved effect was subsequently evaluated in a rat skin infection model [Q. Zhang et al., Proc, Natl. Acad. Sci. U.S.A. 119, e2119417119 (2022)]. Here, a 1.25 ml aliquot of 4.0×10¹¹ CFU MRSA suspension was sprayed onto a skin wound 0 cm×3 cm) of each anesthetized rat to build skin infection model, followed by treatment with sterile medical gauze soaked with normal saline, tedizolid phosphate (mg/kg rat in total) or lysozyme (5 mg/kg rat in total), or their combination (FIG. 35A). At 168 hours post-inoculation, the bacterial load in the wound % was significantly reduced (>10⁶-fold) if these infected rats were treated with combination therapy (FIG. 35B and FIG. 54). Hematoxylin and Cosin (H-E) staining revealed that their inflammatory symptoms were also effectively relived. As shown in FIG. 35C, denser inflammatory cells with blue nuclei were observable in the control, tedizolid phosphate, and lysozyme groups, but not in the combination therapy group. In addition, the smooth edges of slides from rats in the combination therapy group also indicated the better recovery, which was supported by their recovering wound images (FIG. 55A to FIG. 55B). The recovery was further confirmed by two inflammatory indices, i. e., interleukin 6 (IL-4) and reactive oxygen species (ROS) [Z. Zhang et al., PLoS One 10, e0138597 (2013)]. Specifically, more than 20% and 70% decreases were observed in the levels of IL-6 and ROS respectively (FIG. 35D to FIG. 35E). These results clearly showed combination therapy effectively relieved the symptoms in the rat Skin infection model, indicating that the combination effect could be successfully transferred into in vivo therapy).

### Combination Therapy Significantly Relieved the Symptoms of Mice in in Ocular Infection Model

Lysozyme is self-secreted antimicrobial and abundant in human secretions such as tears. Thus, the potential of combination therapy was next evaluated in an ocular infection model, By slowly inoculating each ocular surface of C57/BL6 mouse with 2.5 μl of bacterial suspension containing 1.0×10⁷ CFUs MRSA at exponential phase, the ocular infection model was successfully built at 6 hours post-inoculation. Then, these infected mice were treated with PBS (2.5 μl), mono-therapy of tedizolid phosphate (0.125 mg/ml, 2.5 μl) or lysozyme (100 mg/mL, 2.5 μl), or their combination therapy before rinsing each ocular surface with 5 μl of PBS to collect tear fluid from the canthus for culture. Drugs to treat uninfected mice were replaced with 2.5 μl of PBS. All steps were repeated at 24-hour post-inoculation. At 48 hours post-inoculation, all mice were sacrificed to collect their eye tissues, including eyeballs and accessory structures (FIG. 57) [B. D. Kels et al., Clin. Dermatol. 33, 140-146 (2015)]. It was found that combination therapy significantly reduced the bacterial load in both tear fluids and eyeball homogenates (t; 0.01-fold, FIG. 36A to FIG. 36B). In addition, considering the potential risk of tedizolid phosphate or lysozyme alone to strengthen the infectivity of the bacterium itself, the percentage of invaded epithelial cells and the average count of bacteria per epithelial cell in tear fluids were determined, respectively [Z. Zhang et al., PLoS One 10, e0138597 (2015)]. As shown in FIG. 36C and FIG. 57, compared with epithelial ceils from the uninfected group, those from control or mono-therapy groups had higher invasion ratios of >60%. Interestingly, the percentage significantly fell if infected mice were treated with combination therapy. It was also observed that the combination therapy had a seemingly negligible effect on infectivity of the bacterium itself, as judged from its almost proportional reduction in average invasive bacterial counts compared with bacteria in team. Use of surfactant protein-D (SP-D) to determine whether the host innate immune defense against ocular bacterial infection was disrupted [Z. Zhang et al., PLoS One 10, e0138597 (2015)], revealed that combination therapy could significantly strengthen the host defense as the expression keys of SP-D in tear fluids had a recovering trend compared with that in the uninfected group, as evidenced by a 0.61-fold rise in the control group compared with 1.5-fold in the combination group (FIG. 36D). However, the change in ocular homogenates was negligible, which may attribute to the low proportion of SP-D erected into tear fluids to protect eyeballs from ocular infection [Z. Zhong et al., PLoS One 10, e0138597 (2015)] (FIG. 36D). To visualize the injuries to the ocular surfaces of mice under different treatments, each cornea was photographed to score the damage using sodium fluorescein to stain the ocular surface. The results showed that combination therapy led to greater cornea) integrity in infected mice, in which no obvious fluorescin was observed (FIG. 36E and FIG. 58A to FIG. 58D). In addition, the H-E staining analysis on these collected ocular tissues revealed that, compared with smooth edges in the dehydrated vitreous body in uninfected mice, permeating inflammatory cells and “irregular” retina were clearly observed, especially at the edge of the lens in control infected mice (FIG. 36F). Neither mono-therapy of tedizolid phosphate or lysozyme was able to prevent such permeation and irregularities. However, it was observed that mice under combination therapy had the recovered smooth edge in dehydrated vitreous body and retina, in which ganglion cells (GC) were in uniform arrangement (FIG. 36F and FIG. 56). All results indicated the great potential of combination therapy in clinical treatment of ocular infection,

The First Report to Comprehensively Explore Murine Ocular Microbiome, Revealing that Combination Therapy Successfully Re-Built the Ocular Bacterial Community.

Misuse, overuse, and prolonged use of antimicrobials have been consistently shown to cause the disturbance to the usual constituents and balance of the microbiome at various body sites, leading to further dysbiosis and greater susceptibility to (chronic) diseases [M. Teweldemedhin et al., BMC ophthalmology 17, 212 (2017); L. Maier et al., Nature 599, 120-124 (2021); K. A. Willis et al., Sci. Rep. 10, 12035 (2020)]. Interestingly, eye has long been regarded us an Almost sterile microenvironment [K. A. Willis et al., Sci. Rep. 10, 12035 (2020); W. Xue et al., Front. Coll. Infect. Microbiol. 11, 759333 (2021)], and it is far from gut where various bacterial communities arm well-documented and continuously reported [L Maier et al., Nature 599, 120-124 (2021)]. More recently, scientists revise the traditional opinion because of the reported existence of microbiome even in heathy eye, which is hypothesized to be result of “gut-eye axis” [L. Maier et al., Nature 399, 120-124 (2621); W. Xue et al., Infect. Microbiol. 11, 759333 (2021)]. Considering that microbial genomics is ascendant in revealing bacterial communities in the host tissues [J. S. Johnson et al., Nat. Commun. 10, 5029 (2019)], the microbiome analyses were next performed on tear fluids and homogenized eye tissues to evaluate the potential risk of combination treatment on ocular microbiome.

By using 16S rRNA meta-barcoding to characterize the microbiome (FIG. 6A to FIG. 60C) [J. S. Johnson et al., Nat. Commun. 10, 5029 (2019)], maps of the bacterial communities of the ocular surface (tear fluid) and eyeball (ocular homogenate) of healthy C57/BL6 mice were drawn up. To our knowledge, this is the first study to completely analyze ocular microbiome in such tissues (regions) [H. J. Lee et al., Mucosal. Immunol. 15, 1330-1362 (2022); C. S. de Paiva et al., Sci. Rep. 6, 23561(2016)]. A total of 2122 operational taxonomic units (OTUs, bacteria in bacterial communities) were observed in tear fluids from healthy C57/BL6 mice, of which 575 were shared as core OTUs with those observed in tears from infected mice under different treatments (FIG. 37A). Interestingly, it was observed that bacteria in communities (cutoff≥3% proportion, at the genus level) in healthy murine tears were predominantly anaerobic probiotics, including Clostridium_XIVa (8%), Bifidobacterium (6%), Lachnospiracea_incertae_sedis (5%), Blanaia (5%), Bacteroides (4%), Faecalibacterium (4%) and Prevotella (3%). A similar distribution was noted in bacterial communities from infected mice although their proportions varied by treatments (FIG. 378 and FIG. 60A to FIG. 60). MRSA-infection led to an increase in the proportion of Staphylococcus from 0.052% (healthy mice) into ˜3% (infected mice). However, combination therapy significantly reduced this proportion to almost pre-infection levels (˜0.078%). This reduction was not evident with, either tedizolid phosphate or lysozyme monotherapy, which was consistent with the CFUs-based evidence described above. Meanwhile, some anaerobic probiotics, including Anaerostipes, Prevotella and Propionibacterium [S. Selvamani et al., Appl. Sci, 11, 7247 (2021)] were observed to increase in the combination group (FIG. 37C to FIG. 37D). More importantly, the overall bacterial communities successfully recovered, as judged from the heat-maps showing the general composition of bacteria in tears (FIG. 37C and FIG. 61A), which was further confirmed by partial least squares-discriminant analysis (PLS-DA) because closer distance in the PLS-DA map was observed between the uninfected and combination groups (FIG. 37E). To evaluate the bacterial community diversities within one group or among groups, alpha and beta diversity analyses were performed, respectively. As shown in FIG. 37F and FIG. 37G, other than infected mice in the lysozyme group, all others had greater bacterial community diversities than uninfected mice, as judged from their higher Shannon indices and weighted unifrac distances (two indicts had positive correlation with alpha and beta diversities, respectively) Finally, to analyze the differences in bacterial communities at the taxonomy level, the revolutionary branching trees were devised by using Graphlan map (FIG. 37H). It was observed that bacteria in team were concentrated in Firmicutes and Bacteroides genera, although others including Actinobacteria and Bifidobacterium as probiotics also played important roles. Similar trends were observed in the bacterial communities of ocular homogenates although the affected degree differed, especially in proportion of Staphylococcus in infected mice (FIG. 61B to FIG. 61C and FIG. 62A to FIG. 63B), However, as the bacterial community was so complicated that it was difficult to confirm how or whether the growth of bacteria in the network was absolutely inhibited or promoted because of inter-exclusion and co-existence among these bacteria (FIG. 37I). In conclusion, all results indicated that combination therapy not only eliminated pathogens from mice suffering from ocular infection, but also allowed rapid recovery of their ocular bacterial communities.

### DISCUSSION

Intracellular pH is the measure of the acidity or basicity of intracellular fluid, which is critical for both enzyme-dependent cell metabolism and physiology function [W. Aoi et al., BioMed Res. Int. 2014, 598986 (2014)]. Loss of intracellular pH homeostasis results in compromised activity of enzymes, disruption of cell metabolism, inhibition of cell growth and even cell death. In general, bacteria need maintain their cytoplasmic pH in a narrow range (˜7.5-7.7). However, it has been reported that some bacteria can also survive in severe conditions, such as alkaliphilic Bacillus species growing in alkali soil (pH˜9.5). Indeed, to minimize the effect of these extreme living conditions on intracellular pH, organisms have evolved lots of buffer systems to maintain the intracellular pH homeostasis. A typical regular is a trans-membrane transporter, such as a proton pump. By performing energy dependent or independent conformational change, transporters are able to pump in or out H⁺/OH⁻, which facilitates the relatively stable intracellular pH level. Nevertheless, some essential cellular metabolisms, especially urea cycle, produce abundant acid or basic end products, which further increase the cellular burden to maintain pH homeostasis because the abilities of regulatory systems are limited. Thus, such pH homeostasis becomes more frangible if the cell is treated with high dose of charged compound, such as lysozyme.

Human lysozyme has >2.5% basic residues, i.e. arginine, histidine and lysine, on the surface [T. Masuda et al., Chem, Senses 30, 667-681 (2005)], and thus displays a considerable positive charge in physiological conditions. Indeed, as a typical model protein, lysozyme has been studied for over a century [M. Teweldemedhin et al., BMC ophthalmology 17, 212 (2017)], More importantly, as a naturally occurring antibiotic to kill gram-positive bacteria, lysozyme is self-secreted to protect the eye from bacterial infection [J. M. Tiffany, Eye 17, 923-926 (2003)]. Despite this, lysozyme use as an antibiotic agent has been discontinued for a long period, which panty attributes to the extremely common use of traditional antibiotics as well as reported resistance in gram positive pathogens, especially MRSA. Up to now, its application is not considered, despite increasing concern about levels of antibiotic resistance worldwide.

Here, by performing HTS between lysozyme and 1953 FDA-approved drugs, excellent re-sensitization of tedizolid phosphate against broad-spectrum resistant gram-positive pathogens by lysozyme was firstly reported. Investigation of the mechanism revealed that the combination disrupted pH homeostasis and thus increased intracellular accumulation of tedizolid phosphate. Further proteomics studies on MRSA provided additional evidence for effectiveness at the molecular level and a powerful basis for future studies on these drug-resistant pathogens. An important example in this disclosure was the effect on cell division. In bacteria, cell division occurs by the ingrowth of the envelope layers composed of membrane and peptidoglycan (PG) in the cell wall to form a septum that splits the cell into two compartments [D. W. Adams et al., Nat. Rev. Microbiol. 7, 642-653 (2009)]. This separation is orchestrated by a tubulin homologue, FtsZ, which as polymerized to form a ring-like structure (termed as Z-ring) [D, W, Adams et al., Nat. Rev. Microbiol. 7, 642-653 (2009); A. Sogues et al., Nat. Commun. 11, 1641 (2020)]. The Z-ring is a discontinuous structure consisting of dynamic patches of FtsZ. In addition, about a dozen additional essential proteins, such as SepF and FtsA in MRSA, are also recruited to the division site during cell division, and subsequently activated cell wall synthesis to drive cell envelope constriction [A. Sogues et al., Nat. Commun. 11, 1641 (2020); S. Booth et al., Protein Sci. 2, 2042-2054 (2019)]. If accessory proteins are deficient, there would be insufficient separating power to drive the division in septum, leading to two undivided compartments [S. Booth et al., Protein Sci. 28, 2042-2054 (2019)]. Even if cells are successfully separated, bacteria are unable to survive unless it they divide equally. Indeed, correct division also depends on other critical factors including tightly regulated OatA [E Bernard et al., PLoS One 7, e47893 (2012)]. Although tedizolid phosphate as an oxazolidinone class antibiotic is recognized as a bacterial protein synthesis inhibitor, its downstream functional targets have received less attention. The result of the current study clearly showed that combination therapy significantly changed the expression levels of OatA and SepF/FtsA, and thus caused the unequal and unsuccessful cell division. These effects on cell division regulation might be a novel mechanism of tedizolid phosphate anti-bacterial activity, although further studies are required to validate it.

The current study also evaluated the translational abilities of the combination therapy from in vitro to in vivo in skin and ocular infection models. Treatment with the combined therapy led to rapidly resolvent of both experimentally infected wounds and the ocular surface. In addition, ocular microbiome-based analysis was also performed. To our knowledge, this is the first study to completely analyze the murine ocular microbiome in eyeball (ocular homogenate and ocular surface (tear fluid) [11. J. Lee et al., Mucosal, Immunol. 15, 1350-1362 (2022); C. S. de Paiva et al., Sci, Rep. 6, 23561 (2016)]. The study revealed that the microbiome of such ocular tissues had great community diversity, comprising several anaerobic genera [K. A. Willis et al., Sci. Rep. 10, 12035 (2020); W, Xue et al., Front. Cell. Infect, Microbiol, 11, 759333 (2021)]. It was noted that combination therapy allowed a rapid regeneration and re-establishment of the balance of ocular microbiome, indicating its potential in future clinical applications.

Collectively, this disclosure reveals the potential of tedizolid phosphate/lysozyme as a novel adjuvant against broad-spectrum gram-positive pathogens and provides critical referred bases for future drug development.

Although the foregoing disclosure has been described in some detail by way of illustration and example for purposes of clarity of understanding, one of skill in the an will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A combination of one or more enzymes and one or more anti-microorganism drugs.

2. The combination of claim 1, wherein compared with the anti-microorganism drug alone, said combination results in at least a 10-fold, 50-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold increase in the sensitivity of a microorganism to the anti-microorganism drug.

3. The combination of claim 1, wherein the fractional inhibitory concentration index (FICI) value of the combination is not higher than 0.5; preferably not higher than 0.3; more preferably not higher than 0.1; most preferably not higher than 0.03.

4. The combination of claim 1, wherein the microorganism is a bacterium, fungus, virus, or parasite; preferably, a bacterium.

5. The combination of claim 4, wherein the microorganism is a drug-resistant bacterium.

6. The combination of claim 3, wherein the bacterium is a Gram-positive bacterium or Gram-negative bacterium; and preferably, the bacterium is a Gram-positive bacterium.

7. The combination of claim 6, wherein the Gram-positive bacterium includes but is not limited to methicillin-resistant Staphylococcus aureus (MRSA), methicillin-resistant Staphylococcus epidermis (MRSE), Enterococcus faecalis, Enterococcus faecium, Streptococcus mutans, Corynebacterium pseudodiphtheriticum, Streptococcus pyogenes or Streptococcus pneumoniae, Propionibacterium acnes.

8. The combination of claim 7, wherein the Gram-positive bacterium is methicillin-resistant Staphylococcus aureus (MRSA) or methicillin-resistant Staphylococcus epidermis (MRSE).

9. The combination of claim 1, wherein the enzyme is a hydrolase.

10. The combination of claim 9, wherein the hydrolase is selected from the group consisting of lysozyme, an isoform and homologue thereof, lysin and lysostaphin; and preferably, the hydrolase is lysozyme or an isoform or homologue thereof.

11. The combination of claim 10, wherein the lysozyme is of C-type (from, for example human or chicken), G-type (from, for example goose), T-type (from, for example T4 bacteriophage) or bacterial-type (from, for example bacteria, such as Bacillus subtilis).

12. The combination of claim 1, wherein the anti-microorganism drug is a drug with an inhibitory or killing activity against the microorganism.

13. The combination of claim 12, wherein the anti-microorganism drug is a drug approved by a local drug regulatory authority.

14. The combination of claim 13, wherein the local drug regulatory authority is the FDA in the USA, EMA in EP, PMDA in JP, or NMPA in CN.

15. The combination of claim 14, wherein the anti-microorganism drug is selected from the group consisting of FDA-approved drugs.

16. The combination of claim 12, wherein anti-microorganism drug is selected from an oxazolidinone class antibiotic, a β-lactam class antibiotic or anti-bacterial compound.

17. The combination of claim 16, wherein the oxazolidinone class antibiotic is an oxazolidinone with following core structure: 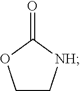
preferably, the oxazolidinone class antibiotic is an oxazolidinone with following core structure:

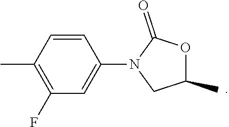

18. The combination of claim 12, wherein the anti-microorganism drug is selected from the group consisting of Auranofin, Cefdinir, Tedizolid Phosphate, Chlorhexidine 2HCl, Tilmicosin, Mezlocillin Sodium, Bardoxolone Methyl, Cefdinir, Cefoperazone, Nitazoxanide, Carmofur, Doripenem Hydrate, Sultamicillin, Cefmetazole sodium, Benzylpenicillin potassium, Mitomycin C, Ceftriaxone Sodium, Cefradine, Meropenem Trihydrate, Ceftazidime Pentahydrate, Cefoselis Sulfate, Luliconazole, Cefcapene Pivoxil Hydrochloride, Cefoselis Sulfate, TD52 (dihydrochloride), SCP1-IN-1, Rifamycin S, Paroxypropione, Ethyl 3,4-dihydroxybenzoate, Delafloxacin (meglumine), 2,4-Diacetylphloroglucinol, Sertaconazole (nitrate), delpazolid phosphate (DP), eperezolid phosphate (EP) and a pharmaceutically acceptable salt thereof.

19. The combination of claim 18, wherein the anti-microorganism drug is selected from the group consisting of Auranofin, Cefdinir, Tedizolid Phosphate, Chlorhexidine 2HCl, Tilmicosin, Mezlocillin Sodium, Bardoxolone Methyl, Cefoperazone, Nitazoxanide, Carmofur, Doripenem Hydrate, delpazolid phosphate (DP), eperezolid phosphate (EP) and a pharmaceutically acceptable salt thereof.

20. The combination of claim 19, wherein the anti-microorganism drug is selected from the group consisting of Auranofin, Cefdinir, Tedizolid Phosphate, delpazolid phosphate (DP), and eperezolid phosphate (EP).

21. The combination of claim 1, wherein the combination further comprises an autolysis promoter or simulator, including but not limited to a walR inhibitor or antagonist, or a N-acetylmuramoyl-L-alanine amidase domain-containing protein agonist; or
an LTA/WTA synthesis promoter or simulator, including but not limited to a promoter or simulator up regulating the expression of an enzyme selected from the group consisting of TarA, TarB, TarD, TarF, TarL, TarS, TagH and FmtA.

22. A pharmaceutical composition, wherein the pharmaceutical composition comprises the combination of any one of claims 1-21 and a pharmaceutically acceptable excipient.

23. The pharmaceutical composition of claim 22, wherein the pharmaceutical composition is in a dosage form for systemic or topical administration.

24. The pharmaceutical composition of claim 23, wherein the systemic dosage form is selected from the group consisting of a tablet, granule, capsule, pill, solutions, emulsion, suspension, injection.

25. The pharmaceutical composition of claim 23, wherein the topical dosage form is selected from the group consisting of a drop, cream, ointment, lotion, liniment, suppository, paste, and patch.

26. A kit, wherein the kit comprises one or more container for accommodating the combination of any one of claims 1-21 or the pharmaceutical composition of any one of claims 22-25.

27. The kit of claim 26, wherein the kit further comprises an instruction on how to use the combination or composition for treating or preventing microorganism infections.

28. The kit of claim 27, wherein the microorganism infection is an eye infection, skin infection, or an infection in wound.

29. A method for treating a microorganism infection, wherein the method includes a step of giving a therapeutically effective amount of the combination of any one of claims 1-21 or the pharmaceutical composition of any one of claims 22-25 or the kit of any one of claims 26-28 to a subject in need thereof.

30. The method of claim 29, wherein the subject is a mammal.

31. The method of claim 30, wherein the subject is a house pet, a racing animal, a domestic animal, or a primate.

32. The method of claim 30, wherein the subject is a cat, dog, horse, pig, cattle, cow, goat, sheep, rabbit, or human; preferably, a human.

33. The method of claim 29, wherein the microorganism infection is an eye infection, skin infection, an oral infection, a respiratory system infection, a digestive system infection or an infection in wound.
